SEQUENCE LISTING (130 sequences)

SEQ ID NO: 1 - protein, 250 aa, synthetic construct
  REGION 1..250: note = CD58
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 2 - DNA, 750 bp, synthetic construct
  misc_feature 1..750: note = CD58
  source 1..750: mol_type = other DNA
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaat

SEQ ID NO: 3 - protein, 288 aa, synthetic construct
  REGION 1..288: note = CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 4 - DNA, 864 bp, synthetic construct
  misc_feature 1..864: note = CD80
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 5 - protein, 329 aa, synthetic construct
  REGION 1..329: note = CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 6 - DNA, 987 bp, synthetic construct
  misc_feature 1..987: note = CD86
  source 1..987: mol_type = other DNA
atggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaactctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaactcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaaggtctgatgaagcccagcgtgtttttaaaagttcgaagacatcttcatgcgacaaaagtgatacatgtttt

SEQ ID NO: 7 - protein, 208 aa, synthetic construct [sequence omitted]

SEQ ID NO: 8 - protein, 224 aa, synthetic construct
  REGION 1..224: note = CD86 Extracellular Domain
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 9 - protein, 92 aa, synthetic construct
  REGION 1..92: note = CD58 Extracellular Domain
FSQQIYGVVYGNVTFHVPSNVPLKEVLWKKQKDKVAELENSEFRAFSSFKNRVYLDTVSGSLTIYNLTSSDEDEYEMESPNITDTMKFFLYV

SEQ ID NO: 10 - protein, 453 aa, synthetic construct
  REGION 1..453: note = HHLA2 Extracellular Domain
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYE

SEQ ID NO: 11 - protein, 133 aa, synthetic construct
  REGION 1..133: note = ICAM-1 Extracellular Domain
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 12 - protein, 184 aa, synthetic construct
  REGION 1..184: note = OX40-L Extracellular Domain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 173 aa, synthetic construct
  REGION 1..173: note = 4-1BBL Extracellular Domain
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 14 - protein, 323 aa, synthetic construct
  REGION 1..323: note = CD40 Extracellular Domain
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 15 - protein, 155 aa, synthetic construct
  REGION 1..155: note = CD155 Extracellular Domain
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 16 - protein, 161 aa, synthetic construct
  REGION 1..161: note = CD70 Extracellular Domain
LPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSS

SEQ ID NO: 17 - protein, 128 aa, synthetic construct
  REGION 1..128: note = HVEM Extracellular Domain
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 18 - protein, 172 aa, synthetic construct
  REGION 1..172: note = GITRL Extracellular Domain
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 19 - protein, 193 aa, synthetic construct
  REGION 1..193: note = CD30L Extracellular Domain
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLAR

SEQ ID NO: 20 - protein, 407 aa, synthetic construct
  REGION 1..407: note = SLAMF2 Extracellular Domain
KDSAPTVVSGILGGSVTLPLNISVDTEIENVIWIGPKNALAFARPKENVTIMVKSYLGRLDITKWSYSLCISNLTLNDAGSYKAQINQRNFEVTTEEEFTLFVYEQLQEPQVTMKSVKVSENFSCNITLMCSVKGAEKSVLYSWTPREPHASESNGGSILTVSRTPCDPDLPYICTAQNPVSQRSSLPVHVGQFCTDPGASRGGTTGETVVGVLGEPVTLPLALPACRDTEKVVWLFNTSIISKEREEAATADPLIKSRDPYKNRVWVSSQDCSLKISQLKIEDAGPYHAYVCSEASSVTSMTHVTLLIYRRLRKPKITWSLRHSEDGICRISLTCSVEDGGNTVMYTWTPLQKEAVVSQGESHLNVSWRSSENHPNLTCTASNPVSRSSHQFLSENICSGPERNTK

SEQ ID NO: 21 - protein, 204 aa, synthetic construct
  REGION 1..204: note = Ly-9 Extracellular Domain
KDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTG

SEQ ID NO: 22 - protein, 204 aa, synthetic construct
  REGION 1..204: note = CD84 Extracellular Domain
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTK

SEQ ID NO: 23 - protein, 283 aa, synthetic construct
  REGION 1..283: note = Ly108 Extracellular Domain
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSH

SEQ ID NO: 24 - protein, 243 aa, synthetic construct
  REGION 1..243: note = MICA Extracellular Domain
EPHSLRYNLMVLSQDGSVQSGFLAEGHLDGQPFLRYDRQKRRAKPQGQWAEDVLGAETWDTETEDLTENGQDLRRTLTHIKDQKGVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTD

SEQ ID NO: 25 - protein, 244 aa, synthetic construct
  REGION 1..244: note = MICB Extracellular Domain
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 26 - protein, 246 aa, synthetic construct
  REGION 1..246: note = ULBP1
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 27 - protein, 244 aa, synthetic construct
  REGION 1..244: note = ULBP2
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 28 - protein, 195 aa, synthetic construct
  REGION 1..195: note = ULBP3
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPD

SEQ ID NO: 29 - protein, 198 aa, synthetic construct
  REGION 1..198: note = ULBP4 Extracellular Domain
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 30 - protein, 246 aa, synthetic construct
  REGION 1..246: note = ULBP5 Extracellular Domain
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 31 - protein, 238 aa, synthetic construct [sequence omitted]

SEQ ID NO: 32 - protein, 238 aa, synthetic construct
  REGION 1..238: note = B7-H2 Extracellular Domain
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 33 - protein, 162 aa, synthetic construct
  REGION 1..162: note = B7-H6 Extracellular Domain
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 34 - protein, 220 aa, synthetic construct
  REGION 1..220: note = B7-H5 Extracellular Domain
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 35 - protein, 244 aa, synthetic construct
  REGION 1..244: note = B7-H3 Extracellular Domain
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASTTENLYFQG

SEQ ID NO: 36 - protein, 128 aa, synthetic construct
  REGION 1..128: note = B7x Extracellular Domain
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPG

SEQ ID NO: 37 - protein, 133 aa, synthetic construct [sequence omitted]

SEQ ID NO: 38 - protein, 152 aa, synthetic construct
  REGION 1..152: note = IL-2
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 39 - protein, 197 aa, synthetic construct
  REGION 1..197: note = IL-7
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 40 - protein, 306 aa, synthetic construct
  REGION 1..306: note = IL-12 subunit alpha
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 114 aa, synthetic construct
  REGION 1..114: note = IL-12 subunit beta
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 42 - protein, 157 aa, synthetic construct
  REGION 1..157: note = IL-15
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 138 aa, synthetic construct
  REGION 1..138: note = IL-18
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 44 - protein, 453 aa, synthetic construct; identical to SEQ ID NO: 10
  REGION 1..453: note = IL-21

SEQ ID NO: 45 - DNA, 327 bp, synthetic construct
  misc_feature 1..327: note = E2 scFv VL nucleotide
  source 1..327: mol_type = other DNA
tccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctg

SEQ ID NO: 46 - protein, 500 aa, synthetic construct
  REGION 1..500: note = Gag polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 47 - protein, 109 aa, synthetic construct
  REGION 1..109: note = E2 scFv VL polypeptide
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 48 - DNA, 381 bp, synthetic construct
  misc_feature 1..381: note = E2 scFv VH nucleotide
  source 1..381: mol_type = other DNA
caggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctcc

SEQ ID NO: 49 - protein, 1003 aa, synthetic construct
  REGION 1..1003: note = Pol polypeptide
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 50 - protein, 127 aa, synthetic construct
  REGION 1..127: note = E2 scFv VH polypeptide
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 51 - DNA, 54 bp, synthetic construct
  misc_feature 1..54: note = E2 scFv linker nucleotide
  source 1..54: mol_type = other DNA
ggctctacaagcggatccggcaagccaggatctggagagggcagcacaaaggga

SEQ ID NO: 52 - DNA, 4307 bp, synthetic construct
  misc_feature 1..4307: note = Gag-pol nucleotide
  source 1..4307: mol_type = other DNA
atgggcgcccgcgccagcgtgctttctggcggcgagctggacaggtgggagaagattcgcctgcggcctggaggaaagaaaaagtacaagctgaagcacatcgtgtgggcttctcgggaactggaaagattcgccgtgaaccctggactgctagagacctccgaaggctgcagacagatcctgggacagctgcaacctagcctgcagaccggcagcgaggagctgagaagcctgtacaacaccgtcgccaccctgtattgtgtgcaccaaagaatcgagatcaaggacaccaaggaggctctggataagatcgaggaagagcagaacaagagcaagaaaaaagcccagcaggccgccgctgataccggccattctaatcaggtgtcccagaactaccccattgtgcaaaatatccagggccagatggtccaccaggccatcagccctagaaccctgaatgcctgggtgaaggtggtggaagagaaggccttttctccagaggtgatccctatgttcagcgccctgagcgagggcgctacccctcaggacctgaacacaatgctgaacaccgtgggcggccaccaggccgccatgcagatgctgaaagaaaccatcaacgaggaggccgccgaatgggaccgggtgcaccccgttcacgccggcccaatcgcccctggccagatgcgggaacctagaggcagcgacatcgccggcacaaccagcacactgcaagagcagatcggatggatgacacacaacccccccatccccgtgggcgaaatctacaagcggtggatcattctgggactgaacaaaatcgttagaatgtacagccctaccagcatcctggatatcagacagggcccaaaggagcctttccgggactacgtggacagattttacaagaccctgagagccgaacaggcctcccaagaggtgaagaactggatgacagagaccctgctggtgcagaatgccaaccctgattgtaagacaatcctgaaggccctcggacctggcgctacactggaagaaatgatgaccgcctgccagggcgtgggcggccccggccacaaggccagagtgctggccgaggctatgagccaggtgacaaaccccgccacaatcatgatccagaagggcaacttcagaaaccagcggaagaccgtgaaatgcttcaactgcggcaaggaaggccacatcgcaaagaactgcagagcccctaggaagaaaggctgttggaagtgcggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggcggacagctgaaagaggccttgctggacaccggagccgatgacaccgtgctggaagaaatgaacctgcctggaagatggaaacctaagatgatcggtggcatcggcggatttatcaaagtgcgacagtatgaccagatcctgatcgagatttgcggccacaaagctatcggaacagtgctggtcggcccgacccccgtgaacatcattggccgcaacctgctgacacagatcggttgtacactgaactttcctatcagccctatcgaaaccgtgccggtcaagctgaagcccggcatggatggccctaaggtgaagcagtggcccctgacagaggaaaagatcaaggcactggtggaaatctgcacagaaatggaaaaagagggcaagatttctaaaatcggcccagagaacccctacaacacccctgttttcgccatcaagaagaaagattccaccaagtggaggaagctggtggactttcgggaactgaacaagcggacccaggatttctgggaggtgcagctgggcatcccccaccctgccggcctgaaacaaaaaaaaagcgtgaccgtgctggacgtgggcgacgcctatttcagcgtgcctctggataaggacttccggaaatacaccgcctttaccatccctagcatcaacaacgagacccctggcatccggtaccagtacaacgtgctcccacagggctggaagggctcacccgccatcttccagtgcagcatgaccaagatcctggagcctttcagaaagcagaatcctgacatcgtgatctaccagtacatggacgacctgtacgtgggctctgatctggagatcggacagcacagaacaaagatcgaagagctgagacagcatctgctgagatggggtttcaccacccccgacaagaagcaccagaaggaacctccttttctgtggatgggctacgagctgcaccccgataagtggacagtgcagcccatcgtgctgcccgagaaggactcctggaccgtgaacgacattcagaagctggtcggaaagctgaattgggcttcccaaatctacgccggcatcaaggtgcggcagctgtgcaagcttctgcgcggcacaaaggccctgacggaagtcgtgccactgaccgaggaagccgaattagagctggccgaaaacagagaaattctgaaagaacctgtgcacggcgtttactacgacccttctaaggacctgatcgccgaaatccagaaacaaggccagggccagtggacttaccaaatctaccaggagcctttcaaaaacctcaagaccggcaagtacgccagaatgaagggagcccatacaaacgacgtgaagcagctgacagaggctgttcagaagatcgccacagaaagcatcgtgatctggggcaagaccccaaagttcaagctgcctatccaaaaggaaacctgggaggcctggtggaccgagtactggcaggccacctggattcctgaatgggagttcgtgaacacaccacctcttgtgaagctgtggtaccagctggaaaaggagccaatcatcggcgccgagacattctacgtggacggcgccgccaaccgggagaccaaactgggaaaggccggatatgtgaccgacagaggcagacaaaaggtggtgcctctgaccgatacaactaaccagaaaacagagctgcaggccattcacctggccctgcaagacagcggcctggaagtgaatatcgtgacagacagtcagtacgccctgggcatcatccaggctcagcctgacaagagcgagagcgagctggtgtcccagatcatcgagcagctgatcaaaaaggagaaggtttatctggcctgggtgcccgcccacaagggcatcggaggcaacgagcaggtggacaagctggtcagcgccggcatccggaaggtgctgttcctggacggcatcgacaaggcccaggaggaacacgagaagtaccacagcaactggcgggccatggccagcgacttcaacctgccacctgtggttgctaaggagatcgtcgcctcttgtgataagtgccagctgaagggcgaggccatgcacggccaagtggattgcagccctgggatctggcagctagactgtacccacctggagggcaaggtgatcctggtggcagtgcacgtggccagcggctacatcgaggctgaggtgatccccgccgaaacgggccaggagaccgcctactttctgctgaagctagccggccggtggcctgtgaagaccgtgcacaccgataacggcagcaatttcaccagcacaaccgtgaaggctgcctgctggtgggctggaatcaagcaggagttcggcatcccatacaatcctcagtctcagggcgtgatcgagagcatgaacaaggaactgaagaagatcattggtcaggtcagagatcaggccgagcacctgaaaaccgccgttcaaatggctgtgttcatccataacttcaaaagaaaaggcggcatcggcggctacagcgccggcgaaagaatcgtggatatcatcgcgaccgacatccaaacaaaagagctgcaaaagcaaatcaccaagatccagaacttcagagtgtactacagagatagcagagatcctgtgtggaagggacctgccaagctgctgtggaagggcgagggcgccgtggtgatccaggacaatagcgacatcaaggtcgtgcccagaagaaaggctaaaatcattagagactacggcaaacagatggccggagatgattgcgtggcttctagacaggacgaggactga

SEQ ID NO: 53 - protein, 18 aa, synthetic construct
  REGION 1..18: note = E2 scFv linker polypeptide
GSTSGSGKPGSGEGSTKG

SEQ ID NO: 54 - DNA, 762 bp, synthetic construct
  misc_feature 1..762: note = E2 scFv nucleotide
  source 1..762: mol_type = other DNA
tccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctcc

SEQ ID NO: 55 - DNA, 762 bp, synthetic construct; identical to SEQ ID NO: 54
  misc_feature 1..762: note = E2 scFv nucleoti de
  source 1..762: mol_type = other DNA

SEQ ID NO: 56 - protein, 254 aa, synthetic construct
  REGION 1..254: note = E2 scFv polypeptide
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 57 - DNA, 141 bp, synthetic construct
  misc_feature 1..141: note = CD8alpha
  source 1..141: mol_type = other DNA
accaccacacctgctcctagaccacctacacccgctcctaccatcgccagccagcctctgtctctgagacctgaggcctgtagacctgccgctggaggcgctgtgcacaccagaggactggatttcgcctgcgacatctac

SEQ ID NO: 58 - protein, 47 aa, synthetic construct
  REGION 1..47: note = CD8alpha
TTTPAPRPPTPAPTIASQPLSLRPEACRPAAGGAVHTRGLDFACDIY

SEQ ID NO: 59 - protein, 13 aa, synthetic construct
  REGION 1..13: note = CD8 Hinge
GAVHTRGLDFACD

SEQ ID NO: 60 - protein, 13 aa, synthetic construct
  REGION 1..13: note = Modified IgG4 Hinge
ESKYGPPSPPSPA

SEQ ID NO: 61 - protein, 12 aa, synthetic construct
  REGION 1..12: note = Modified IgG4 Hinge
ESKYGPPSPPSP

SEQ ID NO: 62 - protein, 12 aa, synthetic construct
  REGION 1..12: note = IgG1 Hinge
EPKSCDKTHTCP

SEQ ID NO: 63 - protein, 13 aa, synthetic construct
  REGION 1..13: note = PD1 Hinge
PSPRPAGQFQTLV

SEQ ID NO: 64 - protein, 13 aa, synthetic construct
  REGION 1..13: note = CD28 Hinge
LCPSPLFPGPSKP

SEQ ID NO: 65 - DNA, 66 bp, synthetic construct
  misc_feature 1..66: note = CD8 transmembrane nucleotide
  source 1..66: mol_type = other DNA
atctgggctcctctggctggaacatgcggcgtgctgctcctgagcctggtgatcacactgtactgc

SEQ ID NO: 66 - protein, 22 aa, synthetic construct
  REGION 1..22: note = CD8 transmembrane polypeptide
IWAPLAGTCGVLLLSLVITLYC

SEQ ID NO: 67 - DNA, 126 bp, synthetic construct
  misc_feature 1..126: note = 41BB nucleotide
  source 1..126: mol_type = other DNA
aagcgcggccggaagaagctgctctacatctttaagcagccattcatgcgccccgtgcagaccacacaggaggaggacggctgctcctgtcggtttccagaggaggaggagggaggatgtgagctg

SEQ ID NO: 68 - protein, 42 aa, synthetic construct
  REGION 1..42: note = 41BB polypeptide
KRGRKKLLYIFKQPFMRPVQTTQEEDGCSCRFPEEEEGGCEL

SEQ ID NO: 69 - DNA, 336 bp, synthetic construct
  misc_feature 1..336: note = CD3zeta nucleotide
  source 1..336: mol_type = other DNA
agagtgaagttctctaggagcgccgatgcccctgcctatcagcagggacagaaccagctgtacaacgagctgaatctgggccggagagaggagtacgacgtgctggataagaggaggggaagagacccagagatgggaggcaagcctcggagaaagaacccacaggagggcctgtataatgagctgcagaaggacaagatggccgaggcctactccgagatcggcatgaagggagagaggcgccggggcaagggacacgatggcctgtatcagggcctgagcaccgccacaaaggacacatacgatgccctgcacatgcaggccctgcctccaagg

SEQ ID NO: 70 - protein, 112 aa, synthetic construct
  REGION 1..112: note = CD3zeta polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 1380 bp, synthetic construct
  misc_feature 1..1380: note = CD19 CAR nucleotide
  source 1..1380: mol_type = other DNA
ctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcggcgctgtgcacaccagaggactggatttcgcctgcgacttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 72 - protein, 460 aa, synthetic construct
  REGION 1..460: note = CD19 CAR polypeptide
LLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - DNA, 735 bp, synthetic construct
  misc_feature 1..735: note = CD19 scFv nucleotide
  source 1..735: mol_type = other DNA
gacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagc

SEQ ID NO: 74 - protein, 245 aa, synthetic construct
  REGION 1..245: note = CD19 scFv polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 75 - DNA, 318 bp, synthetic construct
  misc_feature 1..318: note = CD19 scFv VL nucleotide
  source 1..318: mol_type = other DNA
gacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatc

SEQ ID NO: 76 - protein, 106 aa, synthetic construct
  REGION 1..106: note = CD19 scFv VL polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEI

SEQ ID NO: 77 - DNA, 57 bp, synthetic construct
  misc_feature 1..57: note = CD19 scFv linker nucleotide
  source 1..57: mol_type = other DNA
accggatccacatctggaagcggcaagccaggaagcggagagggatccacaaaggga

SEQ ID NO: 78 - protein, 19 aa, synthetic construct
  REGION 1..19: note = CD19 scFv linker polypeptide
TGSTSGSGKPGSGEGSTKG

SEQ ID NO: 79 - DNA, 360 bp, synthetic construct
  misc_feature 1..360: note = CD19 scFv VH nucleotide
  source 1..360: mol_type = other DNA
gaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagc

SEQ ID NO: 80 - DNA, 360 bp, synthetic construct; identical to SEQ ID NO: 79
  misc_feature 1..360: note = CD19 scFv VH polypeptide
  source 1..360: mol_type = other DNA

SEQ ID NO: 81 - DNA, 39 bp, synthetic construct
  misc_feature 1..39: note = CD8 hinge nucleotide
  source 1..39: mol_type = other DNA
ggcgctgtgcacaccagaggactggatttcgcctgcgac

SEQ ID NO: 82 - protein, 13 aa, synthetic construct
  REGION 1..13: note = CD8 hinge polypeptide
GAVHTRGLDFACD

SEQ ID NO: 83 - DNA, 81 bp, synthetic construct
  misc_feature 1..81: note = CD28 transmembrane domain nucleotide
  source 1..81: mol_type = other DNA
ttctgggtgctggtggtggtgggaggcgtgctggcctgttactccctgctggtgaccgtggcctttatcatcttctgggtg

SEQ ID NO: 84 - protein, 27 aa, synthetic construct
  REGION 1..27: note = CD28 transmembrane domain nucleotide
FWVLVVVGGVLACYSLLVTVAFIIFWV

SEQ ID NO: 85 - DNA, 126 bp, synthetic construct
  misc_feature 1..126: note = 41BB nucleotide
  source 1..126: mol_type = other DNA
aagagaggcaggaagaagctgctgtatatctttaagcagcccttcatgcgccctgtgcagaccacacaggaggaggacggctgcagctgtcggtttccagaggaggaggagggaggatgcgagctg

SEQ ID NO: 86 - DNA, 336 bp, synthetic construct
  misc_feature 1..336: note = CD3zeta domain nucleotide
  source 1..336: mol_type = other DNA
cgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 87 - DNA, 1548 bp, synthetic construct
  misc_feature 1..1548: note = CD20 CAR nucleotide (with flag)
  source 1..1548: mol_type = other DNA
atgctgctgctggtgacaagcctgctgctgtgcgagctgcctcacccagcctttctgctgatccccgatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaagggctctaccagcggaggaggaagcggaggaggatccggaggcggcggctctagcgaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagcgctgcaggaggcggaggatctggaggcggcggcggagactacaaagacgatgacgacaagttcgaagcaaagccaaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccccctcgg

SEQ ID NO: 88 - protein, 516 aa, synthetic construct
  REGION 1..516: note = CD20 CAR polypeptide (with flag sequence)
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSAAGGGGSGGGGGDYKDDDDKFEAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 738 bp, synthetic construct
  misc_feature 1..738: note = CD20 scFv nucleotide
  source 1..738: mol_type = other DNA
gatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaagggctctaccagcggaggaggaagcggaggaggatccggaggcggcggctctagcgaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagc

SEQ ID NO: 90 - protein, 246 aa, synthetic construct
  REGION 1..246: note = CD20 scFv polypeptide
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 91 - DNA, 318 bp, synthetic construct
  misc_feature 1..318: note = CD20 scFv VL nucleotide
  source 1..318: mol_type = other DNA
gatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaag

SEQ ID NO: 92 - protein, 106 aa, synthetic construct
  REGION 1..106: note = CD20 scFv VL polypeptide
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 93 - DNA, 54 bp, synthetic construct
  misc_feature 1..54: note = 218 linker nucleotide
  source 1..54: mol_type = other DNA
ggctctaccagcggaggaggaagcggaggaggatccggaggcggcggctctagc

SEQ ID NO: 94 - protein, 18 aa, synthetic construct
  REGION 1..18: note = 218 linker polypeptide
GSTSGGGSGGGSGGGGSS

SEQ ID NO: 95 - DNA, 366 bp, synthetic construct
  misc_feature 1..366: note = CD20 scFv VH nucleotide
  source 1..366: mol_type = other DNA
gaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagc

SEQ ID NO: 96 - protein, 122 aa, synthetic construct
  REGION 1..122: note = CD20 scFv VH polypeptide
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 97 - DNA, 24 bp, synthetic construct
  misc_feature 1..24: note = Flag nucleotide
  source 1..24: mol_type = other DNA
gactacaaagacgatgacgacaag

SEQ ID NO: 98 - protein, 8 aa, synthetic construct
  REGION 1..8: note = Flag polypeptide
DYKDDDDK

SEQ ID NO: 99 - DNA, 216 bp, synthetic construct
  misc_feature 1..216: note = CD8 HTM nucleotide
  source 1..216: mol_type = other DNA
gcaaagccaaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgc

SEQ ID NO: 100 - protein, 72 aa, synthetic construct
  REGION 1..72: note = CD8 HTM polypeptide
AKPTTTPAPRPPTPAPTIASQPLSLRPEACRPAAGGAVHTRGLDFACDIYIWAPLAGTCGVLLLSLVITLYC

SEQ ID NO: 101 - DNA, 336 bp, synthetic construct
  misc_feature 1..336: note = CD3zeta nucleotide
  source 1..336: mol_type = other DNA
cgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccccctcgg

SEQ ID NO: 102 - DNA, 1497 bp, synthetic construct
  misc_feature 1..1497: note = TagCAR nucleotide
  source 1..1497: mol_type = other DNA
ctgctgctggtgacaagcctgctgctgtgcgagctgcctcacccagcctttctgctgatcccctccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctccaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagcgcggccggaagaagctgctctacatctttaagcagccattcatgcgccccgtgcagaccacacaggaggaggacggctgctcctgtcggtttccagaggaggaggagggaggatgtgagctgagagtgaagttctctaggagcgccgatgcccctgcctatcagcagggacagaaccagctgtacaacgagctgaatctgggccggagagaggagtacgacgtgctggataagaggaggggaagagacccagagatgggaggcaagcctcggagaaagaacccacaggagggcctgtataatgagctgcagaaggacaagatggccgaggcctactccgagatcggcatgaagggagagaggcgccggggcaagggacacgatggcctgtatcagggcctgagcaccgccacaaaggacacatacgatgccctgcacatgcaggccctgcctccaaggtga

SEQ ID NO: 103 - DNA, 1497 bp, synthetic construct
  misc_feature 1..1497: note = TagCAR nucleotide
  source 1..1497: mol_type = other DNA
atgctgctgctggtgacaagcctgctgctgtgcgagctgcctcacccagcctttctgctgatcccctccgtgctgacccagcctagctccgtgtctgccgcaccaggacagaaggtgacaatcagctgttccggctctaccagcaacatcggcaacaattacgtgagctggtaccagcagcaccctggcaaggccccaaagctgatgatctacgacgtgtccaagaggccatctggagtgcctgatcggttctccggctctaagagcggcaattccgcctctctggacatcagcggactgcagtccgaggacgaggcagattactattgcgccgcctgggacgatagcctgtccgagtttctgttcggcaccggcacaaagctgaccgtgctgggctctacaagcggatccggcaagccaggatctggagagggcagcacaaagggacaggtgcagctggtggagagcggaggaaacctggtgcagccaggaggctccctgcgcctgtcttgtgccgccagcggctttaccttcggctcttttagcatgtcctgggtgcgccaggcacctggaggaggactggagtgggtggccggcctgagcgcccggtctagcctgacacactatgccgactccgtgaagggccgcttcaccatctcccgggataacgccaagaatagcgtgtacctgcagatgaatagcctgcgggtggaggacacagccgtgtactattgcgccaggcgctcctatgattcctctggctactggggccacttttactcttatatggacgtgtggggacagggcaccctggtgacagtgagctccaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagcgcggccggaagaagctgctctacatctttaagcagccattcatgcgccccgtgcagaccacacaggaggaggacggctgctcctgtcggtttccagaggaggaggagggaggatgtgagctgagagtgaagttctctaggagcgccgatgcccctgcctatcagcagggacagaaccagctgtacaacgagctgaatctgggccggagagaggagtacgacgtgctggataagaggaggggaagagacccagagatgggaggcaagcctcggagaaagaacccacaggagggcctgtataatgagctgcagaaggacaagatggccgaggcctactccgagatcggcatgaagggagagaggcgccggggcaagggacacgatggcctgtatcagggcctgagcaccgccacaaaggacacatacgatgccctgcacatgcaggccctgcctccaagg

SEQ ID NO: 104 - protein, 498 aa, synthetic construct
  REGION 1..498: note = TagCAR polypeptide
LLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 499 aa, synthetic construct
  UNSURE 1..499: note = TagCAR polypeptidee
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 762 bp, synthetic construct; identical to SEQ ID NO: 54
  misc_feature 1..762: note = E2 scFv nucleotide
  source 1..762: mol_type = other DNA

SEQ ID NO: 107 - protein, 254 aa, synthetic construct; identical to SEQ ID NO: 56
  REGION 1..254: note = E2 scFv polypeptide

SEQ ID NO: 108 - DNA, 327 bp, synthetic construct; identical to SEQ ID NO: 45
  misc_feature 1..327: note = E2 scFv VL nucleotide
  source 1..327: mol_type = other DNA

SEQ ID NO: 109 - protein, 109 aa, synthetic construct; identical to SEQ ID NO: 47
  REGION 1..109: note = E2 scFv VL polypeptide

SEQ ID NO: 110 - DNA, 54 bp, synthetic construct; identical to SEQ ID NO: 51
  misc_feature 1..54: note = E2 scFv linker nucleotide
  source 1..54: mol_type = other DNA

SEQ ID NO: 111 - protein, 18 aa, synthetic construct
  REGION 1..18: note = E2 scFv linker polypeptide
GSTSGSGKPGSGEGSTKG

SEQ ID NO: 112 - DNA, 381 bp, synthetic construct; identical to SEQ ID NO: 48
  misc_feature 1..381: note = E2 scFv VH nucleotide
  source 1..381: mol_type = other DNA

SEQ ID NO: 113 - protein, 127 aa, synthetic construct; identical to SEQ ID NO: 50
  REGION 1..127: note = E2 scFv VH polypeptide

SEQ ID NO: 114 - DNA, 207 bp, synthetic construct
  misc_feature 1..207: note = CD8 HTM nucleotide
  source 1..207: mol_type = other DNA
accaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgc

SEQ ID NO: 115 - protein, 69 aa, synthetic construct
  REGION 1..69: note = CD8 HTM polypeptide
TTTPAPRPPTPAPTIASQPLSLRPEACRPAAGGAVHTRGLDFACDIYIWAPLAGTCGVLLLSLVITLYC

SEQ ID NO: 116 - DNA, 141 bp, synthetic construct; identical to SEQ ID NO: 57
  misc_feature 1..141: note = CD8 Hinge nucleotide
  source 1..141: mol_type = other DNA

SEQ ID NO: 117 - protein, 47 aa, synthetic construct; identical to SEQ ID NO: 58
  REGION 1..47: note = CD8 Hinge polypeptide

SEQ ID NO: 118 - DNA, 66 bp, synthetic construct; identical to SEQ ID NO: 65
  misc_feature 1..66: note = CD8 transmembrane nucleotide
  source 1..66: mol_type = other DNA

SEQ ID NO: 119 - protein, 22 aa, synthetic construct
  REGION 1..22: note = CD8 transmembrane polypeptide
IWAPLAGTCGVLLLSLVITLYC

SEQ ID NO: 120 - protein, 248 aa, synthetic construct
  REGION 1..248: note = Anti-CD3 scFv (VL-G4S x 3 linker - VH)
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAAAKP

SEQ ID NO: 121 - DNA, 744 bp, synthetic construct
  misc_feature 1..744: note = Anti-CD3 scFv (VL-G4S x 3 linker - VH)
  source 1..744: mol_type = other DNA
gatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcct

SEQ ID NO: 122 - protein, 245 aa, synthetic construct
  REGION 1..245: note = Anti-CD3 scFv VL-G4S x 3 linker-VH
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAS

SEQ ID NO: 123 - DNA, 735 bp, synthetic construct
  misc_feature 1..735: note = Anti-CD3 scFv VL-G4S x 3 linker-VH
  source 1..735: mol_type = other DNA
gacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctcc

SEQ ID NO: 124 - protein, 511 aa, synthetic construct
  REGION 1..511: note = Cocal envelope polypeptide
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 125 - DNA, 1539 bp, synthetic construct
  misc_feature 1..1539: note = Cocal envelope nucleotide
  source 1..1539: mol_type = other DNA
atgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtga

SEQ ID NO: 126 - DNA, 1533 bp, synthetic construct
  misc_feature 1..1533: note = Cocal envelope nucleotide
  source 1..1533: mol_type = other DNA
aattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaag

SEQ ID NO: 127 - protein, 116 aa, synthetic construct
  REGION 1..116: note = Rev polypeptide
MAGRSGDSDEDLLKAVRLIKFLYQSNPPPNPEGTRQARRNRRRRWRERQRQIHSISERILSTYLGRSAEPVPLQLPPLERLTLDCNEDCGTSGTQGVGSPQILVESPTILESGAKE

SEQ ID NO: 128 - DNA, 351 bp, synthetic construct
  misc_feature 1..351: note = Rev nucleotide
  source 1..351: mol_type = other DNA
atggccggcagaagcggcgacagcgacgaggatctgctgaaagccgtgcggctgatcaagttcctgtaccagagcaaccctcctcctaaccccgagggcaccagacaggctagacggaaccgcagaagaaggtggcgggaacggcaaagacagatccactctatcagcgagagaatcctgagcacctacctgggaagatccgccgagcctgtccccctgcagctgcctccactggaaagactgaccctggattgtaatgaggactgcggcacaagcggaacccagggcgtgggcagcccccagattctggtggaatcccctacaatcctcgagtctggcgccaaggaatga

SEQ ID NO: 129 - DNA, 1482 bp, synthetic construct
  source 1..1482: mol_type = other DNA
  unsure 1..1482: note = Anti-CD20 CAR (without flag) nucleotide
atgctgctgctggtgacaagcctgctgctgtgcgagctgcctcacccagcctttctgctgatccccgatatcgtgctgacccagtcccccgccatcctgtccgcctctcctggagagaaggtgaccatgacatgtcgggccagctcctctgtgaactacatggactggtatcagaagaagcctggcagctcccccaagccttggatctacgccacctccaatctggcctctggagtgccagcaagattcagcggatccggatctggcacaagctattccctgaccatctccagggtggaggcagaggatgcagcaacatactattgccagcagtggtctttcaacccccctacatttggcggcggcaccaagctggagatcaagggctctaccagcggaggaggaagcggaggaggatccggaggcggcggctctagcgaggtgcagctgcagcagtccggagcagagctggtgaagcctggagcctctgtgaagatgagctgtaaggcctccggctacaccttcacatcttataatatgcactgggtgaagcagacaccaggacagggactggagtggatcggagcaatctaccctggcaacggcgacaccagctataatcagaagtttaagggcaaggccaccctgacagccgataagtcctctagcacagcctacatgcagctgtcctctctgaccagcgaggactccgccgattactattgcgcccggtccaactactatggcagctcctattggttctttgacgtgtggggagcaggaacaaccgtgaccgtgtctagcgcaaagccaaccaccacacctgctcctagaccacctacacccgctcctaccatcgccagccagcctctgtctctgagacctgaggcctgtagacctgccgctggaggcgctgtgcacaccagaggactggatttcgcctgcgacatctacatctgggctcctctggctggaacatgcggcgtgctgctcctgagcctggtgatcacactgtactgcaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccccctcgg

SEQ ID NO: 130 - protein, 494 aa, synthetic construct
  UNSURE 1..494: note = Anti-CD20 CAR (without flag) polypeptide
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0382586 A1
(43) Pub. Date: Dec. 18, 2025

(54) MANUFACTURING VIRAL PARTICLES

(71) Applicant: Umoja Biopharma, Inc., Seattle, WA (US)

(72) Inventors: John Jeffrey PLOMER, Seattle, WA (US); Sarah GOULD, Seattle, WA (US); Branden SALINAS, Seattle, WA (US); Kim BALLINGER, Seattle, WA (US); Molly DEPOY, Seattle, WA (US); Jessica FREEMAN, Seattle, WA (US); Janice JIN, Seattle, WA (US); Mason MUHONEN, Seattle, WA (US); Mark PANKAU, Seattle, WA (US); Amber TURNER, Seattle, WA (US)

(73) Assignee: Umoja Biopharma, Inc., Seattle, WA (US)

(21) Appl. No.: 18/866,424

(22) PCT Filed: May 17, 2023
(86) PCT No.: PCT/US2023/067136

§ 371 Date: Aug. 29, 2025

Related U.S. Application Data
(60) Provisional application No. 63/342,975, filed on May 17, 2022.
(60) Provisional application No. 63/371,756, filed on Aug. 17, 2022.
(60) Provisional application No. 63/371,864, filed on Aug. 18, 2022.
(60) Provisional application No. 63/440,093, filed on Jan. 19, 2023.

(51) Int. Cl. C12N 7/00; C12N 15/86

(52) U.S. Cl. CPC C12N 7/00; C12N 15/86; C12N 2740/16043; C12N 2740/16052; C12N 2740/16211

(57) ABSTRACT

Provided are methods for large scale production of viral particles, and compositions and methods for using said viral particles.

### CROSS-REFERENCE TO RELATED APPLICATIONS

This application claims priority from U.S. provisional application No. 63/342,975, filed May 17, 2022, U.S. provisional application No. 63/371,756, filed Aug. 17, 2022, U.S. provisional application No. 63/371,864, filed Aug. 18, 2022, U.S. provisional application No. 63/440,093, filed Jan. 19, 2023, the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 260132000440SeqList.xml created May 17, 2023 which is 207,707 bytes in size. The information in the electronic format of the Sequence Listing is incorporated by reference in its entirety.

### FIELD

The present disclosure provides methods for large scale production of viral particles, and compositions and methods for using said viral particles.

### BACKGROUND

Gene therapies have shown significant clinical success in treating cancers and other diseases. However, access to these lifesaving therapeutics has been limited due to the critical challenges in cost, supply chain, and manufacturing. Retroviruses are often used as a delivery the transfer of one or more nucleotides of interest to one or more sites of interest. Among retroviruses, lentiviral vector systems are of considerable interest because lentiviruses are able to infect non-dividing cells. In addition, lentiviral vectors allow stable long-term expression of the gene of interest. In most small-scale applications, vectors can be concentrated and purified by relatively simple methods using centrifugation techniques. However, scaling up the purification methods for large-scale production for clinical use represents a major challenge.

In particular, when considering production of a viral vector for human use, the vector purification process is directly linked to safety in terms of purity. There remains a need for a large-scale retroviral vector purification method that yields a product of high purity.

### SUMMARY

The disclosure is based, at least in part, on the discovery of a method for manufacturing viral particles (e.g., lentiviral particles) for in vivo administration to a subject. Specifically, as demonstrated herein, subjecting a mixture of host cells and viral particles to at least two filtration steps reduces contaminants (e.g., host cell DNA and protein) prior to concentrating the mixture via chromatography and ultrafiltration. Without wishing to be bound by theory, the filtration steps described herein reduce contaminants to an amount sufficient for in vivo administration. Accordingly, in some aspects, the disclosure provides a method for preparing a lentivirus formulation, comprising:
(i) filtering a suspension mixture comprising a population of host cells and lentiviral particles to remove contaminants, wherein filtering comprises:
   (a) filtering the mixture with a first filter, wherein the first filter is a depth filter, resulting in a first filtrate,
   (b) filtering the first filtrate with a second filter, wherein the second filter has a retention threshold smaller than the first filter, resulting in a second filtrate, and
   (c) filtering the second filtrate, with a third filter, wherein the third filter has a retention threshold smaller than the second filter, thereby producing a filtered formulation of lentiviral particles; and
(ii) concentrating the filtered formulation of lentiviral particles, wherein concentrating comprises chromatography and ultrafiltration.

In other aspects, the disclosure provides a method for preparing a lentivirus formulation, comprising
(i) contacting a population of host cells in suspension with at least one plasmid encoding a lentiviral protein;
(ii) culturing the population of host cells of step (i) for a period of time sufficient to produce a suspension mixture comprising a population of host cells and lentiviral particles;
(iii) filtering the suspension mixture to remove contaminants, comprising:
   (a) contacting the mixture with an endonuclease,
   (b) filtering the mixture with a first filter, wherein the first filter is a depth filter, resulting in a first filtrate
   (c) filtering the first filtrate with a second filter, wherein the second filter has a retention threshold smaller than the first filter, resulting in a second filtrate, and
   (d) filtering the second filtrate with a third filter, wherein the third filter has a retention threshold smaller than the second filter, thereby producing a filtered formulation of lentiviral particles; and
(iv) concentrating the filtered formulation of lentiviral particles, wherein concentrating comprises chromatography and ultrafiltration.

In some aspects, the host cell comprises a human cell. In some aspects, the human cell comprises a HEK293 cell, a HEK293T cell, a HEK293F cell, a HEK293FT cell, a Te671 cell, a HT1080 cell, or a CEM cell. In some aspects, the cell comprises a HEK293 cell. In some aspects, the cell comprises a HEK293T cell.

In some aspects, the first filter has a retention threshold of 1-60 μm. In some aspects the first filter has a retention threshold of 60 μm. In some aspects, the second filter has a retention threshold of 0.4-4 μm. In some aspects, the second filter has a retention threshold of 0.45 μm. In some embodiments, the third filter has a retention threshold of 0.45 μm±0.2 μm. In some aspects the third filter has a retention threshold of 0.2-0.3 μm. In some aspects, the third filter has a retention threshold of 0.2 μm. In some aspects, the first filter has a retention threshold of 60 μm, the second filter has a retention threshold of 0.45 μm, and the third filter has a retention threshold of 0.2 μm.

In some aspects, the second filter and the third filter are two layers within a dual-layer filter component. In some aspects, the third filter is a dual-layer filter comprising a first layer filter and a second layer filter, wherein the second layer filter has a retention threshold smaller than the first layer filter. In some aspects, the retention threshold of the first filter is 60 μm, the retention threshold of the second filter is 0.45 μm, the retention threshold of the first layer filter is 0.45 μm, and the retention threshold of the second layer filter is 0.2 μm.

In some aspects, an endonuclease is present through steps (i)(a)-(i)(c). In some aspects, the endonuclease is present through steps (iii)(b)-(iii)(d).

In some aspects, the chromatography is anion exchange chromatography. In some embodiments, the AEX chromatography comprises eluting the lentiviral particles with a salt buffer. In some embodiments, the salt buffer comprises NaCl. In some embodiments, the NaCl is at a concentration from about 0.5M to 3M. In some embodiments, the NaCl is at a concentration from about 0.5 M to 1 M. In some embodiments, the NaCl is or about 0.75M. In some embodiments, the NaCl is at a concentration from about 1M to 3 M. In some embodiments, the NaCl is at a concentration from about 1.5 M to 2.5 M. In some embodiments, the NaCl is about 2M.

In some aspects, the chromatography is performed before ultrafiltration. In some aspects, the ultrafiltration is ultrafiltration/diafiltration (UF/DF). In some aspects, the UF/DF is by one or more tangential flow filtration (TFF) steps. In some aspects, the filter of the one or more TFF is a hollow fiber filter. In some aspects, the nominal molecular weight cutoff (NMWC) of the hollow fiber filter is or is about 500 kDa. In some aspects, the TFF comprises a first tangential flow filtration (TFF) step and second tangential flow filtration (TFF) step. In some aspects, the first TFF is performed with a first hollow fiber filter and the second TFF is performed with a second hollow fiber filter. In some aspects, the first and second hollow fiber filter have the same nominal molecular weight cutoff (NMWC). In some aspects, the NMWC is 500 kDa. In some aspects, the first hollow fiber filter has a greater nominal molecular weight cutoff (NMWC) than the second hollow fiber filter. In some aspects, the first hollow fiber filter is 500 kDa. In some aspects, the first hollow fiber filter has a greater surface area than the second hollow fiber filter. In some aspects, the first hollow fiber filter is between 790 cm2 to 1600 cm2. In some aspects, the first hollow fiber filter holds a larger volume than the second hollow fiber filter. In some aspects, the volume of the first hollow fiber filter is 300 mL.

In some aspects, the method comprises sterilizing filtration of the filtered formulation after concentration, thereby producing a sterilized formulation. In some aspects, sterilizing filtration comprises filtering the filtered formulation with a fourth filter. In some aspects, the fourth filter has a retention threshold of 0.2 μm. In some aspects, the method comprises formulating the sterilized formulation in a buffer, thereby producing a drug substance.

In some aspects, the method occurs at a pH of 6-8. In some aspects, the lentivirus formulation is for in vivo administration to a subject.

In some aspects, the amount of contaminants in the filtered formulation is reduced compared to the amount of contaminants in the second filtrate. In some aspects, the amount of contaminants in the sterilized formulation is at a level acceptable for in vivo administration to a subject. In some aspects, the contaminants comprise host cells, host cell DNA (hcDNA), and/or host cell proteins (HCP). In some aspects, the amount of hcDNA is less than 10%, less than 9%, less than 8%, less than 7%, less than 6%, less than 5%, less than 4%, less than 3%, less than 2%, less than 1% or less than 0.5% of the sterilized formulation. In some aspects, the amount of hcDNA is reduced greater than 90%, greater than 91%, greater than 92%, greater than 93%, greater than 94%, greater than 95%, greater than 96%, greater than 97%, greater than 98%, or greater than 99% in the sterilized formulation. In some aspects, the amount of HCP is less than 10%, less than 9%, less than 8%, less than 7%, less than 6%, less than 5%, less than 4%, less than 3%, less than 2%, less than 1% or less than 0.5% of the sterilized formulation. In some aspects, the amount of HCP is reduced greater than 90%, greater than 91%, greater than 92%, greater than 93%, greater than 94%, greater than 95%, greater than 96%, greater than 97%, greater than 98%, or greater than 99% in the sterilized formulation.

In some aspects, the hcDNA amount in the filtered formulation is less than about 2500 ng/1E9 TU. In some aspects, the hcDNA amount in the filtered formulation is at least about 80-fold lower compared to the hcDNA amount in the suspension mixture. In some aspects, the hcDNA amount in the filtered formulation is at least about 5-fold lower compared to the hcDNA amount in the second filtrate. In some aspects, the HCP amount after chromatography is less than about 3000 μg/1E9 TU. In some aspects, the HCP amount after chromatography is at least about 40-fold lower compared to the HCP amount before chromatography. In some aspects, the HCP amount after chromatography is at least about 99% lower compared to the HCP amount before chromatography. In some aspects, the HCP amount is less than about 1500 μg/1E9 TU after a first UF/DF step. In some aspects, the HCP amount is not detectable after a second UF/DF step.

In some aspects, the suspension mixture comprises a media, provided the media does not contain serum and/or animal by-products. In some aspects, the culturing of step (ii) is for 40-48 hours. In some aspects, the filtering and concentrating occurs over a time period of 5-8 hours. In some aspects, the suspension mixture has a volume of 3-50 liters. In some aspects, the suspension mixture has a volume of 5 L to 200 L. In some aspects, the suspension mixture has a volume of 100 L to 200 L. In some aspects, the suspension mixture has a volume of at or about 180 L to at or about 200 L.

In some aspects, the lentiviral particle comprises at least one payload. In some aspects, the payload is at least one nucleic acid. In some aspects, the at least one nucleic acid is a non-coding nucleic acid, optionally wherein the non-coding nucleic acid is an siRNA, a miRNA, or a shRNA. In some aspects, the at least one nucleic acid is a polynucleotide encoding a polypeptide of interest. In some aspects, the at least one plasmid is a polynucleotide encoding a polypeptide of interest. In some aspects, the polypeptide of interest is a chimeric antigen receptor (CAR). In some aspects, the CAR is specific for a tumor-associated antigen. In some aspects, the tumor-associate antigen is CD19, BCMA, GPRC5D, ROR1, FcRL5, alpha-fetoprotein, or Her2. In some aspects, the CAR is a universal CAR. In some aspects, the universal CAR comprises a tag binding domain. In some aspects, the tag is a fluorescein. In some aspects, the CAR comprises a hapten binding domain.

In some aspects, the lentiviral particle comprises a surface engineered fusion protein exposed on the surface of the lentiviral particle, optionally wherein the surface engineered protein is embedded in the lipid bilayer. In some aspects, the surface engineered protein is composed of a single binding domain protein that binds to a target molecule on a target cell. In some aspects, the surface engineered protein is composed of a multiple binding domain protein, wherein each binding domain binds to a target molecule on a target cell, optionally wherein each binding domain binds to a different target molecule. In some aspects, the single binding domain protein or the multiple binding domain protein is an immune-cell activating protein. In some aspects, the surface engineered protein is a fusion protein comprising an immune cell-activating protein and a viral envelope protein.

In some aspects, the lentiviral particle comprises a viral envelope comprising an immune cell-activating protein and a viral envelope protein. In some aspects, the at least one plasmid is a plasmid encoding an immune cell-activating protein and a plasmid encoding a viral envelope protein. In some aspects, the immune-cell activating protein is a protein that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, TCR α chain, TCR β chain, TCR ζ chain, TCR γ chain, TCR δ chain, CD3 ε TCR subunit, CD3 γ TCR subunit, CD3 δ TCR subunit, or NKp80.

In some aspects, the immune-cell activating protein comprises at least one binding domain that binds a target molecule selected from the group consisting of a T cell activation receptor, a costimulatory molecule or an adhesion molecule. In some aspects, the immune-cell activating protein comprises a single binding domain that binds to one target molecule selected from the group consisting of a T cell activation receptor, a costimulatory molecule or an adhesion molecule. In some aspects, the immune-cell activating protein comprises multiple binding domains that bind to two or more target molecules selected from the group consisting of a T cell activation receptor, a costimulatory molecule and an adhesion molecule. In some aspects, the immune-cell activating protein comprises multiple binding domains that each bind to a different target molecule that is a T cell activation receptor, a costimulatory molecule and an adhesion molecule. In some aspects, the immune cell activating protein comprises at least one binding domain that binds at least one costimulatory molecule. In some aspects, the T cell activation receptor is CD3; the costimulatory molecule is CD28, CD137 or CD134; and/or the adhesion molecule is CD58 or CD2. In some aspects, each of the at least one binding domain is independently selected from an antibody or antigen-binding fragment or an ectodomain of a native ligand of the target molecule. In some aspects, the viral envelope protein is a VSV-G envelope protein, a measles virus envelope protein, a nipha virus envelope protein, or a cocal virus G protein.

In some aspects, the viral envelope protein is a VSV-G envelope protein, a measles virus envelope protein, a nipha virus envelope protein, or a cocal virus G protein. In some aspects, the viral envelope protein comprises at least one co-stimulatory molecule. In some aspects, the at least one plasmid is a plasmid encoding a co-stimulatory molecule. In some aspects, the at least one co-stimulatory molecule is CD45, CD2, CD4, CD5, CD8, CD9, CD16, CD22, CD33, CD28, CD37, CD64, CD80, CD86, CD134, CD137, CD154, OX40, 4-1BB, CD40L, or any combination thereof. In some aspects, the at least one plasmid is a plasmid encoding a helper viral protein. In some aspects, the helper viral protein is rev and/or gagpol.

In some aspects, the population of host cells is contacted with a mixture of plasmids comprising (i) a plasmid encoding a gene of interest; (ii) a plasmid encoding a rev viral protein; (iii) a plasmid encoding a gagpol viral protein; and (iv) a plasmid encoding a viral envelope protein. In some aspects, the mixture of plasmids comprises (v) a plasmid encoding an immune cell-activating protein, (vi) a plasmid encoding a co-stimulatory molecule, or (vii) any combination of (v)-(vi).

In some aspects, the disclosure provides a lentiviral formulation produced by a method described herein. In some aspects, the lentiviral formulation comprises an infectious titer of 2.0 to 6×10⁸ TU/mL. In some aspects, the formulation has an infectious titer of 2.5 to 4.7×10⁸ TU/mL. In some aspects, the total number of infectious units in the formulation is 4×1010 TU to 8×1010 TU. In some aspects, the total number of infectious units in the formulation is 5×1010 TU to 7×1010 TU, optionally at or about 6×1010 TU. In some aspects, the formulation comprises less than 10%, less than 9%, less than 8%, less than 7%, less than 6%, less than 5%, less than 4%, less than 3%, less than 2%, less than 1% or less than 0.5% of HCP. In some aspects, the formulation comprises less than 10%, less than 9%, less than 8%, less than 7%, less than 6%, less than 5%, less than 4%, less than 3%, less than 2%, less than 1% or less than 0.5% of hcDNA. In some aspects, the formulation comprises greater than 90%, greater than 91%, greater than 92%, greater than 93%, greater than 94%, greater than 95%, greater than 96%, greater than 97%, greater than 98%, or greater than 99% reduction of HCP. In some aspects, the formulation comprises greater than 90%, greater than 91%, greater than 92%, greater than 93%, greater than 94%, greater than 95%, greater than 96%, greater than 97%, greater than 98%, or greater than 99% reduction of hcDNA, optionally reduced compared to the filtered formulation prior to the concentrating. In some aspects, the formulation comprises greater than 99% reduction of hcDNA and greater than 99% reduction in HCP, optionally reduced compared to the filtered formulation prior to the concentrating. In some aspects, the formulation comprises less than 1% hcDNA and less than 1% HCP. In some aspects, a lentiviral formulation comprises a lentiviral vector at a titer of 2.5 to 4.7×10⁸ TU/mL, wherein the formulation comprises less than 1% hcDNA and less than 1% HCP. In some aspects, the volume of the formulation is 1 mL to 500 mL, optionally 10 mL to 100 mL.

### BRIEF DESCRIPTION OF THE DRAWINGS

Various objects and advantages and a more complete understanding of the disclosure are apparent and more readily appreciated by reference to the following Detailed Description and to the appended claims when taken in conjunction with the accompanying Drawing wherein:

FIGS. 1A-1C provide schematic diagrams depicting the upstream, downstream or both the upstream and downstream manufacturing processes of lentiviral particles. FIG. 1A shows the upstream process of viral particles production with host cells in suspension, and FIG. 1B shows the downstream process of viral particles purification. FIG. 1C shows an exemplary lentiviral manufacturing process.

FIG. 2 provides a box plot showing the scaling up of the upstream process. Consistent product titers were achieved when the bioreactor was scaled up from 3 liters to 10 liters to 40 liters.

FIG. 3 shows the number of harvested surface engineered lentiviral vectors (LV) produced by either HEK 293 cells or HEK 293T in bioreactors capable of holding 3 L, 10 L, 40 L or 50 L of cell culture medium. The LVs produced by HEK 293 cells were surface engineered to express a single domain fusion protein, which is referred to as LV-1 in FIG. 3. The LVs produced by the HEK 293 T cells were surface engineered to express a multi-domain fusion protein, which is referred to as LV-5 in FIG. 3.

FIGS. 4A-4B provide a schematic depicting host cell protein (HCP) and host cell DNA (hcDNA) present in the viral particle formulation following the upstream and downstream processes provided herein. LV-1 refers to a lentivirus expressing a single domain fusion protein. LV-5 refers to a lentivirus expressing a multi-domain fusion protein.

FIG. 5 provides a table showing the run conditions of seven different downstream processes.

FIG. 6 provides a table showing viral particle titer and impurities. Titer was measured by ddPCR, p24, and particles per transducing unit (TU). Impurities include residual HCP, hcDNA, E1A DNA, benzonase, and endotoxin.

FIG. 7 provides a bar graph showing the amount of host cell DNA after the second filtration step, with or without an endonuclease, and the third filtration step in the downstream process.

FIG. 8 provides a bar graph showing the amount of host cell protein after harvest clarification, ion exchange chromatography and subsequent ultrafiltration/diafiltration in the downstream process.

FIG. 9 provides a bar graph showing residual DNA and host cell protein per dose calculated for individual steps in Process No. 6.

FIG. 10 provides a schematic diagram showing a candidate downstream process. TFF refers to tangential flow filtration. Ultrafiltration/diafiltration (UF/DF) is performed in two steps, referred to as TFF-A and TFF-B.

FIGS. 11A-11B provide schematic diagrams depicting the amount of host cell protein after the downstream process. FIG. 11A provides a box plot showing HCP (μg/TU) in a clarified harvest, a chromatography purified harvest and a UF/DF purified harvest. Protein was measured by an ELISA assay in four different runs, which are labeled. FIG. 11B provides a box plot showing host cell protein in the clarified harvest, chromatography purified harvest and UF/DF purified harvest, as measured by LC-MS.

FIG. 12 provides a schematic diagram depicting host cell DNA present after the downstream process. Host cell DNA was measured by qPCR in four different runs, which are labeled.

FIG. 13 provides a schematic depicting lentiviral particle yield as a percentage of the amount of virus loaded to the column in the chromatography step following 0.75M NaCl elution compared to 2M NaCl elution, in a lentivirus expressing a single domain protein (LV-1), a lentivirus expressing three different single domain proteins+GFP (LV-2), a lentivirus expressing three different single domain proteins+CAR (LV-3), a lentivirus expressing a multi-domain fusion protein+CAR (LV-4), or a lentivirus expressing a multi-domain fusion protein (LV-5).

### DETAILED DESCRIPTION

In some embodiments, the present disclosure provides methods of preparing a formulation of infectious viral particles suitable for use in drug product manufacturing and/or in vivo use. In some embodiments, the present disclosure provides methods of preparing a formulation of viral particles for direct in vivo administration. In some embodiments, preparing a formulation of viral particles comprises subjecting a mixture of host cells and viral particles to at least two filtration steps to clarify the mixture. In some embodiments, preparing a formulation of viral particles comprises subjecting a mixture of host cells and viral particles to at least two filtration steps to clarify the mixture and subsequently concentrating the clarified mixture via chromatography and ultrafiltration.

In some embodiments, preparing a formulation of viral particles comprises subjecting a mixture of host cells and viral particles to three filtration steps to clarify the mixture. In some embodiments, preparing a formulation of viral particles comprises subjecting a mixture of host cells and viral particles to three filtration steps to clarify the mixture and subsequently concentrating the clarified mixture via chromatography and ultrafiltration.

Embodiments of the disclosure may employ conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, NY; B. Roe, J. Crabtree, and A. Kahn (1996) DNA Isolation and Sequencing: Essential Techniques, John Wiley & Sons; J. M. Polak and James O'D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA; Methods in Enzymology, Academic Press. Roe, Simon, ed. Protein Purification Techniques. 2nd ed. Oxford: Oxford University Press, 2001, Sofer, Gail and Hagel, Lars. Handbook of Process Chromatography: A Guide to Optimization, Scale-up, and Validation. London and San Diego: Academic Press, 1997, Janson, Jan-Christer and Ryden, Lars, eds. Protein Purification: Principles, High Resolution Methods, and Applications. 2nd ed. New York: John Wiley & Sons, Inc., 1998, Masters, John R. W., ed. Animal Cell Culture: A Practical Approach. 3rd ed. Oxford: Oxford University Press, 2000. Each of these general texts is herein incorporated by reference.

### I. METHODS FOR PRODUCING VIRAL PARTICLES

Scaling up virus particle production for large-scale manufacturing for clinical use represents a challenge in terms of purity and yield. The disclosure aims to overcome this challenge by providing scalable methods of manufacturing viral particles that are sufficiently pure to be used in in vivo direct injection for human subjects.

In some embodiments, the disclosure provides a scalable, suspension cell culture-based manufacture process. Some embodiments of the methods of the disclosure provide consistent cell culture titers for bioreactor scale up from 3 L to 10 L to 40 L.

In some embodiments, the upstream and downstream processes in producing and purifying viral vector particles for in vivo use are described. The disclosure also provides methods of effectively removing host cell DNA and protein, which are critical quality attributes that directly correlate with drug product safety. The disclosure provides methods that may be transferred to clinical and commercial scale manufacturing of viral particles used for in vivo administration.

### A. Upstream Process

In some embodiments, the disclosure provides a method for preparing a viral particle formulation.

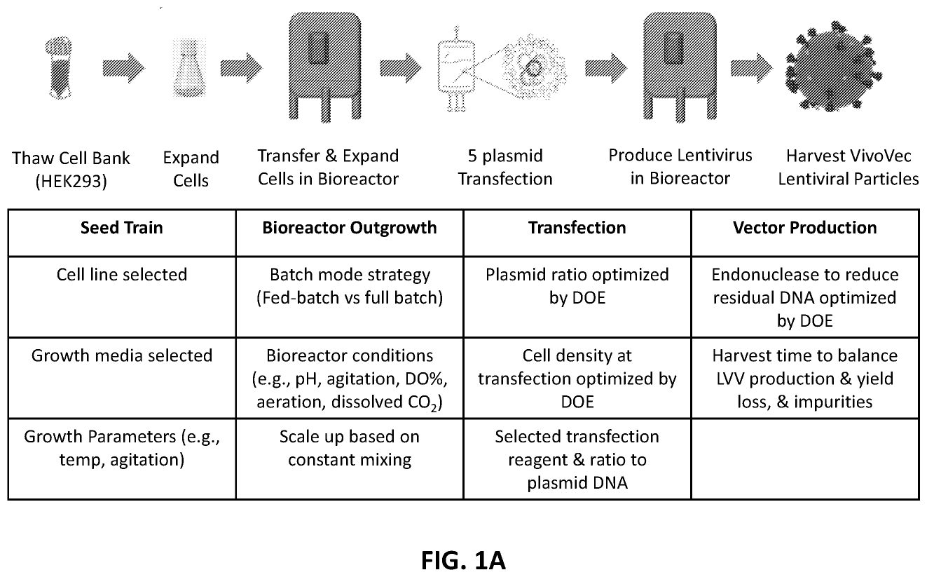

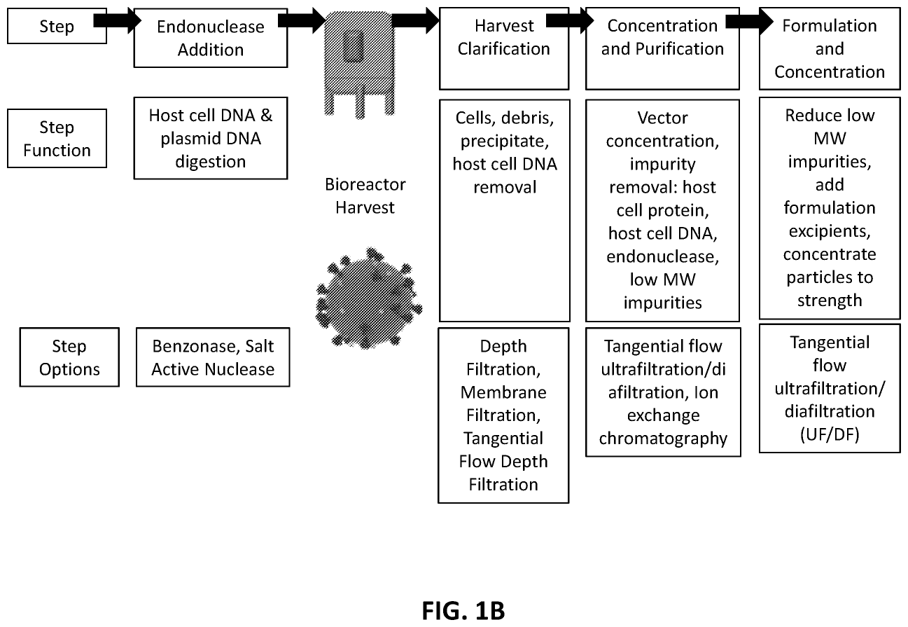

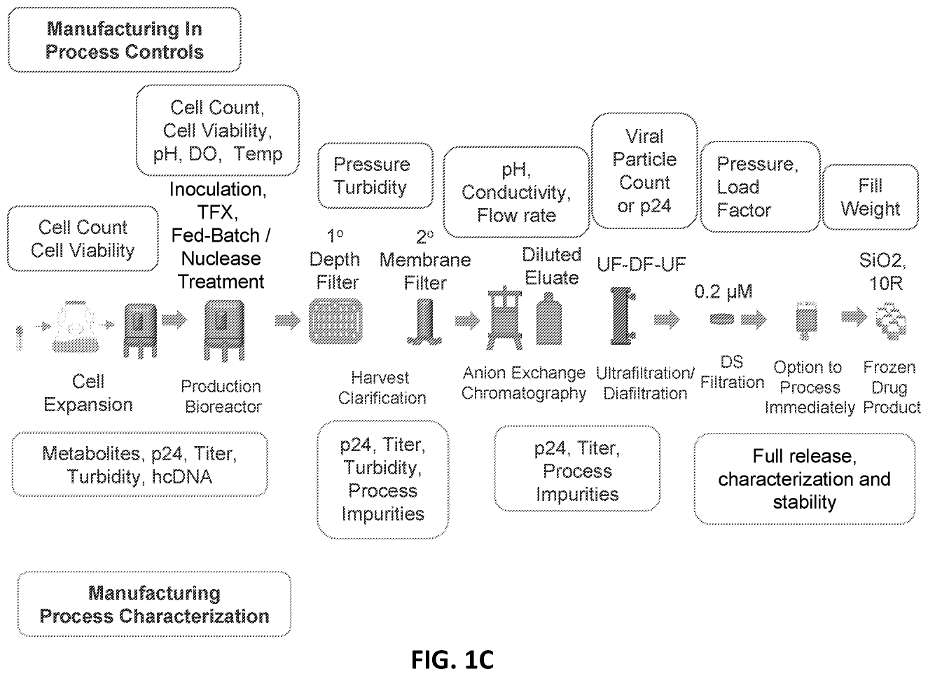

In some embodiments, the method for preparing a viral particle formulation comprises a seed train, a bioreactor outgrowth, transfection with a vector and vector production, referred to herein as an upstream process. The goal of the upstream process is to increase cell yield. In some embodiments, the upstream process includes the upstream process steps demonstrated in FIG. 1A.

In some embodiments, the methods described herein comprise an upstream process to generate viral particles. In some embodiments, the upstream process comprises (i) host cell expansion; (ii) plasmid transfection; (iii) harvesting of viral particles; or (iv) any combination of (i)-(iii). In some embodiments, the methods described herein generate retroviral particles. In some embodiments, the methods described herein generate lentiviral particles.

In some embodiments, the disclosure provides a method for preparing a viral particle formulation, comprising (i) contacting a population of host cells in suspension with at least one plasmid encoding a viral protein; (ii) culturing the population of host cells of step (i) for a period of time sufficient to produce a suspension mixture comprising a population of host cells and viral particles; (iii) filtering the suspension mixture to remove contaminants, comprising: (a) contacting the mixture with an endonuclease, (b) filtering the mixture with a first filter, wherein the first filter is a depth filter, resulting in a first filtrate (c) filtering the first filtrate with a second filter, wherein the second filter has a retention threshold smaller than the first filter, resulting in a second filtrate, and (d) filtering the second filtrate with a third filter, wherein the third filter has a retention threshold smaller than the second filter, thereby producing a filtered formulation of viral particles; and (iv) concentrating the filtered formulation of viral particles, wherein concentrating comprises chromatography and ultrafiltration.

In some embodiments, the disclosure provides a method for preparing a retroviral particle formulation, comprising (i) contacting a population of host cells in suspension with at least one plasmid encoding a retroviral protein; (ii) culturing the population of host cells of step (i) for a period of time sufficient to produce a suspension mixture comprising a population of host cells and retroviral particles; (iii) filtering the suspension mixture to remove contaminants, comprising: (a) contacting the mixture with an endonuclease, (b) filtering the mixture with a first filter, wherein the first filter is a depth filter, resulting in a first filtrate (c) filtering the first filtrate with a second filter, wherein the second filter has a retention threshold smaller than the first filter, resulting in a second filtrate, and (d) filtering the second filtrate with a third filter, wherein the third filter has a retention threshold smaller than the second filter, thereby producing a filtered formulation of retroviral particles; and (iv) concentrating the filtered formulation of viral particles, wherein concentrating comprises chromatography and ultrafiltration.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter, resulting in a first filtrate (c) filtering the first filtrate with a second filter, wherein the second filter has a retention threshold smaller than the first filter, resulting in a second filtrate, and (d) filtering the second filtrate with a third filter, wherein the third filter has a retention threshold smaller than the second filter, thereby producing a filtered formulation of lentiviral particles; and (iv) concentrating the filtered formulation of lentiviral particles, wherein concentrating comprises chromatography and ultrafiltration.

### 1. Host Cell Expansion

Viral vectors can be suitably propagated in cells (also referred to as “host cells”). A cell according to the disclosure can be any cell wherein a desired viral vector can be propagated. By using stable producer/packaging cell lines, it is possible to propagate quantities of viral vector particles (e.g., to prepare suitable titers of a viral vector) for subsequent purification.

As used herein, the term “producer cell” or “host cell” refers to a cell which contains all the elements necessary for production of lentiviral vector particles. As used herein, the term “packaging cell” refers to a cell which contains those elements necessary for production of infectious recombinant virus which are lacking in the RNA genome. Typically, such packaging cells contain one or more producer plasmids which are capable of expressing viral structural proteins (such as codon optimized gag-pol and env) but they do not contain a packaging signal.

In some embodiments, host cells/packaging cells of the disclosure are derived from a mammalian cell and. Any type of cell that is capable of supporting replication of the virus would be acceptable in the practice of methods of the disclosure.

In some embodiments, the host cell may be selected from any cell allowing production of an enveloped virus. In some embodiments, the host cell is selected from a human cell (HEK293, HEK293T, HEK293F, HEK293FT, Te671, HT1080, CEM), a muridae cell (NIH-3T3), a mustelidae cell (Mpf), and a canid cell (D17) (Miller and Chen 1996; Miller 2001; Merten 2004; Rodrigues et al. 2011; Stacey and Merten 2011). Other non-limiting examples of host cells include, but are not limited to, Vero, MDBK, BK-21, CV-1 cells, and mammalian fibroblast or cultured epithelial cells. In some embodiments, the host cells are readily available from commercial Sources (e.g., ATCC, Rockville, Md.).

In some embodiments, host cells/packaging cells of the disclosure are derived from a primate cell, such as human embryonic kidney cell. In some embodiments, the cell may be derived from an existing cell line, e.g., from a HEK 293 cell line. In some embodiments, the host cell is an HEK 293 cell line. In some embodiments, the cell may be derived from an existing cell line, e.g., from a HEK 293T cell line. In some embodiments, the host cell is an HEK 293T cell line. In some embodiments, the cells are adapted for suspension culture.

In some embodiments, the host cells are cultivated in a medium suitable for cultivation of mammal cells and for producing an enveloped virus. The medium may be supplemented with additives well known in the field such as antibiotics and serum (notably fetal calf serum, etc.) added in suitable concentrations. The medium used may notably comprise serum or be serum-free. The culture media for mammal cells are well known in the field, including but not limited to, DMEM (Dulbecco's Modified Eagle's Medium), RPMI1640 or a mixture of various culture media, including for example DMEM/F12, or a serum-free medium like optiMEM®, optiPRO®, optiPRO-SFM®, CD293®, Freestyle F17® (Life Technologies) or Ex-Cell® 293 (Sigma-Aldrich), and LV-MAX™ medium (ThermoFisher Scientific #A3583402).

In some embodiments, the host cells are cultivated in a medium that does not contain serum. In some embodiments, the host cells are cultivated in a medium that does not contain animal products.

In some embodiments, host cells are first diluted into a suitable medium before centrifuging to remove the medium. In some embodiments, seed train expansion of the host cells is performed following the removal of the medium. In some embodiments, seed train expansion is performed to achieve a target cell number. In some embodiments, the series of target cell numbers are achieved. In some embodiments, different target cell numbers correspond to containers of different sizes.

In some embodiments, the seed expansion is first carried out in a plurality of containers until a sufficient number of cells for a bioreactor inoculation is achieved.

In some embodiments, the seed expansion comprises a target inoculation cell density and a target passage cell density. In some embodiments, seed train expansion of the host cells is performed to achieve a target inoculation cell density. In some embodiments, seed train expansion of the host cells is performed to achieve a target passage cell density. In some embodiments, the seed expansion is initiated by inoculating a container with a target inoculation cell density. In some embodiments, the target passage cell density is achieved after cultivating the host cells in the container for their expansion. In some embodiments, once the target passage cell density is achieved, the host cells are passaged into another container, one or more times (e.g., serially). In some embodiments, each passage into a new container is at a target inoculation density and cultivation is maintained under conditions to achieve the target passage density. In some embodiments, the seed expansion is performed to achieve a target number of cells for bioreactor inoculation.

In some embodiments, cells are inoculated in a container or containers for cultivation in the medium until the target passage cell density is achieved. In some embodiments, the container comprises a tissue culture flask, dish, or roller bottle. In some embodiments, the container is a tissue culture flask. In some embodiments, the tissue culture flask is a shake flask. In some embodiments, the tissue culture flask is appropriate for suspension cells. In some embodiments, the tissue culture flask is a non-treated flask. In some embodiments, the container is a bioreactor. In some embodiments, the bioreactor is appropriate for suspension cells.

In some embodiments, the seed train expansion is performed serially in a plurality of containers. In some embodiments, the plurality of containers comprises different containers. In some embodiments, the different containers are different sizes. In some embodiments, the size of the containers can hold a volume between 0.125 L to 50 L, such as 0.125 L to 10 L or 0.125 L to 5 L, each with an increasing size compared to the prior container in a series of containers for expansion. In some embodiments, the size of the containers can hold a volume between 0.125 L to 5 L, each with an increasing size compared to the prior container in a series of containers for expansion. In some embodiments, the container can hold a volume of 0.125 L, 0.25 L, 0.5 L, 1 L, 1.6 L, 2 L, or 5 L. In some embodiments, the plurality of containers comprise two or more shake flasks. In some embodiments, the plurality of containers is for serially passaging the cells. In some embodiments, the cells are inoculated in a container (e.g. shake flask) at a target inoculation density of between 2×10⁵ cells/mL to 5×10⁵ cells/mL, such as at or about 3×10⁵ cells/mL to 5×10⁵ cells/mL, and are serially passaged for inoculation of a larger container when the cells reach a target passage density of about 3.5 to 6×10⁶ cells/mL, such as 4.0 to 6×10⁶ cells/mL. In some embodiments, the methods for seed expansion before bioreactor inoculation involves 2, 3, 4, 5 or more passages of the cells. In some embodiments, the methods for seed expansion involves passaging the cells at least 5 times. In some embodiments, the cells are passaged into containers for increasing size, such as containers that can hold a volume of 0.125 L, 0.5 L, 1 L, 1.6 L, and 5 L.

In some embodiments, the seed train expansion is performed in a plurality of shake flasks. In some embodiments, seed train expansion is performed in two or more, three or more, or four or more shake flasks. In some embodiments, seed train expansion is performed in at least two, at least three, at least four, or at least five shake flasks.

In some embodiments, the seed train expansion is initiated by inoculating host cells that achieve a target inoculation cell density into a first shake flask. In some embodiments, the first shake flask holds a volume of 0.125 L. In some embodiments, the target inoculation cell density for the first shake flask is no lower than 5×10⁵ cells/mL.

In some embodiments, once the inoculated cells achieve a target passage cell density in the first shake flask, the host cells are passaged into a second shake flask at a target inoculation cell density. In some embodiments, the second shake flask holds a volume of 0.5 L. In some embodiments, the target inoculation cell density for the second shake flask is about 2×10⁵ cells/mL to 5×10⁵ cells/mL, such as at or about 3×10⁵ cells/mL to 5×10⁵ cells/mL. In some embodiments, the target inoculation cell density for the second shake flask is no lower than 5×10⁵ cells/mL. In some embodiments, the target inoculation cell density for the second shake flask is about 5×10⁵ cells/mL. In some embodiments, the target passage cell density of the first flask is about 3.5 to 6×10⁶ cells/mL. In some embodiments, the target passage cell density for the first shake flask is 4×10⁶ cells/mL. In some embodiments, the target passage cell density for the first shake flask is 5×10⁶ cells/mL. In some embodiments, the target passage cell density for the first shake flask is 6×10⁶ cells/mL.

In some embodiments, once the inoculated cells achieve a target passage cell density in the second shake flask, the host cells are passaged into a third shake flask at a target inoculation cell density. In some embodiments, the third shake flask holds a volume of 1 L. In some embodiments, the target inoculation cell density for the third shake flask is about 2×10⁵ cells/mL to 5×10⁵ cells/mL, such as at or about 3×10⁵ cells/mL to 5×10⁵ cells/mL. In some embodiments, the target inoculation cell density for the third shake flask is no lower than 5×10⁵ cells/mL. In some embodiments, the target inoculation cell density for the third shake flask is about 5×10⁵ cells/mL. In some embodiments, the target passage cell density of the second shake flask is between about 3.5 to 6×10⁶ cells/mL. In some embodiments, the target passage cell density for the second shake flask is 4×10⁶ cells/mL. In some embodiments, the target passage cell density for the second shake flask is 5×10⁶ cells/mL. In some embodiments, the target passage cell density for the second shake flask is 6×10⁶ cells/mL.

In some embodiments, once the inoculated cells achieve a target passage cell density in the third shake flask, the host cells are passaged into a fourth shake flask at a target inoculation cell density. In some embodiments, the fourth shake flask holds a volume of 1.6 L. In some embodiments, the target inoculation cell density for the fourth shake flask is about 2×10⁵ cells/mL to 5×10⁵ cells/mL, such as at or about 3×10⁵ cells/mL to 5×10⁵ cells/mL. In some embodiments, the target inoculation cell density for the fourth shake flask is no lower than 5×10⁵ cells/mL. In some embodiments, the target inoculation cell density for the fourth shake flask is about 5×10⁵ cells/mL. In some embodiments, the target passage cell density of the third shake flask is between about 3.5 to 6×10⁶ cells/mL. In some embodiments, the target passage cell density for the third shake flask is 4×10⁶ cells/mL. In some embodiments, the target passage cell density for the third shake flask is 5×10⁶ cells/mL. In some embodiments, the target passage cell density for the third shake flask is 6×10⁶ cells/mL.

In some embodiments, once the inoculated cells achieve a target passage cell density in the fourth shake flask, the host cells are passaged into a fifth shake flask at a target inoculation cell density. In some embodiments, the fifth shake flask holds a volume of 5 L. In some embodiments, the target inoculation cell density for the fifth shake flask is about 2×10⁵ cells/mL to 5×10⁵ cells/mL, such as at or about 3×10⁵ cells/mL to 5×10⁵ cells/mL. In some embodiments, the target inoculation cell density for the fifth shake flask is no lower than 5×10⁵ cells/mL. In some embodiments, the target inoculation cell density for the fifth shake flask is about 5×10⁵ cells/mL. In some embodiments, the target passage cell density of the fourth shake flask is between about 3.5 to 6×10⁶ cells/mL. In some embodiments, the target passage cell density for the fourth shake flask is 4×10⁶ cells/mL. In some embodiments, the target passage cell density for the fourth shake flask is 5×10⁶ cells/mL. In some embodiments, the target passage cell density for the fourth shake flask is 6×10⁶ cells/mL.

In some embodiments, the seed train expansion is completed when the host cells in the fifth shake flask achieve a target passage cell density. In some embodiments, the target passage cell density of the fifth shake flask is between about 3.5 to 6×10⁶ cells/mL. In some embodiments, the target passage cell density for the fifth shake flask is 4×10⁶ cells/mL. In some embodiments, the target passage cell density for the fifth shake flask is 5×10⁶ cells/mL. In some embodiments, the target passage cell density for the fifth shake flask is 6×10⁶ cells/mL. In some embodiments, the target passage cell density of the fifth shake flask is between about 6 to 7×10⁹ cells/mL. In some embodiments, when the seed train expansion is complete, host cells are passaged into a bioreactor at a target inoculation cell density. In some embodiments, the target inoculation cell density for the bioreactor is no lower than 5×10⁵ cells/mL. In some embodiments, the target inoculation cell density for the bioreactor is about 2×10⁵ cells/mL to 5×10⁵ cells/mL, such as at or about 3×10⁵ cells/mL to 5×10⁵ cells/mL. In some embodiments, the target inoculation cell density for the second shake flask is about 5×10⁵ cells/mL.

In some embodiments, the bioreactor can hold a volume between 3 L to 200 L. In some embodiments, the bioreactor can hold a volume of 3 L, 10 L, 40 L, 50 L or 200 L. In some embodiments, the bioreactor can hold a volume of 40 L. In some embodiments, the bioreactor can hold a volume of 200 L. In some embodiments, the bioreactor has a volume of from about 20 mL to about 1500 mL, such as from about 20 mL to about 1000 mL, about 20 mL to about 500 mL, about 20 mL to about 200 mL, about 20 mL to about 100 mL or about 20 mL to about 40 mL. In some embodiments, the bioreactor has a volume of about 40 mL to about 200 mL. In some embodiments, the bioreactor has a volume of at or about 40 mL. In some embodiments, the bioreactor has a volume of at or about 50 mL. In some embodiments, the bioreactor has a volume of at or about 100 mL. In some embodiments, the bioreactor has a volume of at or about 200 mL.

In some embodiments, the host cells are inoculated and grown in each of the one or more bioreactor under dissolved oxygen and pH control. In some embodiments, the host cells are inoculated in a first bioreactor and grown under dissolved oxygen and pH control. In some embodiments, the host cells are transferred from a first bioreactor to a second bioreactor and grown under dissolved oxygen and pH control. In some embodiments, the bioreactor is suitable for transfection.

For instance, in some embodiments, cells are first inoculated into a shake flask (e.g. 125 mL shake flask) and then are transferred to one or more larger shake flasks (e.g. 500 mL)—each with an increasing volume—until such time as a sufficient number of cells is achieved for bioreactor inoculation. In some embodiments, the cell number sufficient for bioreactor inoculation is 6 to 7×10⁹ cells. In some embodiments, the cell density sufficient for bioreactor inoculation is between 3.5 to 6×10⁶ cells/mL.

In some embodiments, the seed train expansion is initiated with no lower than 5×10⁵ cells/mL. In some embodiments, the cells are seeded into a 125 mL flask. In some embodiments, the cells are seeded into a 500 mL shake flask. In some embodiments, the cells are seeded into a 1 L shake flask. In some embodiments, the cells are seeded into a 1.6 L shake flask. In some embodiments, the cells are seeded into a 5 L shake flask.

In some embodiments, the bioreactor is inoculated after seed train expansion is performed up to the 5 L shake flask.

In some embodiments, a series of target cell numbers are achieved from the cultivation in each of the plurality of containers. In some embodiments, different target cell numbers correspond to containers of different sizes.

In some embodiments, a series of target cell densities are achieved from the cultivation in each of the plurality of containers. In some embodiments, different target cell densities correspond to containers of different sizes.

In some embodiments, the expansion is continued in one or more bioreactors until a final target cell density of about 4×10⁶ cells/mL to 6×10⁶ cells/mL is achieved in a bioreactor at the desired scale for the expansion. In some embodiments, the bioreactor has a size of 40 mL to 500 mL, such as at or about 40 mL to 200 mL. In some embodiments, the bioreactor is a size of at or about 40 mL. In some embodiments, the bioreactor is a size of at or about 50 mL. In some embodiments, the bioreactor is a size of at or about 100 mL. In some embodiments, the bioreactor is a size of at or about 200 mL.

In some embodiments, the seed expansion is initiated with a first target cell density of cells of at or about 5×10⁵ cells/mL. In some embodiments, a second target cell density is 4 to 6×10⁶ cells/mL. In some embodiments, the second target cell density is achieved in a container of 125-500 mL. In some embodiments, a third target cell number is 6 to 7×10⁹ cells. In some embodiments, the third target cell number is achieved at a density of the cells that is at or about 5×10⁵ cells/mL. In some embodiments, the third target cell number is achieved in a container of 1-5 L. In some embodiments, the third target cell number is suitable for inoculation in a container of about 50 L, optionally where the container is a bioreactor. In some embodiments, the bioreactor is a first bioreactor and the cells are transferred to a second bioreactor for full scale expansion until a final target cell density is achieved. In some embodiments, the host cells are transfected with plasmid when the final target cell density is achieved.

In some embodiments, the host cells are inoculated and grown in a first bioreactor under dissolved oxygen and pH control. In some embodiments, the first bioreactor is about 50 L. In some embodiments, the target cell density in the first bioreactor is 4 to 6×10⁶ cells/mL.

In some embodiments, the host cells are transferred to a second bioreactor once the target cell density is reached in the first bioreactor. In some embodiments, the second bioreactor is about 200 L. In some embodiments, the initial cell density in the second bioreactor upon transfer is 1-5×10⁵ cells/mL. In some embodiments, the host cells are grown in the second bioreactor until a final target cell density is reached. In some embodiments, the final target cell density in the second bioreactor is 4 to 6×10⁶ cells/mL. In some embodiments, the final target cell density in the second bioreactor is about 5×10⁶ cells/mL. In some embodiments, the final target cell density in the second bioreactor is suitable for transfection.

In some embodiments, the seed expansion is initiated with a first cell density that is about 5×10⁵ cells/mL into a first container. In some embodiments, the cells are cultivated in the first container until a second target cell density is achieved that is 4 to 6×10⁶ cells/mL. In some embodiments, cells from the first container are transferred to a second container, and the cells are cultivated in the second container until expansion of a target number of cells for bioreactor inoculation that is 6 to 7×10⁹ cells/mL. In some embodiments, each of the first and second container are containers of increasing size. In some embodiments, the first and second containers are shake flasks. In some embodiments, the first container (e.g. shake flask) has a volume of 125-500 mL. In some embodiments the second container (e.g. shake flask) has a volume of 1-5 L. In some embodiments, the first shake flask holds a volume of 125 mL. In some embodiments the first shake flask holds a volume of 500 mL. In some embodiments, the second shake flask holds a volume of 1 L. In some embodiments, the second shake flask holds a volume of 1.6 L. In some embodiments, the second shake flask holds a volume of 5 L.

In some embodiments, the target number of cells expanded from the seed train expansion in shake flasks is used to inoculate a bioreactor. In some embodiments, cells from the target number of cells from the second container are inoculated into a first bioreactor, and the cells are cultivated in the bioreactor until a target cell density is achieved between the range of 4 to 6×10⁶ cells/mL. In some embodiments, the cells from the first bioreactor are inoculated into a second bioreactor at scale, and the cells are cultivated in the second bioreactor until a final target cell density is achieved between the range of 4 to 7×10⁶ cells/mL. In some embodiments, the final target cell density in the second bioreactor is about 5×10⁶ cells/mL. In some embodiments, the first bioreactor has a smaller volume than the second bioreactor. In some embodiments, the first bioreactor has a volume of 3-50 L. In some embodiments, the second bioreactor has a volume of 40-200 L. In some embodiments, the first bioreactor holds a volume of 3 L. In some embodiments, the first bioreactor holds a volume of 10 L. In some embodiments, the first bioreactor holds a volume of 50 L. In some embodiments, the first bioreactor holds a volume of 40 L. In some embodiments, In some embodiments, a second bioreactor holds a volume of 200 L.

In some embodiments, the host cells are transfected with plasmid when the final target cell density is achieved.

### 2. Plasmid Transfection

In some embodiments, the methods described herein comprise transfecting a host cell with at least one plasmid to generate a viral particle. In some embodiments, the methods described herein comprise contacting a population of host cells with at least one plasmid encoding a viral particle for a period of time sufficient to produce a viral particle. In some embodiments, contacting a population of host cells with at least one plasmid encoding a viral protein is performed by transfection.

In some embodiments, transfection is performed in a bioreactor once a target transfection density is reached. In some embodiments, the target transfection density is the final target density achieved by the seed expansion method for host cell expansion as described above. In some cases, fresh media may be added to the bioreactor to dilute the final target density to the target transfection density. In some embodiments, the target transfection density is about 0.5 to about 10×10⁶ cells/mL. In some embodiments, the target transfection density is about 1 to about 3×10⁶ cells/mL. In some embodiments, the target transfection density is about 3 to 6×10⁶ cells/mL. In some embodiments, the target transfection density is about 4 to about 7×10⁶ cells/mL. In some embodiments, the target transfection density is about 1×10⁶ cells/mL, about 2×10⁶ cells/mL, about 3×10⁶ cells/mL, about 4×10⁶ cells/mL, about 5×10⁶ cells/mL or about 6×10⁶ cells/mL, or any value between any of the foregoing. In some embodiments, the target transfection density is 4×10⁶ cells/mL.

In some embodiments, the target transfection density is determined by or is dependent on the host cell. In some cases, certain host cells have higher productivity. For instance, HEK293T cells can exhibit higher productivity than HEK293 cells, such that the target transfection density when using HEK293T cells can be 3-fold or lower, such as 2-3-fold lower, than the target transfection density when using HEK293 cells. The ability to achieve a desired yield while using a lower cell density improves the process by providing a lower residual impurity burden to the downstream steps.

In some embodiments, the host cells are HEK293T cells. In some embodiments, the target transfection density is about 1 to about 3×10⁶ cells/mL. In some embodiments, the target transfection density is about 1×10⁶ cells/mL. In some embodiments, the target transfection density is about 2×10⁶ cells/mL. In some embodiments, the target transfection density is about 3×10⁶ cells/mL.

In some embodiments, the host cells are HEK293 cells. In some embodiments, the target transfection density is about 3 to 6×10⁶ cells/mL. In some embodiments, the target transfection density is about 4×10⁶ cells/mL. In some embodiments, the target transfection density is about 5×10⁶ cells/mL. In some embodiments, the target transfection density is about 6×10⁶ cells/mL.

In some embodiments, the contents of the bioreactor are diluted with fresh media to reach the target transfection density. In some embodiments, the target transfection density is 4×10⁶ cells/mL. In some embodiments, a bolus of glucose is added to the bioreactor with the fresh media to a target glucose level. In some embodiments, glucose is supplemented in the media for transfection. In some embodiments, the target glucose level in the media for the transfection is 1 g/L to 10 g/L, such as 2.5 g/L to 7.5 g/L. In some embodiments, the target glucose level is or is about 5.5 g/L. In some embodiments, glucose is supplemented at a concentration to target 5.5 g/L.

In some embodiments, transfection is performed shortly after the target transfection density is reached. The transfection methods may be performed using methods well known in the art. For example, the transfection process may be performed using calcium phosphate or commercially available formulations such as Lipofectamine™ 2000CD (Invitrogen, CA) or polyethylenimine (PEI).

Commercially available instruments and reagents may be used for transfection. In some embodiments, the instruments and reagents used for transfection is specific for large-scale virus production. Illustrative examples of such commercially available reagents include PEIpro® (Polyplus-Transfection®).

In some embodiments, at least one plasmid used for transfection comprises a non-coding nucleic acid. In some embodiments, the non-coding nucleic acid is a siRNA, a miRNA, or a shRNA.

Exemplary genes of interest include any payloads as described in Section II.A.2.b.v. In some embodiments, at least one plasmid used for transfection comprises a polynucleotide encoding a polypeptide or gene of interest (e.g. also referred to as a payload gene). In some embodiments, the at least one plasmid comprising a polynucleotide encoding a gene or polypeptide of interest is a transfer plasmid. In some embodiments, the gene of interest is not expressed by the host cell and the transfer plasmid is incorporated into the viral particle as a nucleic acid. In some embodiments, the polypeptide of interest is a chimeric antigen receptor (CAR). In some embodiments, the chimeric antigen receptor is specific for a tumor-associated antigen. In some embodiments, the tumor-associate antigen is CD19, BCMA, GPRC5D, ROR1, FcRL5, alpha-fetoprotein, or Her2. In some embodiments, the chimeric antigen receptor is specific for a tag. In some embodiments, the tag is a hapten. In some embodiments, exemplary haptens include: DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin.

In some embodiments, at least one plasmid used for transfection encodes a viral envelope protein. In some embodiments, the viral envelope protein is a VSV-G envelope protein, a measles virus envelope protein, a nipah virus envelope protein, or a cocal virus G protein. In some embodiments, the viral envelope protein is a cocal virus G protein.

In some embodiments, the Cocal virus G envelope protein comprises an amino acid sequence at least 80%, 85%, 90%, 95%, 96%, 97%, 98%, 99% or 100% identical to the amino acid sequence of SEQ ID NO:124. In some embodiments, the Cocal virus G envelope protein comprises an amino acid sequence at least 80% identical to the amino acid sequence of SEQ ID NO:124. In some embodiments, the Cocal virus G envelope protein comprises an amino acid sequence at least 85% identical to the amino acid sequence of SEQ ID NO:124. In some embodiments, the Cocal virus G envelope protein comprises an amino acid sequence at least 90% identical to the amino acid sequence of SEQ ID NO:124. In some embodiments, the Cocal virus G envelope protein comprises an amino acid sequence at least 95% identical to the amino acid sequence of SEQ ID NO:124. In some embodiments, the Cocal virus G envelope protein comprises an amino acid sequence at least 96% identical to the amino acid sequence of SEQ ID NO:124. In some embodiments, the Cocal virus G envelope protein comprises an amino acid sequence at least 97% identical to the amino acid sequence of SEQ ID NO:124. In some embodiments, the Cocal virus G envelope protein comprises an amino acid sequence at least 98% identical to the amino acid sequence of SEQ ID NO:124. In some embodiments, the Cocal virus G envelope protein comprises an amino acid sequence at least 99% identical to the amino acid sequence of SEQ ID NO:124. In some embodiments, the Cocal virus G envelope protein comprises an amino acid sequence at least 100% identical to the amino acid sequence of SEQ ID NO:124. In some embodiments, the Cocal virus G envelope protein comprises the amino acid sequence of SEQ ID NO:124. In some embodiments, the Cocal virus G envelope protein consists the amino acid sequence of SEQ ID NO:124.

In some embodiments, the Cocal virus G envelope protein is encoded by a nucleotide sequence at least 80%, 85%, 90%, 95%, 96%, 97%, 98%, 99% or 100% identical to the nucleotide sequence of SEQ ID NO:125. In some embodiments, the Cocal virus G envelope protein is encoded by a nucleotide sequence at least 80% identical to the nucleotide sequence of SEQ ID NO:125. In some embodiments, the Cocal virus G envelope protein is encoded by a nucleotide sequence at least 85% identical to the nucleotide sequence of SEQ ID NO:125. In some embodiments, the Cocal virus G envelope protein is encoded by a nucleotide sequence at least 90% identical to the nucleotide sequence of SEQ ID NO:125. In some embodiments, the Cocal virus G envelope protein is encoded by a nucleotide sequence at least 95% identical to the nucleotide sequence of SEQ ID NO:125. In some embodiments, the Cocal virus G envelope protein is encoded by a nucleotide sequence at least 96% identical to the nucleotide sequence of SEQ ID NO:125. In some embodiments, the Cocal virus G envelope protein is encoded by a nucleotide sequence at least 97% identical to the nucleotide sequence of SEQ ID NO:125. In some embodiments, the Cocal virus G envelope protein is encoded by a nucleotide sequence at least 98% identical to the nucleotide sequence of SEQ ID NO:125. In some embodiments, the Cocal virus G envelope protein is encoded by a nucleotide sequence at least 99% identical to the nucleotide sequence of SEQ ID NO:125. In some embodiments, the Cocal virus G envelope protein is encoded by a nucleotide sequence at least 100% identical to the nucleotide sequence of SEQ ID NO:125. In some embodiments, the Cocal virus G envelope protein is encoded by a nucleotide comprising the nucleotide sequence of SEQ ID NO:125. In some embodiments, the Cocal virus G envelope protein is encoded by a nucleotide consisting of the nucleotide sequence of SEQ ID NO:125.

In some embodiments, the Cocal virus G envelope protein is encoded by a nucleotide sequence at least 80%, 85%, 90%, 95%, 96%, 97%, 98%, 99% or 100% identical to the nucleotide sequence of SEQ ID NO:126. In some embodiments, the Cocal virus G envelope protein is encoded by a nucleotide sequence at least 80% identical to the nucleotide sequence of SEQ ID NO:126. In some embodiments, the Cocal virus G envelope protein is encoded by a nucleotide sequence at least 85% identical to the nucleotide sequence of SEQ ID NO:126. In some embodiments, the Cocal virus G envelope protein is encoded by a nucleotide sequence at least 90% identical to the nucleotide sequence of SEQ ID NO:126. In some embodiments, the Cocal virus G envelope protein is encoded by a nucleotide sequence at least 95% identical to the nucleotide sequence of SEQ ID NO:126. In some embodiments, the Cocal virus G envelope protein is encoded by a nucleotide sequence at least 96% identical to the nucleotide sequence of SEQ ID NO:126. In some embodiments, the Cocal virus G envelope protein is encoded by a nucleotide sequence at least 97% identical to the nucleotide sequence of SEQ ID NO:126. In some embodiments, the Cocal virus G envelope protein is encoded by a nucleotide sequence at least 98% identical to the nucleotide sequence of SEQ ID NO:126. In some embodiments, the Cocal virus G envelope protein is encoded by a nucleotide sequence at least 99% identical to the nucleotide sequence of SEQ ID NO:126. In some embodiments, the Cocal virus G envelope protein is encoded by a nucleotide sequence at least 100% identical to the nucleotide sequence of SEQ ID NO:126. In some embodiments, the Cocal virus G envelope protein is encoded by a nucleotide comprising the nucleotide sequence of SEQ ID NO:126. In some embodiments, the Cocal virus G envelope protein is encoded by a nucleotide consisting of the nucleotide sequence of SEQ ID NO:126.

The envelope expression cassette may include one of a number of envelopes such as VSV-G or various murine retrovirus envelopes such as 4070A. In some embodiments, the viral envelope protein is a fusion protein with an immune cell-activating protein. The immune-activating protein can be a single binding domain protein or a multiple binding domain protein that binds to one or more target molecules on immune cells to stimulate or activate immune cell activity. In some embodiments, the immune cell-activating protein contains at least one binding domain that binds to a primary T cell receptor (e.g. CD3), a costimulatory molecule or an adhesion molecule. In some embodiments, the viral envelope protein comprises a binding domain that binds to at least one co-stimulatory molecule.

In some embodiments, at least one plasmid used for transfection encodes a protein that binds to a co-stimulatory molecule. In some embodiments, at least one co-stimulatory molecule is CD45, CD2, CD4, CD5, CD8, CD9, CD16, CD22, CD33, CD28, CD37, CD64, CD80, CD86, CD134, CD137, CD154, OX40, 4-1BB, CD40L, or any combination thereof. In some embodiments, the costimulatory molecule is any of the costimulatory molecules described in Section II.A.1.b.

In some embodiments, at least one plasmid used for transfection encodes an immune cell activating protein. In some embodiments, the immune-cell activating protein is a protein that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, TCR α chain, TCR β chain, TCR ζ chain, TCR γ chain, TCR δ chain, CD3 ε TCR subunit, CD3 γ TCR subunit, CD3 δ TCR subunit, or NKp80. In some embodiments, the costimulatory molecule is any of the costimulatory molecules described in Section II.A.1.a.

In some embodiments, at least one plasmid used for transfection encodes a protein that binds to an adhesion molecule. In some embodiments, at least one adhesion molecule is CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, B7-H6, SLAMF2, B7-H2, B7-H5, B7-H3, B7x, and TMIGD2 or any combination thereof. In some embodiments, the adhesion molecule is any of the adhesion molecules described in Section II.A.1.c.

In some embodiments, to improve safety, host cells have been engineered wherein the 3′LTR of the provirus is deleted. In such cells, two recombination events would be necessary to produce a wild type virus. In some embodiments, further safety improvements involve the introduction of the gag-pol genes and the env gene on separate constructs. These constructs can be introduced sequentially to prevent recombination during transfection. In these split-construct cell lines, a further reduction in recombination may be achieved by changing the codons. This technique, based on the redundancy of the genetic code, aims to reduce homology between the separate constructs, for example between the regions of overlap in the gag-pol and env open reading frames.

In some embodiments, at least one plasmid used for transfection encodes a helper viral protein. In some embodiments, the helper viral protein is rev and/or gagpol.

In some embodiments, the gag protein comprises an amino acid sequence at least 80%, 85%, 90%, 95%, 96%, 97%, 98%, 99% or 100% identical to the amino acid sequence of SEQ ID NO:46. In some embodiments, the gag protein comprises an amino acid sequence at least 80% identical to the amino acid sequence of SEQ ID NO:46. In some embodiments, the gag protein comprises an amino acid sequence at least 85% identical to the amino acid sequence of SEQ ID NO:46. In some embodiments, the gag protein comprises an amino acid sequence at least 90% identical to the amino acid sequence of SEQ ID NO:46. In some embodiments, the gag protein comprises an amino acid sequence at least 95% identical to the amino acid sequence of SEQ ID NO:46. In some embodiments, the gag protein comprises an amino acid sequence at least 96% identical to the amino acid sequence of SEQ ID NO:46. In some embodiments, the gag protein comprises an amino acid sequence at least 97% identical to the amino acid sequence of SEQ ID NO:46. In some embodiments, the gag protein comprises an amino acid sequence at least 98% identical to the amino acid sequence of SEQ ID NO:46. In some embodiments, the gag protein comprises an amino acid sequence at least 99% identical to the amino acid sequence of SEQ ID NO:46. In some embodiments, the gag protein comprises an amino acid sequence at least 100% identical to the amino acid sequence of SEQ ID NO:46. In some embodiments, the gag protein comprises the amino acid sequence of SEQ ID NO:46. In some embodiments, the gag protein consists the amino acid sequence of SEQ ID NO:46.

In some embodiments, the pol protein comprises an amino acid sequence at least 80%, 85%, 90%, 95%, 96%, 97%, 98%, 99% or 100% identical to the amino acid sequence of SEQ ID NO:49. In some embodiments, the pol protein comprises an amino acid sequence at least 80% identical to the amino acid sequence of SEQ ID NO:49. In some embodiments, the pol protein comprises an amino acid sequence at least 85% identical to the amino acid sequence of SEQ ID NO:49. In some embodiments, the pol protein comprises an amino acid sequence at least 90% identical to the amino acid sequence of SEQ ID NO:49. In some embodiments, the pol protein comprises an amino acid sequence at least 95% identical to the amino acid sequence of SEQ ID NO:49. In some embodiments, the pol protein comprises an amino acid sequence at least 96% identical to the amino acid sequence of SEQ ID NO:49. In some embodiments, the pol protein comprises an amino acid sequence at least 97% identical to the amino acid sequence of SEQ ID NO:49. In some embodiments, the pol protein comprises an amino acid sequence at least 98% identical to the amino acid sequence of SEQ ID NO:49. In some embodiments, the pol protein comprises an amino acid sequence at least 99% identical to the amino acid sequence of SEQ ID NO:49. In some embodiments, the pol protein comprises an amino acid sequence at least 100% identical to the amino acid sequence of SEQ ID NO:49. In some embodiments, the pol protein comprises the amino acid sequence of SEQ ID NO:49. In some embodiments, the pol protein consists the amino acid sequence of SEQ ID NO:49.

In some embodiments, the gag and pol protein are encoded by a nucleotide sequence at least 80%, 85%, 90%, 95%, 96%, 97%, 98%, 99% or 100% identical to the nucleotide sequence of SEQ ID NO:52. In some embodiments, the gag and pol protein are encoded by a nucleotide sequence at least 80% identical to the nucleotide sequence of SEQ ID NO:52. In some embodiments, the gag and pol protein are encoded by a nucleotide sequence at least 85% identical to the nucleotide sequence of SEQ ID NO:52. In some embodiments, the gag and pol protein are encoded by a nucleotide sequence at least 90% identical to the nucleotide sequence of SEQ ID NO:52. In some embodiments, the gag and pol protein are encoded by a nucleotide sequence at least 95% identical to the nucleotide sequence of SEQ ID NO:52. In some embodiments, the gag and pol protein are encoded by a nucleotide sequence at least 96% identical to the nucleotide sequence of SEQ ID NO:52. In some embodiments, the gag and pol protein are encoded by a nucleotide sequence at least 97% identical to the nucleotide sequence of SEQ ID NO:52. In some embodiments, the gag and pol protein are encoded by a nucleotide sequence at least 98% identical to the nucleotide sequence of SEQ ID NO:52. In some embodiments, the gag and pol protein are encoded by a nucleotide sequence at least 99% identical to the nucleotide sequence of SEQ ID NO:52. In some embodiments, the gag and pol protein are encoded by a nucleotide sequence at least 100% identical to the nucleotide sequence of SEQ ID NO:52. In some embodiments, the gag and pol protein are encoded by the nucleotide sequence of SEQ ID NO:52. In some embodiments, the gag and pol protein consist of the nucleotide sequence of SEQ ID NO:52.

In some embodiments, the rev protein comprises an amino acid sequence at least 80%, 85%, 90%, 95%, 96%, 97%, 98%, 99% or 100% identical to the amino acid sequence of SEQ ID NO:127. In some embodiments, the rev protein comprises an amino acid sequence at least 80% identical to the amino acid sequence of SEQ ID NO: 127. In some embodiments, the rev protein comprises an amino acid sequence at least 85% identical to the amino acid sequence of SEQ ID NO: 127. In some embodiments, the rev protein comprises an amino acid sequence at least 90% identical to the amino acid sequence of SEQ ID NO: 127. In some embodiments, the rev protein comprises an amino acid sequence at least 95% identical to the amino acid sequence of SEQ ID NO:127. In some embodiments, the rev protein comprises an amino acid sequence at least 96% identical to the amino acid sequence of SEQ ID NO:127. In some embodiments, the rev protein comprises an amino acid sequence at least 97% identical to the amino acid sequence of SEQ ID NO:127. In some embodiments, the rev protein comprises an amino acid sequence at least 98% identical to the amino acid sequence of SEQ ID NO:127. In some embodiments, the rev protein comprises an amino acid sequence at least 99% identical to the amino acid sequence of SEQ ID NO:127. In some embodiments, the rev protein comprises an amino acid sequence at least 100% identical to the amino acid sequence of SEQ ID NO:127. In some embodiments, the rev protein comprises the amino acid sequence of SEQ ID NO:127. In some embodiments, the rev protein consists the amino acid sequence of SEQ ID NO:127.

In some embodiments, the rev protein is encoded by a nucleotide sequence comprising a nucleotide sequence at least 80%, 85%, 90%, 95%, 96%, 97%, 98%, 99% or 100% identical to the nucleotide sequence of SEQ ID NO:128. In some embodiments, the rev protein is encoded by a nucleotide sequence comprising a nucleotide sequence at least 80% identical to the nucleotide sequence of SEQ ID NO:128. In some embodiments, the rev protein is encoded by a nucleotide sequence comprising a nucleotide sequence at least 85% identical to the nucleotide sequence of SEQ ID NO:128. In some embodiments, the rev protein is encoded by a nucleotide sequence comprising a nucleotide sequence at least 90% identical to the nucleotide sequence of SEQ ID NO: 128. In some embodiments, the rev protein is encoded by a nucleotide sequence comprising a nucleotide sequence at least 95% identical to the nucleotide sequence of SEQ ID NO:128. In some embodiments, the rev protein is encoded by a nucleotide sequence comprising a nucleotide sequence at least 96% identical to the nucleotide sequence of SEQ ID NO:128. In some embodiments, the rev protein is encoded by a nucleotide sequence comprising a nucleotide sequence at least 97% identical to the nucleotide sequence of SEQ ID NO: 128. In some embodiments, the rev protein is encoded by a nucleotide sequence comprising a nucleotide sequence at least 98% identical to the nucleotide sequence of SEQ ID NO:128. In some embodiments, the rev protein is encoded by a nucleotide sequence comprising a nucleotide sequence at least 99% identical to the nucleotide sequence of SEQ ID NO:128. In some embodiments, the rev protein is encoded by a nucleotide sequence comprising a nucleotide sequence at least 100% identical to the nucleotide sequence of SEQ ID NO: 128. In some embodiments, the rev protein is encoded by a nucleotide sequence comprising the nucleotide sequence of SEQ ID NO: 128. In some embodiments, the rev protein is encoded by a nucleotide sequence consisting of the nucleotide sequence of SEQ ID NO: 128.

In some embodiments, a mixture of plasmids is used for transfection. In some embodiments, the mixture of plasmids comprises a plasmid encoding a gene of interest; a plasmid encoding a rev viral protein; and a plasmid encoding a gagpol viral protein.

In some embodiments, the mixture of plasmids comprises a plasmid encoding a gene of interest; a plasmid encoding a rev viral protein; a plasmid encoding a gagpol viral protein; and a plasmid encoding a viral envelope protein.

In some embodiments, the mixture of plasmids comprises a plasmid encoding a gene of interest; a plasmid encoding a rev viral protein; a plasmid encoding a gagpol viral protein; a plasmid encoding a viral envelope protein; and a plasmid encoding an immune cell activating protein.

In some embodiments, the mixture of plasmids comprises a plasmid encoding a gene of interest; a plasmid encoding a rev viral protein; a plasmid encoding a gagpol viral protein; and a plasmid encoding a viral envelope protein.

In some embodiments, the mixture of plasmids comprises a plasmid encoding a gene of interest; a plasmid encoding a rev viral protein; a plasmid encoding a gagpol viral protein; a plasmid encoding a viral envelope protein; and a plasmid encoding a co-stimulatory molecule.

In some embodiments, the mixture of plasmids comprises a plasmid encoding a gene of interest; a plasmid encoding a rev viral protein; a plasmid encoding a gagpol viral protein; a plasmid encoding a viral envelope protein; and a plasmid encoding an immune cell activating protein.

In some embodiments, the mixture of plasmids comprises a plasmid encoding a gene of interest; a plasmid encoding a rev viral protein; a plasmid encoding a gagpol viral protein; a plasmid encoding a viral envelope protein; a plasmid encoding an immune cell activating protein; and a plasmid encoding a co-stimulatory molecule.

In some embodiments, the mixture of plasmids comprises a plasmid encoding a gene of interest; a plasmid encoding a rev viral protein; a plasmid encoding a gagpol viral protein; a plasmid encoding a viral envelope protein; and a plasmid encoding a co-stimulatory molecule.

In some embodiments, the mixture of plasmids comprises a plasmid encoding a gene of interest; a plasmid encoding a rev viral protein; a plasmid encoding a gagpol viral protein; a plasmid encoding a viral envelope protein; a plasmid encoding an immune cell activating protein; and a plasmid encoding a co-stimulatory molecule.

In some embodiments, a 5-plasmid system is used for transfection. In some embodiments, the 5-plasmid system includes a plasmid expressing a gene of interest, a plasmid expressing the lentiviral gagpol gene, a plasmid expressing the lentiviral rev gene, a plasmid expressing a viral envelope protein, and a plasmid expressing an immune-activating protein.

In some embodiments, a plasmid system is used for transfection that comprises more than 5 plasmids. In some embodiments, the plasmid system includes a plasmid expressing a gene of interest, a plasmid expressing the lentiviral gagpol gene, a plasmid expressing the lentiviral rev gene, a plasmid expressing a viral envelope protein, a plasmid expressing an immune-activating protein, and at least one additional plasmid expressing a protein for incorporation into the viral envelope (e.g. a costimulatory molecule or other viral particle surface molecule described herein).

In some embodiments, transient transfection is used to generate the viral producing cells of the disclosure. Transient transfection avoids the longer time required to generate stable vector-producing cell lines and can be used if the vector genome or lentiviral packaging components are toxic to cells. In the processes using transiently transfected cells, any agent allowing transfection of plasmids may be used. Illustrative examples include calcium phosphate or polyethylenimine, although other agents may be contemplated by one skilled in the art (Ansorge et al. 2010). The conditions (e.g., the number of plasmids, ratio between the plasmids, ratio between the plasmids and the transfection agent, the type of medium, etc.) and the transfection time may be adapted by one skilled in the art according to the characteristics of the produced virus and/or of the transgene introduced into the transfer plasmid.

### 3. Harvest of Viral Particles

In some embodiments, cells transfected with the viral vector (also referred to herein as vector producing cells) are cultured to increase cell and virus numbers and/or virus titers. Cell culturing can be accomplished by methods well known to persons skilled in the art, and includes but is not limited to providing nutrients for the cells, for instance in the appropriate culture media. The methods may comprise growth adhering to surfaces, growth in suspension, or combinations thereof.

In some embodiments, culturing is performed in tissue culture flasks, dishes, roller bottles, or in bioreactors, using batch, fed-batch, continuous systems, hollow fiber, and the like, in order to achieve large scale production of virus through cell culture. In some embodiments, the vector-producing cells are capable of growing in suspension. Suitable conditions for culturing cells are known to one of skill in the art (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

In some embodiments, vector producing cells are cultured to a target density suitable for harvest. In some embodiments, harvest occurs 40-50 hours after transfection. In some embodiments, harvest occurs about 48 hours after transfection.

In some embodiments, an endonuclease is added to the vector producing cells before harvest. In some embodiments, an endonuclease is added to the vector producing cells 1-5 hours before harvest. In some embodiments, an endonuclease is added to the vector producing cells about 2 hours before harvest. In some embodiments, the endonuclease is a nuclease. In some embodiments, the endonuclease is a salt active nuclease. Various endonucleases are known and can be used. In some embodiments, the endonuclease is benzonase. In some embodiments, the endonuclease is denarase.

In some embodiments, the endonuclease is added to the bioreactor to achieve a concentration of between 1 U/mL and 100 U/mL, such as 1 U/mL and 50 U/mL, 1 U/mL and 20 U/mL, 1 U/mL and 10 U/mL, 10 U/mL and 100 U/mL, 10 U/mL and 50 U/mL, 10 U/mL and 20 U/mL, 20 U/mL and 100 U/mL, 20 U/mL and 50 U/mL or 50 U/mL and 100 U/mL. In some embodiments, the concentration is between 5 U/mL and 20 U/mL. In some embodiments, the concentration is 20 U/mL.

In some embodiments, a MgCl₂ supplementation is added to the vector producing cells before harvest. In some embodiments, a MgCl₂ supplementation is added to the vector producing cells 1-5 hours before harvest. In some embodiments, a MgCl₂ supplementation is added to the vector producing cells about 2 hours before harvest. In some embodiments, MgCl₂ is added to the bioreactor to achieve a concentration of between 0.5 mM and 5 mM, such as 0.5 mM to 4 mM, 0.5 mM to 2 mM, 0.5 mM to 1 mM, 1 mM to 5 mM, 1 mM to 4 mM, 1 mM to 2 mM, 2 mM to 5 mM, 2 mM to 4 mM or 4 mM to 5 mM. In some embodiments, the concentration is between 0.5 mM to 4 mM. In some embodiments, the concentration is 2 mM.

In some embodiments, the harvested cell culture may also be referred to as a suspension mixture. In some embodiments, the suspension mixture has a volume of about 3-50 liters. In some embodiments, the suspension mixture has a volume of about 50-500 liters. In some embodiments, the suspension mixture has a volume of about 200 liters.

In some embodiments, the upstream process produces a suspension mixture with a harvest of infectious titer comprising 0.5×10⁵ TU/mL to 5×10⁶ TU/mL. In some embodiments, the infectious titer comprises 1×10⁵ TU/mL to 3×10⁵ TU/mL, 2×10⁵ TU/mL to 4×10⁵ TU/mL, 3×10⁵ TU/mL to 5×10⁵ TU/mL, 4×10⁵ TU/mL to 6×10⁵ TU/mL, 5×10⁵ TU/mL to 7×10⁵ TU/mL, 6×10⁵ TU/mL to 8×10⁵ TU/mL, 7×10⁵ TU/mL to 9×10⁵ TU/mL, 8×10⁵ TU/mL to 1×10⁶ TU/mL, 9×10⁵ TU/mL to 2×10⁶ TU/mL, 1×10⁶ TU/mL to 3×10⁶ TU/mL, 2×10⁶ TU/mL to 4×10⁶ TU/mL, 3×10⁶ TU/mL to 5×10⁶ TU/mL. In some embodiments, the infectious titer is 2×10⁶ TU/mL to 4×10⁶ TU/mL.

In some embodiments, the infectious titer comprises at least about 2×10⁶ TU/mL. In some embodiments, the infectious titer comprises at least about 3×10⁶ TU/mL. In some embodiments, the infectious titer comprises at least about 4×10⁶ TU/mL. In some embodiments, the infection titer is 2×10⁶ TU/mL. In some embodiments, the infection titer is 3×10⁶ TU/mL. In some embodiments, the infection titer is 4×10⁶ TU/mL.

In some embodiments, the suspension mixture is about 3 L to 200 L. In some embodiments, the suspension mixture comprises a volume between about 1 to 10 L, 5 to 15 L, 10 to 20 L, 15 to 25 L, 20 to 30 L, 25 to 35 L, 30 to 40 L, 35 to 45 L, 40 to 50 L, 45 to 55 L, 50 to 60 L, 55 to 65 L, 60 to 70 L, 65 to 75 L, 70 to 80 L, 75 to 85 L, 80 to 90 L, 85 to 95 L, 90 to 100 L, 95 to 105 L or 100 to 200 L. In some embodiments, the suspension mixture comprises at least 3 L, 10 L, 40 L, 50 L, 100 L or 200 L, or any value between any of the foregoing. In some embodiments, the suspension mixture comprises at least 3 L, 10 L, 40 L, 50 L, 100 L or 200 L. In some embodiments, the suspension mixture is about 40 L. In some embodiments, the suspension mixture is about 200 L. In some embodiments, the suspension mixture is about 160 L to 200 L, such as at or about 160 L, 170 L, 180 L, 190 L or 200 L, or any value between any of the foregoing.

In some embodiments, the upstream process produces a suspension mixture with a harvest of infectious titer of 2×10⁹ TU to 2×10¹¹ TU. In some embodiments, the infectious titer of 4×10⁹ TU to 12×10⁹ TU, 8×10⁹ TU to 16×10⁹ TU, 12×10⁹ TU to 2×10¹⁰ TU, 16×10⁹ TU to 2.4×10¹⁰ TU, 2×10¹⁰ TU to 2.8×10¹⁰ TU, 2.4×10¹⁰ TU to 3.2×10¹⁰ TU, 2.8×10¹⁰ TU to 3.6×10¹⁰ TU, 3.2×10¹⁰ TU to 4×10¹⁰ TU, 3.6×10¹⁰ TU to 8×10¹⁰ TU, 4×10¹⁰ TU to 12×10¹⁰ TU, 8×10¹⁰ TU to 16×10¹⁰ TU, 12×10¹⁰ TU to 2×10¹¹ TU. In some embodiments, the upstream process produces a suspension mixture with a harvest of infectious titer of 8×10¹⁰ TU to 16×10¹⁰ TU,

In some embodiments, the infectious titer comprises at least about 8×10¹⁰ TU. In some embodiments, the infectious titer comprises at least about 12×10¹⁰ TU. In some embodiments, the infectious titer comprises at least about 16×10¹⁰ TU. In some embodiments, the infection titer is 8×10¹⁰ TU. In some embodiments, the infection titer is 12×10¹⁰ TU. In some embodiments, the infection titer is 16×10¹⁰ TU.

In some embodiments, the upstream process produces a suspension mixture with a harvest of infectious titer of 1×10¹⁰ TU to 1×10¹² TU. In some embodiments, the infectious titer is 2×10¹⁰ TU to 6×10¹⁰ TU, 4×10¹⁰ TU to 8×10¹⁰ TU, 6×10¹⁰ TU to 10×10¹⁰ TU, 8×10¹⁰ TU to 12×10¹⁰ TU, 10×10¹⁰ TU to 14×10¹⁰ TU, 12×10¹⁰ TU to 16×10¹⁰ TU, 14×10¹⁰ TU to 18×10¹⁰ TU, 16×10¹⁰ TU to 2×10¹¹ TU, 18×10¹⁰ TU to 4×10¹¹ TU, 2×10¹¹ TU to 6×10¹¹ TU, 4×10¹¹ TU to 8×10¹¹ TU, 6×10¹¹ TU to 10×10¹¹ TU. In some embodiments, the upstream process produces a suspension mixture with a harvest of infectious titer of 4×10¹¹ TU to 8×10¹¹ TU.

In some embodiments, the infectious titer comprises at least about 4×10¹¹ TU. In some embodiments, the infectious titer comprises at least about 6×10¹¹ TU. In some embodiments, the infectious titer comprises at least about 8×10¹¹ TU. In some embodiments, the infectious titer is about 4×10¹¹ TU. In some embodiments, the infection titer is about 6×10¹¹ TU. In some embodiments, the infection titer is about 8×10¹¹ TU.

### B. Downstream Process

In some embodiments, the disclosure provides a method for preparing a viral particle formulation.

In some embodiments, the method for preparing a viral particle formulation comprises introduction of a nuclease, harvest clarification, concentration and purification of the harvest and formulation and concentration of the harvest, referred to herein as a downstream process. The goal of the downstream process is to increase viral particle yield and increase viral particle purity. In some embodiments, the downstream process comprises the downstream process demonstrated in FIG. 1B.

In some embodiments, the disclosure provides a method for preparing a viral particle formulation from a suspension mixture of a population of host cells and viral particles. In some embodiments, the suspension mixture is prepared from the upstream processes as described in sections above. In some embodiments, preparing a viral particle formulation comprises (i) clarifying the mixture of host cells and viral particles to produce a filtered formulation of viral particles, and (ii) concentrating the filtered formulation to produce a concentrated formulation of viral particles. In some embodiments, the method comprises (iii) sterile filtering the concentrated formulation of viral particles to produce the viral particle formulation. In some embodiments, the viral particle formulation is suitable for in vivo administration. In some embodiments, the process of preparing the viral particle formulation purifies the viral vectors to remove impurities.

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter has a retention threshold smaller than the second filter, thereby producing a filtered formulation of viral particles; and (ii) concentrating the filtered formulation of viral particles, wherein concentrating comprises chromatography and ultrafiltration.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter has a retention threshold smaller than the second filter, thereby producing a filtered formulation of retroviral particles; and (ii) concentrating the filtered formulation of retroviral particles, wherein concentrating comprises chromatography and ultrafiltration.

In some embodiments, the disclosure provides a method for preparing a retrovirus (e.g., lentivirus) formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter has a retention threshold smaller than the second filter, thereby producing a filtered formulation of lentiviral particles; and (ii) concentrating the filtered formulation of lentiviral particles, wherein concentrating comprises chromatography and ultrafiltration.

### 1. Clarification of Bioreactor Harvest

In some embodiments, the methods described herein comprise filtering a mixture of a population of host cells and viral particles to remove contaminants. This filtering step is also referred to herein as “clarifying” or “clarification”. In some embodiments, the clarifying or clarification comprises using a filter. In some embodiments, the filter is a depth filter.

In some embodiments, the clarifying the suspension mixture of host cells and viral particles is carried out to remove host cells, cell debris and precipitated impurities from the suspension mixture from the bioreactor harvest while retaining the viral vector particles. In some embodiments, clarification can be carried out using filters or by centrifugation. In some embodiments, centrifugation can comprise density gradient centrifugation, ultracentrifugation and differential centrifugation. In some embodiments, the clarifying or clarification comprises centrifugation and depth filtration. In some embodiments, the clarification is carried out using depth filtration or membrane filtration. In some embodiments, one or more of the filters is a hollow fiber filter.

In some embodiments, the clarification process step is carried out using a series of filters with progressively smaller pore sizes. In some embodiments, at least one filter is a depth filter, which captures contaminants within its structure and are useful to filter turbid process pools with high throughputs. In some embodiments, at least one filter is a membrane filter which typically traps contaminants larger than the pore size on the addressed surface of the membrane. In some embodiments, the processing steps include a series of filters where the first filter is a depth filter, and is followed by filtering with a membrane filter. In some embodiments, using depth filters to reduce the total number of large particles in the pool allows for improved throughput across membrane filters with smaller pore sizes.

In some embodiments, the clarification process uses primary, secondary and tertiary filtration.

In some embodiments, the clarifying or clarification comprises centrifugation. In some embodiments, centrifugation can comprise density gradient centrifugation, ultracentrifugation and differential centrifugation. In some embodiments, the clarifying or clarification comprises centrifugation and depth filtration.

In some embodiments, filtering the mixture comprises (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter has a retention threshold smaller than the second filter, thereby producing a filtered formulation of viral particles.

In some embodiments, the methods comprise filtering a mixture of a population of host cells and retroviral particles. In some embodiments, filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter has a retention threshold smaller than the second filter, thereby producing a filtered formulation of retroviral particles.

In some embodiments, the methods comprise filtering a mixture of a population of host cells and lentiviral particles. In some embodiments filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter has a retention threshold smaller than the second filter, thereby producing a filtered formulation of lentiviral particles.

In some embodiments, the first filter has a retention threshold of about 1-60 μm, about 5-60 μm, about 10-60 μm, about 15-60 μm, about 20-60 μm, about 25-60 μm, about 30-60 μm, about 35-60 μm, about 40-60 μm, about 45-60 μm, about 50-60 μm, about 55-60 m, or above 60 μm. In some embodiments, the first filter has a retention threshold of about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 m, about 70 μm, about 75 μm, about 80 μm, about 85 μm, about 90 μm, about 95 μm, or about 100 μm. In some embodiments, the first filter has a retention threshold of 60 μm.

In some embodiments, the first filter is a depth filter. In some embodiments, the first filter is a commercially available depth filter. An illustrative example of a commercially available first filter includes but is not limited to, a Clarisolve® Depth Filter (Millipore®).

In some embodiments, the second filter has a retention threshold of about 0.2-4 μm, about 0.3-4 μm, about 0.4-4 μm, about 0.5-4 μm, about 0.6-4 μm, about 0.7-4 μm, about 0.8-4 μm, about 0.9-4 μm, about 1-4 μm, about 2-4 μm, or about 3-4 μm. In some embodiments, the second filter has a retention threshold of about 0.4-3 μm, about 0.4-2 μm, about 0.4-1 μm, about 0.4-0.9 μm, about 0.4-0.8 μm, about 0.4-0.7 μm, about 0.4-0.6 μm, or about 0.4-0.5 μm. In some embodiments, the second filter has a retention threshold of about 0.2 μm, about 0.25 μm, about 0.3 μm, about 0.35 μm, about 0.4 μm, about 0.45 μm, about 0.5 μm, about 0.55 μm, about 0.6 μm, about 0.65 μm, about 0.7 μm, about 0.75 μm, about 0.8 μm, about 0.85 μm, about 0.9 μm, about 0.95 μm, about 1 μm, about 2 μm, about 3 μm, or about 4 μm. In some embodiments, the second filter has a retention threshold of 0.45 μm.

In some embodiments, the second filter is a commercially available filter. An illustrative example of a commercially available second filter includes but is not limited to, a Sartopure® PP3 filter (Sartorius).

In some embodiments, the third filter has a retention threshold of about 0.2-1 μm, about 0.2-0.9 μm, about 0.2-0.8 μm, about 0.2-0.7 μm, about 0.2-0.6 μm, about 0.2-0.5 μm, about 0.2-0.4 μm, or about 0.2-0.3 μm. In some embodiments, the third filter has a retention threshold of about 0.2 μm, about 0.25 μm, about 0.3 μm, about 0.35 μm, about 0.4 μm, about 0.45 μm, or about 0.5 μm. In some embodiments, the third filter has a retention threshold of 0.2 μm.

In some embodiments, the third filter is a commercially available filter. An illustrative example of a commercially available third filter includes but is not limited to, a Sartopure®2 HF filter (Sartorius).

In some embodiments, the first filter has a retention threshold of 60 μm, the second filter has a retention threshold of 0.45 μm, and the third filter has a retention threshold of 0.2 μm.

In some embodiments, the second filter and the third filter are two layers within a dual-layer filter component. In some embodiments, the third filter is a dual-filter comprising a first layer filter and a second layer filter. In some embodiments, each layer of the dual-layer filter component has a different retention threshold. In some embodiments, a first layer has a retention threshold of about 0.4 μm, about 0.45 μm, about 0.5 μm, about 0.55 μm, about 0.6 μm, about 0.65 μm, about 0.7 μm, about 0.75 μm, about 0.8 μm, about 0.85 μm, about 0.9 μm, about 0.95 μm, about 1 μm, about 2 μm, about 3 μm, or about 4 μm. In some embodiments, a second layer has a retention threshold of about 0.2 μm, about 0.25 μm, about 0.3 μm, about 0.35 μm, about 0.4 μm, about 0.45 μm, or about 0.5 μm. In some embodiments, a first layer has a retention threshold of about 0.45 μm and a second layer has a retention threshold of about 0.2 μm.

In some embodiments, the dual-layer filter component is commercially available. Illustrative examples of commercially available dual-layer filter components include but are not limited to, Sartopure®2 (pore size: 0.45|0.2 μm), Sartopure®2 XLG (pore size: 0.8|0.2 μm), and Sartopure®2 XLI (pore size: 0.35|0.2 μm).

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a dual-layer filter component comprising a second filter and a third filter, wherein the second filter has a retention threshold smaller than the first filter and the third filter has a retention threshold smaller than the second filter, thereby producing a filtered formulation of viral particles.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a dual-layer filter component comprising a second filter and a third filter, wherein the second filter has a retention threshold smaller than the first filter and the third filter has a retention threshold smaller than the second filter, thereby producing a filtered formulation of retroviral particles.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a dual-layer filter component comprising a second filter and a third filter, wherein the second filter has a retention threshold smaller than the first filter and the third filter has a retention threshold smaller than the second filter, thereby producing a filtered formulation of lentiviral particles.

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, (c) filtering the second filtrate with a dual-layer filter component comprising the second filter and a third filter, wherein the third filter has a retention threshold smaller than the second filter, thereby producing a filtered formulation of viral particles.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, (c) filtering the second filtrate with a dual-layer filter component comprising the second filter and a third filter, wherein the third filter has a retention threshold smaller than the second filter, thereby producing a filtered formulation of retroviral particles.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, (c) filtering the second filtrate with a dual-layer filter component comprising the second filter and a third filter, wherein the third filter has a retention threshold smaller than the second filter, thereby producing a filtered formulation of lentiviral particles.

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filter, and (c) filtering the second filtrated with a third filter, wherein the third filter is a dual-layer filter component comprising a first layer filter and a second layer filter, wherein the second layer filter has a retention threshold smaller than the first layer filter, thereby producing a filtered formulation of viral particles.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filter, and (c) filtering the second filtrated with a third filter, wherein the third filter is a dual-layer filter component comprising a first layer filter and a second layer filter, wherein the second layer filter has a retention threshold smaller than the first layer filter, thereby producing a filtered formulation of retroviral particles.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filter, and (c) filtering the second filtrated with a third filter, wherein the third filter is a dual-layer filter component comprising a first layer filter and a second layer filter, wherein the second layer filter has a retention threshold smaller than the first layer filter, thereby producing a filtered formulation of lentiviral particles.

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filter, and (c) filtering the second filtrated with a third filter, wherein the third filter is a dual-layer filter component comprising a first layer filter and a second layer filter, wherein the first layer filter has the same retention threshold as the second filter, and wherein the second layer filter has a retention threshold smaller than the first layer filter, thereby producing a filtered formulation of viral particles.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filter, and (c) filtering the second filtrated with a third filter, wherein the third filter is a dual-layer filter component comprising a first layer filter and a second layer filter, wherein the first layer filter has the same retention threshold as the second filter, and wherein the second layer filter has a retention threshold smaller than the first layer filter, thereby producing a filtered formulation of retroviral particles.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filter, and (c) filtering the second filtrated with a third filter, wherein the third filter is a dual-layer filter component comprising a first layer filter and a second layer filter, wherein the first layer filter has the same retention threshold as the second filter, and wherein the second layer filter has a retention threshold smaller than the first layer filter, thereby producing a filtered formulation of lentiviral particles.

In some embodiments, one or more filter is a hollow fiber filter (also described below as a hollow fiber module). In some embodiments, the hollow fiber filter has a nominal molecular weight cutoffs (NMWC) between 100 kDa and 1000 kDa. In some embodiments, the NMWC is between 100 kDa and 500 kDa. In some embodiments, the NMWC is between 300 kDa and 750 kDa. In some embodiments, the NMWC is between 500 kDa and 1000 kDa.

In some embodiments, the NMWC is 500 kDa. In some embodiments, the NMWC is 300 kDa. In some embodiments, the NMWC is 100 kDa.

In some embodiments, the hollow fiber filter has a pore size between 0.1 μm and 0.65 μm. In some embodiments, the pore size is 0.1 μm. In some embodiments, the pore size is 0.2 μm. In some embodiments, the pore size is 0.45 μm. In some embodiments, the pore size is 0.65 μm.

In some embodiments, the hollow fiber filter has a length of 20 cm, 41.5 cm, 65 cm, 50 cm, 68 cm, or 108 cm. In some embodiments, the hollow fiber filter has an inner lumen of 0.50 mm, 0.63 mm, 0.75 mm, 1 mm, 2 mm or 3 mm.

In some embodiments, the hollow fiber filter has a surface area between 235 cm² and 1600 cm². In some embodiments, the surface area is 235 cm². In some embodiments, the surface area is 790 cm². In some embodiments, the surface area is 1600 cm².

In some embodiments, the hollow fiber filter holds a volume of 300 mL.

In some embodiments, the hollow fiber filter is a commercially available hollow fiber filter. An illustrative example of a commercially available hollow fiber filter includes but is not limited to, a Spectrum® Hollow Fiber Filter (Repligen).

In some embodiments, the filtering is carried out under sterile conditions. In some embodiments, each of the filter steps are performed in a closed system. In some embodiments, one or more of the filters can be operably connected in-line in a closed system. In some embodiments, the first filter is connected in-line to the bioreactor. In some embodiments, the second or third filter are connected in-line to the first filter, in which all filters are operably connected. In some embodiments, the filtering is in a closed system and is operably connected in-line to the bioreactor, in which the suspension harvest from the bioreactor is pumped directly through the first filter, and optionally further pumped directly through the second filter and third filter all in a closed system. In some embodiments, the closed system does not allow transfer of any material out of the system, such as does not allow exposure of material to air or the outside environment.

In some embodiments, an endonuclease is added prior to filtering the suspension mixture. In some embodiments, an endonuclease is added prior to filtering the mixture with a first filter. In some embodiments, an endonuclease is added prior to filtering the mixture with a second filter. In some embodiments, an endonuclease is added prior to filtering the mixture with a third filter. In some embodiments, an endonuclease is added after filtering the mixture with a third filter. In some embodiments, an endonuclease is added after filtering the mixture with a third filter and prior to concentrating the filtered formulation.

In some embodiments, an endonuclease is present through the steps of filtering the mixture with a third filter and concentrating the filtered formulation. In some embodiments, an endonuclease is present through the steps of filtering the mixture with a second filter and concentrating the filtered formulation. In some embodiments, an endonuclease is present through the steps of filtering the mixture with a first filter and concentrating the filtered formulation.

In some embodiments, residual host cell DNA (hcDNA) is measured after the filtration steps. hcDNA may be measured by qPCR and normalized to the amount of virus particles. In some embodiments, hcDNA is measured in units per dose, where one dose is defined using the transducing unit (TU) of the virus. In some embodiments, hcDNA is measured in ng per dose, where one dose is 1E9 TU. A unit of 1E9 is the same as 1×10⁹.

In some embodiments, hcDNA is less than about 5000 ng/1E9 TU, less than about 4500 ng/1E9 TU, less than about 4000 ng/1E9 TU, less than about 3500 ng/1E9 TU, less than about 3000 ng/1E9 TU, less than about 2500 ng/1E9 TU, less than about 2000 ng/1E9 TU, less than about 1500 ng/1E9 TU, or less than about 1000 ng/1E9 TU after the third filter. In some embodiments, hcDNA is less than about 2500 ng/1E9 TU after the third filter. In some embodiments, hcDNA is not detectable after the third filter.

In some embodiments, hcDNA after the third filter is at least 5-fold, at least 10-fold, at least 15-fold, at least 20-fold, at least 30-fold, at least 40-fold, at least 50-fold, at least 60-fold, at least 70-fold, at least 80-fold, at least 90-fold, or at least 100-fold lower compared to hcDNA at harvest (in the suspension mixture). In some embodiments, hcDNA after the third filter is at least 80-fold lower compared to hcDNA at harvest.

In some embodiments, hcDNA after the third filter is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold lower compared to hcDNA after the second filter. In some embodiments, hcDNA after the third filter is at least 5-fold lower compared to hcDNA after the second filter.

### 2. Chromatography

In some embodiments, the methods of the disclosure comprise concentrating a filtered formulation of viral particles. In some embodiments, concentrating can be by chromatography. In some embodiments, chromatography also acts to remove impurities, such as HCP and HcDNA, from the filtered formulation of particles. In some embodiments, the methods of the disclosure include clarifying a suspension mixture by using a series of filters as described above, followed by a step of chromatography. In some embodiments, the chromatography is used to further remove impurities, such as HCP and hcDNA, from the filtered formulation of particles, as well as concentrate the viral particles in the formulation.

Chromatography techniques well known to those of skill in the art may be used in the methods of the disclosure. These techniques may involve the separation of the vector particles from the cellular milieu and, if necessary, the further purification of the vector particles. One or more of a variety of chromatographic methods may be used for purification. In some embodiments, chromatography is performed after clarification of the harvest.

In some embodiments, residual host cell protein (HCP) is measured after chromatography. HCP may be measured by ELISA and normalized to the amount of virus particles. In some embodiments, HCP is measured in units per dose, where one dose is defined using the transducing unit (TU) of the virus. In some embodiments, HCP is measured in μg per dose, where one dose is 1E9 TU.

In some embodiments, HCP is less than about 5000 μg/1E9 TU, less than about 4500 μg/1E9 TU, less than about 4000 μg/1E9 TU, less than about 3500 μg/1E9 TU, less than about 3000 μg/1E9 TU, less than about 2500 μg/1E9 TU, less than about 2000 μg/1E9 TU, less than about 1500 μg/1E9 TU, or less than about 1000 μg/1E9 TU after chromatography. In some embodiments, HCP is less than about 3000 μg/1E9 TU after chromatography.

In some embodiments, HCP is at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, or at least 50-fold lower after chromatography compared to before chromatography. In some embodiments, HCP is at least about 40-fold lower after chromatography compared to before chromatography.

Illustrative chromatographic techniques are described below.

a. Ion-Exchange Chromatography

In some embodiments, the chromatography includes ion-exchange chromatography. Ion-exchange chromatography utilizes the fact that charged species, such as biomolecules and viral vectors, can bind reversibly to a stationary phase (such as a membrane, or else the packing in a column) that has, fixed on its surface, groups that have an opposite charge. There are two types of ion exchangers. Anion exchangers are stationary phases that bear groups having a positive charge and hence can bind species with a negative charge. Cation exchangers bear groups with a negative charge and hence can bind species with positive charge. The pH of the medium has an important influence on this, as it can alter the charge on a species. Thus, for a species such as a protein, if the pH is above the pi, the net charge will be negative, whereas below the pi, the net charge will be positive.

Displacement (elution) of the bound species can be affected by the use of suitable buffers. In general, the ionic concentration of the buffer is increased until the species is displaced through competition of buffer ions for the ionic sites on the stationary phase. An alternative method of elution entails changing the pH of the buffer until the net charge of the species no longer favors biding to the stationary phase. An illustrative example would be reducing the pH until the species assumes a net positive charge and will no longer bind to an anion exchanger.

Some purification can be achieved if impurities are uncharged, or else if they bear a charge of opposite sign to that of the desired species, but the same sign to that on the ion exchanger. This is because uncharged species and those having a charge of the same sign to that an ion exchanger, will not normally bind. For different bound species, the strength of the binding varies with factors such as the charge density and the distribution of charges on the various species. Thus, by applying an ionic or pH gradient (as a continuous gradient, or as a series of steps), the desired species might be eluted separately from impurities.

In some embodiments, ion exchange chromatography is performed after clarification of the harvest. In some embodiments, anion exchange chromatography is performed after clarification of the harvest.

In some embodiments, the chromatography is anion exchange (AEX) chromatography. In some embodiments, AEX is carried out with a membrane having a surface coating of positively charged functional groups, which allows for convective flow of negatively charged viral vectors across the membrane where they can bind with the positively charged coated surface. In some embodiments, the membrane has a nominal pore size of about 0.8 μM.

In some embodiments, the AEX utilizes a commercially available membrane. An illustrative example of a commercially available for AEX includes but is not limited to, a Mustang® Q chromatography membrane. In particular embodiments, the AEX membrane binds the lentivirus while impurities do not bind. The lentivirus is then able to be eluted by increasing the salt (e.g. NaCl) concentration.

In some embodiments, using AEX allows for larger impurities as well as impurities with different binding capacities than that of the viral vector, primarily positively charged impurities, to flow through the membrane during loading. Loosely bound impurities are then removed with a wash step. In some embodiments, the wash step is carried out using a salt buffer, which typically includes the same pH and conductivity as that of the filtered formulation added as the bulk load.

In some embodiments, the ion exchange chromatography, such as anion exchange chromatography, includes a step of eluting the viral vector bound to the membrane. In some embodiments, elution can be carried out by increasing the salt concentration across the membrane resulting in dissociation of viral vector from the membrane. In some embodiments, a low concentration of salt (e.g. NaCl) is used during equilibration, loading and wash steps of the anion exchange chromatography and then the concentration of salt (e.g. NaCl) is increased in the buffer added for elution.

In some embodiments, the method provided herein involves eluting the surface engineered lentiviral particle by adding a high salt buffer, such as containing sodium chloride (NaCl), potassium chloride (KCl), sodium acetate (NaOAc), or tetramethylammonium chloride. In some embodiments, the method of eluting the surface engineered lentiviral particle comprises NaCl.

In some embodiments, the NaCl concentration in the salt buffer for elution is from about 0.5M to 3M. In some embodiments, the NaCl concentration in the salt buffer for elution is from about 0.75M to 2M. In some embodiments, the NaCl concentration in the salt buffer for elution is about 0.75M. In some embodiments, the NaCl concentration in the salt buffer for elution is about 1M. In some embodiments, the NaCl concentration in the salt buffer for elution is about 2M.

In some embodiments, the present disclosure herein surprisingly demonstrates that the concentration of salt buffer for elution can impact the yield of lentivirus produced by the process. In particular, results show that there is a decreasing trend in the percent yield with the changes to the surface engineering and/or the transgene payload. In some embodiments, an improved yield of lentivirus can be achieved by using a higher salt concentration for elution when the surface engineered protein is larger or the transgene payload sequence is longer.

In one exemplary method, a lentivirus particle generated to express a surface engineered protein containing a single domain fusion protein can be eluted in a buffer containing 0.5M to 1.5 M NaCl, such as 0.5 M to 0.75 M NaCl. In some embodiments, a lentivirus particle generated to express a surface engineered protein containing a single domain fusion protein can be eluted in a buffer containing 0.75 M NaCl.

In another exemplary method, a lentivirus particle generated to express a surface engineered protein containing a multiple domain fusion protein can be eluted in a buffer containing 1.5M NaCl to 2.5 M NaCl, such as 1.75 M to 2.25 M NaCl. In some embodiments, a lentivirus particle generated to express a multi-domain fusion protein is eluted in a buffer containing 2M NaCl.

b. Size Exclusion Chromatography

In some embodiments, the chromatography includes size exclusion chromatography Size exclusion chromatography is a technique that separates species according to their size. Typically, it is performed by the use of a column packed with particles having pores of a well-defined size. For the chromatographic separation, particles are chosen that have pore sizes that are appropriate with regard to the sizes of the species in the mixture to be separated. When the mixture is applied, as a solution (or suspension, in the case of a virus), to the column and then eluted with buffer, the largest particles will elute first as they have limited (or no) access to the pores. Smaller particles will elute later as they can enter the pores and hence take a longer path through the column.

Thus, in considering the use of size exclusion chromatography for the purification of viral vectors, it would be expected that the vector would be eluted before smaller impurities such as proteins.

In some embodiments, size exclusion chromatography is performed after clarification of the harvest.

c. Hydrophobic Interaction Chromatography (HIC)

In some embodiments, the chromatography includes hydrophobic interaction chromatography. Species, such as proteins, have on their surfaces, hydrophobic regions that can bind reversibly to weakly hydrophobic sites on a stationary phase. In media having a relatively high salt concentration, this binding is promoted. Typically in HIC, the sample to be purified is bound to the stationary phase in a high salt environment. Elution is then achieved by the application of a gradient (continuous, or as a series of steps) of decreasing salt concentration. A salt that is commonly used is ammonium sulfate. Species having differing levels of hydrophobicity will tend to be eluted at different salt concentrations and so the target species can be purified from impurities. Other factors, such as pH, temperature and additives to the elution medium such as detergents, chaotropic salts and organics can also influence the strength of binding of species to HIC stationary phases. One, or more, of these factors can be adjusted or utilized to optimize the elution and purification of product.

Viral vectors have on their surface, hydrophobic moieties such as proteins, and thus HIC could be employed as a means of purification.

In some embodiments, HIC is performed after clarification of the harvest.

d. Reversed-Phase Chromatography (RPC)

In some embodiments, the chromatography includes reversed-phase chromatography Like HIC, RPC separates species according to differences in their hydrophobicity levels. A stationary phase of higher hydrophobicity than that employed in HIC is used. The stationary phase often consists of a material, typically silica, to which are bound hydrophobic moieties such as alkyl groups or phenyl groups. Alternatively, the stationary phase might be an organic polymer, with no attached groups. The sample-containing the mixture of species to be resolved is applied to the stationary phase in an aqueous medium of relatively high polarity which promotes binding. Elution is then achieved by reducing the polarity of the aqueous medium by the addition of an organic solvent such as isopropanol or acetonitrile. Commonly a gradient (continuous, or as a series of steps) of increasing organic solvent concentration is used and the species are eluted in order of their respective hydrophobicity levels.

Other factors, such as the pH of the elution medium, and the use of additives, can also influence the strength of binding of species to RPC stationary phases. One, or more, of these factors can be adjusted or utilized to optimize the elution and purification of product. A common additive is trifluoroacetic acid (TFA). This suppresses the ionization of acidic groups such as carboxyl moieties in the sample. It also reduces the pH in the eluting medium, and this suppresses the ionization of free silanol groups that may be present on the surface of stationary phases having a silica matrix. TFA is one of a class of additives known as ion pairing agents. These interact with ionic groups, present on species in the sample, that bear an opposite charge. The interaction tends to mask the charge, increasing the hydrophobicity of the species. Anionic ion pairing agents, such as TFA and pentafluoropropionic acid interact with positively charged groups on a species. Cationic ion pairing agents such, as triethylamine, interact with negatively charged groups.

Viral vectors have on their surface, hydrophobic moieties such as proteins, and thus RPC, potentially, could be employed as a means of purification.

In some embodiments, RPC is performed after clarification of the harvest.

e. Affinity Chromatography

In some embodiments, the chromatography includes affinity chromatography Affinity chromatography utilizes the fact that certain ligands that bind specifically with biomolecules such as proteins or nucleotides, can be immobilized on a stationary phase. The modified stationary phase can then be used to separate the relevant biomolecule from a mixture. Examples of highly specific ligands are antibodies, for the purification of target antigens and enzyme inhibitors for the purification of enzymes. More general interactions can also be utilized such as the use of the protein A ligand for the isolation of a wide range of antibodies.

Typically, affinity chromatography is performed by application of a mixture, containing the species of interest, to the stationary phase that has the relevant ligand attached. Under appropriate conditions this will lead to the binding of the species to the stationary phase. Unbound components are then washed away before an eluting medium is applied. The eluting medium is chosen to disrupt the binding of the ligand to the target species. This is commonly achieved by choice of an appropriate ionic strength, pH or by the use of substances that will compete with the target species for ligand sites. For some bound species, a chaotropic agent such as urea is used to effect displacement from the ligand. This, however, can result in irreversible denaturation of the species.

Viral vectors have on their surface, moieties such as proteins, which might be capable of binding specifically to appropriate ligands. This means that, potentially, affinity chromatography could be used in their isolation.

In some embodiments, affinity chromatography is performed after clarification of the harvest.

f. Immobilized Metal Ion Affinity Chromatography (IMAC)

In some embodiments, the chromatography includes immobilized metal ion affinity chromatography Biomolecules, such as proteins, can have on their surface, electron donating moieties that can form coordinate bonds with metal ions. This can facilitate their binding to stationary phases carrying immobilized metal ions such as Ni2+, Cu2+, Zn2+ or Fe3+. The stationary phases used in IMAC have chelating agents, typically nitriloacetic acid or iminodiacetic acid covalently attached to their surface and it is the chelating agent that holds the metal ion. It is necessary for the chelated metal ion to have at least one coordination site left available to form a coordinate bond to a biomolecule. Potentially there are several moieties on the surface of biomolecules that might be capable of bonding to the immobilized metal ion. These include histidine, tryptophan and cysteine residues as well as phosphate groups. For proteins, however, the predominant donor appears to be the imidazole group of the histidine residue. Native proteins can be separated using IMAC if they exhibit suitable donor moieties on their surface. IMAC can also be used for the separation of recombinant proteins bearing a chain of several linked histidine residues.

Typically, IMAC is performed by application of a mixture, containing the species of interest, to the stationary phase. Under appropriate conditions this will lead to the coordinate bonding of the species to the stationary phase. Unbound components are then washed away before an eluting medium is applied. For elution, gradients (continuous, or as a series of steps) of increasing salt concentration or decreasing pH may be used. Also, a commonly used procedure is the application of a gradient of increasing imidazole concentration. Biomolecules having different donor properties, for example having histidine residues in differing environments, can be separated by the use of gradient elution.

Viral vectors have on their surface, moieties such as proteins, which might be capable of binding to IMAC stationary phases. This means that, potentially, IMAC could be used in their isolation.

In some embodiments, IMAC is performed after clarification of the harvest.

### 3. Ultrafiltration/Diafiltration

In some embodiments, the methods of the disclosure comprise concentrating a filtered formulation of viral particles, wherein concentrating comprises ultrafiltration. In some embodiments, the methods of the disclosure comprise purifying a filtered formulation of viral particles, wherein purifying comprises ultrafiltration. In some embodiments, ultrafiltration is performed before chromatography. In some embodiments, ultrafiltration is performed after chromatography. In some embodiments, ultrafiltration also acts to remove impurities, such as HCP and HcDNA, from the filtered formulation of particles. In some embodiments, the ultrafiltration is used to further remove impurities, such as HCP and hcDNA, from the filtered formulation of particles and/or from the chromatography eluate, as well as concentrate the viral particles in the formulation. In some embodiments, the methods of the disclosure include clarifying a suspension mixture by using a series of filters as described above, followed by a step of chromatography, and then a step of ultrafiltration.

According to embodiments of disclosure, the filtered formulation of viral particles is subjected to ultrafiltration (also referred to as diafiltration when used for buffer exchange) at least once during the process, e.g., for concentrating the vector and/or buffer exchange.

The process used to concentrate the viral particle according to the method of the disclosure can include any filtration process (e.g., ultrafiltration (UF)) where the concentration of viral particle is increased by forcing diluent to be passed through a filter in such a manner that the diluent is removed from the viral particle preparation whereas the viral particle is unable to pass through the filter and thereby remains, in concentrated form, in the viral particle preparation. UF is described in detail in, e.g., Microfiltration and Ultrafiltration: Principles and Applications, L. Zeman and A. Zydney (Marcel Dekker, Inc., New York, NY, 1996); and in: Ultrafiltration Handbook, Munir Cheryan (Technomic Publishing, 1986; ISBN No. 87762-456-9).

In some embodiments, Tangential Flow Filtration (“TFF”) as described in, e.g., MILLIPORE catalogue entitled “Pharmaceutical Process Filtration Catalogue” pp. 177-202 (Bedford, Massachusetts, 1995/96) is used. TFF is widely used in the bioprocessing industry for cell harvesting, clarification, purification and concentration of products including viruses. The system is composed of three distinct process streams: the feed solution, the permeate and the retentate. Depending on application, filters with different pore sizes may be used in TFF. In some embodiments, the retentate contains the product (e.g., lentiviral particle).

In some embodiments, TFF is used to purify and concentrate viral particles using ultrafiltration and diafiltration (UF/DF). In embodiments using TFF, viral vector particles are retained in the system, while smaller components and impurities permeate the membrane and are removed from the system.

In some embodiments, the particular ultrafiltration membrane selected will have a pore size sufficiently small to retain viral particle but large enough to effectively clear impurities. Depending on the manufacturer and membrane type, nominal molecular weight cutoffs (NMWC) between 100 and 1000 kDa may be appropriate, for instance membranes with 300 kDa or 500 kDa NMWC. The membrane composition may be, but is not limited to, regenerated cellulose, polyethersulfone, polysulfone, or derivatives thereof. The membranes can be flat sheets (also called flat screens) or hollow fibers. UF is generally referred to filtration using filters with a pore size of smaller than 0.1 μm. Products are generally retained, while volume can be reduced through permeation (or be kept constant during diafiltration by adding buffer with the same speed as the speed with which the permeate, containing buffer and impurities, is removed at the permeate side).

The two most widely used geometries for TFF in the biopharmaceutical industry are plate & frame (flat screens) and hollow fiber modules. The hollow fiber modules are composed of an array of self-supporting fibers with a dense skin layer. Fiber diameters range from 0.5 mm-3 mm. An advantage of hollow fiber modules is the availability of filters from small membrane areas (ca. 16 cm2) to very large membrane areas (ca. 20 m2) allowing linear and simple scale-up.

In some embodiments, hollow fibers are used for TFF. These are reported to give less shear and a better viral particle/infectious unit (VP/IU) ratio than flat screen membranes. Further, the trans membrane pressure is generally lower in hollow fibers than with flat screens.

In certain embodiments, hollow fibers of 500 kDa (0.05 μm) pore size are used. Ultrafiltration may comprise diafiltration (DF), using ultrafilters and is an ideal way for removal and exchange of salts, sugars, non-aqueous solvents, separation of free from bound species, removal of material of low molecular weight, or rapid change of ionic and/or pH environments. Microsolutes are removed most efficiently by adding solvent to the solution being ultrafiltered at a rate equal to the UF rate. This washes microspecies from the solution at a constant volume, purifying the retained viral particle.

UF/DF can be used to concentrate and/or buffer exchange the viral particle suspensions according to the present disclosure in different stages of the concentration process. In some embodiments, methods of the disclosure utilize a DF step to exchange the buffer of the supernatant after chromatography or other purification steps. In some embodiments, the eluate from the chromatography step according to the disclosure is concentrated and further purified by ultrafiltration-diafiltration. During this process the viral particle is exchanged into formulation buffer.

In some embodiments, the ultrafiltration/diafiltration may be tangential flow diafiltration, stirred cell diafiltration and dialysis. In some embodiments, the ultrafiltration/diafiltration may comprise a plurality of tangential flow diafiltrations (TFF). In some embodiments, the plurality can be 1, 2, 3, 4 or more separate tangential flow diafiltrations. In some embodiments, the process includes two TFF steps and systems. In some embodiments, a first TFF step can be used to concentrate the chromatography eluate (e.g. AEX pool) and exchange the buffer to the diafiltration buffer, and a concentration. In some embodiments, a second TFF can be used to further concentrate the pool from the first TFF, such as to achieve a final target titer of the drug substance. In some embodiments, the first TFF and the second TFF are the same. In some embodiments, the first and second TFF are different. In some embodiments, the first TFF and second TFF have the same NMWC but the first TFF has a larger hold up volume or surface area.

In some embodiments, TFF, such as each of the TFF individually, be performed with a hollow fiber filter. In some embodiments, the hollow fiber filter has a nominal molecular weight cutoffs (NMWC) between 100 kDa and 1000 kDa. In some embodiments, the NMWC is between 100 kDa and 500 kDa. In some embodiments, the NMWC is between 300 kDa and 750 kDa. In some embodiments, the NMWC is between 500 kDa and 1000 kDa.

In some embodiments, the NMWC is 500 kDa. In some embodiments, the NMWC is 300 kDa. In some embodiments, the NMWC is 100 kDa.

In some embodiments, the hollow fiber filter has a pore size between 0.1 μm and 0.65 μm. In some embodiments, the pore size is 0.1 μm. In some embodiments, the pore size is 0.2 μm. In some embodiments, the pore size is 0.45 μm. In some embodiments, the pore size is 0.65 μm.

In some embodiments, the hollow fiber filter has a length of 20 cm, 41.5 cm, 65 cm, 50 cm, 68 cm, or 108 cm. In some embodiments, the hollow fiber filter has an inner lumen of 0.50 mm, 0.63 mm, 0.75 mm, 1 mm, 2 mm or 3 mm.

In some embodiments, the hollow fiber filter has a surface area between 235 cm² and 1600 cm². In some embodiments, the surface area is 235 cm². In some embodiments, the surface area is 790 cm². In some embodiments, the surface area is 1600 cm².

In some embodiments, the hollow fiber filter holds a volume of 300 mL.

In some embodiments, the hollow fiber filter is a commercially available hollow fiber filter. An illustrative example of a commercially available hollow fiber filter includes but is not limited to, a Spectrum® Hollow Fiber Filter (Repligen).

In some embodiments, each of the TFF is carried out with a hollow fiber filter comprising a different cassette or module that differ in one or more of the batch volume, pore size, membrane cutoff, inner lumen diameter or length. In some embodiments, the hollow fiber filter of each TFF can have the same nominal molecular weight cutoff (NMWC) or pore size, but differ in the surface area or volume. In some embodiments, the hollow fiber filter of each TFF can have a different NMWC or pore size and also differ in the surface area or volume.

In some embodiments, HCP is measured after the UF/DF steps. HCP may be measured by ELISA and normalized to the amount of virus particles. In some embodiments, HCP is measured in units per dose, where one dose is defined using the transducing unit (TU) of the virus. In some embodiments, HCP is measured in μg per dose, where one dose is 1E9 TU.

In some embodiments, HCP is less than about 5000 μg/1E9 TU, less than about 4500 μg/1E9 TU, less than about 4000 μg/1E9 TU, less than about 3500 μg/1E9 TU, less than about 3000 μg/1E9 TU, less than about 2500 μg/1E9 TU, less than about 2000 μg/1E9 TU, less than about 1500 μg/1E9 TU, or less than about 1000 μg/1E9 TU after UF/DF. In some embodiments, HCP is less than about 1500 μg/1E9 TU after a first UF/DF step. In some embodiments, HCP is not detectable after a second UF/DF step.

### 4. Filter Sterilization

In some embodiments, the methods of the disclosure comprise sterile filtering a filtered and concentration viral particle formulation as described herein. Filter-sterilization is common in processes for pharmaceutical grade materials, and known to the person skilled in the art. Filter-sterilization renders the resulting formulation substantially free of contaminants. The level of contaminants following filter-sterilization is such that the formulation is suitable for clinical use. Suitable sterilizing filters for use according to the disclosure are well known to those skilled in the art.

In some embodiments, the concentrated mixture after UF/DF is filter sterilized to produce a sterilized formulation. In some embodiments, the sterilizing filter has a maximum pore size of 0.22 μm. In some embodiments, the sterilizing filter has a retention threshold of 0.2 μm.

In some embodiments, the sterilized formulation is formulated in a buffer, thereby producing a drug substance. In some embodiments, the amount of contaminants in the drug substance is at a level acceptable for in vivo administration to a subject. In some embodiments, contaminants comprise host cells. In some embodiments, contaminants comprise host cell DNA. In some embodiments, contaminants comprise host cell proteins. In some embodiments, contaminants comprise host cell DNA and host cell proteins.

In some embodiments, the clarification, chromatography, UF/DF, and filter sterilization steps occur over a time period of about 5-10 hours. In some embodiments, the clarification, chromatography, UF/DF, and filter sterilization steps occur over a time period of about 5-8 hours. In some embodiments, the clarification, chromatography, UF/DF, and filter sterilization steps occur over a time period of about 5-6 hours. In some embodiments, the clarification, chromatography, UF/DF, and filter sterilization steps occur over a time period of about 6-7 hours. In some embodiments, the clarification, chromatography, UF/DF, and filter sterilization steps occur over a time period of about 6 hours.

In some embodiments, each of the clarification, chromatography, UF/DF, and filter sterilization steps occurs at a pH of 6-8. In some embodiments, each of the clarification, chromatography, UF/DF, and filter sterilization steps occurs at a pH equal to 6.0, 6.1 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. In some embodiments, each of the clarification, chromatography, UF/DF, and filter sterilization steps occurs at a pH equal to 7.0, 7.1, 7.2, 7.3, 7.4, 7.5, 7.6, 7.7, 7.8, 7.9 or 8.0.

In some embodiments, the viral vector formulation prepared by the above methods lacks impurities. In some embodiments, impurities comprise host cell DNA (hcDNA). In some embodiments, impurities comprise host cell protein (HCP). In some embodiments, the viral vector formulation is substantially pure. In some embodiments, viral vector formulation provided herein contains less than 10%, less than 9%, less than 8%, less than 7%, less than 6%, less than 5%, less than 4%, less than 3%, less than 2%, less than 1% or less than 0.5% of hcDNA. In some embodiments, viral vector formulation provided herein contains less than 1% of hcDNA. In some embodiments, a the methods used to prepare the viral vector formulation achieve greater than 90%, greater than 91%, greater than 92%, greater than 93%, greater than 94%, greater than 95%, greater than 96%, greater than 97%, greater than 98%, greater than 99% reduction in hcDNA, such as relative to the suspension mixture. In some embodiments, the methods used to prepare the viral vector formulation achieve a 99% reduction in hcDNA, such as compared to the suspension mixture.

In some embodiments, viral vector formulation provided herein contains less than 10%, less than 9%, less than 8%, less than 7%, less than 6%, less than 5%, less than 4%, less than 3%, less than 2%, less than 1% or less than 0.5% of HCP. In some embodiments, a viral vector formulation described herein contains less than 1% HCP. In some embodiments, the methods used to prepare the viral vector formulation achieve greater than 90%, greater than 91%, greater than 92%, greater than 93%, greater than 94%, greater than 95%, greater than 96%, greater than 97%, greater than 98%, greater than 99% reduction in HCP, such as relative to the suspension mixture. In some embodiments, the methods used to prepare the viral vector formulation achieve a 99% reduction in HCP, such as relative to the suspension mixture.

In some embodiments, the yield of the viral vector achieved by the above methods of preparing the viral vector formulation is at least at or about 10%, at least at or about 12%, at least at or about 15%, at least at or about 18%, at least at or about 20%, at least at or about 22%, at least at or about 25%, at least at or about 28% or at least at or about 30%, In some embodiments, the yield of the viral vector achieved by the above methods of preparing the viral vector formulation is 10% to 30%, such as at or about 10%, 12%, 15%, 18%, 20%, 22%, 25%, 28% or 30%, or any value between any of the foregoing. In some embodiments, the yield of viral vector achieved by the above methods of preparing the viral vector formulation is at least at or about 15% or is about 15%.

In some embodiments, the downstream process produces a viral vector formulation (which may be called a drug substance) with a viral vector titer of 0.5×10⁷ TU/mL to 5×10⁸ TU/mL. In some embodiments, the viral vector titer is 1×10⁷ TU/mL to 3×10⁷ TU/mL, 2×10⁷ TU/mL to 4×10⁷ TU/mL, 3×10⁷ TU/mL to 5×10⁷ TU/mL, 4×10⁷ TU/mL to 6×10⁷ TU/mL, 5×10⁷ TU/mL to 7×10⁷ TU/mL, 6×10⁷ TU/mL to 8×10⁷ TU/mL, 7×10⁷ TU/mL to 9×10⁷ TU/mL, 8×10⁷ TU/mL to 1×10⁸ TU/mL, 9×10⁷ TU/mL to 2×10⁸ TU/mL, 1×10⁸ TU/mL to 3×10⁸ TU/mL, 2×10⁸ TU/mL to 4×10⁸ TU/mL, 3×10⁸ TU/mL to 5×10⁸ TU/mL. In some embodiments, the viral vector titer is 2.5×10⁸ TU/mL to 4.7×10⁸ TU/mL. In some embodiments, the downstream process produces a viral vector formulation (or drug substance) with a viral vector titer of 2.5×10⁸ TU/mL to 4.7×10⁸ TU/mL. In some embodiments, the viral vector titer comprises at least about 2.5×10⁸ TU/mL. In some embodiments, the viral vector titer comprises at least about 4.7×10⁸ TU/mL. In some embodiments, the viral vector titer is 2.5×10⁸ TU/mL. In some embodiments, the viral vector titer is 4.7×10⁸ TU/mL.

In some embodiments, the viral vector formulation is at a volume between about 1 to 500 mL. In some embodiments, the viral vector formulation comprises a volume between about 1 to 10 mL, 5 to 15 mL, 10 to 20 mL, 15 to 25 mL, 20 to 30 mL, 25 to 35 mL, 30 to 40 mL, 35 to 45 mL, 40 to 50 mL, 50 to 100 mL, 75 to 125 mL, 100 to 300 mL, 200 to 400 mL, or 300 to 500. In some embodiments, the viral vector formulation comprises at least 10 mL, 50 mL, 100 mL, 200 mL, or 300 mL. In some embodiments, the viral vector formulation is 10 mL. In some embodiments, the viral vector formulation is 100 mL. In some embodiments, the viral vector formulation is 200 mL. In some embodiments, the viral vector formulation is 300 mL. In some embodiments, the viral vector formulation is 500 mL. In some embodiments, the viral vector is a retrovirus (e.g., a lentivirus).

In some embodiments, the downstream process produces a viral vector formulation or drug substance with a viral vector titer of 5×10¹⁰ TU to 5×10¹² TU. In some embodiments, the viral vector titer is 1×10¹¹ TU to 3×10¹¹ TU, 2×10¹¹ TU to 4×10¹¹ TU, 3×10¹¹ TU to 5×10¹¹ TU, 4×10¹¹ TU to 6×10¹¹ TU, 5×10¹¹ TU to 7×10¹¹ TU, 6×10¹¹ TU to 8×10¹¹ TU, 7×10¹¹ TU to 9×10¹¹ TU, 8×10¹¹ TU to 1×10¹² TU, 9×10¹¹ TU to 2×10¹² TU, 1×10¹² TU to 3×10¹² TU, 2×10¹² TU to 4×10¹² TU, 3×10¹² TU to 5×10¹² TU. In some embodiments, the viral vector titer is 2.5×10¹² TU to 4.7×10¹² TU. In some embodiments, the viral vector titer is at least about 2.5×10¹² TU. In some embodiments, the viral vector titer is at least about 4.7×10¹² TU. In some embodiments, the viral vector titer is 2.5×10¹² TU. In some embodiments, the viral vector titer is 4.7×10¹² TU.

In some embodiments, the downstream process produces a viral vector formulation or drug substance with a viral vector titer of 5×10⁸ TU to 5×10¹⁰ TU. In some embodiments, the viral vector titer is 1×10⁹ TU to 3×10⁹ TU, 2×10⁹ TU to 4×10⁹ TU, 3×10⁹ TU to 5×10⁹ TU, 4×10⁹ TU to 6×10⁹ TU, 5×10⁹ TU to 7×10⁹ TU, 6×10⁹ TU to 8×10⁹ TU, 7×10⁹ TU to 9×10⁹ TU, 8×10⁹ TU to 1×10¹⁰ TU, 9×10⁹ TU to 2×10¹⁰ TU, 1×10¹⁰ TU to 3×10¹⁰ TU, 2×10¹⁰ TU to 4×10¹⁰ TU, 3×10¹⁰ TU to 5×10¹⁰ TU. In some embodiments, the viral vector titer is 2.5×10¹⁰ TU to 4.7×10¹⁰ TU. In some embodiments, the viral vector titer comprises at least about 2.5×10¹⁰ TU. In some embodiments, the viral vector titer comprises at least about 4.7×10¹⁰ TU. In some embodiments, the viral vector titer is 2.5×10¹⁰ TU. In some embodiments, the viral vector titer is 4.7×10¹⁰ TU. In some embodiments, the viral vector titer is 6×10¹⁰ TU.

In some embodiments, the downstream process produces a viral vector formulation or drug substance with a viral vector titer of 1×10⁹ TU to 1×10¹¹ TU. In some embodiments, the viral vector titer is 2×10⁹ TU to 6×10⁹ TU, 4×10⁹ TU to 8×10⁹ TU, 6×10⁹ TU to 1×10¹⁰ TU, 8×10⁹ TU to 1.2×10¹⁰ TU, 1×10¹⁰ TU to 1.4×10¹⁰ TU, 1.2×10¹⁰ TU to 1.6×10¹⁰ TU, 1.4×10¹⁰ TU to 1.8×10¹⁰ TU, 1.6×10¹⁰ TU to 2×10¹⁰ TU, 1.8×10¹⁰ TU to 4×10¹⁰ TU, 2×10¹⁰ TU to 6×10¹⁰ TU, 4×10¹⁰ TU to 8×10¹⁰ TU, 6×10¹⁰ TU to 1×10¹¹ TU. In some embodiments, the viral vector titer is 5×10¹⁰ TU to 9.4×10¹⁰ TU. In some embodiments, the viral vector titer comprises at least about 5×10¹⁰ TU. In some embodiments, the viral vector titer comprises at least about 9.4×10¹⁰ TU. In some embodiments, the viral vector titer is 5×10¹⁰ TU. In some embodiments, the viral vector titer is 9.4×10¹⁰ TU. In some embodiments, the viral vector titer is 6×10¹⁰ TU.

In some embodiments, the downstream process produces a viral vector formulation or drug substance with a viral vector titer of 1.5×10⁹ TU to 1.5×10¹¹ TU. In some embodiments, the viral vector titer is 3×10⁹ TU to 9×10⁹ TU, 6×10⁹ TU to 1.2×10¹⁰ TU, 9×10⁹ TU to 1.5×10¹⁰ TU, 1.2×10¹⁰ TU to 1.8×10¹⁰ TU, 1.5×10¹⁰ TU to 2.1×10¹⁰ TU, 1.8×10¹⁰ TU to 2.4×10¹⁰ TU, 2.1×10¹⁰ TU to 2.7×10¹⁰ TU, 2.4×10¹⁰ TU to 3×10¹⁰ TU, 2.7×10¹⁰ TU to 6×10¹⁰ TU, 3×10¹⁰ TU to 9×10¹⁰ TU, 6×10¹⁰ TU to 1.2×10¹¹ TU, 9×10¹⁰ TU to 1.5×10¹¹ TU. In some embodiments, the viral vector titer is 7.5×10¹⁰ TU to 1.4×10¹¹ TU. In some embodiments, the viral vector titer comprises at least about 7.5×10¹⁰ TU. In some embodiments, the viral vector titer comprises at least about 1.4×10¹¹ TU.

In some embodiments, the viral vector titer is 7.5×10¹⁰ TU. In some embodiments, the viral vector titer is 1.4×10¹¹ TU. In some embodiments, the viral vector titer is 6×10¹⁰ TU.

### C. Process Controls and Characterization

In some embodiments, provided herein is a method for preparing a viral particle formulation. In some embodiments, the method for preparing a viral particle formulation comprises a manufacturing process. In some embodiments, an exemplary manufacturing process is shown in the upstream and downstream processes in FIG. 1C. In some embodiments, the manufacturing process provided herein includes assessment of one or more manufacturing process controls as shown in FIG. 1C. In some embodiments, the manufacturing process controls, include measurement of one or more characteristics during the manufacturing process with the purpose of ensuring consistency between different manufacturing runs. For example, determining cell count at the beginning of the manufacturing process can ensure that similar cell yields will be consistent between different manufacturing runs, which will ultimately lead to consistent, predictable and stable virus production.

In some embodiments, the manufacturing process provided herein includes assessment of one or more manufacturing process characterizations as shown in FIG. 1C. In some embodiments, a manufacturing process characterization is a characteristic measured to provide information about the performance of the manufacturing process in retrospect. For example, measuring process impurities (i.e., impurities introduced due to reagents or other materials used in the manufacturing process) indicates how well certain steps of the manufacturing process are working. Measuring process impurities at the harvest clarification step or the anion exchange chromatography step indicate how well materials (e.g., columns or membranes) are working to remove process impurities.

In some embodiments, the manufacturing process controls in the upstream process include measuring: cell count or cell viability during cell expansion (e.g., seed train); or cell count, cell viability, pH, dissolved oxygen (DO), or temperature in the production bioreactor. In some embodiments, cell count is used to determine whether cells are growing and also to enable cell passage at non-limiting densities during seed train expansion. In some cases, higher cell densities may induce stress on the cells. In some embodiments, cell viability is used to confirm whether the cell count primarily contains viable, non-dead cells. In some embodiments, pH is used to ensure cells remain viable. In some cases, a pH<6.8 negatively impacts viral producer cell growth and transfection. In some embodiments, DO represents the total amount of oxygen available to viral producer cells.

In some embodiments, the manufacturing process controls in the downstream process include measuring: pressure or turbidity of the viral particle product in the harvest clarification step; pH, conductivity or flow rate of the viral particle product in the AEX chromatography step; viral particle count or p24 of the viral particle product in the UF/DF step; pressure or load factor of the viral particle product during the drug substance (DS) filtration step; or fill weight of the frozen drug product. In some embodiments, turbidity is measured to indicate the presence of cell or other contamination. In some embodiments, pressure is measured to determine whether larger impurities need to be removed. In some cases, high pressures can cause issues in the membrane chromatography step. P24 is a capsid protein expressed by viruses. Thus, in some embodiments, measuring p24 indicates the presence of cells that are producing vial particles. In some embodiments, fill weight is the amount of drug product in a given container (e.g., a vial). In some embodiments, a drug product is assessed for whether it conforms to an acceptable weight range, otherwise the drug product could be rejected during clinical use.

In some embodiments, the manufacturing process characterizations in the upstream process include measuring metabolites, p24, titer, turbidity, hcDNA during cell expansion (e.g., seed train) and production bioreactor steps. In some embodiments, metabolites are used to determine whether cells are healthy.

In some embodiments, the manufacturing process characterizations in the downstream process include measuring: p24, titer, turbidity, or process impurities during the harvest clarification step; p24, titer or process impurities during the AEX and UF/DF steps; or full release, characterization or stability during the DS filtration step.

In addition to the step-wise manufacturing process controls and characterizations, an analytical platform can be used to support a rapid scale up of the lentiviral particle formulation. In some embodiments, the analytical platform comprises methods to assess the: (i) identity of the viral particle formulation; (ii) purity of the viral particle formulation; (iii) potency of the viral particle formulation; and (iv) safety of the viral particle formulation.

In some embodiments, methods employed in identifying the viral particle formulation include sequencing to detect the presence of transgenes expressed by the virus or western blot/ELISA to detect the presence of proteins expressed by the virus.

In some embodiments, methods employed to assess purity of the viral particle formulation include detection of impurities such as endonuclease (a process related impurity), host cell protein, host cell DNA, and polyethyleneimine (PEI; a process related impurity). In some embodiments, viral particle formulation components that indicate purity include the presence of plasmid DNA, E1A DNA, or SV40 DNA.

In some embodiments, methods employed to assess potency of the viral particle formulation includes physical titer, which is determined through the measurement of p24, RNA genomes, and the number of physical particles. In some embodiments, another method used to assess potency includes the transducing titer required to generate the viral particle product. In some embodiments, the transducing titer or infectious titer is any of the infectious titers provided herein. In some embodiments, further methods used to assess potency include measuring the expression of cell surface markers on the virus particles or measuring cell surface marker functionality (e.g., CAR functionality).

In some embodiments, methods employed to assess safety of the viral particle product includes measuring endotoxins, bioburden (i.e., the number of contaminated organisms found in the viral particle formulation prior to sterilization), sterility, subvisible particles (i.e., particles that are too large for analysis by size exclusion chromatography (SEC) (e.g., ˜>0.1 μm), but too small to be visible to the unaided eye (e.g., <100 μm)), mycoplasma, adventitious viruses, and replication competent lentivirus.

### D. Exemplary Methods

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a dual-layer filter component comprising a second filter and a third filter, wherein the second filter has a retention threshold smaller than the first filter and the third filter has a retention threshold smaller than the second filter, thereby producing a filtered formulation of viral particles; (ii) purifying the filtered formulation of viral particles; and (iii) concentrating the filtered formulation of viral particles, wherein concentrating comprises chromatography and ultrafiltration.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a dual-layer filter component comprising a second filter and a third filter, wherein the second filter has a retention threshold smaller than the first filter and the third filter has a retention threshold smaller than the second filter, thereby producing a filtered formulation of retroviral particles; (ii) purifying the filtered formulation of retroviral particles; and (iii) concentrating the filtered formulation of retroviral particles, wherein concentrating comprises chromatography and ultrafiltration.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a dual-layer filter component comprising a second filter and a third filter, wherein the second filter has a retention threshold smaller than the first filter and the third filter has a retention threshold smaller than the second filter, thereby producing a filtered formulation of lentiviral particles; (ii) purifying the filtered formulation of lentiviral particles; and (iii) concentrating the filtered formulation of lentiviral particles, wherein concentrating comprises chromatography and ultrafiltration.

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, (c) filtering the second filtrate with a dual-layer filter component comprising the second filter and a third filter, wherein the third filter has a retention threshold smaller than the second filter, thereby producing a filtered formulation of viral particles; (ii) purifying the filtered formulation of viral particles; and (iii) concentrating the filtered formulation of viral particles, wherein concentrating comprises chromatography and ultrafiltration.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, (c) filtering the second filtrate with a dual-layer filter component comprising the second filter and a third filter, wherein the third filter has a retention threshold smaller than the second filter, thereby producing a filtered formulation of viral particles; (ii) purifying the filtered formulation of retroviral particles; and (iii) concentrating the filtered formulation of retroviral particles, wherein concentrating comprises chromatography and ultrafiltration.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, (c) filtering the second filtrate with a dual-layer filter component comprising the second filter and a third filter, wherein the third filter has a retention threshold smaller than the second filter, thereby producing a filtered formulation of viral particles; (ii) purifying the filtered formulation of lentiviral particles; and (iii) concentrating the filtered formulation of lentiviral particles, wherein concentrating comprises chromatography and ultrafiltration.

In some embodiments, the disclosure provides a method for preparing a viral particle formulation, comprising (i) filtering a suspension mixture comprising a population of host cells and viral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter is a dual-layer filter comprising a first layer filter and a second layer filter, wherein the first layer filter has the same retention threshold as the second filter, and wherein the second layer filter has a retention threshold smaller than the first layer filter; (ii) purifying the filtered formulation of viral particles; and (iii) concentrating the filtered formulation of viral particles, wherein concentrating comprises chromatography and ultrafiltration.

In some embodiments, the disclosure provides a method for preparing a retroviral particle formulation, comprising (i) filtering a suspension mixture comprising a population of host cells and retroviral particles to remove contaminants, wherein filtering comprises: (a) filtering the mixture with a first filter, wherein the first filter is a depth filter, resulting in a first filtrate, (b) filtering the first filtrate with a second filter, wherein the second filter has a retention threshold smaller than the first filter, resulting in a second filtrate, and (c) filtering the second filtrate with a third filter, wherein the third filter is a dual-layer filter comprising a first layer filter and a second layer filter, wherein the first layer filter has the same retention threshold as the second filter, and wherein the second layer filter has a retention threshold smaller than the first layer filter; (ii) purifying the filtered formulation of retroviral particles; and (iii) concentrating the filtered formulation of retroviral particles, wherein concentrating comprises chromatography and ultrafiltration.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter, resulting in a first filtrate, ((b) filtering the first filtrate with a second filter, wherein the second filter has a retention threshold smaller than the first filter, resulting in a second filtrate, and (c) filtering the second filtrate with a third filter, wherein the third filter is a dual-layer filter comprising a first layer filter and a second layer filter, wherein the first layer filter has the same retention threshold as the second filter, and wherein the second layer filter has a retention threshold smaller than the first layer filter, thereby producing a filtered formulation of lentiviral particles; (ii) purifying the filtered formulation of lentiviral particles; and (iii) concentrating the filtered formulation of lentiviral particles, wherein concentrating comprises chromatography and ultrafiltration.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter with a retention threshold of at least 8 μm, resulting in a first filtrate (c) filtering the first filtrate with a second filter, wherein the second filter has a retention threshold of 0.45 μm, resulting in a second filtrate, and (d) filtering the second filtrate with a third filter, wherein the third filter has a retention threshold of 0.2 μm, thereby producing a filtered formulation of lentiviral particles; (iv) purifying the filtered formulation of lentiviral particles; and (v) concentrating the filtered formulation of lentiviral particles, wherein concentrating comprises chromatography and ultrafiltration.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter with a retention threshold of at least 8 μm, resulting in a first filtrate (c) filtering the first filtrate with a second filter, wherein the second filter has a retention threshold of 0.45 μm, resulting in a second filtrate, and (d) filtering the second filtrate with a third filter, wherein the third filter has a retention threshold of 0.2 μm, thereby producing a filtered formulation of lentiviral particles; (iv) purifying the filtered formulation of lentiviral particles; and (v) concentrating the filtered formulation of lentiviral particles, wherein concentrating comprises ion exchange chromatography and two steps of ultrafiltration.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter with a retention threshold of at least 8 μm, resulting in a first filtrate (c) filtering the first filtrate with a second filter, wherein the second filter has a retention threshold of 0.45 μm, resulting in a second filtrate, and (d) filtering the second filtrate with a third filter, wherein the third filter has a retention threshold of 0.2 μm, thereby producing a filtered formulation of lentiviral particles; (iv) purifying the filtered formulation of lentiviral particles; (v) concentrating the filtered formulation of lentiviral particles, wherein concentrating comprises ion exchange chromatography and two steps of ultrafiltration; and (vi) filter sterilizing the concentrated formulation to produce a sterile drug substance.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter with a retention threshold of at least 8 μm, (b) filtering the first filtrate with a dual-layer filter component comprising a second filter and a third filter, wherein the second filter has a retention threshold of 0.45 μm and the third filter has a retention threshold of 0.2 μm, thereby producing a filtered formulation of lentiviral particles; (ii) purifying the filtered formulation of lentiviral particles; and (iii) concentrating the filtered formulation of lentiviral particles, wherein concentrating comprises chromatography and ultrafiltration.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter with a retention threshold of at least 8 μm, (b) filtering the first filtrate with a dual-layer filter component comprising a second filter and a third filter, wherein the second filter has a retention threshold of 0.45 μm and the third filter has a retention threshold of 0.2 μm, thereby producing a filtered formulation of lentiviral particles; (ii) purifying the filtered formulation of lentiviral particles; and (iii) concentrating the filtered formulation of lentiviral particles, wherein concentrating comprises ion exchange chromatography and two steps of ultrafiltration.

In some embodiments, the disclosure provides a method for preparing a lentivirus formulation, comprising (i) filtering a suspension mixture comprising a population of host cells and lentiviral particles to remove contaminants, wherein filtering comprises: (a) filtering the mixture with a first filter, wherein the first filter is a depth filter with a retention threshold of at least 8 μm, (b) filtering the first filtrate with a dual-layer filter component comprising a second filter and a third filter, wherein the second filter has a retention threshold of 0.45 μm and the third filter has a retention threshold of 0.2 μm, thereby producing a filtered formulation of lentiviral particles; (ii) purifying the filtered formulation of lentiviral particles; and (ii) concentrating the filtered formulation of lentiviral particles, wherein concentrating comprises ion exchange chromatography and two steps of ultrafiltration; and (iii) filter sterilizing the concentrated formulation to produce a sterile drug substance.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter with a retention threshold of at least 8 μm, resulting in a first filtrate (c) filtering the first filtrate with a second filter, wherein the second filter has a retention threshold of 0.45 μm, resulting in a second filtrate, and (d) filtering the second filtrate with a third filter, wherein the third filter has a first layer filter having a retention threshold of 0.45 μm and a second layer filter having a retention threshold of 0.2 μm, thereby producing a filtered formulation of lentiviral particles; (iv) purifying the filtered formulation of lentiviral particles; and (v) concentrating the filtered formulation of lentiviral particles, wherein concentrating comprises chromatography and ultrafiltration.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter with a retention threshold of at least 8 μm, resulting in a first filtrate (c) filtering the first filtrate with a second filter, wherein the second filter has a retention threshold of 0.45 μm, resulting in a second filtrate, and (d) filtering the second filtrate with a third filter, wherein the third filter has a first layer filter having a retention threshold of 0.45 μm and a second layer filter having a retention threshold of 0.2 μm; (iv) purifying the filtered formulation of lentiviral particles; and (v) concentrating the filtered formulation of lentiviral particles, wherein concentrating comprises ion exchange chromatography and two steps of ultrafiltration.

In some embodiments, the disclosure provides a method for preparing a lentivirus formulation, comprising (i) contacting a population of host cells in suspension with at least one plasmid encoding a lentiviral protein; (ii) culturing the population of host cells of step (i) for a period of time sufficient to produce a suspension mixture comprising a population of host cells and lentiviral particles; (iii) filtering the suspension mixture to remove contaminants, comprising: (a) contacting the mixture with an endonuclease, (b) filtering the mixture with a first filter, wherein the first filter is a depth filter with a retention threshold of at least 8 μm, resulting in a first filtrate (c) filtering the first filtrate with a second filter, wherein the second filter has a retention threshold of 0.45 μm, resulting in a second filtrate, and (d) filtering the second filtrate with a third filter, wherein the third filter has a first layer filter having a retention threshold of 0.45 μm and a second layer filter having a retention threshold of 0.2 μm; (iv) purifying the filtered formulation of lentiviral particles; (v) concentrating the filtered formulation of lentiviral particles, wherein concentrating comprises ion exchange chromatography and two steps of ultrafiltration; and (vi) filter sterilizing the concentrated formulation to produce a sterile drug substance.

In some embodiments, the disclosure provides a method for preparing a viral particle formulation comprising (i) expanding a population of host cells in one or more seed trains in a shake flask; (ii) inoculating a bioreactor with the population of host cells; (iii) transfecting the population of host cells in the bioreactor; (iv) adding an endonuclease (e.g., benzonase); (v) harvesting cell culture media containing the population of host cells from the bioreactor; (vi) clarifying the cell culture media one or more times; (vii) purifying and concentrating the cell culture media with AEX chromatography, comprising an elution step; (viii) filtrating the cell culture media (e.g., tangential flow filtration); (ix) performing drug substance filtration; and (x) storing the drug substance, i.e., the viral particle formulation.

In some embodiments, disclosure provides a method for preparing a lentivirus formulation comprising (i) expanding a population of host cells in one or more seed trains in a shake flask; (ii) inoculating a bioreactor with the population of host cells; (iii) transfecting the population of host cells in the bioreactor; (iv) adding an endonuclease (e.g., benzonase); (v) harvesting cell culture media containing the population of host cells from the bioreactor; (vi) clarifying the cell culture media one or more times; (vii) purifying and concentrating the cell culture media with AEX chromatography, comprising an elution step; (viii) filtrating the cell culture media (e.g., tangential flow filtration); (ix) performing drug substance filtration; and (x) storing the drug substance, i.e., the lentivirus formulation.

In some embodiments, the disclosure provides a method for preparing a retrovirus formulation comprising (i) expanding a population of host cells in one or more seed trains in a shake flask; (ii) inoculating a bioreactor with the population of host cells; (iii) transfecting the population of host cells in the bioreactor; (iv) adding an endonuclease (e.g., benzonase); (v) harvesting cell culture media containing the population of host cells from the bioreactor; (vi) clarifying the cell culture media one or more times; (vii) purifying and concentrating the cell culture media with AEX chromatography, comprising an elution step; (viii) filtrating the cell culture media (e.g., tangential flow filtration); (ix) performing drug substance filtration; and (x) storing the drug substance, i.e., the retrovirus formulation.

### II. VIRUS PARTICLES

As it is well known in the art, a viral particle is a tool that allows or facilitates the transfer of an entity from one environment to another. In accordance with the disclosure, and by way of example, some viral particles used in recombinant DNA techniques allow entities, such as a segment of DNA, to be transferred into a host cell. Examples of vectors used in recombinant DNA techniques include but are not limited to, plasmids, chromosomes, artificial chromosomes or viruses. The term “expression vector” means a construct capable of in vivo or in vitro/ex vivo expression.

### A. Retroviral Particles

In some embodiments, the disclosure provides a method for preparing a viral formulation. In some embodiments, the virus is a retrovirus. A large number of different retroviruses have been identified. Examples of retrovirus include but are not limited to: murine leukemia virus (MLV), human immunodeficiency virus (HIV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “Retroviruses”, Cold Spring Harbor Laboratory Press Eds: J M Coffin, S M Hughes, H E Varmus pp 758-763.

Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

A lentiviral vector of the disclosure may be derived from or may be derivable from any suitable lentivirus. A recombinant retroviral vector particle is capable of transducing a recipient cell with a nucleotide of interest (NOI). Once within the cell, the RNA genome from the vector particle is reverse transcribed into DNA and integrated into the DNA of the recipient cell. In some embodiments of the disclosure, at least part of one or more protein coding regions essential for replication may be removed from the virus. This makes the viral vector replication defective. Portions of the viral genome may also be replaced by an NOI in order to generate a vector comprising an NOI which is capable of transducing a target non-dividing host cell and/or integrating its genome into a host genome.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an illustrative third-generation system, the Env gene is VSV-G and the promoter is the CMV promoter. The third-generation system uses two packaging plasmids, one encoding gag and pol and the other encoding rev as a further safety feature; an improvement over the single packaging plasmid of so-called second-generation systems. Although safer, the third-generation system can be more cumbersome to use and result in lower viral titers due to the addition of an additional plasmid. Illustrative packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, containing a retroviral or lentiviral envelope including one or more of the env encoded glycoproteins, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to the gating adaptor. In some embodiments, a sequence encoding a receptor that specifically binds to the gating adaptor is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to a ligand, and (iv) a 3′ LTR or UTR.

In some embodiments, the retroviral particles comprise a cell surface receptor that binds to a surface marker on a target host cell, allowing host cell transduction. The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof.

In some embodiments, the viral envelope comprises a viral envelope protein. In some embodiments, a viral envelope protein is a VSV-G envelope protein, a measles virus envelope protein, a nipah virus envelope protein, or a cocal virus G protein. In some embodiments, the viral particle comprises a modified VSV G protein that lacks LDLR binding affinity. In some embodiments, these mutations comprise mutations at positions 47 (for example, K47Q) and/or 354 (for example, R354A).

In some embodiments, the viral envelope protein is a VSV G protein from the Cocal strain (Cocal glycoprotein). In some embodiments, the VSV G protein is a Cocal envelope protein containing a mutation at position 354 (R354). In some embodiments, the VSV G protein is a Cocal envelope protein containing a mutation at position 47 (K47). In some embodiments, the VSV G protein is a Cocal envelope variant containing a R354Q mutation. In some embodiments, the VSV G protein is a Cocal envelope variant containing a K47Q mutation. In some embodiments, this variant may be referred to as “blinded” Cocal envelope. Illustrative Cocal envelope variants are provided in, e.g., US 2020/0216502 A1, which is incorporated herein by reference in its entirety.

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, innate lymphoid cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors. In some embodiments, the vector may be a viral vector, a retroviral vector, a lentiviral vector, a gamma-retroviral vector. In some embodiments, the viral vector comprises a VSV G-protein or functional variant thereof. In some embodiments, the viral vector comprises a Cocal G-protein or functional variant thereof.

### 1. Engineered Viral Envelope

In some embodiments, the viral envelope protein is engineered to express a surface engineered protein. In some embodiments, the surface engineered protein is exposed on the surface of the lentiviral particle. In some embodiments, the surface engineered protein is embedded in the lipid bilayer. In some embodiments, the surface engineered protein is composed of a single binding domain protein that binds to a target molecule on a target cell. In some embodiments, the surface engineered protein is composed of a multiple binding domain protein, wherein each binding domain binds to a target molecule on a target cell. In some embodiments, each binding domain of the multiple binding protein binds to a different target molecule. In some embodiments, the binding domains of the multiple binding protein are connected by a linker. In some embodiments, the target cell is an immune cell, such as a T cell. In some embodiments, the surface engineered protein is a transduction enhancer. In some embodiments, the surface engineered protein part of the viral envelope as a fusion protein with a viral envelope protein.

In some embodiments, the viral envelope comprises a transduction enhancer. In some embodiments, the viral envelope comprises an immune cell-activating protein. In some embodiments, the viral envelope comprises a co-stimulation molecule. In some embodiments, the viral envelope comprises an immune cell-activating protein, and a co-stimulation molecule. In some embodiments, the viral envelope comprises an adhesion molecule.

In some embodiments, the transduction enhancer is a single domain binding protein or is a multiple domain binding protein. In some embodiments, the transduction enhancer comprises at least one binding domain that binds a target molecule selected from an immune cell activating receptor, a T cell costimulatory receptor or an adhesion molecule. In some embodiments, the immune cell activating receptor is a T cell activating receptor or an NK cell activating receptor.

In some embodiments, the transduction enhancer comprises a single binding domain that binds to one target molecule selected from an immune cell activating receptor (e.g. T cell activating receptor), a T cell costimulatory receptor or an adhesion molecule. In some embodiments, the single domain fusion protein is encoded by a nucleic acid sequence of 600-900 nucleotides. In some embodiments, the single domain fusion protein is encoded by a nucleic acid sequence of about 720 nucleotides. In some embodiments, the single fusion protein is 200-300 amino acids in length. In some embodiments, the single fusion protein is about 240 amino acids in length.

In some embodiments, the transduction enhancer comprises multiple binding domains that bind to two or more target molecules selected from an immune cell activating receptor (e.g. a T cell activating receptor), a T cell costimulatory receptor or an adhesion molecule. In some embodiments, the transduction enhancer comprises multiple binding domains that each bind to a different target molecule that is an immune cell activating receptor (e.g. T cell activating receptor), a T cell costimulatory receptor and an adhesion molecule. In some embodiments, the viral envelope comprises an immune cell-activating protein, a co-stimulation molecule and an adhesion molecule. In some embodiments, the multi-domain fusion protein is encoded by a nucleic acid sequence of 1,000 to 3,000 nucleotides. In some embodiments, the multi-domain fusion protein is encoded by a protein that is at least 2,000, nucleotides. In some embodiments, the multi-domain fusion protein is at least 700 amino acids in length.

In some embodiments, the viral envelope comprises one or more transduction enhancers. In some embodiments, the transduction enhancers include T cell activation receptors, NK cell activation receptors, and/or co-stimulatory molecules. In some embodiments, one or more transduction enhancers comprise one or more of anti-CD3scFv, CD86, CD80, and/or CD58. In some embodiments, the transduction enhancers comprise at least an anti-CD3 scFv, and CD58. In some embodiments, the transduction enhancers comprise at least an anti-CD3 scFv, and CD80. In some embodiments, the transduction enhancers comprise at least an anti-CD3 scFv, and CD86. In some embodiments, the transduction enhancers comprise at least an anti-CD3 scFv, a CD80, and CD58. In some embodiments, the transduction enhancers comprise at least an anti-CD3 scFv, a CD86, and CD58.

In some embodiments, the viral particle is surface engineered with a protein that binds to a target molecule on a target cell. In some embodiments, the surface engineered protein is a fusion of one or more binding domains that bind to a target molecule on a target cell with a viral envelope protein. In some embodiments, the viral envelope protein is a heterologous viral envelope protein. In some embodiments, the viral particle comprises a cell surface receptor that binds to a ligand on a target host cell, allowing host cell transduction. In some embodiments, the viral particle comprises a heterologous viral envelope glycoprotein yielding a pseudotyped viral particle. For example, the viral envelope glycoprotein may be derived from RD114 or one of its variants, VSV-G, Gibbon-ape leukemia virus (GALV), or is the Amphotropic envelope, Measles envelope or baboon retroviral envelope glycoprotein. In some embodiments, the viral envelope glycoprotein is a VSV G protein from the Cocal strain (Cocal glycoprotein) or a functional variant thereof.

In some embodiments, the viral envelope comprises more than one polypeptide on the surface. In some embodiments, the more than one polypeptide binds to a target immune cells and replicates an immunological synapse. In some embodiments, the viral envelope comprises an immune cell-activating protein, a co-stimulatory molecule, and an adhesion molecule, wherein the immune cell-activating protein, co-stimulatory molecule, and adhesion molecule each bind a target immune cell.

a. Immune Cell-Activating Agents

In some embodiments, the transduction enhancer comprises a mitogenic stimulus, which is incorporated into a retroviral or lentiviral capsid, such that the virus both activates and transduces T cells. This removes the need to add vector and mitogen. In some embodiments, the transduction enhancer comprises a mitogenic transmembrane protein and/or one or more costimulatory molecules, which get(s) incorporated into the retrovirus when it buds from the producer/packaging cell membrane. In some embodiments, the transduction enhancers are expressed as separate cell surface molecules on the producer cell rather than being part of the viral envelope glycoprotein.

In some embodiments, the viral vector described herein comprises a mitogenic transduction enhancer in the viral envelope. In some embodiments, the mitogenic transduction enhancer is derived from the host cell during retroviral vector production. In some embodiments, the mitogenic transduction enhancer is made by the packaging cell and expressed at the cell surface. When the nascent retroviral vector buds from the host cell membrane, the mitogenic transduction enhancer may be incorporated in the viral envelope as part of the packaging cell-derived lipid bilayer. In some embodiments, the mitogenic enhancer is an antibody or fragment thereof. In some embodiments, the mitogenic enhancer is a single domain antibody, for example, a camelid antibody. In some embodiments, the mitogenic enhancer is an scFv. In some embodiments, the mitogenic enhancer is a nanobody.

In some embodiments, the transduction enhancer is host-cell derived. The term “host-cell derived” indicates that the mitogenic transduction enhancer is derived from the host cell as described above and is not produced as a fusion or chimera from one of the viral genes, such as gag, which encodes the main structural proteins; or env, which encodes the envelope protein.

Envelope proteins are formed by two subunits, the transmembrane (TM) that anchors the protein into the lipid membrane and the surface (SU) which binds to the cellular receptors. In some embodiments, the packaging-cell derived mitogenic transduction enhancer of the present invention does not comprise the surface envelope subunit (SU).

In some embodiments, the mitogenic transduction enhancer has the structure: M-S-TM, in which M is a mitogenic domain; S is an optional spacer domain and TM is a transmembrane domain.

The mitogenic domain is the part of the mitogenic transduction enhancer which causes T-cell activation. It may bind or otherwise interact, directly or indirectly, with a T cell, leading to T cell activation. In some embodiments, the mitogenic domain binds a T cell surface antigen, such as CD3, CD28, CD134 and CD137.

CD3 is a T-cell co-receptor. It is a protein complex composed of four distinct chains. In mammals, the complex contains a CD3y chain, a CD35 chain, and two CD3e chains. These chains associate with the T-cell receptor (TCR) and the z-chain to generate an activation signal in T lymphocytes. The TCR, z-chain, and CD3 molecules together comprise the TCR complex. In some embodiments, the mitogenic domain binds to a CD3 e chain.

In some embodiments, the mitogenic domain comprises all or part of an antibody or other molecule which specifically binds a T-cell surface antigen. In some embodiments, the antibody activates the TCR or CD28. In some embodiments, the antibody binds the TCR, CD3 or CD28. Examples of such antibodies include: OKT3, 15E8 and TGN1412. Other suitable antibodies include:
Anti-CD28: CD28.2, 10F3
Anti-CD3/TCR: UCHT1, YTH12.5, TR66

In some embodiments, the mitogenic domain comprises the binding domain from OKT3, 15E8, TGN1412, CD28.2, 10F3, UCHT1, YTH12.5 or TR66.

In some embodiments, the mitogenic domain comprises all or part of a co-stimulatory molecule such as OX40L and 41BBL. For example, the mitogenic domain may comprise the binding domain from OX40L or 41BBL.

OKT3, also known as Muromonab-CD3 is a monoclonal antibody targeted at the CD3e chain. It is clinically used to reduce acute rejection in patients with organ transplants. It was the first monoclonal antibody to be approved for clinical use in humans.

In some embodiments, the viral envelope comprises an immune cell-activating protein. In some embodiments, the immune cell-activating protein specifically binds a receptor on an immune cell. In some embodiments, the immune cell-activating protein provides signal one for T cell activation.

In some embodiments, the immune cell-activating protein specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, or NKp80. In some embodiments, the immune cell-activating protein specifically binds CD3γ, CD3δ, or CD3ε. In some embodiments, the immune cell-activating protein specifically binds CD3γ, CD3δ, CD3ε, CD9, CD5, CD22, CD33, CD37, CD64, CD45, CD28H, LFA-1, DNAM-1, CD27, ICOS, LIGHT, GITR, CD30, SLAM, Ly-9, CD84, Ly108, CD16, CD56, NKG2D, NKp46, NKp44, NKp30, CD244, NKp80, TCRα chain, TCRβ chain, TCRγ chain, or TCRδ chain. In some embodiments, the immune cell-activating protein specifically binds CD3γ, CD3δ, or CD3ε. In some embodiments, the immune cell-activating protein specifically binds CD3.

In some embodiments, the immune cell-activating protein is an antibody or antigen binding fragment thereof that specifically binds a receptor on an immune cell. In some embodiments, the immune cell-activating protein is an antibody or antigen binding fragment thereof that specifically binds CD28, CD2, CD3, CD28H, LFA-1, OX40, 4-1BB, CD40L, DNAM-1, CD27, ICOS, LIGHT, GITR, CD30, SLAM, Ly-9, CD84, Ly108, NKG2D, NKp46, NKp44, NKp30, CD244, or NKp80. In some embodiments, the immune cell-activating protein is an antibody or antigen binding fragment thereof that specifically binds CD28, CD2, CD3γ, CD3δ, CD3ε, CD4, CD8, CD9, CD5, CD22, CD33, CD37, CD64, CD45, CD28H, LFA-1, OX40, 4-1BB, CD40L, DNAM-1, CD27, ICOS, LIGHT, GITR, CD30, SLAM, Ly-9, CD84, Ly108, CD16, CD56, NKG2D, NKp46, NKp44, NKp30, CD244, NKp80, TCRα chain, TCRβ chain, TCRγ chain, or TCRδ chain. In some embodiments, the immune cell-activating protein is an antibody or antigen binding fragment thereof that specifically binds CD3γ, CD3δ, or CD3ε. In some embodiments, immune cell-activating protein is an antibody or antigen binding fragment thereof that specifically binds CD3.

Antibodies targeting the polypeptides described herein are known to those of skill in the art. Methods for generating antibodies are known to those of skill in the art.

In some embodiments, the viral envelope comprises an anti-CD3F antibody, or antigen-binding fragment thereof. In some embodiments, the anti-CD3F antibody, or antigen-binding fragment thereof is coupled to a transmembrane domain. An illustrative anti-CD3F antibody is OKT3. OKT3, also known as Muromonab-CD3, is a monoclonal antibody targeted at the CD3ε chain.

In some embodiments, the viral envelope comprises a single chain Fv fragment (scFv) of an anti-CD3 antibody.

In some embodiments, the anti-CD3 scFv comprises an amino acid sequence at least 80%, 85%, 90%, 95%, 96%, 97%, 98%, 99% or 100% identical to the amino acid sequence of SEQ ID NO:120. In some embodiments, the anti-CD3 scFv comprises an amino acid sequence at least 80% identical to the amino acid sequence of SEQ ID NO:120. In some embodiments, the anti-CD3 scFv comprises an amino acid sequence at least 85% identical to the amino acid sequence of SEQ ID NO:120. In some embodiments, the anti-CD3 scFv comprises an amino acid sequence at least 90% identical to the amino acid sequence of SEQ ID NO:120. In some embodiments, the anti-CD3 scFv comprises an amino acid sequence at least 95% identical to the amino acid sequence of SEQ ID NO:120. In some embodiments, the anti-CD3 scFv comprises an amino acid sequence at least 96% identical to the amino acid sequence of SEQ ID NO: 120. In some embodiments, the anti-CD3 scFv comprises an amino acid sequence at least 97% identical to the amino acid sequence of SEQ ID NO:120. In some embodiments, the anti-CD3 scFv comprises an amino acid sequence at least 98% identical to the amino acid sequence of SEQ ID NO:120. In some embodiments, the anti-CD3 scFv comprises an amino acid sequence at least 99% identical to the amino acid sequence of SEQ ID NO:120. In some embodiments, the anti-CD3 scFv comprises an amino acid sequence at least 100% identical to the amino acid sequence of SEQ ID NO:120. In some embodiments, the anti-CD3 scFv comprises the amino acid sequence of SEQ ID NO:120. In some embodiments, the anti-CD3 scFv consists the amino acid sequence of SEQ ID NO:120.

In some embodiments, the anti-CD3 scFv comprises an amino acid sequence at least 80%, 85%, 90%, 95%, 96%, 97%, 98%, 99% or 100% identical to the amino acid sequence of SEQ ID NO:122. In some embodiments, the anti-CD3 scFv comprises an amino acid sequence at least 80% identical to the amino acid sequence of SEQ ID NO:122. In some embodiments, the anti-CD3 scFv comprises an amino acid sequence at least 85% identical to the amino acid sequence of SEQ ID NO:122. In some embodiments, the anti-CD3 scFv comprises an amino acid sequence at least 90% identical to the amino acid sequence of SEQ ID NO:122. In some embodiments, the anti-CD3 scFv comprises an amino acid sequence at least 95% identical to the amino acid sequence of SEQ ID NO:122. In some embodiments, the anti-CD3 scFv comprises an amino acid sequence at least 96% identical to the amino acid sequence of SEQ ID NO:122. In some embodiments, the anti-CD3 scFv comprises an amino acid sequence at least 97% identical to the amino acid sequence of SEQ ID NO:122. In some embodiments, the anti-CD3 scFv comprises an amino acid sequence at least 98% identical to the amino acid sequence of SEQ ID NO:122. In some embodiments, the anti-CD3 scFv comprises an amino acid sequence at least 99% identical to the amino acid sequence of SEQ ID NO:122. In some embodiments, the anti-CD3 scFv comprises an amino acid sequence at least 100% identical to the amino acid sequence of SEQ ID NO:122. In some embodiments, the anti-CD3 scFv comprises the amino acid sequence of SEQ ID NO:122. In some embodiments, the anti-CD3 scFv consists the amino acid sequence of SEQ ID NO:122.

In some embodiments, the anti-CD3 scFV is encoded by a nucleotide sequence at least 80%, 85%, 90%, 95%, 96%, 97%, 98%, 99% or 100% identical to the nucleotide sequence of SEQ ID NO:121. In some embodiments, the anti-CD3 scFv is encoded by a nucleotide sequence at least 80% identical to the nucleotide sequence of SEQ ID NO:121. In some embodiments, the anti-CD3 scFv is encoded by a nucleotide sequence at least 85% identical to the nucleotide sequence of SEQ ID NO:121. In some embodiments, the anti-CD3 scFv is encoded by a nucleotide sequence at least 90% identical to the nucleotide sequence of SEQ ID NO:121. In some embodiments, the anti-CD3 scFv is encoded by a nucleotide sequence at least 95% identical to the nucleotide sequence of SEQ ID NO:121. In some embodiments, the anti-CD3 scFv is encoded by a nucleotide sequence at least 96% identical to the nucleotide sequence of SEQ ID NO:121. In some embodiments, the anti-CD3 scFv is encoded by a nucleotide sequence at least 97% identical to the nucleotide sequence of SEQ ID NO:121. In some embodiments, the anti-CD3 scFv is encoded by a nucleotide sequence at least 98% identical to the nucleotide sequence of SEQ ID NO:121. In some embodiments, the anti-CD3 scFv is encoded by a nucleotide sequence at least 99% identical to the nucleotide sequence of SEQ ID NO:121. In some embodiments, the anti-CD3 scFv is encoded by a nucleotide sequence at least 100% identical to the nucleotide sequence of SEQ ID NO:121. In some embodiments, the anti-CD3 scFv is encoded by a nucleotide sequence comprising the nucleotide sequence of SEQ ID NO:121. In some embodiments, the anti-CD3 scFv is encoded by a nucleotide sequence consisting of the nucleotide sequence of SEQ ID NO:121.

In some embodiments, the anti-CD3 scFV is encoded by a nucleotide sequence at least 80%, 85%, 90%, 95%, 96%, 97%, 98%, 99% or 100% identical to the nucleotide sequence of SEQ ID NO:123. In some embodiments, the anti-CD3 scFv is encoded by a nucleotide sequence at least 80% identical to the nucleotide sequence of SEQ ID NO:123. In some embodiments, the anti-CD3 scFv is encoded by a nucleotide sequence at least 85% identical to the nucleotide sequence of SEQ ID NO:123. In some embodiments, the anti-CD3 scFv is encoded by a nucleotide sequence at least 90% identical to the nucleotide sequence of SEQ ID NO:123. In some embodiments, the anti-CD3 scFv is encoded by a nucleotide sequence at least 95% identical to the nucleotide sequence of SEQ ID NO:123. In some embodiments, the anti-CD3 scFv is encoded by a nucleotide sequence at least 96% identical to the nucleotide sequence of SEQ ID NO:123. In some embodiments, the anti-CD3 scFv is encoded by a nucleotide sequence at least 97% identical to the nucleotide sequence of SEQ ID NO:123. In some embodiments, the anti-CD3 scFv is encoded by a nucleotide sequence at least 98% identical to the nucleotide sequence of SEQ ID NO:123. In some embodiments, the anti-CD3 scFv is encoded by a nucleotide sequence at least 99% identical to the nucleotide sequence of SEQ ID NO:123. In some embodiments, the anti-CD3 scFv is encoded by a nucleotide sequence at least 100% identical to the nucleotide sequence of SEQ ID NO:123. In some embodiments, the anti-CD3 scFv is encoded by a nucleotide sequence comprising the nucleotide sequence of SEQ ID NO:123. In some embodiments, the anti-CD3 scFv is encoded by a nucleotide sequence consisting of the nucleotide sequence of SEQ ID NO:123.

b. Co-Stimulatory Molecules

In some embodiments, the viral envelope comprises at least one co-stimulatory molecule. In some embodiments, the co-stimulatory molecule specifically binds a receptor on an immune cell. In some embodiments, the co-stimulatory provides signal two for cell activation.

As used herein, the term “costimulatory molecule” refers to a molecule capable of generating a costimulatory signal to T cells. Lymphocytes, such as T cells and natural killer (NK) cells, typically require several signals and interactions with antigen presenting cells (APCs) for optimal priming to gain full effector functions. For T cells these include signaling through the T cell receptor (TCR), costimulatory molecules (such as CD28 and CD2), cytokines, as well as various adhesion molecules necessary to allow sufficient time for proper synapse formation and signal transduction. NK cells require similar types of stimulation but may rely on different activating receptors, such as NKG2D, NKp46, and DNAM-1. For T cells, proper costimulation, in addition to TCR stimulation, is especially important for effective priming and many studies have shown that TCR stimulation alone can lead to functional anergy and unresponsiveness. Costimulatory signals augment T and NK cell function by enhancing cell metabolism, cytokine production, differentiation, and long-term persistence. Costimulation is an important factor for cell proliferation, differentiation and survival. In some embodiments, costimulatory molecules include, but are not limited to, CD45, CD2, CD4, CD5, CD8, CD9, CD16, CD22, CD33, CD28, CD37, CD64, CD80, CD86, CD134, CD137, and CD154. In some embodiments, the costimulatory molecule includes, but is not limited to, binding agents, such as scFvs, antibodies, single-domain antibodies, antibody fragments, nanobodies that bind to any of the costimulatory molecules described herein. In some embodiments, these binding agents may include anti-CD28, anti-CD2, anti-CD45, anti-CD4, anti-CD5, anti-CD8, anti-CD9, anti-CD16, anti-CD22, anti-CD33, anti-CD37, anti-CD64, anti-CD80, anti-CD86, anti-CD137, anti-CD154, anti-CD28H, anti-LFA-1, anti-OX40, anti-4-1BB, anti-CD40L, anti-DNAM-1, anti-CD27, anti-ICOS, anti-LIGHT, anti-GITR, anti-CD30, anti-SLAM, anti-Ly-9, anti-CD84, anti-Ly108, anti-NKG2D, anti-NKp46, anti-NKp44, anti-NKp30, anti-CD244, anti-NKp80, anti-TCRα chain, anti-TCRβ chain, anti-TCRγ chain, and anti-TCRδ chain agents.

In some embodiments, the co-stimulation molecule is a ligand for CD28. CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). In some embodiments, the co-stimulation molecule is an antibody, or fragment thereof, that binds to CD28. Examples of such antibodies include: 15E8 and TGN1412. Other suitable antibodies include: CD28.2 and 10F3.

In some embodiments, the co-stimulation molecule is CD86. CD86, also known as B7-2, is a ligand for CD28. In some embodiments, the ligand for CD28 is CD86. In some embodiments, the co-stimulation molecule is CD80. CD80 is an additional ligand for CD28. In some embodiments, the ligand for CD28 is CD80. In some embodiments, the ligand for CD28 is an anti-CD28 antibody or an anti-CD28 scFv. In some embodiments, the anti-CD28 antibody or an anti-CD28 scFv is coupled to a transmembrane domain for display on the surface of the viral envelope.

In some embodiments, the co-stimulation molecule is a CD86 polypeptide comprising the amino acid sequence of SEQ ID NO: 5. In some embodiments, the co-stimulation molecule is a CD86 polypeptide comprising an amino acid sequence at least 80%, at least 85%, at least 90%, at least 95%, at least 96%, at least 97%, at least 98% or at least 99% identical to SEQ ID NO: 5.

In some embodiments, the CD86 polypeptide is encoded by the nucleotide sequence of SEQ ID NO: 6. In some embodiments, the CD86 polypeptide is encoded by a nucleotide sequence at least 80%, at least 85%, at least 90%, at least 95%, at least 96%, at least 97%, at least 98% or at least 99% identical to SEQ ID NO: 6.

In some embodiments, the co-stimulation molecule is a CD80 polypeptide comprising the amino acid sequence of SEQ ID NO: 3. In some embodiments, the co-stimulation molecule is a CD80 polypeptide comprising an amino acid sequence at least 80%, at least 85%, at least 90%, at least 95%, at least 96%, at least 97%, at least 98% or at least 99% identical to SEQ ID NO: 3.

In some embodiments, the CD80 polypeptide is encoded by the nucleotide sequence of SEQ ID NO: 4. In some embodiments, the CD80 polypeptide is encoded by a nucleotide sequence at least 80%, at least 85%, at least 90%, at least 95%, at least 96%, at least 97%, at least 98% or at least 99% identical to SEQ ID NO: 4.

In some embodiments, the co-stimulation molecule is a CD80 extracellular domain polypeptide comprising the amino acid sequence of SEQ ID NO: 7. In some embodiments, the co-stimulation molecule is a CD80 extracellular domain polypeptide comprising an amino acid sequence at least 80%, at least 85%, at least 90%, at least 95%, at least 96%, at least 97%, at least 98% or at least 99% identical to SEQ ID NO: 7. In some embodiments, the co-stimulation molecule is a CD86 extracellular domain polypeptide comprising the amino acid sequence of SEQ ID NO: 8. In some embodiments, the co-stimulation molecule is a CD86 extracellular domain polypeptide comprising an amino acid sequence at least 80%, at least 85%, at least 90%, at least 95%, at least 96%, at least 97%, at least 98% or at least 99% identical to SEQ ID NO: 8.

CD134, also known as OX40, is a member of the TNFR-superfamily of receptors which is expressed on activated T cells. OX40 may promote cell division and survival. OX40 is a secondary costimulatory molecule, expressed after 24 to 72 hours following activation; its ligand, OX40L, is also not expressed on resting antigen presenting cells, but is following their activation. In some embodiments, the viral particle comprises a ligand for OX40, or functional fragment thereof, coupled to its native transmembrane domain or a heterologous transmembrane domain.

CD137, also known as 4-1BB, is a member of the tumor necrosis factor (TNF) receptor family. CD137 is expressed on activated T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity. In some embodiments, the viral particle comprises a ligand for 4-1BB, or functional fragment thereof, coupled to its native transmembrane domain or a heterologous transmembrane domain. 4-1BBL is a cytokine that belongs to the tumor necrosis factor (TNF) ligand family. This transmembrane cytokine is a bidirectional signal transducer that acts as a ligand for 4-1BB, which is a costimulatory receptor molecule in T lymphocytes. 4-1BBL has been shown to reactivate anergic T lymphocytes in addition to promoting T lymphocyte proliferation.

Viral particles comprising one or more activation or co-stimulation molecule(s) may be made by engineering the packaging cell line by methods provided by WO 2016/139463; or by expression of the T-cell activation or co-stimulation molecule(s) from a polycistronic helper vector as described in Int'l Pat. Pub. No. WO 2020/106992 A1, both of which are incorporated herein by reference in their entireties.

c. Adhesion Molecules

In some embodiments, the viral particle comprises an adhesion molecule. As used herein, the term “adhesion molecule” refers to a subset of cell surface molecules involved in the binding of cells with other cells. Adhesion cells may help to form more stable interactions, such as an immunological synapse, between immune cells. The immunological synapse is a stable adhesive junction between a polarized immune effector cell and an antigen-bearing cell. In some embodiments, the adhesion molecule may provide a costimulatory signal to the target cell. In some embodiments, adhesion molecules include, but are not limited to, CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, and B7-H6.

In some embodiments, the adhesion molecule is a fragment or ectodomain of CD58, HHLA2, ICAM-1, OX40L, 4-1BBL, CD40, CD155, CD70, HVEM, GITRL, ICOSL, CD30L, SLAM, Ly-9, CD84, Ly108, MICA, MICB, ULBP1, ULBP2, ULBP3, ULBP4, ULBP5, ULBP6, and B7-H6. In some embodiments, adhesion molecules include but are not limited to SLAMF2, B7-H2, B7-H5, B7-H3, B7x, and TMIGD2. In some embodiments, the adhesion molecule is a fragment or ectodomain of SLAMF2, B7-H2, B7-H5, B7-H3, B7x, and TMIGD2.

In some embodiments, the adhesion molecule includes, but is not limited to, binding agents, such as scFvs, antibodies, single-domain antibodies, antibody fragments, and nanobodies that bind to any of the adhesion or costimulatory molecules described herein. In some embodiments, these binding agents may include anti-CD28, anti-CD2, anti-CD28H, anti-LFA-1, anti-OX40, anti-4-1BB, anti-CD40L, anti-DNAM-1, anti-CD27, anti-ICOS, anti-LIGHT, anti-GITR, anti-CD30, anti-SLAM, anti-Ly-9, anti-CD84, anti-Ly108, anti-NKG2D, anti-NKp46, anti-NKp44, anti-NKp30, anti-CD244, anti-NKp80, anti-TCRα chain, anti-TCRβ chain, anti-TCRγ chain, and anti-TCRδ chain agents.

In some embodiments, the adhesion molecule binds to CD2. CD2 is also known as T11, LFA-2, and the erythrocyte rosette receptor and is a surface protein expressed on T lymphocytes and NK cells. CD2 is a natural ligand for CD58. In addition to performing adhesion functions, engagement of CD2 provides a costimulatory signal that may enhance activation and effector functions. In some embodiments, the lentiviral particle comprises a molecule that binds to CD2. In some embodiments, the lentiviral particle comprises an antibody, single domain antibody, antibody fragment, and/or nanobody specific for CD2. In some embodiments, the lentiviral particle comprises CD58, or a functional portion thereof, that binds to CD2.

In some embodiments, the adhesion molecule is CD58. In some embodiments, the adhesion molecule is a CD58 polypeptide comprising the amino acid sequence of SEQ ID NO: 1. In some embodiments, the adhesion molecule is a CD58 polypeptide comprising an amino acid sequence at least 80%, at least 85%, at least 90%, at least 95%, at least 96%, at least 97%, at least 98% or at least 99% identical to SEQ ID NO: 1.

In some embodiments, the CD58 polypeptide is encoded by the nucleotide sequence of SEQ ID NO: 2. In some embodiments, the CD58 polypeptide is encoded by a nucleotide sequence at least 80%, at least 85%, at least 90%, at least 95%, at least 96%, at least 97%, at least 98% or at least 99% identical to SEQ ID NO: 2.

In some embodiments, the adhesion molecule is a CD58 extracellular domain polypeptide comprising the amino acid sequence of SEQ ID NO: 9. In some embodiments, the adhesion molecule is a CD58 extracellular domain polypeptide comprising an amino acid sequence at least 80%, at least 85%, at least 90%, at least 95%, at least 96%, at least 97%, at least 98% or at least 99% identical to SEQ ID NO: 9.

d. Additional Non-Viral Proteins

In some embodiments, the viral particle comprises at least one non-viral protein. In some embodiments, the viral particle comprises at least one non-viral protein in addition to those described supra.

In some embodiments, the viral particle comprises a targeting ligand. In some embodiments, the viral particle comprises CD19, or a functional fragment thereof, coupled to its native transmembrane domain or a heterologous transmembrane domain. In some embodiments, CD19 acts as a ligand for blinatumomab, thus providing an adapter for coupling the particle to T-cells via the anti-CD3 moiety of blinatumomab. In some embodiments, another type of particle surface ligand can serve to couple an appropriately surface engineered lentiviral particle to a T-cell using a multispecific antibody comprising a binding moiety for the particle surface ligand. In some embodiments, the multispecific antibody is a bispecific antibody, for example, a Bispecific T-cell engager (BiTE).

In some embodiments, the non-viral protein is a cytokine. In some embodiments, the cytokine may be selected from the group consisting of IL-2, IL-7, IL-12, IL-15, IL-18, IL-21, and any combination thereof. In some embodiments, the cytokine is IL-12α. In some embodiments, the cytokine is IL-12β. Where the non-viral protein used is a soluble protein (such as an scFv or a cytokine) it may be tethered to the surface of the viral particle by fusion to a transmembrane domain, such as the transmembrane domain of CD8. Alternatively, it may be indirectly tethered to the lentiviral particle by use of a transmembrane protein engineered to bind the soluble protein. Further inclusion of one or more cytoplasmic residues may increase the stability of the fusion protein.

The mitogenic transduction enhancer and/or cytokine-based transduction enhancer may comprise a “spacer sequence” to connect the antigen-binding domain with the transmembrane domain. A flexible spacer allows the antigen-binding domain to orient in different directions to facilitate binding. As used herein, the term “coupled to” refers to a chemical linkage, a direct C-terminal to N-terminal fusion of two protein; chemical linkage to a non-peptide space; chemical linkage to a polypeptide space; and C-terminal to N-terminal fusion of two protein via peptide bonds to a polypeptide spacer, e.g., a spacer sequence.

The spacer sequence may, for example, comprise an lgG1 Fc region, an lgG1 hinge or a human CD8 stalk or the mouse CD8 stalk. The spacer may alternatively comprise an alternative linker sequence which has similar length and/or domain spacing properties as an lgG1 Fc region, an lgG1 hinge or a CD8 stalk. A human lgG1 spacer may be altered to remove Fc binding motifs. In some embodiments, the spacer sequence may be derived from a human protein.

In some embodiments, the spacer sequence comprises a CD8 derived hinge.

In some embodiments, the spacer sequence comprises a ‘short’ hinge. The short hinge is described as hinge region comprising fewer nucleotides relative to CAR hinge regions known in the art.

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

The viral particle of the present invention may comprise a cytokine-based transduction enhancer in the viral envelope. In some embodiments, the cytokine-based transduction enhancer is derived from the host cell during viral particle production. In some embodiments, the cytokine-based transduction enhancer is made by the host cell and expressed at the cell surface. When the nascent viral particle buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain (e.g., a spacer sequence) and TM is the transmembrane domain. The spacer domain and transmembrane domains are as defined above.

The cytokine domain may comprise a T-cell activating cytokine, such as from IL2, IL7 and IL15, or a functional fragment thereof. As used herein, a “functional fragment” of a cytokine is a fragment of a polypeptide that retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding.

### 2. Payload

In some embodiments, the viral particle comprises a payload. In some embodiments, the payload is conjugated to the surface of the particle. In some embodiments, the payload is encapsulated by the particle. In some embodiments, the viral particle delivers a payload to a target cell.

In some embodiments, the payload is a nucleic acid. In some embodiments, the nucleic acid is a coding nucleic acid. In some embodiments, the nucleic acid encodes a polypeptide of interest. In some embodiments, the polypeptide of interest is a therapeutic polypeptide. In some embodiments, the polypeptide of interest is a chimeric antigen receptor. In some embodiments, the nucleic acid is transduced into a target cell and the polypeptide of interest is expressed in the target cell. In some embodiments, the nucleic acid is a non-coding nucleic acid. In some embodiments, then nucleic acid is a therapeutic non-coding nucleic acid. Non-coding nucleic acids are known to those of skill in the art and include but are not limited to siRNA, miRNA, and shRNA.

In some embodiments, expression of a payload is driven by a promoter. In some embodiments, the promoter is the MND promoter (myeloproliferative sarcoma virus enhancer, negative control region deleted, dl587rev primer-binding site substituted), which is a viral-derived synthetic promoter that contains the U3 region of a modified Moloney murine leukemia virus (MoMuLV) LTR with myeloproliferative sarcoma virus enhancer13 and has high expression in human CD34+ stem cells, lymphocytes, and other tissues. In some embodiments, separate proteins are expressed, separated by 2A peptide sequences that induce ribosomal skipping and cleavage during translation. In some embodiments, the promoter is a CMV promoter. In some embodiments, the promoter is the EF1a promoter. In other embodiments, the promoter is an HTLV promoter.

a. Synthetic Cytokine Receptor Complex

In some embodiments, the retroviral vector comprises a nucleotide sequence encoding a synthetic cytokine receptor complex. The synthetic cytokine receptors of the present disclosure comprise a synthetic gamma chain and a synthetic beta chain, each comprising a dimerization domain. The dimerization domains controllable dimerize in the present of a non-physiological ligand, thereby activating signaling the synthetic cytokine receptor. In a preferred embodiment, the non-physiological ligand is rapamycin or a rapalog, such synthetic cytokine receptor termed a rapamycin-activated cytokine receptor (RACR).

The synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2G intracellular domain.

The synthetic beta chain polypeptide comprises a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. The synthetic gamma chain polypeptide comprises a first dimerization domain, a first transmembrane domain, and an interleukin-2 receptor subunit gamma (IL-2RG) intracellular domain). The dimerization domain may be extracellular (N-terminal to the transmembrane domain) or intracellular (C-terminal to the transmembrane domain and N- or C-terminal to the IL-2RB or IL-7RB intracellular domain).

### i) Intracellular Domain

In some embodiments, the intracellular signaling domain of the first transmembrane receptor protein comprises an interleukin-2 receptor subunit gamma (IL2Rg) domain.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

### ii) Dimerization Domain

The dimerization domains may be heterodimerization domains, including but not limited to FK506-Binding Protein of size 12 kD (FKBP) and a FKBP12-rapamycin binding (FRB) domain, which are known in the art to dimerize in the presence of rapamycin or a rapalog.

Alternatively, the first dimerization domain and the second dimerization domain may be a FK506-Binding Protein of size 12 kD (FKBP) and a calcineurin domain, which are known in the art to dimerize in the presence of FK506 or an analogue thereof.

In some embodiments the dimerization domains are homodimerization domains selected from:
i) FK506-Binding Protein of size 12 kD (FKBP);
ii) cyclophiliA (CypA); or
iii) gyrase B (CyrB);
with the corresponding non-physiological ligands being, respectively
i) FK1012, AP1510, AP1903, or AP20187;
ii) cyclosporin-A (CsA); or
iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYR1-like 1 (PYL1) and abscisic acid insensitive 1 (ABI1).

iii) Transmembrane Domains

The transmembrane domain is the sequence of the synthetic cytokine receptor that spans the membrane. The transmembrane domain may comprise a hydrophobic alpha helix. In some embodiments, the transmembrane domain is derived from a human protein.

### iv) Cytosolic FRB

The FRB domain is an approximately 100 amino acid domain derived from the mTOR protein kinase. It may be expressed in the cytosol as a freely diffusible soluble protein. Advantageously, the FRB domain reduces the inhibitory effects of rapamycin on mTOR in the transduced cells and promote consistent activation of transduced cells giving the cells a proliferative advantage over native cells.

In some embodiments, synthetic cytokine receptor complex comprises a cytosolic polypeptide that binds to the ligand or a complex comprising the ligand.

In some embodiments, the cytosolic polypeptide comprises an FRB domain. In some embodiments, the cytosolic polypeptide comprises an FRB domain and the ligand is rapamycin. Advantageously, the cytosolic FRB confers resistance to the immunosuppressive effect of the non-physiological ligand (e.g., rapamycin or rapalog).

b. Chimeric Antigen Receptor

In some embodiments, the viral particles described herein are used to transduce a nucleic acid sequence (polynucleotide) encoding one or more chimeric antigen receptor (CARs) into a cell (e.g., a T lymphocyte). In some embodiments, the transduction of the viral particle results in expression of one or more CARs in the transduced cells.

Conventionally, CARs are generated by fusing a polynucleotide encoding a VL, VH, or scFv to the 5′ end of a polynucleotide encoding transmembrane and intracellular domains, and transducing cells with that polynucleotide as well as with the corresponding VH or VL, if needed. Numerous variations on CARs well known in the art and the disclosure contemplates using any of the known variations. Additionally, VL/VH pairs and scFvs for innumerable haptens are known in the art or can be generated by conventional methods routinely. Accordingly, the present disclosure contemplates using any known hapten-binding domain.

In some embodiments, the binding portion of the CAR can be, for example, a single chain fragment variable region (scFv) of an antibody, a Fab, Fv, Fc, or (Fab′)2 fragment, and the like. The use of unaltered (i.e., full size) antibodies, such as IgG, IgM, IgA, IgD or IgE, in the CAR or as the CAR is excluded from the scope of the invention.

In some embodiments, a co-stimulation domain serves to enhance the proliferation and survival of the lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, but are not limited to: CD28 (see, e.g., Alvarez-Vallina, L. et al., Eur J Immunol. 1996. 26(10):2304-9); CD137 (4-1BB), a member of the tumor necrosis factor (TNF) receptor family (see, e.g., Imai, C. et al., Leukemia. 2004. 18:676-84); and CD134 (OX40), a member of the TNFR-superfamily of receptors (see, e.g., Latza, U. et al., Eur. J. Immunol. 1994. 24:677). A skilled artisan will understand that sequence variants of these co-stimulation domains can be used, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In some embodiments of the invention, the CAR constructs comprise two co-stimulation domains. While the particular combinations include all possible variations of the four noted domains, specific examples include: 1) CD28+CD137 (4-1BB) and 2) CD28+CD134 (OX40).

In some embodiments, the activation signaling domain serves to activate cells upon binding of the CAR to a targeted moiety. The identity of the activation signaling domain is limited only in that it has the ability to induce activation of the selected cell upon binding of the targeted moiety by the CAR. Suitable activation signaling domains include the CD3ζ chain and Fc receptor γ. The skilled artisan will understand that sequence variants of these noted activation signaling domains can be used without adversely impacting the invention, where the variants have the same or similar activity as the domain on which they are modeled. Such variants may have at least about 80%, at least about 90%, at least about 95%, at least about 97%, at least about 98%, at least about 99%, or at least about 99.5% sequence identity to the amino acid sequence of the domain from which they are derived.

In some embodiments, the CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

In some embodiments, the payload may comprise a CAR-like construct. For example, the payload may comprise a nucleic acid that encodes both a receptor and a reporter. In some embodiments, this construct may comprise the structure S-ETD-MBD-IRES-R where S is a signal sequence, ETD comprises an extracellular targeting domain, MBD comprises a membrane-bound domain, IRES comprises an internal ribosome entry site, and R encodes a reporter. In some embodiments, the reporter may be a fluorescent protein or an antibiotic resistance marker. In some embodiments, the extracellular targeting domain may comprise an antibody, an antibody fragment, a small molecule ligand, or peptide. In some embodiments, the peptide may comprise some or all of a cytokine or interleukin.

Illustrative CAR constructs suitable for CAR-NK cells are provided below:
(1) scFv-CD8_{TM}-4-1BB_{IC}-CD3ζs (see, e.g., Liu E, Tong Y, Dotti G, et al., Leukemia. 2018; 32: 520-531);
(2) scFv-CD28_{TM+IC}-CD3ζs (see, e.g., Han J, Chu J, Keung C W et al., Sci Rep. 2015; 5: 11483; Kruschinski A, Moosmann A, Poschke I et al., Proc Natl Acad Sci USA. 2008; 105: 17481-17486; and Chu J, Deng Y, Benson D M et al., Leukemia. 2014; 28: 917-927);
(3) scFv-DAP12_{TM+IC} (see, e.g., Muller N, Michen S, Tietze S et al., J Immunother. 2015; 38: 197-210);
(4) scFv-CD8_{TM}-2B4_{IC}-CD3ζs (see, e.g., Xu Y, Liu Q, Zhong M et al., J Hematol Oncol. 2019; 12: 49);
(5) scFv-2B4_{TM+IC}-CD3ζs (see, e.g., Altvater B, Landmeier S, Pscherer S et al., Clin Cancer Res. 2009; 15: 4857-4866);
(6) scFv-CD28_{TM+IC}-4-1BB_{IC}-CD3ζs (see, e.g., Kloss S, Oberschmidt O, Morgan M et al., Hum Gene Ther. 2017; 28: 897-913);
(7) scFv-CD16_{TM}-2B4_{IC}-CD3ζs (see, e.g., Li Y, Hermanson D L, Moriarity B S Kaufman D S, Cell Stem Cell. 2018; 23: 181-192);
(8) scFv-NKp44_{TM}-DAP10_{IC}-CD3ζs (see, e.g., Li Y, Hermanson D L, Moriarity B S Kaufman D S, Cell Stem Cell. 2018; 23: 181-192);
(9) scFv-NKp46_{TM}-2B4_{IC}-CD3ζs (see, e.g., Li Y, Hermanson D L, Moriarity B S Kaufman D S, Cell Stem Cell. 2018; 23: 181-192);
(10) scFv-NKG2D_{TM}-2B4_{IC}-CD3ζs (see, e.g., Li Y, Hermanson D L, Moriarity B S Kaufman D S, Cell Stem Cell. 2018; 23: 181-192);
(11) scFv-NKG2D_{TM}-4-1BB_{IC}-CD3ζs (see, e.g., Li Y, Hermanson D L, Moriarity B S Kaufman D S, Cell Stem Cell. 2018; 23: 181-192);
(12) scFv-NKG2D_{TM}-2B4_{IC}-DAP12_{IC}-CD3ζs (see, e.g., Li Y, Hermanson D L, Moriarity B S Kaufman D S, Cell Stem Cell. 2018; 23: 181-192);
(13) scFv-NKG2D_{TM}-2B4_{IC}-DAP10_{IC}-CD3ζs (see, e.g., Li Y, Hermanson D L, Moriarity B S Kaufman D S, Cell Stem Cell. 2018; 23: 181-192);
(14) scFv-NKG2D_{TM}-4-1BB_{IC}-2B4_{IC}-CD3ζS (see, e.g., Li Y, Hermanson D L, Moriarity B S Kaufman D S, Cell Stem Cell. 2018; 23: 181-192); and
(15) scFv-NKG2D_{TM}-CD3ζS (see, e.g., Li Y, Hermanson D L, Moriarity B S Kaufman D S, Cell Stem Cell. 2018; 23: 181-192).

While the affinity at which the CARs, expressed by the lymphocytes, bind to the targeted moiety can vary, and in some cases low affinity binding may be preferable (such as about 50 nM), the binding affinity of the CARs to the targeted ligand will generally be at least about 100 nM, 1 μM, or 10 μM, preferably at least about 100 μM, 1 fM or 10 fM, even more preferably at least about 100 fM.

### i) CAR Extracellular Domain

In some embodiments, the CAR comprises an extracellular domain that binds to an antigen of interest. In some embodiments, the extracellular domain comprises a receptor, or a portion of a receptor, that binds to said antigen. In some embodiments, the extracellular domain comprises, or is, an antibody or an antigen-binding portion thereof. In some embodiments, the extracellular domain comprises, or is, a single-chain Fv domain. The single-chain Fv domain can comprise, for example, a VL linked to VH by a flexible linker, wherein said VL and VH are from an antibody that binds said antigen.

In some embodiments, the extracellular domain of CAR may contain any polypeptide that binds the desired antigen (e.g. prostate neoantigen). The extracellular domain may comprise a scFv, a portion of an antibody or an alternative scaffold. CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In some embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma. In some embodiments, in which the cancer is chronic lymphocytic leukemia (CLL), the B cells of the CLL have a normal karyotype. In some embodiments, in which the cancer is chronic lymphocytic leukemia (CLL), the B cells of the CLL carry a 17p deletion, an 11q deletion, a 12q trisomy, a 13q deletion or a p53 deletion.

In some embodiments, the antigen is expressed on a B-cell malignancy cell, relapsed/refractory CD19-expressing malignancy cell, diffuse large B-cell lymphoma (DLBCL) cell, Burkitt's type large B-cell lymphoma (B-LBL) cell, follicular lymphoma (FL) cell, chronic lymphocytic leukemia (CLL) cell, acute lymphocytic leukemia (ALL) cell, mantle cell lymphoma (MCL) cell, hematological malignancy cell, colon cancer cell, lung cancer cell, liver cancer cell, breast cancer cell, renal cancer cell, prostate cancer cell, ovarian cancer cell, skin cancer cell, melanoma cell, bone cancer cell, brain cancer cell, squamous cell carcinoma cell, leukemia cell, myeloma cell, B cell lymphoma cell, kidney cancer cell, uterine cancer cell, adenocarcinoma cell, pancreatic cancer cell, chronic myelogenous leukemia cell, glioblastoma cell, neuroblastoma cell, medulloblastoma cell, or a sarcoma cell.

In some embodiments, the antigen is a tumor-associated antigen (TAA) or a tumor-specific antigen (TSA). In some embodiments, without limitation, the tumor-associated antigen or tumor-specific antigen is B cell maturation antigen (BCMA), B cell Activating Factor (BAFF), GPRC5D, FCRL5, ROR1, L1-CAM, CD22, folate receptor, carboxy anhydrase IX (CAIX), claudin 18.2, FAP, mesothelin, IL13Ra2, Lewis Y, CCNA1, WT-1, TACI, CD38, SLAMF7, CD138, DLL3, transmembrane 4 L six family member 1 (TM4SF1), epithelial cell adhesion molecule (EpCAM), PD-1, PD-L1, CTLA-4, AXL, ROR2, glypican-3 (GPC3), CD133, CD147, EGFR, MUC1, GD2, Her2, prostate stem cell antigen (PSCA), prostate-specific membrane antigen (PSMA) alpha-fetoprotein (AFP), carcinoembryonic antigen (CEA), EGFRvIII, cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, vascular endothelial growth factor receptor (VEGFR), the dimeric form of the pyruvate kinase isoenzyme type M2 (tumor M2-PK), an abnormal ras protein, or an abnormal p53 protein.

In other embodiments, the CAR is a universal CAR and does not itself specifically target a tumor antigen. For example, the CAR could comprise a tag-specific scFv such that an exogenous agent comprising the tag and a tumor-targeting domain could direct the universal CAR T cell to the target tumor.

In some embodiments, the CAR is a second-generation CAR comprised of an anti-fluorescein scFv linked to the 4-1BB costimulatory domain and the CD3zeta intracellular signaling domain.

In some embodiments, the antigen is CD19. CARs targeting CD19 are described, for example, in US Publication No. 20160152723, U.S. Pat. Nos. 10,736,918, 10,357,514, and 7,446,190, each incorporated by reference.

In some embodiments, a CAR comprises an extracellular domain comprising a FMC63 scFv binding domain for CD19 binding. In some embodiments, the CAR is a second-generation CAR comprised of the FMC63 mouse anti-human CD19 scFv linked to the 4-1BB costimulatory domain and the CD3zeta intracellular signaling domain. In some embodiments, a CAR comprises a binding domain for CD19, a CD8a hinge, a CD8a transmembrane domain, a 4-1BB costimulatory domain, and a CD3zeta signaling domain. In some embodiments, a CAR comprises a binding domain for CD19, an IgG4 hinge, a CD28 transmembrane domain, a 4-1BB costimulatory domain, and a CD3zeta signaling domain. In some embodiments, a CAR comprises a binding domain for CD19, a CD28 hinge, a CD28 transmembrane domain, a CD28 costimulatory domain, and CD3zeta signaling domain. In some embodiments, a CAR comprises an extracellular domain comprising a FMC63 scFv binding domain for CD19 binding, a CD8a hinge, a CD8a transmembrane domain, a 4-1BB costimulatory domain, and a CD3zeta signaling domain. In some embodiments, a CAR comprises an extracellular domain comprising a FMC63 scFv binding domain for CD19 binding, an IgG4 hinge, a CD28 transmembrane domain, a 4-1BB costimulatory domain, and a CD3zeta signaling domain. In some embodiments, a CAR comprises an extracellular domain comprising a FMC63 scFv binding domain for CD19 binding, a CD28 hinge, a CD28 transmembrane domain, a CD28 costimulatory domain, and CD3zeta signaling domain.

In some embodiments, the CAR is a second-generation CAR comprised of the FMC63 mouse anti-human CD19 scFv linked to the CD28 costimulatory domain and the CD3zeta intracellular signaling domain. In some embodiments, the CAR is a second-generation CAR comprised of the FMC63 mouse anti-human CD19 scFv linked to a CD8 transmembrane domain, 4-1BB costimulatory domain, and the CD3zeta intracellular signaling domain.

In some embodiments, the antigen is BCMA. CAR T therapies targeting BCMA have been approved by the FDA and include Abecma and Carvykti. CARs targeting BCMA are described, for example, in US Publication No. 2020/0246381; U.S. Pat. No. 10,918,665; US Publication No. 2019/0161553, each of which is herein incorporated by reference. In some embodiments, a CAR comprises a binding domain for BCMA, a CD8a hinge, a CD8a transmembrane domain, a 4-1BB costimulatory domain, and a CD3zeta signaling domain. In some embodiments, a CAR comprises a binding domain for BCMA, an IgG4 hinge, a CD28 transmembrane domain, a 4-1BB costimulatory domain, and a CD3zeta signaling domain. In some embodiments, a CAR comprises a binding domain for BCMA, a CD28 hinge, a CD28 transmembrane domain, a CD28 costimulatory domain, and CD3zeta signaling domain.

In some embodiments, the antigen is G protein-coupled receptor class C group 5 member D (GPRC5D). CARs targeting GRC5D are described, for example, in US Publication Nos. 2018/0118803 and 2021/10393689, each of which is herein incorporated by reference. In some embodiments, a CAR comprises a binding domain for GRC5D, a CD8a hinge, a CD8a transmembrane domain, a 4-1BB costimulatory domain, and a CD3zeta signaling domain. In some embodiments, a CAR comprises a binding domain for GRC5D, an IgG4 hinge, a CD28 transmembrane domain, a 4-1BB costimulatory domain, and a CD3zeta signaling domain. In some embodiments, a CAR comprises a binding domain for GRC5D, a CD28 hinge, a CD28 transmembrane domain, a CD28 costimulatory domain, and CD3zeta signaling domain.

In some embodiments, the antigen is Fe Receptor-like 5 (FcRL5). CARs targeting FcRL5 are described, for example, in US Publication No. US 2017/0275362, which is herein incorporated by reference. In some embodiments, a CAR comprises a binding domain for FcRL5, a CD8a hinge, a CD8a transmembrane domain, a 4-1BB costimulatory domain, and a CD3zeta signaling domain. In some embodiments, a CAR comprises a binding domain for FcRL5, an IgG4 hinge, a CD28 transmembrane domain, a 4-1BB costimulatory domain, and a CD3zeta signaling domain. In some embodiments, a CAR comprises a binding domain for FcRL5, a CD28 hinge, a CD28 transmembrane domain, a CD28 costimulatory domain, and CD3zeta signaling domain.

In some embodiments, the antigen is receptor tyrosine kinase-like orphan receptor 1 (ROR1). CARs targeting ROR1 are described, for example, in US Publication No. 2022/0096651, which is herein incorporated by reference. In some embodiments, a CAR comprises a binding domain for ROR1, a CD8a hinge, a CD8a transmembrane domain, a 4-1BB costimulatory domain, and a CD3zeta signaling domain. In some embodiments, a CAR comprises a binding domain for ROR1, an IgG4 hinge, a CD28 transmembrane domain, a 4-1BB costimulatory domain, and a CD3zeta signaling domain. In some embodiments, a CAR comprises a binding domain for ROR1, a CD28 hinge, a CD28 transmembrane domain, a CD28 costimulatory domain, and CD3zeta signaling domain.

In some embodiments, the CAR is a second-generation CAR comprised an anti-BCMA scFv linked to the 4-1BB costimulatory domain and the CD3zeta intracellular signaling domain. In some embodiments, the CAR is a second-generation CAR comprised an anti-GPRC5D scFv linked to the 4-1BB costimulatory domain and the CD3zeta intracellular signaling domain. In some embodiments, the CAR is a second-generation CAR comprised an anti-ROR1 scFv linked to the 4-1BB costimulatory domain and the CD3zeta intracellular signaling domain.

In some embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In some embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In some embodiments, the TAA or TSA is alpha-actinin-4, Bage-1, BCR-ABL, Bcr-Abl fusion protein, beta-catenin, CA 125, CA 15-3 (CA 27.29BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAAO205, Mart2, Mum-1, 2, and 3, neo-PAP, myosin class I, OS-9, pml-RARa fusion protein, PTPRK, K-ras, N-ras, triosephosphate isomerase, Gage 3, 4, 5, 6, 7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, pi5(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68KP1, CO-029, FGF-5, G250, Ga733 (EpCAM), HTgp-175, M344, MA-50, MG7-Ag, MOV18, NB70K, NY-CO-1, RCAS1, SDCCAG16, TA-90, TAAL6, TAG72, TLP, TPS, CD19, CD20, CD22, CD27, CD30, CD70, CD123, CD133, B-cell maturation antigen, CS1, GPCR5, GD2 (ganglioside G2), EGFRvIII (epidermal growth factor variant III), sperm protein 17 (Spi7), mesothelin, PAP (prostatic acid phosphatase), prostein, TARP (T cell receptor gamma alternate reading frame protein), Trp-p8, STEAPi (six-transmembrane epithelial antigen of the prostate 1), an abnormal ras protein, or an abnormal p53 protein. In some embodiments, said tumor-associated antigen or tumor-specific antigen is integrin αvβ3 (CD61), galactin, K-Ras (V-Ki-ras2 Kirsten rat sarcoma viral oncogene), or Ral-B. Other tumor-associated and tumor-specific antigens are known to those in the art.

Antibodies, and scFvs, that bind to TSAs and TAAs include antibodies and scFVs that are known in the art, as are nucleotide sequences that encode them.

In some embodiments, the antigen is an antigen not considered to be a TSA or a TAA, but which is nevertheless associated with tumor cells, or damage caused by a tumor. In some embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in some embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In some embodiments, therefore, the antigen is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

In some embodiments of the polypeptides described herein, the extracellular domain is joined to said transmembrane domain directly or by a linker, spacer or hinge polypeptide sequence, e.g., a sequence from CD28 or a sequence from CTLA4.

In some embodiments, the extracellular domain that binds the desired antigen may be derived from antibodies or their antigen binding fragments generated using the technologies described herein.

An exemplary anti-CD19 CAR is shown in Table 1 with its different portions including the extracellular domain.

In some embodiments, the CAR is an anti-CD20 CAR and the extracellular binding domain of the CD20 CAR is specific to CD20, for example, human CD20. In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the VH, the VL, and/or one or more CDRs of any of the antibodies. An exemplary anti-CD20 CAR is shown in Table 2 and Table 3 with its different portions including its extracellular domain.

### Universal CARs

In some embodiments, the CAR targets a moiety that is not produced or expressed by cells of the subject being treated. This CAR thus allows for focused targeting of the cells to target cells, such as cancer cells. By administration of a small conjugate molecule the CAR cell response can be targeted to only those cells expressing the tumor receptor, thereby reducing off-target toxicity, and the activation of CAR cells can be more easily controlled due to the rapid clearance of the small conjugate molecule. As an added advantage, the CAR-expressing cells can be used as a “universal” cytotoxic cell to target a wide variety of tumors without the need to prepare separate CAR constructs. The targeted moiety recognized by the CAR may also remain constant. It is only the ligand portion of the small conjugate molecule that needs to be altered to allow the system to target cancer cells of different identity.

Various methods to target CARs and CAR-expressing cells have been described in the art, including, for example in US 2020/0123224, the disclosure of which is incorporated by reference herein. For example, a fluorescein or fluorescein isothiocyanate (FITC) moiety may be conjugated to an agent that binds to a desired target cell (such as a cancer cell), and thereby a CAR cell expressing an anti-fluorescein/FITC chimeric antigen receptor may be selectively targeted to the target cell labeled by the conjugate. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

In some embodiments, the CAR is a anti-FITC CAR and the ligand is composed of a fluorescein or fluorescein isothiocyanate (FITC) moiety conjugated to an agent that binds to a desired target cell (such as a cancer cell). Exemplary ligands are described below. In some embodiments, the ligand is FITC-folate.

An exemplary anti-FITC CAR is shown in Table 4 and its different portions.

In some embodiments, the CAR comprises an scFv domain. In some embodiments, the scFv domain comprises anti-fluorescein isothiocyanate (FITC) E2. In some embodiments, the scFv domain comprises a light chain variable domain (VL), a linker, and a heavy chain variable domain (VH).

In some embodiments, the E2 scFv VL comprises a nucleotide sequence at least 80%, 85%, 90%, 95%, 96%, 97%, 98%, 99% or 100% identical to the nucleotide sequence of SEQ ID NO:45. In some embodiments, the E2 scFv VL comprises a nucleotide sequence at least 80% identical to the nucleotide sequence of SEQ ID NO:45. In some embodiments, the E2 scFv VL comprises a nucleotide sequence at least 85% identical to the nucleotide sequence of SEQ ID NO:45. In some embodiments, the E2 scFv VL comprises a nucleotide sequence at least 90% identical to the nucleotide sequence of SEQ ID NO:45. In some embodiments, the E2 scFv VL comprises a nucleotide sequence at least 95% identical to the nucleotide sequence of SEQ ID NO:45. In some embodiments, the E2 scFv VL comprises a nucleotide sequence at least 96% identical to the nucleotide sequence of SEQ ID NO:45. In some embodiments, the E2 scFv VL comprises a nucleotide sequence at least 97% identical to the nucleotide sequence of SEQ ID NO:45. In some embodiments, the E2 scFv VL comprises a nucleotide sequence at least 98% identical to the nucleotide sequence of SEQ ID NO:45. In some embodiments, the E2 scFv VL comprises a nucleotide sequence at least 99% identical to the nucleotide sequence of SEQ ID NO:45. In some embodiments, the E2 scFv VL comprises a nucleotide sequence at least 100% identical to the nucleotide sequence of SEQ ID NO:45. In some embodiments, the E2 scFv VL comprises the nucleotide sequence of SEQ ID NO:45. In some embodiments, the E2 scFv VL consists of the nucleotide sequence of SEQ ID NO:45.

In some embodiments, the E2 scFv VL comprises an amino acid sequence at least 80%, 85%, 90%, 95%, 96%, 97%, 98%, 99% or 100% identical to the amino acid sequence of SEQ ID NO:47. In some embodiments, the E2 scFv VL comprises an amino acid sequence at least 80% identical to the amino acid sequence of SEQ ID NO:47. In some embodiments, the E2 scFv VL comprises an amino acid sequence at least 85% identical to the amino acid sequence of SEQ ID NO:47. In some embodiments, the E2 scFv VL comprises an amino acid sequence at least 90% identical to the amino acid sequence of SEQ ID NO:47. In some embodiments, the E2 scFv VL comprises an amino acid sequence at least 95% identical to the amino acid sequence of SEQ ID NO:47. In some embodiments, the E2 scFv VL comprises an amino acid sequence at least 96% identical to the amino acid sequence of SEQ ID NO:47. In some embodiments, the E2 scFv VL comprises an amino acid sequence at least 97% identical to the amino acid sequence of SEQ ID NO:47. In some embodiments, the E2 scFv VL comprises an amino acid sequence at least 98% identical to the amino acid sequence of SEQ ID NO:47. In some embodiments, the E2 scFv VL comprises an amino acid sequence at least 99% identical to the amino acid sequence of SEQ ID NO:47. In some embodiments, the E2 scFv VL comprises an amino acid sequence at least 100% identical to the amino acid sequence of SEQ ID NO:47. In some embodiments, the E2 scFv VL comprises the amino acid sequence of SEQ ID NO:47. In some embodiments, the E2 scFv VL consists the amino acid sequence of SEQ ID NO:47.

In some embodiments, the E2 scFv VH comprises a nucleotide sequence at least 80%, 85%, 90%, 95%, 96%, 97%, 98%, 99% or 100% identical to the nucleotide sequence of SEQ ID NO:48. In some embodiments, the E2 scFv VH comprises a nucleotide sequence at least 80% identical to the nucleotide sequence of SEQ ID NO:48. In some embodiments, the E2 scFv VH comprises a nucleotide sequence at least 85% identical to the nucleotide sequence of SEQ ID NO:48. In some embodiments, the E2 scFv VH comprises a nucleotide sequence at least 90% identical to the nucleotide sequence of SEQ ID NO:48. In some embodiments, the E2 scFv VH comprises a nucleotide sequence at least 95% identical to the nucleotide sequence of SEQ ID NO:48. In some embodiments, the E2 scFv VH comprises a nucleotide sequence at least 96% identical to the nucleotide sequence of SEQ ID NO:48. In some embodiments, the E2 scFv VH comprises a nucleotide sequence at least 97% identical to the nucleotide sequence of SEQ ID NO:48. In some embodiments, the E2 scFv VH comprises a nucleotide sequence at least 98% identical to the nucleotide sequence of SEQ ID NO:48. In some embodiments, the E2 scFv VH comprises a nucleotide sequence at least 99% identical to the nucleotide sequence of SEQ ID NO:48. In some embodiments, the E2 scFv VH comprises a nucleotide sequence at least 100% identical to the nucleotide sequence of SEQ ID NO:48. In some embodiments, the E2 scFv VH comprises the nucleotide sequence of SEQ ID NO:48. In some embodiments, the E2 scFv VH consists of the nucleotide sequence of SEQ ID NO:48.

In some embodiments, the E2 scFv VH comprises an amino acid sequence at least 80%, 85%, 90%, 95%, 96%, 97%, 98%, 99% or 100% identical to the amino acid sequence of SEQ ID NO:50. In some embodiments, the E2 scFv VH comprises an amino acid sequence at least 80% identical to the amino acid sequence of SEQ ID NO:50. In some embodiments, the E2 scFv VH comprises an amino acid sequence at least 85% identical to the amino acid sequence of SEQ ID NO:50. In some embodiments, the E2 scFv VH comprises an amino acid sequence at least 90% identical to the amino acid sequence of SEQ ID NO:50. In some embodiments, the E2 scFv VH comprises an amino acid sequence at least 95% identical to the amino acid sequence of SEQ ID NO:50. In some embodiments, the E2 scFv VH comprises an amino acid sequence at least 96% identical to the amino acid sequence of SEQ ID NO:50. In some embodiments, the E2 scFv VH comprises an amino acid sequence at least 97% identical to the amino acid sequence of SEQ ID NO:50. In some embodiments, the E2 scFv VH comprises an amino acid sequence at least 98% identical to the amino acid sequence of SEQ ID NO:50. In some embodiments, the E2 scFv VH comprises an amino acid sequence at least 99% identical to the amino acid sequence of SEQ ID NO:50. In some embodiments, the E2 scFv VH comprises an amino acid sequence at least 100% identical to the amino acid sequence of SEQ ID NO:50. In some embodiments, the E2 scFv VH comprises the amino acid sequence of SEQ ID NO:50. In some embodiments, the E2 scFv VH consists the amino acid sequence of SEQ ID NO:50.

In some embodiments, the E2 scFv linker comprises a nucleotide sequence at least 80%, 85%, 90%, 95%, 96%, 97%, 98%, 99% or 100% identical to the nucleotide sequence of SEQ ID NO:51. In some embodiments, the E2 scFv linker comprises a nucleotide sequence at least 80% identical to the nucleotide sequence of SEQ ID NO:51. In some embodiments, the E2 scFv linker comprises a nucleotide sequence at least 85% identical to the nucleotide sequence of SEQ ID NO:51. In some embodiments, the E2 scFv linker comprises a nucleotide sequence at least 90% identical to the nucleotide sequence of SEQ ID NO:51. In some embodiments, the E2 scFv linker comprises a nucleotide sequence at least 95% identical to the nucleotide sequence of SEQ ID NO:51. In some embodiments, the E2 scFv linker comprises a nucleotide sequence at least 96% identical to the nucleotide sequence of SEQ ID NO:51. In some embodiments, the E2 scFv linker comprises a nucleotide sequence at least 97% identical to the nucleotide sequence of SEQ ID NO:51. In some embodiments, the E2 scFv linker comprises a nucleotide sequence at least 98% identical to the nucleotide sequence of SEQ ID NO:51. In some embodiments, the E2 scFv linker comprises a nucleotide sequence at least 99% identical to the nucleotide sequence of SEQ ID NO:51. In some embodiments, the E2 scFv linker comprises a nucleotide sequence at least 100% identical to the nucleotide sequence of SEQ ID NO:51. In some embodiments, the E2 scFv linker comprises the nucleotide sequence of SEQ ID NO:51. In some embodiments, the E2 scFv linker consists the nucleotide sequence of SEQ ID NO:51.

In some embodiments, the E2 scFv linker comprises an amino acid sequence at least 80%, 85%, 90%, 95%, 96%, 97%, 98%, 99% or 100% identical to the amino acid sequence of SEQ ID NO:53. In some embodiments, the E2 scFv linker comprises an amino acid sequence at least 80% identical to the amino acid sequence of SEQ ID NO:53. In some embodiments, the E2 scFv linker comprises an amino acid sequence at least 85% identical to the amino acid sequence of SEQ ID NO:53. In some embodiments, the E2 scFv linker comprises an amino acid sequence at least 90% identical to the amino acid sequence of SEQ ID NO:53. In some embodiments, the E2 scFv linker comprises an amino acid sequence at least 95% identical to the amino acid sequence of SEQ ID NO:53. In some embodiments, the E2 scFv linker comprises an amino acid sequence at least 96% identical to the amino acid sequence of SEQ ID NO:53. In some embodiments, the E2 scFv linker comprises an amino acid sequence at least 97% identical to the amino acid sequence of SEQ ID NO:53. In some embodiments, the E2 scFv linker comprises an amino acid sequence at least 98% identical to the amino acid sequence of SEQ ID NO:53. In some embodiments, the E2 scFv linker comprises an amino acid sequence at least 99% identical to the amino acid sequence of SEQ ID NO:53. In some embodiments, the E2 scFv linker comprises an amino acid sequence at least 100% identical to the amino acid sequence of SEQ ID NO:53. In some embodiments, the E2 scFv linker comprises the amino acid sequence of SEQ ID NO:53. In some embodiments, the E2 scFv linker consists the amino acid sequence of SEQ ID NO:53.

In some embodiments, the E2 scFv comprises a nucleotide sequence at least 80%, 85%, 90%, 95%, 96%, 97%, 98%, 99% or 100% identical to the nucleotide sequence of SEQ ID NO:54. In some embodiments, the E2 scFv comprises a nucleotide sequence at least 80% identical to the nucleotide sequence of SEQ ID NO:54. In some embodiments, the E2 scFv comprises a nucleotide sequence at least 85% identical to the nucleotide sequence of SEQ ID NO:54. In some embodiments, the E2 scFv comprises a nucleotide sequence at least 90% identical to the nucleotide sequence of SEQ ID NO:54. In some embodiments, the E2 scFv comprises a nucleotide sequence at least 95% identical to the nucleotide sequence of SEQ ID NO:54. In some embodiments, the E2 scFv comprises a nucleotide sequence at least 96% identical to the nucleotide sequence of SEQ ID NO:54. In some embodiments, the E2 scFv comprises a nucleotide sequence at least 97% identical to the nucleotide sequence of SEQ ID NO:54. In some embodiments, the E2 scFv comprises a nucleotide sequence at least 98% identical to the nucleotide sequence of SEQ ID NO:54. In some embodiments, the E2 scFv comprises a nucleotide sequence at least 99% identical to the nucleotide sequence of SEQ ID NO:54. In some embodiments, the E2 scFv comprises a nucleotide sequence at least 100% identical to the nucleotide sequence of SEQ ID NO:54. In some embodiments, the E2 scFv comprises the nucleotide sequence of SEQ ID NO:54. In some embodiments, the E2 scFv consists of the nucleotide sequence of SEQ ID NO:54.

In some embodiments, the E2 scFv comprises an amino acid sequence at least 80%, 85%, 90%, 95%, 96%, 97%, 98%, 99% or 100% identical to the amino acid sequence of SEQ ID NO:56. In some embodiments, the E2 scFv comprises an amino acid sequence at least 80% identical to the amino acid sequence of SEQ ID NO:56. In some embodiments, the E2 scFv comprises an amino acid sequence at least 85% identical to the amino acid sequence of SEQ ID NO:56. In some embodiments, the E2 scFv comprises an amino acid sequence at least 90% identical to the amino acid sequence of SEQ ID NO:56. In some embodiments, the E2 scFv comprises an amino acid sequence at least 95% identical to the amino acid sequence of SEQ ID NO:56. In some embodiments, the E2 scFv comprises an amino acid sequence at least 96% identical to the amino acid sequence of SEQ ID NO:56. In some embodiments, the E2 scFv comprises an amino acid sequence at least 97% identical to the amino acid sequence of SEQ ID NO:56. In some embodiments, the E2 scFv comprises an amino acid sequence at least 98% identical to the amino acid sequence of SEQ ID NO:56. In some embodiments, the E2 scFv comprises an amino acid sequence at least 99% identical to the amino acid sequence of SEQ ID NO:56. In some embodiments, the E2 scFv comprises an amino acid sequence at least 100% identical to the amino acid sequence of SEQ ID NO:56. In some embodiments, the E2 scFv comprises the amino acid sequence of SEQ ID NO:56. In some embodiments, the E2 scFv consists of the amino acid sequence of SEQ ID NO:56.

In some embodiments, the CAR system utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275(11):26684-90), centyrins, and DARPins (see Reichert, J. M. MAbs 2009. 1(3):190-209).

In some embodiments, the cell-targeting moiety is DUPA (DUPA-(99m) Tc), a ligand bound by PSMA-positive human prostate cancer cells with nanomolar affinity (K_{D}14 nM; see Kularatne, S. A. et al., Mol Pharm. 2009. 6(3):780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013. ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega, E. I. et al., Cancer Metastasis Rev. 2008. 27(4):655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9. 10, 11, 12, 13, 14, 15, 16, 17, 18, 19, 20, 21, 22, 23, 24, 25, 26, 27, 28, 29, 30, 31, 32, 33, 34, 35, 36, 37. 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

Illustrative conjugates of the disclosure include the following molecules: FITC-(PEG)₁₂-Folate, FITC-(PEG)₂₀-Folate, FITC-(PEG)₁₀₈-Folate, FITC-DUPA, FITC-(PEG)₁₂-DUPA, FITC-CCK2R ligand, FITC-(PEG)₁₂-CCK2R ligand, FITC-(PEG)₁₁-NK1R ligand and FITC-(PEG)₂-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 μM or 10 μM, even more preferably at least about 100 μM.

Examples of conjugates and methods of making them are provided in U.S. patent applications US 2017/0290900, US 2019/0091308, and US 2020/0023009, all of which are incorporated herein by reference.

In some embodiments, the viral particle described herein comprises a nucleotide sequence encoding a universal, modular, anti-tag chimeric antigen receptor (UniCAR). This system allows for retargeting of UniCAR engrafted immune cells against multiple antigens (see e.g., US Patent Publication US20170240612 A1 incorporated herein by reference in its entirety; Cartellieri et al., (2016) Blood Cancer Journal 6, e458 incorporated herein by reference in its entirety).

In some embodiments, the viral particle described herein comprises a nucleotide sequence encoding a switchable CAR and/or CAR effector cell (CAR-EC) switches. In this system, the CAR-EC switches have a first region that is bound by a CAR on the CAR-EC and a second region that binds a cell surface molecule on a target cell, thereby stimulating an immune response from the CAR-EC that is cytotoxic to the bound target cell. In some embodiments, the CAR-EC switch may act as an “on-switch” for CAR-EC activity. Activity may be “turned off” by reducing or ceasing administration of the switch. These CAR-EC switches may be used with CAR-ECs disclosed herein, as well as existing CAR T-cells, for the treatment of a disease or condition, such as cancer, wherein the target cell is a malignant cell. Such treatment may be referred to herein as switchable immunotherapy (US Patent Publication U.S. Pat. No. 9,624,276 B2 incorporated herein by reference in its entirety).

In some embodiments, the viral particle comprises a nucleotide sequence encoding a universal immune receptor (e.g., switchable CAR, sCAR) that binds a peptide neo-epitope (PNE). In some embodiments, the peptide neo-epitope (PNE) has been incorporated at defined different locations within an antibody targeting an antigen (antibody switch). Therefore, sCAR-T-cell specificity is redirected only against PNE, not occurring in the human proteome, thus allowing an orthogonal interaction between the sCAR-T-cell and the antibody switch. In this way, sCAR-T cells are strictly dependent on the presence of the antibody switch to become fully activated, thus excluding CAR T-cell off-target recognition of endogenous tissues or antigens in the absence of the antibody switch (Arcangeli et al., (2016) Transl Cancer Res 5(Suppl 2):S174-S177 incorporated herein by reference in its entirety). Other examples of switchable CARs is provided by US Patent Application US20160272718A1 incorporated herein by reference in its entirety.

As used herein, the term “tag” encompasses a universal immune receptor, a tag, a switch, or an Fc region of an immunoglobulin as described supra. In some embodiments, a viral particle comprises a nucleotide sequence encoding a CAR comprising a tag binding domain. In some embodiments, the CAR binds fluorescein isothiocyanate (FITC), streptavidin, biotin, dinitrophenol, peridinin chlorophyll protein complex, green fluorescent protein, phycoerythrin (PE), horse radish peroxidase, palmitoylation, nitrosylation, alkalanine phosphatase, glucose oxidase, or maltose binding protein.

### ii) CAR Linker Region (e.g., Spacer)

The optional linker of CAR positioned between the extracellular domain and the transmembrane domain may be a polypeptide of about 2 to 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another.

In some embodiments, the linker is derived from a hinge region or portion of the hinge region of any immunoglobulin. In some embodiments, the linker is derived from IgG4. In some embodiments, the linker is derived from the extracellular domain of CD28. In other embodiments, the linker is derived from the extracellular domain of CD8. In some embodiments, the IgG hinge comprises an IgG1 hinge. In some embodiments, the hinge domain comprises a PD1 hinge.

In some embodiments, the CD8a hinge comprises a nucleotide sequence at least 80%, 85%, 90%, 95%, 96%, 97%, 98%, 99% or 100% identical to the nucleotide sequence of SEQ ID NO:57. In some embodiments, the CD8α hinge comprises a nucleotide sequence at least 80% identical to the nucleotide sequence of SEQ ID NO:57. In some embodiments, the CD8α hinge comprises a nucleotide sequence at least 85% identical to the nucleotide sequence of SEQ ID NO:57. In some embodiments, the CD8α hinge comprises a nucleotide sequence at least 90% identical to the nucleotide sequence of SEQ ID NO:57. In some embodiments, the CD8α hinge comprises a nucleotide sequence at least 95% identical to the nucleotide sequence of SEQ ID NO:57. In some embodiments, the CD8α hinge comprises a nucleotide sequence at least 96% identical to the nucleotide sequence of SEQ ID NO:57. In some embodiments, the CD8α hinge comprises a nucleotide sequence at least 97% identical to the nucleotide sequence of SEQ ID NO:57. In some embodiments, the CD8α hinge comprises a nucleotide sequence at least 98% identical to the nucleotide sequence of SEQ ID NO:57. In some embodiments, the CD8α hinge comprises a nucleotide sequence at least 99% identical to the nucleotide sequence of SEQ ID NO:57. In some embodiments, the CD8α hinge comprises a nucleotide sequence at least 100% identical to the nucleotide sequence of SEQ ID NO:57. In some embodiments, the CD8α hinge comprises the nucleotide sequence of SEQ ID NO:57. In some embodiments, the CD8α hinge consists of the nucleotide sequence of SEQ ID NO:57.

In some embodiments, the CD8α hinge comprises an amino acid sequence at least 80%, 85%, 90%, 95%, 96%, 97%, 98%, 99% or 100% identical to the amino acid sequence of SEQ ID NO:58. In some embodiments, the CD8α hinge comprises an amino acid sequence at least 80% identical to the amino acid sequence of SEQ ID NO:58. In some embodiments, the CD8α hinge comprises an amino acid sequence at least 85% identical to the amino acid sequence of SEQ ID NO:58. In some embodiments, the CD8α hinge comprises an amino acid sequence at least 90% identical to the amino acid sequence of SEQ ID NO:58. In some embodiments, the CD8α hinge comprises an amino acid sequence at least 95% identical to the amino acid sequence of SEQ ID NO:58. In some embodiments, the CD8α hinge comprises an amino acid sequence at least 96% identical to the amino acid sequence of SEQ ID NO:58. In some embodiments, the CD8α hinge comprises an amino acid sequence at least 97% identical to the amino acid sequence of SEQ ID NO:58. In some embodiments, the CD8α hinge comprises an amino acid sequence at least 98% identical to the amino acid sequence of SEQ ID NO:58. In some embodiments, the CD8α hinge comprises an amino acid sequence at least 99% identical to the amino acid sequence of SEQ ID NO:58. In some embodiments, the CD8α hinge comprises an amino acid sequence at least 100% identical to the amino acid sequence of SEQ ID NO:58. In some embodiments, the CD8α hinge comprises the amino acid sequence of SEQ ID NO:58. In some embodiments, the CD8α hinge consists of the amino acid sequence of SEQ ID NO:58.

In some embodiments, the CD8 hinge comprises a nucleotide sequence at least 80%, 85%, 90%, 95%, 96%, 97%, 98%, 99% or 100% identical to the nucleotide sequence of SEQ ID NO:59. In some embodiments, the CD8 hinge comprises a nucleotide sequence at least 80% identical to the nucleotide sequence of SEQ ID NO:59. In some embodiments, the CD8 hinge comprises a nucleotide sequence at least 85% identical to the nucleotide sequence of SEQ ID NO:59. In some embodiments, the CD8 hinge comprises a nucleotide sequence at least 90% identical to the nucleotide sequence of SEQ ID NO:59. In some embodiments, the CD8 hinge comprises a nucleotide sequence at least 95% identical to the nucleotide sequence of SEQ ID NO:59. In some embodiments, the CD8 hinge comprises a nucleotide sequence at least 96% identical to the nucleotide sequence of SEQ ID NO:59. In some embodiments, the CD8 hinge comprises a nucleotide sequence at least 97% identical to the nucleotide sequence of SEQ ID NO:59. In some embodiments, the CD8 hinge comprises a nucleotide sequence at least 98% identical to the nucleotide sequence of SEQ ID NO:59. In some embodiments, the CD8 hinge comprises a nucleotide sequence at least 99% identical to the nucleotide sequence of SEQ ID NO:59. In some embodiments, the CD8 hinge comprises a nucleotide sequence at least 100% identical to the nucleotide sequence of SEQ ID NO:59. In some embodiments, the CD8 hinge comprises the nucleotide sequence of SEQ ID NO:59. In some embodiments, the CD8 hinge consists of the nucleotide sequence of SEQ ID NO:59.

In some embodiments, the modified IgG4 hinge comprises an amino acid sequence at least 80%, 85%, 90%, 95%, 96%, 97%, 98%, 99% or 100% identical to the amino acid sequence of SEQ ID NO:60. In some embodiments, the modified IgG4 hinge comprises an amino acid sequence at least 80% identical to the amino acid sequence of SEQ ID NO:60. In some embodiments, the modified IgG4 hinge comprises an amino acid sequence at least 85% identical to the amino acid sequence of SEQ ID NO:60. In some embodiments, the modified IgG4 hinge comprises an amino acid sequence at least 90% identical to the amino acid sequence of SEQ ID NO:60. In some embodiments, the modified IgG4 hinge comprises an amino acid sequence at least 95% identical to the amino acid sequence of SEQ ID NO:60. In some embodiments, the modified IgG4 hinge comprises an amino acid sequence at least 96% identical to the amino acid sequence of SEQ ID NO:60. In some embodiments, the modified IgG4 hinge comprises an amino acid sequence at least 97% identical to the amino acid sequence of SEQ ID NO:60. In some embodiments, the modified IgG4 hinge comprises an amino acid sequence at least 98% identical to the amino acid sequence of SEQ ID NO:60. In some embodiments, the modified IgG4 hinge comprises an amino acid sequence at least 99% identical to the amino acid sequence of SEQ ID NO:60. In some embodiments, the modified IgG4 hinge comprises an amino acid sequence at least 100% identical to the amino acid sequence of SEQ ID NO:60. In some embodiments, the modified IgG4 hinge comprises the amino acid sequence of SEQ ID NO:60. In some embodiments, the modified IgG4 hinge consists of the amino acid sequence of SEQ ID NO:60.

In some embodiments, the modified IgG4 hinge comprises an amino acid sequence at least 80%, 85%, 90%, 95%, 96%, 97%, 98%, 99% or 100% identical to the amino acid sequence of SEQ ID NO:61. In some embodiments, the modified IgG4 hinge comprises an amino acid sequence at least 80% identical to the amino acid sequence of SEQ ID NO:61. In some embodiments, the modified IgG4 hinge comprises an amino acid sequence at least 85% identical to the amino acid sequence of SEQ ID NO:61. In some embodiments, the modified IgG4 hinge comprises an amino acid sequence at least 90% identical to the amino acid sequence of SEQ ID NO:61. In some embodiments, the modified IgG4 hinge comprises an amino acid sequence at least 95% identical to the amino acid sequence of SEQ ID NO:61. In some embodiments, the modified IgG4 hinge comprises an amino acid sequence at least 96% identical to the amino acid sequence of SEQ ID NO:61. In some embodiments, the modified IgG4 hinge comprises an amino acid sequence at least 97% identical to the amino acid sequence of SEQ ID NO:61. In some embodiments, the modified IgG4 hinge comprises an amino acid sequence at least 98% identical to the amino acid sequence of SEQ ID NO:61. In some embodiments, the modified IgG4 hinge comprises an amino acid sequence at least 99% identical to the amino acid sequence of SEQ ID NO:61. In some embodiments, the modified IgG4 hinge comprises an amino acid sequence at least 100% identical to the amino acid sequence of SEQ ID NO:61. In some embodiments, the modified IgG4 hinge comprises the amino acid sequence of SEQ ID NO:61. In some embodiments, the modified IgG4 hinge consists of the amino acid sequence of SEQ ID NO:61.

In some embodiments, the IgG1 hinge comprises an amino acid sequence at least 80%, 85%, 90%, 95%, 96%, 97%, 98%, 99% or 100% identical to the amino acid sequence of SEQ ID NO:62. In some embodiments, the IgG1 hinge comprises an amino acid sequence at least 80% identical to the amino acid sequence of SEQ ID NO:62. In some embodiments, the IgG1 hinge comprises an amino acid sequence at least 85% identical to the amino acid sequence of SEQ ID NO:62. In some embodiments, the IgG1 hinge comprises an amino acid sequence at least 90% identical to the amino acid sequence of SEQ ID NO:62. In some embodiments, the IgG1 hinge comprises an amino acid sequence at least 95% identical to the amino acid sequence of SEQ ID NO:62. In some embodiments, the IgG1 hinge comprises an amino acid sequence at least 96% identical to the amino acid sequence of SEQ ID NO:62. In some embodiments, the IgG1 hinge comprises an amino acid sequence at least 97% identical to the amino acid sequence of SEQ ID NO:62. In some embodiments, the IgG1 hinge comprises an amino acid sequence at least 98% identical to the amino acid sequence of SEQ ID NO:62. In some embodiments, the IgG1 hinge comprises an amino acid sequence at least 99% identical to the amino acid sequence of SEQ ID NO:62. In some embodiments, the IgG1 hinge comprises an amino acid sequence at least 100% identical to the amino acid sequence of SEQ ID NO:62. In some embodiments, the IgG1 hinge comprises the amino acid sequence of SEQ ID NO:62. In some embodiments, the IgG1 hinge consists of the amino acid sequence of SEQ ID NO:62.

In some embodiments, the PD1 hinge comprises an amino acid sequence at least 80%, 85%, 90%, 95%, 96%, 97%, 98%, 99% or 100% identical to the amino acid sequence of SEQ ID NO:63. In some embodiments, the PD1 hinge comprises an amino acid sequence at least 80% identical to the amino acid sequence of SEQ ID NO:63. In some embodiments, the PD1 hinge comprises an amino acid sequence at least 85% identical to the amino acid sequence of SEQ ID NO:63. In some embodiments, the PD1 hinge comprises an amino acid sequence at least 90% identical to the amino acid sequence of SEQ ID NO:63. In some embodiments, the PD1 hinge comprises an amino acid sequence at least 95% identical to the amino acid sequence of SEQ ID NO:63. In some embodiments, the PD1 hinge comprises an amino acid sequence at least 96% identical to the amino acid sequence of SEQ ID NO:63. In some embodiments, the PD1 hinge comprises an amino acid sequence at least 97% identical to the amino acid sequence of SEQ ID NO:63. In some embodiments, the PD1 hinge comprises an amino acid sequence at least 98% identical to the amino acid sequence of SEQ ID NO:63. In some embodiments, the PD1 hinge comprises an amino acid sequence at least 99% identical to the amino acid sequence of SEQ ID NO:63. In some embodiments, the PD1 hinge comprises an amino acid sequence at least 100% identical to the amino acid sequence of SEQ ID NO:63. In some embodiments, the PD1 hinge comprises the amino acid sequence of SEQ ID NO:63. In some embodiments, the PD1 hinge consists of the amino acid sequence of SEQ ID NO:63.

In some embodiments, the CD28 hinge comprises an amino acid sequence at least 80%, 85%, 90%, 95%, 96%, 97%, 98%, 99% or 100% identical to the amino acid sequence of SEQ ID NO:64. In some embodiments, the CD28 hinge comprises an amino acid sequence at least 80% identical to the amino acid sequence of SEQ ID NO:64. In some embodiments, the CD28 hinge comprises an amino acid sequence at least 85% identical to the amino acid sequence of SEQ ID NO:64. In some embodiments, the CD28 hinge comprises an amino acid sequence at least 90% identical to the amino acid sequence of SEQ ID NO:64. In some embodiments, the CD28 hinge comprises an amino acid sequence at least 95% identical to the amino acid sequence of SEQ ID NO:64. In some embodiments, the CD28 hinge comprises an amino acid sequence at least 96% identical to the amino acid sequence of SEQ ID NO:64. In some embodiments, the CD28 hinge comprises an amino acid sequence at least 97% identical to the amino acid sequence of SEQ ID NO:64. In some embodiments, the CD28 hinge comprises an amino acid sequence at least 98% identical to the amino acid sequence of SEQ ID NO:64. In some embodiments, the CD28 hinge comprises an amino acid sequence at least 99% identical to the amino acid sequence of SEQ ID NO:64. In some embodiments, the CD28 hinge comprises an amino acid sequence at least 100% identical to the amino acid sequence of SEQ ID NO:64. In some embodiments, the CD28 hinge comprises the amino acid sequence of SEQ ID NO:64. In some embodiments, the CD28 hinge consists of the amino acid sequence of SEQ ID NO:64

iii) CAR Transmembrane Region

The transmembrane region can be any transmembrane region that can be incorporated into a functional CAR, e.g., a transmembrane region from a CD28, CD4, or a CD8 molecule.

In some embodiments, the transmembrane domain of CAR may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of CAR may be derived from the transmembrane domain of CD28. In some embodiments, the transmembrane domain of a CAR may be derived from the transmembrane domain of CD8, for example, CD8α.

In some embodiments, the CD8 transmembrane domain comprises a nucleotide sequence at least 80%, 85%, 90%, 95%, 96%, 97%, 98%, 99% or 100% identical to the nucleotide sequence of SEQ ID NO:65. In some embodiments, the CD8 transmembrane domain comprises a nucleotide sequence at least 80% identical to the nucleotide sequence of SEQ ID NO:65. In some embodiments, the CD8 transmembrane domain comprises a nucleotide sequence at least 85% identical to the nucleotide sequence of SEQ ID NO:65. In some embodiments, the CD8 transmembrane domain comprises a nucleotide sequence at least 90% identical to the nucleotide sequence of SEQ ID NO:65. In some embodiments, the CD8 transmembrane domain comprises a nucleotide sequence at least 95% identical to the nucleotide sequence of SEQ ID NO:65. In some embodiments, the CD8 transmembrane domain comprises a nucleotide sequence at least 96% identical to the nucleotide sequence of SEQ ID NO:65. In some embodiments, the CD8 transmembrane domain comprises a nucleotide sequence at least 97% identical to the nucleotide sequence of SEQ ID NO:65. In some embodiments, the CD8 transmembrane domain comprises a nucleotide sequence at least 98% identical to the nucleotide sequence of SEQ ID NO:65. In some embodiments, the CD8 transmembrane domain comprises a nucleotide sequence at least 99% identical to the nucleotide sequence of SEQ ID NO:65. In some embodiments, the CD8 transmembrane domain comprises a nucleotide sequence at least 100% identical to the nucleotide sequence of SEQ ID NO:65. In some embodiments, the CD8 transmembrane domain comprises the nucleotide sequence of SEQ ID NO:65. In some embodiments, the CD8 transmembrane domain consists of the nucleotide sequence of SEQ ID NO:65.

In some embodiments, the CD8 transmembrane domain comprises an amino acid sequence at least 80%, 85%, 90%, 95%, 96%, 97%, 98%, 99% or 100% identical to the amino acid sequence of SEQ ID NO:66. In some embodiments, the CD8 transmembrane domain comprises an amino acid sequence at least 80% identical to the amino acid sequence of SEQ ID NO:66. In some embodiments, the CD8 transmembrane domain comprises an amino acid sequence at least 85% identical to the amino acid sequence of SEQ ID NO:66. In some embodiments, the CD8 transmembrane domain comprises an amino acid sequence at least 90% identical to the amino acid sequence of SEQ ID NO:66. In some embodiments, the CD8 transmembrane domain comprises an amino acid sequence at least 95% identical to the amino acid sequence of SEQ ID NO:66. In some embodiments, the CD8 transmembrane domain comprises an amino acid sequence at least 96% identical to the amino acid sequence of SEQ ID NO:66. In some embodiments, the CD8 transmembrane domain comprises an amino acid sequence at least 97% identical to the amino acid sequence of SEQ ID NO:66. In some embodiments, the CD8 transmembrane domain comprises an amino acid sequence at least 98% identical to the amino acid sequence of SEQ ID NO:66. In some embodiments, the CD8 transmembrane domain comprises an amino acid sequence at least 99% identical to the amino acid sequence of SEQ ID NO:66. In some embodiments, the CD8 transmembrane domain comprises an amino acid sequence at least 100% identical to the amino acid sequence of SEQ ID NO:66. In some embodiments, the CD8 transmembrane domain comprises the amino acid sequence of SEQ ID NO:66. In some embodiments, the CD8 transmembrane domain consists of the amino acid sequence of SEQ ID NO:66.

### iv) Intracellular Domains

In some embodiments, the intracellular domain of the CAR is or comprises an intracellular domain or motif of a protein that is expressed on the surface of T lymphocytes and triggers activation and/or proliferation of said T lymphocytes. Such a domain or motif is able to transmit a primary antigen-binding signal that is necessary for the activation of a T lymphocyte in response to the antigen's binding to the CAR's extracellular portion. In some embodiments, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In some embodiments, the intracellular domain is a CD3ζ intracellular signaling domain. In some embodiments, the intracellular domain is from a lymphocyte receptor chain, a TCR/CD3 complex protein, an Fc receptor subunit or an IL-2 receptor subunit. In some embodiments, the intracellular signaling domain of CAR may be derived from the signaling domains of for example CD3ζ, CD3ε, CD22, CD79a, CD66d or CD39. “Intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

In some embodiments, the intracellular domain of the CAR is the zeta CD3 chain (CD3zeta).

In some embodiments, the CAR additionally comprises one or more co-stimulatory domains or motifs, e.g., as part of the intracellular domain of the polypeptide. Co-stimulatory molecules are well-known cell surface molecules other than antigen receptors or Fc receptors that provide a second signal required for efficient activation and function of T lymphocytes upon binding to antigen. The one or more co-stimulatory domains or motifs can, for example, be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof. In some embodiments, the one or more co-stimulatory domains are selected from the group consisting of intracellular domains of 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

In some embodiments, the co-stimulatory domain is an intracellular domain of 4-1BB, CD28, or OX40. Exemplary CAR constructs comprising a CD28 signaling domain are disclosed in U.S. Pat. No. 7,446,190, incorporated by reference. Exemplary CAR constructs comprising a 4-1BB signaling domain are disclosed in U.S. Pat. Nos. 9,856,322 and 8,399,964, each incorporated by reference.

In some embodiments, the viral particle encodes a CAR comprising an IgG4 linker operatively linked to a CD28 transmembrane domain operatively linked to a 4-1BB co-stimulatory domain operatively linked to a CD3zeta signaling domain.

In some embodiments, the viral particle encodes a CAR comprising an CD8a linker operatively linked to a CD8a transmembrane domain operatively linked to a 4-1BB co-stimulatory domain operatively linked to a CD3zeta signaling domain.

In some embodiments, the viral particle encodes a CAR comprising an CD28 linker operatively linked to a CD28 transmembrane domain operatively linked to a CD28 co-stimulatory domain operatively linked to a CD3zeta signaling domain.

In some embodiments, the intracellular domain can be further modified to encode a detectable, for example, a fluorescent, protein (e.g., green fluorescent protein) or any variants known thereof.

In some embodiments, the 4-1BB endodomain comprises a nucleotide sequence at least 80%, 85%, 90%, 95%, 96%, 97%, 98%, 99% or 100% identical to the nucleotide sequence of SEQ ID NO:67. In some embodiments, the 4-1BB endodomain comprises a nucleotide sequence at least 80% identical to the nucleotide sequence of SEQ ID NO:67. In some embodiments, the 4-1BB endodomain comprises a nucleotide sequence at least 85% identical to the nucleotide sequence of SEQ ID NO:67. In some embodiments, the 4-1BB endodomain comprises a nucleotide sequence at least 90% identical to the nucleotide sequence of SEQ ID NO:67. In some embodiments, the 4-1BB endodomain comprises a nucleotide sequence at least 95% identical to the nucleotide sequence of SEQ ID NO:67. In some embodiments, the 4-1BB endodomain comprises a nucleotide sequence at least 96% identical to the nucleotide sequence of SEQ ID NO:67. In some embodiments, the 4-1BB endodomain comprises a nucleotide sequence at least 97% identical to the nucleotide sequence of SEQ ID NO:67. In some embodiments, the 4-1BB endodomain comprises a nucleotide sequence at least 98% identical to the nucleotide sequence of SEQ ID NO:67. In some embodiments, the 4-1BB endodomain comprises a nucleotide sequence at least 99% identical to the nucleotide sequence of SEQ ID NO:67. In some embodiments, the 4-1BB endodomain comprises a nucleotide sequence at least 100% identical to the nucleotide sequence of SEQ ID NO:67. In some embodiments, the 4-1BB endodomain comprises the nucleotide sequence of SEQ ID NO:67. In some embodiments, the 4-1BB endodomain consists the nucleotide sequence of SEQ ID NO:67.

In some embodiments, the 4-1BB endodomain comprises an amino acid sequence at least 80%, 85%, 90%, 95%, 96%, 97%, 98%, 99% or 100% identical to the amino acid sequence of SEQ ID NO:68. In some embodiments, the 4-1BB endodomain comprises an amino acid sequence at least 80% identical to the amino acid sequence of SEQ ID NO:68. In some embodiments, the 4-1BB endodomain comprises an amino acid sequence at least 85% identical to the amino acid sequence of SEQ ID NO:68. In some embodiments, the 4-1BB endodomain comprises an amino acid sequence at least 90% identical to the amino acid sequence of SEQ ID NO:68. In some embodiments, the 4-1BB endodomain comprises an amino acid sequence at least 95% identical to the amino acid sequence of SEQ ID NO:68. In some embodiments, the 4-1BB endodomain comprises an amino acid sequence at least 96% identical to the amino acid sequence of SEQ ID NO:68. In some embodiments, the 4-1BB endodomain comprises an amino acid sequence at least 97% identical to the amino acid sequence of SEQ ID NO:68. In some embodiments, the 4-1BB endodomain comprises an amino acid sequence at least 98% identical to the amino acid sequence of SEQ ID NO:68. In some embodiments, the 4-1BB endodomain comprises an amino acid sequence at least 99% identical to the amino acid sequence of SEQ ID NO:68. In some embodiments, the 4-1BB endodomain comprises an amino acid sequence at least 100% identical to the amino acid sequence of SEQ ID NO:68. In some embodiments, the 4-1BB endodomain comprises the amino acid sequence of SEQ ID NO:68. In some embodiments, the 4-1BB endodomain consists of the amino acid sequence of SEQ ID NO:68.

In some embodiments, the CD3ζ endodomain comprises a nucleotide sequence at least 80%, 85%, 90%, 95%, 96%, 97%, 98%, 99% or 100% identical to the nucleotide sequence of SEQ ID NO:69. In some embodiments, the CD3ζ endodomain comprises a nucleotide sequence at least 80% identical to the nucleotide sequence of SEQ ID NO:69. In some embodiments, the CD3ζ endodomain comprises a nucleotide sequence at least 85% identical to the nucleotide sequence of SEQ ID NO:69. In some embodiments, the CD3ζ endodomain comprises a nucleotide sequence at least 90% identical to the nucleotide sequence of SEQ ID NO:69. In some embodiments, the CD3ζ endodomain comprises a nucleotide sequence at least 95% identical to the nucleotide sequence of SEQ ID NO:69. In some embodiments, the CD3ζ endodomain comprises a nucleotide sequence at least 96% identical to the nucleotide sequence of SEQ ID NO:69. In some embodiments, the CD3ζ endodomain comprises a nucleotide sequence at least 97% identical to the nucleotide sequence of SEQ ID NO:69. In some embodiments, the CD3ζ endodomain comprises a nucleotide sequence at least 98% identical to the nucleotide sequence of SEQ ID NO:69. In some embodiments, the CD3ζ endodomain comprises a nucleotide sequence at least 99% identical to the nucleotide sequence of SEQ ID NO:69. In some embodiments, the CD3ζ endodomain comprises a nucleotide sequence at least 100% identical to the nucleotide sequence of SEQ ID NO:69. In some embodiments, the CD3ζ endodomain comprises the nucleotide sequence of SEQ ID NO:69. In some embodiments, the CD3ζ endodomain consists the nucleotide sequence of SEQ ID NO:69.

In some embodiments, the CD3ζ endodomain comprises an amino acid sequence at least 80%, 85%, 90%, 95%, 96%, 97%, 98%, 99% or 100% identical to the amino acid sequence of SEQ ID NO:70. In some embodiments, the CD3ζ endodomain comprises an amino acid sequence at least 80% identical to the amino acid sequence of SEQ ID NO:70. In some embodiments, the CD3ζ endodomain comprises an amino acid sequence at least 85% identical to the amino acid sequence of SEQ ID NO:70. In some embodiments, the CD3ζ endodomain comprises an amino acid sequence at least 90% identical to the amino acid sequence of SEQ ID NO:70. In some embodiments, the CD3ζ endodomain comprises an amino acid sequence at least 95% identical to the amino acid sequence of SEQ ID NO:70. In some embodiments, the CD3ζ endodomain comprises an amino acid sequence at least 96% identical to the amino acid sequence of SEQ ID NO:70. In some embodiments, the CD3ζ endodomain comprises an amino acid sequence at least 97% identical to the amino acid sequence of SEQ ID NO:70. In some embodiments, the CD3ζ endodomain comprises an amino acid sequence at least 98% identical to the amino acid sequence of SEQ ID NO:70. In some embodiments, the CD3ζ endodomain comprises an amino acid sequence at least 99% identical to the amino acid sequence of SEQ ID NO:70. In some embodiments, the CD3ζ endodomain comprises an amino acid sequence at least 100% identical to the amino acid sequence of SEQ ID NO:70. In some embodiments, the CD3ζ endodomain comprises the amino acid sequence of SEQ ID NO:70. In some embodiments, the CD3ζ endodomain consists of the amino acid sequence of SEQ ID NO:70.

### v) Exemplary CARs

In some embodiments the CAR is an anti-CD19 CAR. In some embodiments, the CD19 CAR may comprise a signal peptide, an extracellular binding domain that specifically binds CD19, a hinge domain, a transmembrane domain, an intracellular costimulatory domain, and an intracellular activation signaling domain. In some embodiments, the anti-CD19 CAR comprises the features set forth in Table 1.

TABLE 1
Exemplary sequences of anti-CD19 CAR and components

| | | Nucleotide | Amino Acid |
|---|---|---|---|
| | Component | (SEQ ID NO) | (SEQ ID NO) |
| Full Anti-CD19 CAR | | 71 | 72 |
| Extracellular | CD19 scFv | 73 | 74 |
| binding domain | CD19 scFv VL | 75 | 76 |
| | linker | 77 | 78 |
| | CD19 scFv VH | 79 | 80 |
| Hinge and | CD8 hinge | 81 | 82 |
| transmembrane | CD28 | 83 | 84 |
| domain | transmembrane | | |
| Costimulatory | 4-1BB | 85 | 68 |
| signaling domain | | | |
| Activation | CD3 zeta | 86 | 70 |
| signaling domain | | | |

In some embodiments, the anti-CD19 CAR is encoded by a nucleotide that is at least 80%, 85%, 90%, 95%, 96%, 97%, 98%, 99% or 100% identical to the nucleotide sequence of SEQ ID NO:73. In some embodiments, the anti-CD19 CAR is encoded by a nucleotide sequence that is at least 80% identical to the nucleotide sequence of SEQ ID NO:73. In some embodiments, the anti-CD19 CAR is encoded by a nucleotide sequence that is at least 85% identical to the nucleotide sequence of SEQ ID NO:73. In some embodiments, the anti-CD19 CAR is encoded by a nucleotide sequence that is at least 90% identical to the nucleotide sequence of SEQ ID NO:73. In some embodiments, the anti-CD19 CAR is encoded by a nucleotide sequence that is at least 95% identical to the nucleotide sequence of SEQ ID NO:73. In some embodiments, the anti-CD19 CAR is encoded by a nucleotide sequence that is at least 96% identical to the nucleotide sequence of SEQ ID NO:73. In some embodiments, the anti-CD19 CAR is encoded by a nucleotide sequence that is at least 97% identical to the nucleotide sequence of SEQ ID NO:73. In some embodiments, the anti-CD19 CAR is encoded by a nucleotide sequence that is at least 98% identical to the nucleotide sequence of SEQ ID NO:73. In some embodiments, the anti-CD19 CAR is encoded by a nucleotide sequence that is at least 99% identical to the nucleotide sequence of SEQ ID NO:73. In some embodiments, the anti-CD19 CAR is encoded by a nucleotide sequence that is at least 100% identical to the nucleotide sequence of SEQ ID NO:73. In some embodiments, the anti-CD19 CAR is encoded by a nucleotide sequence comprising the nucleotide sequence of SEQ ID NO:73. In some embodiments, the anti-CD19 CAR is encoded by a nucleotide sequence consisting of the nucleotide sequence of SEQ ID NO:73.

In some embodiments, the anti-CD19 CAR comprises an amino acid sequence at least 80%, 85%, 90%, 95%, 96%, 97%, 98%, 99% or 100% identical to the amino acid sequence of SEQ ID NO:74. In some embodiments, the anti-CD19 CAR comprises an amino acid sequence at least 80% identical to the amino acid sequence of SEQ ID NO:74. In some embodiments, the anti-CD19 CAR comprises an amino acid sequence at least 85% identical to the amino acid sequence of SEQ ID NO:74. In some embodiments, the anti-CD19 CAR comprises an amino acid sequence at least 90% identical to the amino acid sequence of SEQ ID NO:74. In some embodiments, the anti-CD19 CAR comprises an amino acid sequence at least 95% identical to the amino acid sequence of SEQ ID NO:74. In some embodiments, the anti-CD19 CAR comprises an amino acid sequence at least 96% identical to the amino acid sequence of SEQ ID NO:74. In some embodiments, the anti-CD19 CAR comprises an amino acid sequence at least 97% identical to the amino acid sequence of SEQ ID NO:74. In some embodiments, the anti-CD19 CAR comprises an amino acid sequence at least 98% identical to the amino acid sequence of SEQ ID NO:74. In some embodiments, the anti-CD19 CAR comprises an amino acid sequence at least 99% identical to the amino acid sequence of SEQ ID NO:74. In some embodiments, the anti-CD19 CAR comprises an amino acid sequence at least 100% identical to the amino acid sequence of SEQ ID NO:74. In some embodiments, the anti-CD19 CAR comprises an amino acid sequence comprising the sequence of SEQ ID NO:74. In some embodiments, the anti-CD19 CAR comprises an amino acid sequence consisting of the sequence of SEQ ID NO:74.

In some embodiments the CAR is an anti-CD20 CAR. In some embodiments, the CD20 CAR may comprise a signal peptide, an extracellular binding domain that specifically binds CD20, a flag domain, a hinge domain, a transmembrane domain, an intracellular costimulatory domain, and an intracellular activation signaling domain. In some embodiments, the CD20 CAR may comprise a signal peptide, an extracellular binding domain that specifically binds CD20, a hinge domain, a transmembrane domain, an intracellular costimulatory domain, and an intracellular activation signaling domain. In some embodiments, the an anti-CD20 CAR comprises the features set forth in Table 2 (anti-CD2 CAR with flag) or Table 3 (anti-CD20 CAR without flag).

TABLE 2
Exemplary sequences of anti-CD20
CAR and components (with flag)

| | | Nucleotide | Amino Acid |
|---|---|---|---|
| | Component | (SEQ ID NO) | (SEQ ID NO) |
| Full Anti-CD20 CAR | | 87 | 88 |
| Extracellular | CD20 scFv | 89 | 90 |
| binding domain | CD20 scFv VL | 91 | 92 |
| | 218 linker | 93 | 94 |
| | CD20 scFv VH | 95 | 96 |
| Flag domain | Flag | 97 | 98 |
| Hinge and | CD8 hinge and | 99 | 100 |
| transmembrane | transmembrane | | |
| domain | | | |
| Costimulatory | 4-1BB | 85 | 68 |
| signaling domain | | | |
| Activation | CD3 zeta | 101 | 70 |
| signaling domain | | | |

TABLE 3
Exemplary sequences of anti-CD20
CAR and components (without flag)

| | | Nucleotide | Amino Acid |
|---|---|---|---|
| | Component | (SEQ ID NO) | (SEQ ID NO) |
| Full Anti-CD20 CAR | | 129 | 130 |
| Extracellular | CD20 scFv | 89 | 90 |
| binding domain | CD20 scFv VL | 91 | 92 |
| | 218 linker | 93 | 94 |
| | CD20 scFv VH | 95 | 96 |
| Hinge and | CD8 hinge and | 99 | 100 |
| transmembrane | transmembrane | | |
| domain | | | |
| Costimulatory | 4-1BB | 85 | 68 |
| signaling domain | | | |
| Activation | CD3 zeta | 101 | 70 |
| signaling domain | | | |

In some embodiments, the anti-CD20 CAR is encoded by a nucleotide that is at least 80%, 85%, 90%, 95%, 96%, 97%, 98%, 99% or 100% identical to the nucleotide sequence of SEQ ID NO:87. In some embodiments, the anti-CD20 CAR is encoded by a nucleotide sequence that is at least 80% identical to the nucleotide sequence of SEQ ID NO: 87. In some embodiments, the anti-CD20 CAR is encoded by a nucleotide sequence that is at least 85% identical to the nucleotide sequence of SEQ ID NO:87. In some embodiments, the anti-CD20 CAR is encoded by a nucleotide sequence that is at least 90% identical to the nucleotide sequence of SEQ ID NO:87. In some embodiments, the anti-CD20 CAR is encoded by a nucleotide sequence that is at least 95% identical to the nucleotide sequence of SEQ ID NO:87. In some embodiments, the anti-CD20 CAR is encoded by a nucleotide sequence that is at least 96% identical to the nucleotide sequence of SEQ ID NO:87. In some embodiments, the anti-CD20 CAR is encoded by a nucleotide sequence that is at least 97% identical to the nucleotide sequence of SEQ ID NO:87. In some embodiments, the anti-CD20 CAR is encoded by a nucleotide sequence that is at least 98% identical to the nucleotide sequence of SEQ ID NO:87. In some embodiments, the anti-CD20 CAR is encoded by a nucleotide sequence that is at least 99% identical to the nucleotide sequence of SEQ ID NO:87. In some embodiments, the anti-CD20 CAR is encoded by a nucleotide sequence that is at least 100% identical to the nucleotide sequence of SEQ ID NO:87. In some embodiments, the anti-CD20 CAR is encoded by a nucleotide sequence comprising the nucleotide sequence of SEQ ID NO:87. In some embodiments, the anti-CD20 CAR is encoded by a nucleotide sequence consisting of the nucleotide sequence of SEQ ID NO:87.

In some embodiments, the anti-CD20 CAR comprises an amino acid sequence at least 80%, 85%, 90%, 95%, 96%, 97%, 98%, 99% or 100% identical to the amino acid sequence of SEQ ID NO:88. In some embodiments, the anti-CD20 CAR comprises an amino acid sequence at least 80% identical to the amino acid sequence of SEQ ID NO:88. In some embodiments, the anti-CD20 CAR comprises an amino acid sequence at least 85% identical to the amino acid sequence of SEQ ID NO:88. In some embodiments, the anti-CD20 CAR comprises an amino acid sequence at least 90% identical to the amino acid sequence of SEQ ID NO:88. In some embodiments, the anti-CD20 CAR comprises an amino acid sequence at least 95% identical to the amino acid sequence of SEQ ID NO:88. In some embodiments, the anti-CD20 CAR comprises an amino acid sequence at least 96% identical to the amino acid sequence of SEQ ID NO:88. In some embodiments, the anti-CD20 CAR comprises an amino acid sequence at least 97% identical to the amino acid sequence of SEQ ID NO:88. In some embodiments, the anti-CD20 CAR comprises an amino acid sequence at least 98% identical to the amino acid sequence of SEQ ID NO:88. In some embodiments, the anti-CD20 CAR comprises an amino acid sequence at least 99% identical to the amino acid sequence of SEQ ID NO:88. In some embodiments, the anti-CD20 CAR comprises an amino acid sequence at least 100% identical to the amino acid sequence of SEQ ID NO:88. In some embodiments, the anti-CD20 CAR comprises an amino acid sequence comprising the sequence of SEQ ID NO:88. In some embodiments, the anti-CD20 CAR comprises an amino acid sequence consisting of the sequence of SEQ ID NO:88.

In some embodiments, the anti-CD20 CAR is encoded by a nucleotide that is at least 80%, 85%, 90%, 95%, 96%, 97%, 98%, 99% or 100% identical to the nucleotide sequence of SEQ ID NO:129. In some embodiments, the anti-CD20 CAR is encoded by a nucleotide sequence that is at least 80% identical to the nucleotide sequence of SEQ ID NO:129. In some embodiments, the anti-CD20 CAR is encoded by a nucleotide sequence that is at least 85% identical to the nucleotide sequence of SEQ ID NO:129. In some embodiments, the anti-CD20 CAR is encoded by a nucleotide sequence that is at least 90% identical to the nucleotide sequence of SEQ ID NO:129. In some embodiments, the anti-CD20 CAR is encoded by a nucleotide sequence that is at least 95% identical to the nucleotide sequence of SEQ ID NO:129. In some embodiments, the anti-CD20 CAR is encoded by a nucleotide sequence that is at least 96% identical to the nucleotide sequence of SEQ ID NO:129. In some embodiments, the anti-CD20 CAR is encoded by a nucleotide sequence that is at least 97% identical to the nucleotide sequence of SEQ ID NO:129. In some embodiments, the anti-CD20 CAR is encoded by a nucleotide sequence that is at least 98% identical to the nucleotide sequence of SEQ ID NO:129. In some embodiments, the anti-CD20 CAR is encoded by a nucleotide sequence that is at least 99% identical to the nucleotide sequence of SEQ ID NO:129. In some embodiments, the anti-CD20 CAR is encoded by a nucleotide sequence that is at least 100% identical to the nucleotide sequence of SEQ ID NO:129. In some embodiments, the anti-CD20 CAR is encoded by a nucleotide sequence comprising the nucleotide sequence of SEQ ID NO:129. In some embodiments, the anti-CD20 CAR is encoded by a nucleotide sequence consisting of the nucleotide sequence of SEQ ID NO:129.

In some embodiments, the anti-CD20 CAR comprises an amino acid sequence at least 80%, 85%, 90%, 95%, 96%, 97%, 98%, 99% or 100% identical to the amino acid sequence of SEQ ID NO:130. In some embodiments, the anti-CD20 CAR comprises an amino acid sequence at least 80% identical to the amino acid sequence of SEQ ID NO:130. In some embodiments, the anti-CD20 CAR comprises an amino acid sequence at least 85% identical to the amino acid sequence of SEQ ID NO:130. In some embodiments, the anti-CD20 CAR comprises an amino acid sequence at least 90% identical to the amino acid sequence of SEQ ID NO: 130. In some embodiments, the anti-CD20 CAR comprises an amino acid sequence at least 95% identical to the amino acid sequence of SEQ ID NO:130. In some embodiments, the anti-CD20 CAR comprises an amino acid sequence at least 96% identical to the amino acid sequence of SEQ ID NO:130. In some embodiments, the anti-CD20 CAR comprises an amino acid sequence at least 97% identical to the amino acid sequence of SEQ ID NO:130. In some embodiments, the anti-CD20 CAR comprises an amino acid sequence at least 98% identical to the amino acid sequence of SEQ ID NO:130. In some embodiments, the anti-CD20 CAR comprises an amino acid sequence at least 99% identical to the amino acid sequence of SEQ ID NO:130. In some embodiments, the anti-CD20 CAR comprises an amino acid sequence at least 100% identical to the amino acid sequence of SEQ ID NO:130. In some embodiments, the anti-CD20 CAR comprises an amino acid sequence comprising the sequence of SEQ ID NO:130. In some embodiments, the anti-CD20 CAR comprises an amino acid sequence consisting of the sequence of SEQ ID NO:130.

In some embodiments, the CAR is a universal CAR with the features set forth in Table 4. In some embodiments, the CAR is an anti-FITC CAR.

TABLE 4
Exemplary sequences of anti-FITC CAR and components

| | | Nucleotide | Amino Acid |
|---|---|---|---|
| | Component | (SEQ ID NO) | (SEQ ID NO) |
| Full Anti-FITC Tag CAR | | 102, 103 | 104, 105 |
| Extracellular | E2 scFv | 106 | 107 |
| binding domain | E2 scFv VL | 108 | 109 |
| | linker | 110 | 111 |
| | E2 scFv VH | 112 | 113 |
| Hinge and | CD8 hinge and | 114 | 115 |
| transmembrane | transmembrane | | |
| domain | CD8 hinge | 116 | 117 |
| | CD8 | 118 | 119 |
| | transmembrane | | |
| Costimulatory | 4-1BB | 67 | 68 |
| signaling domain | | | |
| Activation | CD3 zeta | 69 | 70 |
| signaling domain | | | |

In some embodiments, the anti-FITC CAR is encoded by a nucleotide that is at least 80%, 85%, 90%, 95%, 96%, 97%, 98%, 99% or 100% identical to the nucleotide sequence of SEQ ID NOs:102 or 103. In some embodiments, the anti-FITC CAR is encoded by a nucleotide sequence that is at least 80% identical to the nucleotide sequence of SEQ ID NOs:102 or 103. In some embodiments, the anti-FITC CAR is encoded by a nucleotide sequence that is at least 85% identical to the nucleotide sequence of SEQ ID NOs:102 or 103. In some embodiments, the anti-FITC CAR is encoded by a nucleotide sequence that is at least 90% identical to the nucleotide sequence of SEQ ID NOs:102 or 103. In some embodiments, the anti-FITC CAR is encoded by a nucleotide sequence that is at least 95% identical to the nucleotide sequence of SEQ ID NOs:102 or 103. In some embodiments, the anti-FITC CAR is encoded by a nucleotide sequence that is at least 96% identical to the nucleotide sequence of SEQ ID NOs:102 or 103. In some embodiments, the anti-FITC CAR is encoded by a nucleotide sequence that is at least 97% identical to the nucleotide sequence of SEQ ID NOs:102 or 103. In some embodiments, the anti-FITC CAR is encoded by a nucleotide sequence that is at least 98% identical to the nucleotide sequence of SEQ ID NOs:102 or 103. In some embodiments, the anti-FITC CAR is encoded by a nucleotide sequence that is at least 99% identical to the nucleotide sequence of SEQ ID NOs:102 or 103. In some embodiments, the anti-FITC CAR is encoded by a nucleotide sequence that is at least 100% identical to the nucleotide sequence of SEQ ID NOs:102 or 103. In some embodiments, the anti-FITC CAR is encoded by a nucleotide sequence comprising the nucleotide sequence of SEQ ID NOs:102 or 103. In some embodiments, the anti-FITC CAR is encoded by a nucleotide sequence consisting of the nucleotide sequence of SEQ ID NOs:102 or 103.

In some embodiments, the anti-FITC CAR comprises an amino acid sequence at least 80%, 85%, 90%, 95%, 96%, 97%, 98%, 99% or 100% identical to the amino acid sequence of SEQ ID NOs:104 or 105. In some embodiments, the anti-FITC CAR comprises an amino acid sequence at least 80% identical to the amino acid sequence of SEQ ID NOs:104 or 105. In some embodiments, the anti-FITC CAR comprises an amino acid sequence at least 85% identical to the amino acid sequence of SEQ ID NOs:104 or 105. In some embodiments, the anti-FITC CAR comprises an amino acid sequence at least 90% identical to the amino acid sequence of SEQ ID NOs:104 or 105. In some embodiments, the anti-FITC CAR comprises an amino acid sequence at least 95% identical to the amino acid sequence of SEQ ID NOs:104 or 105. In some embodiments, the anti-FITC CAR comprises an amino acid sequence at least 96% identical to the amino acid sequence of SEQ ID NOs:104 or 105. In some embodiments, the anti-FITC CAR comprises an amino acid sequence at least 97% identical to the amino acid sequence of SEQ ID NOs:104 or 105. In some embodiments, the anti-FITC CAR comprises an amino acid sequence at least 98% identical to the amino acid sequence of SEQ ID NOs:104 or 105. In some embodiments, the anti-FITC CAR comprises an amino acid sequence at least 99% identical to the amino acid sequence of SEQ ID NOs:104 or 105. In some embodiments, the anti-FITC CAR comprises an amino acid sequence at least 100% identical to the amino acid sequence of SEQ ID NOs:104 or 105. In some embodiments, the anti-FITC CAR comprises an amino acid sequence comprising the sequence of SEQ ID NOs:104 or 105. In some embodiments, the anti-FITC CAR comprises an amino acid sequence consisting of the sequence of SEQ ID NOs:104 or 105.

### III. PHARMACEUTICAL COMPOSITIONS

The disclosure provides a formulation (pharmaceutical composition) for treating an individual by gene therapy, wherein the composition comprises a therapeutically effective amount of a viral particle prepared by the methods disclosed herein. The pharmaceutical composition may be for human or animal usage. Typically, a physician will determine the actual dosage which will be most suitable for an individual subject and it will vary with the age, weight and response of the particular individual. The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise, or in addition to, the carrier, excipient or diluent any suitable binders), lubricant(s), suspending agent(s), coating agent(s), solubilising agent(s), and other carrier agents that may aid or increase the viral entry into the target site (such as for example a lipid delivery system).

The formulations and compositions of the present disclosure may comprise a combination of any number of viral particles, and optionally one or more additional pharmaceutical agents (polypeptides, polynucleotides, compounds etc.) formulated in pharmaceutically acceptable or physiologically-acceptable compositions for administration to a cell, tissue, organ, or an animal, either alone, or in combination with one or more other modalities of therapy. In some embodiments, the one or more additional pharmaceutical agent further increases transduction efficiency of vectors.

In some embodiments, the present disclosure provides compositions comprising a therapeutically-effective amount of a viral particle, as described herein, formulated together with one or more pharmaceutically acceptable carriers (additives) and/or diluents. In some embodiments, the composition further comprises other agents, such as, e.g., cytokines, growth factors, hormones, small molecules or various pharmaceutically active agents.

In some embodiments, compositions and formulations of the viral particles used in accordance with the present disclosure may be prepared for storage by mixing a viral particle having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed. In some embodiments, one or more pharmaceutically acceptable surface-active agents (surfactant), buffers, isotonicity agents, salts, amino acids, sugars, stabilizers and/or antioxidant are used in the formulation.

Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers and phosphate-buffers.

Isotonicity agents are used to provide an isotonic formulation. An isotonic formulation is liquid, or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

Non-limiting examples of salts include salts of any combinations of the cations sodium potassium, calcium or magnesium with anions chloride, phosphate, citrate, succinate, sulphate or mixtures thereof. Non-limiting examples of amino acids comprise arginine, glycine, ornithine, lysine, histidine, glutamic acid, asparagic acid, isoleucine, leucine, alanine, phenylalanine, tyrosine, tryptophane, methionine, serine, proline. Non-limiting examples of sugars according to the invention include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Non-limiting examples of stabilizer includes amino acids and sugars as described above as well as commercially available cyclodextrins and dextrans of any kind and molecular weight as known in the art. Non-limiting examples of antioxidants include excipients such as methionine, benzylalcohol or any other excipient used to minimize oxidation.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the present disclosure is contemplated.

The compositions may further comprise one or more polypeptides, polynucleotides, vectors comprising same, compounds that increase the transduction efficiency of vectors, formulated in pharmaceutically acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules or various pharmaceutically active agents. There is virtually no limit to other components that may also be included in the compositions, provided that the additional agents do not adversely affect the ability of the composition to deliver the intended therapy.

The present disclosure also provides pharmaceutical compositions comprising an expression cassette or vector (e.g., therapeutic vector) disclosed herein and one or more pharmaceutically acceptable carriers, diluents or excipients. In some embodiments, the pharmaceutical composition comprises a lentiviral vector comprising an expression cassette disclosed herein, e.g., wherein the expression cassette comprises one or more polynucleotide sequences encoding one or more chimeric antigen receptor (CARs) and variants thereof.

The pharmaceutical compositions that contain the expression cassette or vector may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The vector can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the vectors according to the present disclosure.

In some embodiments, the viral particle (e.g., lentiviral particle), or a pharmaceutical composition comprising that viral particle, is effective when administered systemically. For example, the viral vectors of the disclosure, in some cases, demonstrate efficacy when administered intravenously to subject (e.g., a primate, such as a non-human primate or a human). In some embodiments, the viral vectors of the disclosure are capable of inducing expression of CAR in various immune cells when administered systemically (e.g., in T-cells, dendritic cells, NK cells).

In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral vectors general contains salts to prevent aggregation and other excipients (e.g., poloxamer) to reduce stickiness of the viral particle. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In some embodiments, the formulation is stable for storage and use when frozen (e.g., at less than 0° C., about −60° C., or about −72° C.). In some embodiments, the formulation is a cryopreserved solution.

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intraperitoneal, and intramuscular administration and formulation.

In certain circumstances, it will be desirable to deliver the compositions disclosed herein parenterally, intravenously, intramuscularly, or intraperitoneally, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In some embodiments, isotonic agents, for example, sugars or sodium chloride, are added. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2005). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

In some embodiments, the present disclosure provides formulations or compositions suitable for the delivery of viral vector systems (i.e., viral-mediated transduction) including, but not limited to, retroviral (e.g., lentiviral) vectors.

The present disclosure further contemplates that one or more additional agents that improve the transduction efficiency of viral particle may be used.

### IV. METHODS OF TREATMENT

In some embodiments, purified viral particles of the disclosure may be administered to a human subject. In some embodiments, purified viral particles of the disclosure is used as a method of treatment for a disease. In some embodiments, purified viral particles of the disclosure is part of one or more anti-cancer therapies. In some embodiments, the lentivirus formulation is for in vivo administration to a subject.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotozumab, ixazomib, melphalan, dexamethasone, vincristine, cyclophosphamide, hydroxy daunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

In some embodiments, the one or more agents to be administered with or after the viral particle comprises one or more adaptor molecules. In some embodiments, these adaptor molecules may comprise a targeting moiety and a hapten. In such embodiments, the viral particle may comprise a sequence encoding a hapten-specific CAR. Exemplary combinations are disclosed in WO 2021/076788 and US 20170290900, each of which is incorporated herein in its entirety.

### A. Ex Vivo Cell Therapies

In some aspects, the viral particles of the disclosure may be used in delivering a nucleic acid to a cell ex vivo. In some embodiments, the disclosure provides viral particles for delivering a nucleic acid to an immune cell ex vivo. In some embodiments, the viral particles of the disclosure activate and transduce an immune cell ex vivo.

In some embodiments, the disclosure provides viral particles for delivering a nucleic acid to a cell in an ex vivo CAR T manufacturing process. Such methods typically involve the isolation of PBMCs from a patient via leukapheresis. These cells are washed and optionally further purified via one or more selection steps to isolate particular T cell populations of interest. In some aspects, these might include CD4+ and/or CD8+ T cells. The washed and/or purified cells may be activated and then transduced using a lentiviral vector. The activation step may comprise contacting the cells with an exogenous activation agent such as anti-CD3 and anti-CD28 antibodies bound to a substrate or using unbound antibodies. Exemplary activation agents include anti-CD3 and anti-CD28-presenting beads and/or soluble polymers. After transduction, the cells may be optionally further washed and cultured until harvest. Methods of manufacturing engineered cell therapies, including CAR T cells, are known in the art (see e.g., Abou-el-Enein, M. et al. Blood Cancer Discov (2021), Vol 2(5): 408-422; Arcangeli, S. et al. Front. Immunol (19 Jun. 2020), Vol. 11 (1217) 1-13; Ghassemi, S. et al. Nat Biomed Eng (February 2022), Vol 6(2): 118-128; Vormittag, P. et al. Curr Opin Biotechnol (October 2018), Vol. 54: 164-181; each of which is herein incorporated by reference). Exemplary methods of autologous CAR T manufacturing are disclosed in US Patent Publication Nos. 2019/0269727, 2016/0122782, 2021/0163893, and US 2017/0037369, each of which is incorporated herein in its entirety.

In some embodiments, the disclosure provides viral particles for delivering a nucleic acid to a cell in an ex-vivo closed-loop manufacturing process. In some embodiments, the lentiviral vectors disclosed herein permit delivery of a nucleic acid to a target cell during a closed-loop process. Exemplary methods of closed-loop processes are disclosed in US Patent Publication No. 2021/0244871 and WO2022072885, both of which are incorporated herein in their entirety.

In some embodiments, the lentiviral vectors as disclosed herein eliminate the need for an ex-vivo activation step. In such embodiments, the isolated cells could be transduced directly after leukapheresis, washing, or selection.

### B. Diseases

The disclosure also provides a viral particle that can be used for treatment of diseases, disorders or conditions. In some embodiments, the disease or disorder is cancer. In some embodiments, the cancer is a hematological malignancy or a solid tumor. In some embodiments, the subject is relapsed or refractory to treatment with a prior anti-cancer therapeutic.

In some embodiments, a therapeutic application of the viral particles disclosed herein is to treat malignancies that have failed other non-CAR T-cell treatment options.

In some embodiments, the cancer is a hematological malignancy.

In some embodiments, the hematological malignancy is lymphoma, a B cell malignancy, Hodgkin's lymphoma, non-Hodgkin's lymphoma, a DLBLC, a FL, a MCL, a marginal zone B-cell lymphoma (MZL), a mucosa-associated lymphatic tissue lymphoma (MALT), a CLL, an ALL, an AML, Waldenstrom's Macroglobulinemia or a T-cell lymphoma.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the at least one genetic abnormality is a translocation between chromosomes 8 and 21, a translocation or an inversion in chromosome 16, a translocation between chromosomes 15 and 17, changes in chromosome 11, or mutation in fins-related tyrosine kinase 3 (FLT3), nucleophosmin (NPM1), isocitrate dehydrogenase 1 (IDH1), isocitrate dehydrogenase 2 (IDH2), DNA (cytosine-5)-methyltransferase 3 (DNMT3A), CCAAT/enhancer binding protein alpha (CEBPA), U2 small nuclear RNA auxiliary factor 1 (U2AF1), enhancer of zeste 2 polycomb repressive complex 2 subunit (EZH2), structural maintenance of chromosomes 1A (SMC1A) or structural maintenance of chromosomes 3 (SMC3).

In some embodiments, the hematological malignancy is an ALL. In some embodiments, the ALL is B-cell lineage ALL, T-cell lineage ALL, adult ALL or pediatric ALL.

In some embodiments, the subject with ALL has a Philadelphia chromosome or is resistant or has acquired resistance to treatment with a BCR-ABL kinase inhibitor. The Ph chromosome is present in about 20% of adults with ALL and a small percentage of children with ALL and is associated with poor prognosis. At a time of relapse, patients with Ph+ positive ALL may be on tyrosine kinase inhibitor (TKI) regimen and may have therefore become resistant to the TKI. The viral particles as described herein may thus be administered to a subject who has become resistant to selective or partially selective BCR-ABL inhibitors. Exemplary BCR-ABL inhibitors are for example imatinib, dasatinib, nilotinib, bosutinib, ponatinib, bafetinib, saracatinib, tozasertib or danusertib.

In some embodiments, the subject has ALL with t(v; 11q23) (MLL rearranged), t(1; 19)(q23; p13.3); TCF3-PBX1 (E2A-PBX1), t(12; 21)(p13; q22); ETV6-RUNX1 (TEL-AML1) or t(5; 14)(q31; q32); IL3-IGH chromosomal rearrangement. Chromosomal rearrangements can be identified using well known methods, for example fluorescent in situ hybridization, karyotyping, pulsed field gel electrophoresis, or sequencing.

In some embodiments, the hematological malignancy is the smoldering multiple myeloma, MGUS, ALL, DLBLC, BL, FL, MCL, Waldenstrom's macroglobulinema, plasma cell leukemia, AL, precursor B-cell lymphoblastic leukemia, precursor B-cell lymphoblastic leukemia, myelodysplastic syndrome (MDS), CLL, B cell malignancy, CML, HCL, blastic plasmacytoid dendritic cell neoplasm, Hodgkin's lymphoma, non-Hodgkin's lymphoma, MZL, MALT, plasma cell leukemia, ALCL, leukemia, or lymphoma.

In some embodiments, the cancer is diffuse large B-cell lymphoma (DLBCL). In some embodiments, the cancer is Burkitt's type large B-cell lymphoma (B-LBL). In some embodiments, the cancer is follicular lymphoma (FL). In some embodiments, the cancer is chronic lymphocytic leukemia (CLL). In some embodiments, the cancer is acute lymphocytic leukemia (ALL). In some embodiments, the cancer is mantle cell lymphoma (MCL).

In some embodiments, the cancer is a solid tumor.

In some embodiments, the solid tumor is a lung cancer, a non-small cell lung cancer (NSCLC), a liver cancer, a cervical cancer, a colon cancer, a breast cancer, an ovarian cancer, an endometrial cancer, a pancreatic cancer, a melanoma, an esophageal cancer, a gastric cancer, a stomach cancer, a renal carcinoma, a bladder cancer, a hepatocellular carcinoma, a renal cell carcinoma, an urothelial carcinoma, a head and neck cancer, a glioma, a glioblastoma, a colorectal cancer, a thyroid cancer, epithelial cancers, or adenocarcinomas. WO2019057124A1 discloses cancers that are amenable to treatment with T cell redirecting therapeutics that bind CD19.

In some embodiments, the solid tumor is a prostate cancer. the prostate cancer is a relapsed prostate cancer. In some embodiments, the prostate cancer is a refractory prostate cancer. In some embodiments, the prostate cancer is a malignant prostate cancer. In some embodiments, the prostate cancer is a castration resistant prostate cancer.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### V. DEFINITIONS

Unless otherwise defined in the disclosure, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described in the disclosure, are those well-known and commonly used in the art.

As used in the disclosure, the following terms have the meanings ascribed to them unless specified otherwise.

The articles “a,” “an,” and “the” are used in the disclosure to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used in the disclosure, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises,” and “comprising” will be understood to imply the inclusion of a stated step or element or group of steps or elements but not the exclusion of any other step or element or group of steps or elements. In some embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used in the disclosure, the term “isolated” means material that is substantially or essentially free from components that normally accompany it in its native state. In some embodiments, the term “obtained” or “derived” is used synonymously with isolated. A “subject,” “individual,” or “patient” as used in the disclosure, includes any animal that exhibits a symptom of a condition that can be detected or identified with compositions of the disclosure. Suitable subjects include laboratory animals (such as mouse, rat, rabbit, or guinea pig), farm animals (such as horses, cows, sheep, pigs), and domestic animals or pets (such as a cat or dog). In some embodiments, the subject is a mammal. In certain embodiments, the subject is a non-human primate and, in preferred embodiments, the subject is a human.

### VI. EXEMPLARY EMBODIMENTS

Among the provided embodiments are:
1. A method for preparing a lentivirus formulation, comprising
   (i) filtering a suspension mixture comprising a population of host cells and lentiviral particles to remove contaminants, wherein filtering comprises:
      (a) filtering the mixture with a first filter, wherein the first filter is a depth filter, resulting in a first filtrate,
      (b) filtering the first filtrate with a second filter, wherein the second filter has a retention threshold smaller than the first filter, resulting in a second filtrate, and
      (c) filtering the second filtrate, with a third filter, wherein the third filter has a retention threshold smaller than the second filter, thereby producing a filtered formulation of lentiviral particles; and
   (ii) concentrating the filtered formulation of lentiviral particles, wherein concentrating comprises chromatography and ultrafiltration.
2. A method for preparing a lentivirus formulation, comprising
   (i) contacting a population of host cells in suspension with at least one plasmid encoding a lentiviral protein;
   (ii) culturing the population of host cells of step (i) for a period of time sufficient to produce a suspension mixture comprising a population of host cells and lentiviral particles;
   (iii) filtering the suspension mixture to remove contaminants, comprising:
      (a) contacting the mixture with an endonuclease,
      (b) filtering the mixture with a first filter, wherein the first filter is a depth filter, resulting in a first filtrate
      (c) filtering the first filtrate with a second filter, wherein the second filter has a retention threshold smaller than the first filter, resulting in a second filtrate, and
      (d) filtering the second filtrate with a third filter, wherein the third filter has a retention threshold smaller than the second filter, thereby producing a filtered formulation of lentiviral particles; and
   (iv) concentrating the filtered formulation of lentiviral particles, wherein concentrating comprises chromatography and ultrafiltration.
3. The method of embodiment 1 or 2, wherein the host cell comprises a human cell.
4. The method of embodiment 3, wherein the human cell comprises a HEK293 cell, a HEK293T cell, a HEK293F cell, a HEK293FT cell, a Te671 cell, a HT1080 cell, or a CEM cell.
5. The method of embodiment 3 or 4, wherein the cell comprises a HEK293 cell.
6. The method of embodiment 3 or 4, wherein the cell comprises a HEK293T cell.
7. The method of any one of embodiments 1-6, wherein the first filter has a retention threshold of 1-60 μm.
8. The method of embodiment 7, wherein the first filter has a retention threshold of 60 μm.
9. The method of any one of embodiments 1-8, wherein the second filter has a retention threshold of 0.4-4 μm.
10. The method of embodiment 9, wherein the second filter has a retention threshold of 0.45 μm.
11. The method of any of embodiments 1-10, wherein the third filter has a retention threshold of 0.45 μm±0.2 μm.
12. The method of any one of embodiments 1-10, wherein the third filter has a retention threshold of 0.2-0.3 μm.
13. The method of embodiment 12, wherein the third filter has a retention threshold of 0.2 μm.
14. The method of any one of embodiments 1-10, 12 and 13, wherein the first filter has a retention threshold of 60 μm, the second filter has a retention threshold of 0.45 μm, and the third filter has a retention threshold of 0.2 μm.
15. The method of any one of embodiments 1 and 3-14, wherein an endonuclease is present through steps (i)(a)-(i)(c).
16. The method of any one of embodiments 2-15, wherein the endonuclease is present through steps (iii)(b)-(iii)(d).
17. The method of any one of embodiments 1-16, wherein the second filter and the third filter are two layers within a dual-layer filter component.
18. The method of any one of embodiments 1-16, wherein the third filter is a dual-layer filter comprising a first layer filter and a second layer filter, wherein the second layer filter has a retention threshold smaller than the first layer filter.
19. The method of embodiment 18, wherein the retention threshold of the first filter is 60 μm, the retention threshold of the second filter is 0.45 μm, the retention threshold of the first layer filter is 0.45 μm, and the retention threshold of the second layer filter is 0.2 μm.
20. The method of any one of embodiments 1-19, wherein the chromatography is anion exchange chromatography (AEX).
21. The method of embodiment 20, wherein the AEX chromatography comprises eluting the lentiviral particles with a salt buffer.
22. The method of embodiment 21, wherein the salt buffer comprises NaCl.
23. The method of embodiment 22, wherein the NaCl is at a concentration from about 0.5M to 3M.
24. The method of embodiment 22, wherein the NaCl is at a concentration from about 0.5 M to 1 M.
25. The method of any of embodiments 22-24, wherein the NaCl is or about 0.75M.
26. The method of embodiment 22, wherein the NaCl is at a concentration from about 1M to 3 M.
27. The method of embodiment 22, wherein the NaCl is at a concentration from about 1.5 M to 2.5 M.
28. The method of any of embodiments 22, 26 or 27, wherein the NaCl is about 2M.
29. The method of any one of embodiments 1-28, wherein the chromatography is performed before ultrafiltration.
30. The method of any one of embodiments 1-29, wherein the ultrafiltration is ultrafiltration/diafiltration (UF/DF).
31. The method of embodiment 30, wherein the UF/DF is by one or more tangential flow filtration (TFF) steps.
32. The method of embodiment 31, wherein the filter of the one or more TFF is a hollow fiber filter.
33. The method of embodiment 32, wherein the nominal molecular weight cutoff (NMWC) of the hollow fiber filter is or is about 500 kDa.
34. The method of any of embodiments 31-33, wherein the TFF comprises a first tangential flow filtration (TFF) step and second tangential flow filtration (TFF) step.
35. The method of embodiment 33, wherein the first TFF is performed with a first hollow fiber filter and the second TFF is performed with a second hollow fiber filter.
36. The method of embodiment 35, wherein the first and second hollow fiber filter have the same nominal molecular weight cutoff (NMWC).
37. The method of embodiment 36, wherein the NMWC is 500 kDa.
38. The method of embodiment 35, wherein the first hollow fiber filter has a greater nominal molecular weight cutoff (NMWC) than the second hollow fiber filter.
39. The method of embodiment 38, wherein the NMWC of the first hollow fiber filter is 500 kDa.
40. The method of any of embodiments 35-39, wherein the first hollow fiber filter has a greater surface area than the second hollow fiber filter.
41. The method of embodiment 40, wherein the surface area of the first hollow fiber filter is between 790 cm² to 1600 cm².
42. The method of any of embodiments 35-41, wherein the first hollow fiber filter holds a larger volume than the second hollow fiber filter.
43. The method of embodiment 42, wherein the volume of the first hollow fiber filter is 300 mL.
44. The method of any one of embodiments 1-43, comprising sterilizing filtration of the filtered formulation after concentration, thereby producing a sterilized formulation.
45. The method of embodiment 44, wherein sterilizing filtration comprises filtering the filtered formulation with a fourth filter.
46. The method of embodiment 45, wherein the fourth filter has a retention threshold of 0.2 μm.
47. The method of any one of embodiments 44-46, comprising formulating the sterilized formulation in a buffer, thereby producing a drug substance.
48. The method of any one of embodiments 1-47, wherein the method occurs at a pH of 6-8.
49. The method of any one of embodiments 1-48, wherein the lentivirus formulation is for in vivo administration to a subject.
50. The method of any one of embodiments 1-49, wherein the amount of contaminants in the filtered formulation is reduced compared to the amount of contaminants in the second filtrate.
51. The method of any one of embodiments 44-50, wherein the amount of contaminants in the sterilized formulation is at a level acceptable for in vivo administration to a subject.
52. The method of any one of embodiments 1-51, wherein the contaminants comprise host cells, host cell DNA (hcDNA), and/or host cell proteins (HCP).
53. The method of embodiment 52, wherein the amount of hcDNA is less than 10%, less than 9%, less than 8%, less than 7%, less than 6%, less than 5%, less than 4%, less than 3%, less than 2%, less than 1% or less than 0.5% of the sterilized formulation.
54. The method of embodiment 52, wherein the amount of hcDNA is reduced greater than 90%, greater than 91%, greater than 92%, greater than 93%, greater than 94%, greater than 95%, greater than 96%, greater than 97%, greater than 98%, or greater than 99% in the sterilized formulation.
55. The method of embodiment 52, wherein the amount of HCP is less than 10%, less than 9%, less than 8%, less than 7%, less than 6%, less than 5%, less than 4%, less than 3%, less than 2%, less than 1% or less than 0.5% of the sterilized formulation.
56. The method of embodiment 52, wherein the amount of HCP is reduced greater than 90%, greater than 91%, greater than 92%, greater than 93%, greater than 94%, greater than 95%, greater than 96%, greater than 97%, greater than 98%, or greater than 99% in the sterilized formulation.
57. The method of any of embodiments 52-56, wherein the hcDNA amount in the filtered formulation is less than about 2500 ng/1E9 TU.
58. The method of any one of embodiments 52-57, wherein the hcDNA amount in the filtered formulation is at least about 80-fold lower compared to the hcDNA amount in the suspension mixture.
59. The method of any one of embodiments 52-58, wherein the hcDNA amount in the filtered formulation is at least about 5-fold lower compared to the hcDNA amount in the second filtrate.
60. The method of any one of embodiments 52-59, wherein the HCP amount after chromatography is less than about 3000 μg/1E9 TU.
61. The method of any one of embodiments 52-60, wherein the HCP amount after chromatography is at least about 40-fold lower compared to the HCP amount before chromatography.
62. The method of any one of embodiments 52-61, wherein the HCP amount after chromatography is at least about 99% lower compared to the HCP amount before chromatography.
63. The method of any one of embodiments 52-62, wherein the HCP amount is less than about 1500 μg/1E9 TU after a first UF/DF step.
64. The method of any one of embodiments 52-63, wherein the HCP amount is not detectable after a second UF/DF step.
65. The method of any one of embodiments 1-64, wherein the suspension mixture comprises a media, wherein the media does not contain serum and/or animal by-products.
66. The method of any one of embodiments 2-65, wherein the culturing of step (ii) is for 40-48 hours.
67. The method of any one of embodiments 1-66, wherein the filtering and concentrating occurs over a time period of 5-8 hours.
68. The method of any one of embodiments 1-67, wherein the suspension mixture has a volume of 3-50 liters.
69. The method of any of embodiments 1-67, wherein the suspension mixture has a volume of 5 L to 200 L.
70. The method of any of embodiments 1-67, wherein the suspension mixture has a volume of 100 L to 200 L.
71. The method of any of embodiments 1-67, wherein the suspension mixture has a volume of at or about 180 L to at or about 200 L.
72. The method of any one of embodiments 1-71, wherein the lentiviral particle comprises at least one payload.
73. The method of embodiment 72, wherein the at least one payload comprises a non-coding nucleic acid, optionally wherein the non-coding nucleic acid is an siRNA, a miRNA, or a shRNA.
74. The method of embodiment 72, wherein the at least payload is a polynucleotide encoding a polypeptide of interest.
75. The method of any one of embodiments 2-74, wherein the at least one plasmid is a polynucleotide encoding a polypeptide of interest.
76. The method of embodiment 74 or 75, wherein the polypeptide of interest is a chimeric antigen receptor (CAR).
77. The method of embodiment 76, wherein the CAR is specific for a tumor-associated antigen.
78. The method of embodiment 77, wherein the tumor-associated antigen is CD19, BCMA, GPRC5D, ROR1, FcRL5, alpha-fetoprotein, or Her2.
79. The method of embodiment 76, wherein the CAR is a universal CAR.
80. The method of embodiment 79, wherein the universal CAR comprises an extracellular domain comprising a tag binding domain.
81. The method of embodiment 80, wherein the tag is a fluorescein.
82. The method of embodiment 76, wherein the CAR comprises an extracellular domain comprising a hapten binding domain.
83. The method of any one of embodiments 1-82, wherein the lentiviral particle comprises a surface engineered fusion protein exposed on the surface of the lentiviral particle, optionally wherein the surface engineered protein is embedded in the lipid bilayer.
84. The method of embodiment 83, wherein the surface engineered protein is composed of a single binding domain protein that binds to a target molecule on a target cell.
85. The method of embodiment 84, wherein the surface engineered protein is composed of a multiple binding domain protein, wherein each binding domain binds to a target molecule on a target cell, optionally wherein each binding domain binds to a different target molecule.
86. The method of embodiment 84 or embodiment 85 wherein the single binding domain protein or the multiple binding domain protein is an immune-cell activating protein.
87. The method of embodiment 83-86, wherein the surface engineered protein is a fusion protein comprising an immune cell-activating protein and a viral envelope protein.
88. The method of any one of embodiments 1-87, wherein the lentiviral particle comprises a viral envelope comprising an immune cell-activating protein and a viral envelope protein.
89. The method of any one of embodiments 2-88, wherein the at least one plasmid is a plasmid encoding a fusion protein comprising an immune cell-activating protein and a viral envelope protein.
90. The method of any one of embodiments 86-89, wherein the immune-cell activating protein comprises at least one binding domain that specifically binds CD2, CD3, CD28H, LFA-1, DNAM-1, CD27, ICOS, LIGHT, GITR, CD30, SLAM, Ly-9, CD84, Ly108, NKG2D, NKp46, NKp44, NKp30, CD244, TCR α chain, TCR β chain, TCR ζ chain, TCR γ chain, TCR δchain, CD3 ε TCR subunit, CD3 γ TCR subunit, CD3 δ TCR subunit, or NKp80, or combinations thereof.
91. The method of any one of embodiments 86-90, wherein the immune-cell activating protein comprises at least one binding domain that binds a target molecule selected from the group consisting of a T cell activation receptor, a costimulatory molecule or an adhesion molecule.
92. The method of any one of embodiments 86-91, wherein the immune-cell activating protein comprises a single binding domain that binds to one target molecule selected from the group consisting of a T cell activation receptor, a costimulatory molecule or an adhesion molecule.
93. The method of any one of embodiments 86-91, wherein the immune-cell activating protein comprises multiple binding domains that bind to two or more target molecules selected from the group consisting of a T cell activation receptor, a costimulatory molecule and an adhesion molecule.
94. The method of any one of embodiments 86-91, wherein the immune-cell activating protein comprises multiple binding domains that each bind to a different target molecule that is a T cell activation receptor, a costimulatory molecule and an adhesion molecule.
95. The method of any one of embodiments 86-94, wherein the immune cell activating protein comprises at least one binding domain that binds at least one costimulatory molecule.
96. The method of any of embodiments 91-95, wherein:
   the T cell activation receptor is CD3;
   the costimulatory molecule is CD28, CD137 or CD134; and/or
   the adhesion molecule is CD58 or CD2.
97. The method of any of embodiments 84-96, wherein each of the at least one binding domain is independently selected from an antibody or antigen-binding fragment or an ectodomain of a native ligand of the target molecule.
98. The method of any one of embodiments 87-97, wherein the viral envelope protein is a VSV-G envelope protein, a measles virus envelope protein, a nipha virus envelope protein, or a cocal virus G protein.
99. The method of any one of embodiments 2-98, wherein the at least one plasmid is a plasmid encoding a helper viral protein.
100. The method of embodiment 99, wherein the helper viral protein is rev and/or gagpol.
101. The method of any one of embodiments 2-100, wherein the population of host cells is contacted with a mixture of plasmids comprising (i) a plasmid encoding a gene of interest; (ii) a plasmid encoding a rev viral protein; (iii) a plasmid encoding a gagpol viral protein; and (iv) a plasmid encoding a viral envelope protein.
102. The method of embodiment 101, wherein the mixture of plasmids comprises (v) a plasmid encoding an immune cell-activating protein, (vi) a plasmid encoding a co-stimulatory molecule, or (vii) any combination of (v)-(vi).
103. A lentiviral formulation produced by the method of any one of embodiments 1-102.
104. The lentiviral formulation of embodiment 103, wherein the formulation has an infectious titer of 2.0 TU/mL to 6×10⁸ TU/mL.
105. The lentiviral formulation of embodiment 103 or embodiment 104, wherein the formulation has an infectious titer of 2.5 TU/mL to 4.7×10⁸ TU/mL.
106. The lentiviral formulation of embodiment 104 or embodiment 105, wherein the total number of infectious units in the formulation is 4×10¹⁰ TU to 8×10¹⁰ TU.
107. The lentiviral formulation of any of embodiments 104-106, wherein the total number of infectious units in the formulation is 5×10¹⁰ TU to 7×10¹⁰ TU, optionally at or about 6×10¹⁰ TU.
108. The lentiviral formulation of any of embodiments 104-107, wherein the formulation comprises less than 10%, less than 9%, less than 8%, less than 7%, less than 6%, less than 5%, less than 4%, less than 3%, less than 2%, less than 1% or less than 0.5% of HCP.
109. The lentiviral formulation of any of embodiments 104-108, wherein the formulation comprises less than 10%, less than 9%, less than 8%, less than 7%, less than 6%, less than 5%, less than 4%, less than 3%, less than 2%, less than 1% or less than 0.5% of hcDNA.
110. The lentiviral formulation of any of embodiments 104-109, wherein the formulation comprises greater than 90%, greater than 91%, greater than 92%, greater than 93%, greater than 94%, greater than 95%, greater than 96%, greater than 97%, greater than 98%, or greater than 99% reduction of HCP.
111. The lentiviral formulation of any of embodiments 104-110, wherein the formulation comprises greater than 90%, greater than 91%, greater than 92%, greater than 93%, greater than 94%, greater than 95%, greater than 96%, greater than 97%, greater than 98%, or greater than 99% reduction of hcDNA, optionally reduced compared to the filtered formulation prior to the concentrating.
112. The lentiviral formulation of any of embodiments 104-111, wherein the formulation comprises greater than 99% reduction of hcDNA and greater than 99% reduction in HCP, optionally reduced compared to the filtered formulation prior to the concentrating.
113. The lentiviral formulation of any of embodiments 104-112, wherein the formulation comprises less than 1% hcDNA and less than 1% HCP.
114. A lentiviral formulation comprising a lentiviral vector at a titer of 2.5 to 4.7×10⁸ TU/mL, wherein the formulation comprises less than 1% hcDNA and less than 1% HCP.
115. The lentiviral formulation of any of embodiments 103-114, wherein the volume of the formulation is 1 mL to 500 mL, optionally 10 mL to 100 mL.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

Furthermore, it is intended that any method described in the disclosure may be rewritten into Swiss-type format for the use of any agent described in the disclosure, for the manufacture of a medicament, in treating any of the disorders described in the disclosure. Likewise, it is intended for any method described in the disclosure to be rewritten as a compound for use claim, or as a use of a compound claim.

All publications, patents, and patent applications described in the disclosure are hereby incorporated by reference in their entireties.

### EXAMPLES

The disclosure is further illustrated by the following examples, which are not to be construed as limiting this disclosure in scope or spirit to the specific procedures described in the disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure.

### Example 1—Illustrative Production Process of a Lentiviral Particle

A process for producing and purifying lentiviral particles in suspension culture for use in a bioreactor was designed.

### Upstream Process

The purpose of the upstream process is to expand lentiviral production through a series of viral producer cell (VPC) expansion cell culture steps.

Briefly, following VPC cell bank vial thaw and seed train inoculation, the VPCs were expanded to 200 L and transiently transfected at an optimized ratio with a 5-plasmid system. After approximately 2 days of virus production, the cell culture was treated with an endonuclease to reduce DNA impurities prior to harvest and transferred to the downstream process for purification and concentration. An exemplary upstream production process is detailed below and depicted in FIG. 1A.

### Step 1: Cell Bank Vial Thaw

HEK293 VPCs were thawed in a cryovial at a 37° C. set point bead bath for a maximum of 2 minutes until a small ice crystal remains. The entire contents of the thawed cryovial were diluted into pre-warmed LV-MAX™ medium (ThermoFisher Scientific #A3583402) before centrifuging at 250×g for 3 minutes to remove the DMSO containing cryopreservation medium.

### Step 2: Seed Train Expansion

Thawed and pelleted VPCs were inoculated into a shake flask seed train targeting 5×10⁵ cells/mL using LV-max media. The seed train was periodically maintained with fresh media. When cells reach 4 to 6×10⁶ cells/mL, the cells are transferred into larger shake flasks (e.g., 1.6 L or 5 L) until the target cell number for a 50 L bioreactor inoculation was achieved. In some embodiments, the transfer occurs when the cells have a target passage density of 4 to 6×10⁶ cells/mL. In some embodiments, the target cell number may optionally be 6 to 7×10⁹ cells.

In some embodiments, the seed train expansion was initiated by inoculating 5×10⁵ cells/mL into a 0.125 L shake flask. The cells were then serially passaged for seed expansion and bioreactor inoculation. In this example, when the cells reached a cell density of 4 to 6×10⁶ cells/mL in the 0.125 L shake flask, cells were passaged into a larger 0.5 L shake flask. Once the cells reached a cell density of 4 to 6×10⁶ cells/mL in the 0.5 L shake flask, cells were passaged into a larger 1 L shake flask. Once the cells reached a cell density of 4 to 6×10⁶ cells/mL in the 1 L shake flask, cells were passaged into a larger 1.6 L shake flask. Once the cells reached a cell density of 4 to 6×10⁶ cells/mL in the 1.6 L shake flask, cells were passaged into a larger 5 L shake flask. In some embodiments, between each step described above, the inoculation of each shake flask, starting with the 0.5 L shake flask, was performed with about 5×10⁵ cells/mL.

### Step 3: 50 L Seed Bioreactor Inoculation and Growth Stage

The 50 L seed bioreactor was inoculated from the seed train and grown under dissolved oxygen and pH control. In some embodiments, the seed bioreactor was a 3 L, 10 L or 40 L seed bioreactor. Once a sufficient cell density was achieved (4 to 6×10⁶ cells/mL), the seed bioreactor was transferred to a 200 L bioreactor for outgrowth stage.

### Step 4: 200 L Production Bioreactor Inoculation and Growth Stage

The 200 L bioreactor was prefilled with fresh media to target 5×10⁵ cells/mL upon transfer of the 50 L seed bioreactor. In some embodiments, the seed bioreactor was a 40 L seed bioreactor. The cells were again grown up to 4 to 6×10⁶ cells/mL using the same conditions described for the 50 L Seed Bioreactor Growth stage.

### Step 5: 200 L Production Bioreactor Transfection

The bioreactor contents were diluted with fresh media to the 4×10⁶ cells/mL target transfection density, including a one-time bolus addition of glucose to replenish the LV-max medium back to its initial 5.5 g/L concentration. The bioreactor was transfected shortly after fresh media addition using PEIpro® (Polyplus-Transfection®) and a 5-plasmid system. The 5-plasmid system included a plasmid expressing a gene of interest, a plasmid expressing the lentiviral gagpol gene, a plasmid expressing the lentiviral rev gene, a plasmid expressing a viral envelope protein, and a plasmid expressing an immune-activating protein.

### Step 6: 200 L Production Bioreactor Endonuclease Addition and Harvest

A one-time bolus addition of an endonuclease and MgCl₂ supplementation was performed 2 hours before batch harvest, targeting 44 to 48 hours after transfection, before proceeding to the first step of the downstream process (harvest clarification).

### Summary of the Upstream Process

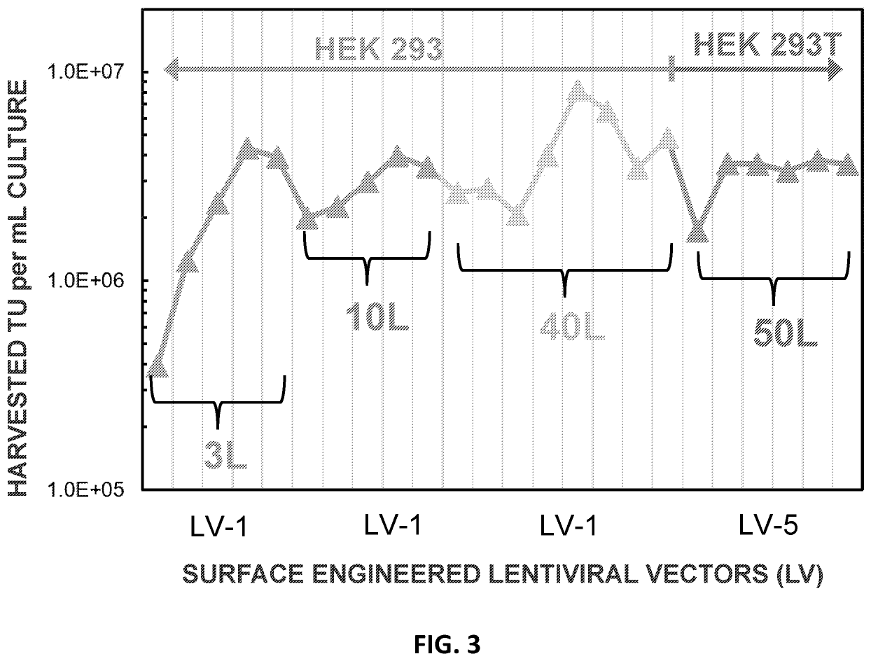

The upstream manufacturing platform described above was used to produce engineered lentiviral vectors (LVs) in different producer cell lines (e.g. HEK293 or HEK293T) with transfer plasmids containing different transgenes encoding either single domain proteins (LV-1) or multi-domain fusion proteins (LV-5). The upstream manufacturing platform also was carried out using different seed bioreactors and utilizing different laboratory sites. FIG. 3 depicts the harvested virus concentration as transduction units (TU) per mL prepared for exemplary upstream process runs under different conditions. The results show production of a potent LV engineered product across varied process runs, and demonstrate consistency of the upstream process whether the processes differed in reagent source (e.g. plasmids), producer cell lines, bioreactor and scale, and particular laboratory site. These results thus demonstrate that the upstream process can be leveraged for different products and cell lines, including at different scales and process sites. Moreover, the results are consistent with a finding that a potent product can be consistently produced, which may reduce dosing requirements and the extent of batch size and scale-up.

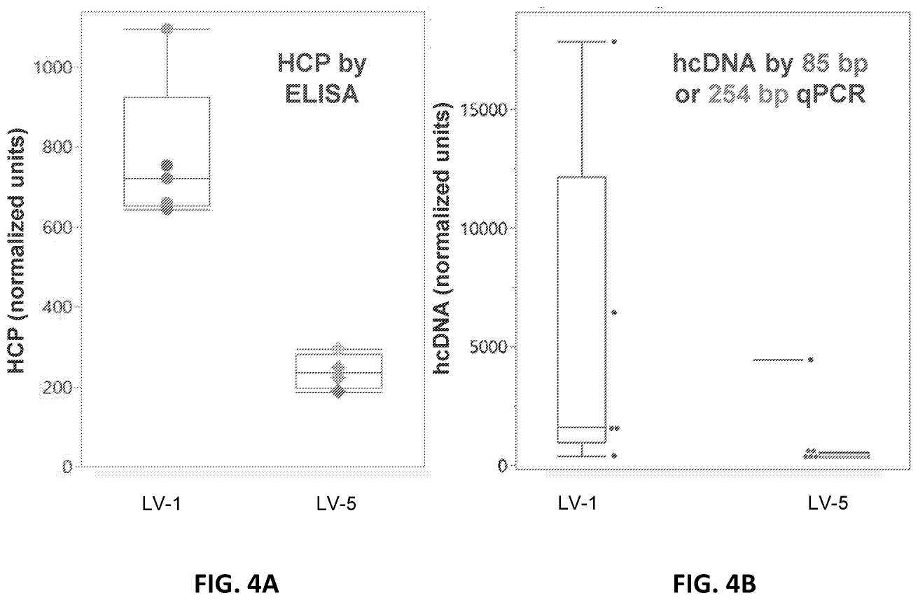

Following the upstream process, host cell impurities including host cell protein (HCP) and host cell DNA (hcDNA) were measured in LV-1 and LV-5 produced by HEK293T cells. hcDNA and HCP were measured as described in Example 3. FIGS. 4A and 4B show that LV production by HEK293T cells significantly decreased impurities of the LV product.

### Downstream Process

The purpose of the downstream process is recovering the lentivirus from the upstream bioreactor through a series of unit operations to purify, concentrate, and formulate the lentivirus to produce a lentiviral drug substance. Briefly, the harvest clarification step removed host cells, cell debris, and precipitated impurities. The clarified harvest pool was processed using anion exchange (AEX) chromatography to concentrate and purify the lentivirus. The AEX chromatography pool was processed using ultrafiltration/diafiltration (UF/DF) to exchange buffer and further concentrate lentivirus to the meet the lentiviral drug substance transducing titer specification. The resulting pool was 0.2 μm-filtered to produce bulk drug substance (BDS), which was stored frozen at ≤−60° C. A general downstream production process is detailed below and depicted in FIG. 1B.

### Step 7: Harvest Clarification

HEK293 host cells, cell debris, and precipitates present in the production bioreactor at harvest were removed using a series of depth and membrane filters. Bioreactor harvest solution was fed through the filters at a controlled flow rate and differential pressure to clarify the harvest. The clarified harvest pool was forward processed to the AEX chromatography step.

### Step 8: Anion Exchange (AEX) Chromatography

The main purification function of this step is host cell protein reduction. The AEX chromatography unit operation captured and concentrated lentivirus from the clarified harvest pool and reduced process-related impurities. The step was performed with a Mustang Q™ (Pall Corporation) AEX membrane chromatography capsule, which is composed of a PES pleated membrane (e.g. 0.8 μm nominal pore size) and a surface coating of positively charged functional groups. This structure allows for convective flow of the negatively charged lentivirus across the membrane where it can then bind with the positively charged coated surface. Larger impurities as well as impurities with different binding capacities than that of the lentivirus, primarily positively charged impurities, flow through the membrane during loading. Loosely bound impurities are then removed with a wash step carried out with a salt buffer with the same pH and conductivity as that of the bulk load. Lentivirus bound to the membrane was then eluted by increasing the salt concentration (e.g. to 0.75 M) across the membrane resulting in dissociation of lentivirus from the membrane. During the salt elution, the lentivirus was collected directly into a lower salt dilution buffer. Prior to starting the step, the clarified harvest pool was conditioned by adding sodium chloride and adjusting to approximately pH 8 with Tris. The conditioned clarified harvest was loaded onto the AEX capsule to bind lentivirus while unbound or loosely bound impurities flowed through the capsule. Following the load, impurities were further removed using a buffer wash step with a pH 8 wash buffer. Following the wash step, lentivirus was eluted from the capsule using a pH 8 elution buffer. The eluted lentivirus was diluted five-fold with a pH 8 buffer to reduce the sodium chloride, and the diluted AEX pool was forward processed to the UF/DF step.

### Step 9: Ultrafiltration/Diafiltration (UF/DF)

The UF/DF unit operation concentrated and performed buffer exchange (diafiltration) of the diluted AEX chromatography pool to achieve the pH, osmolality, and transducing titer specified for the BDS. This step used a ≤500 kD molecular-weight cut-off tangential flow ultrafiltration membrane to produce a UF/DF pool in a pH neutral buffer. Following diafiltration and final concentration to the desired transducing titer, lentivirus was recovered, and the UF/DF pool was forward processed to the drug substance filtration step.

### Step 10 and 11: Drug Substance Filtration and Storage

The UF/DF pool in the pH neutral buffer (10 mM Tris, 10% sucrose, pH 7.1) was filtered through a 0.2 μm pore-size, sterilizing grade filter into a container. The lentiviral drug substance was then stored at ≤−60° C. until further processing to drug product.

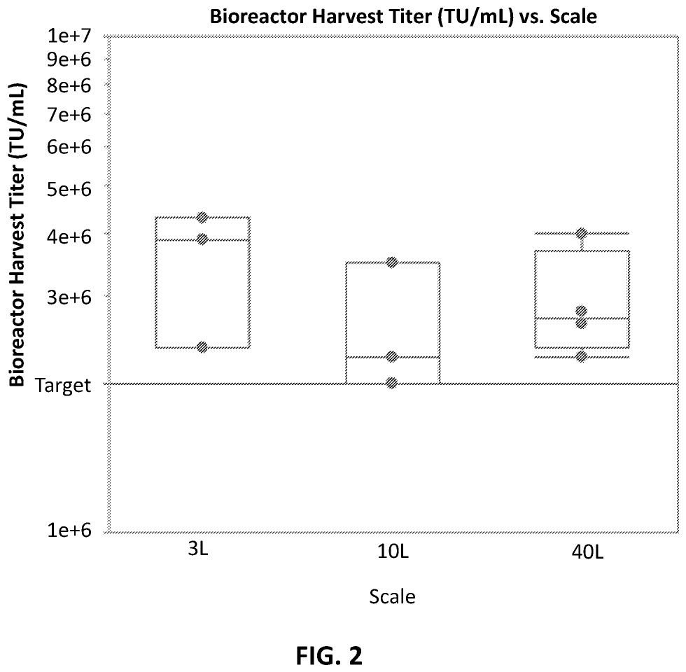

FIG. 2 provides a box plot showing the scaling up of the upstream process. These data demonstrate that the upstream and downstream processing steps provided herein result in consistent titer production when seed train expansion was performed in 3 L, 10 L or 40 L bioreactors.

### Example 2—Comparison of Multiple Downstream Production Processes

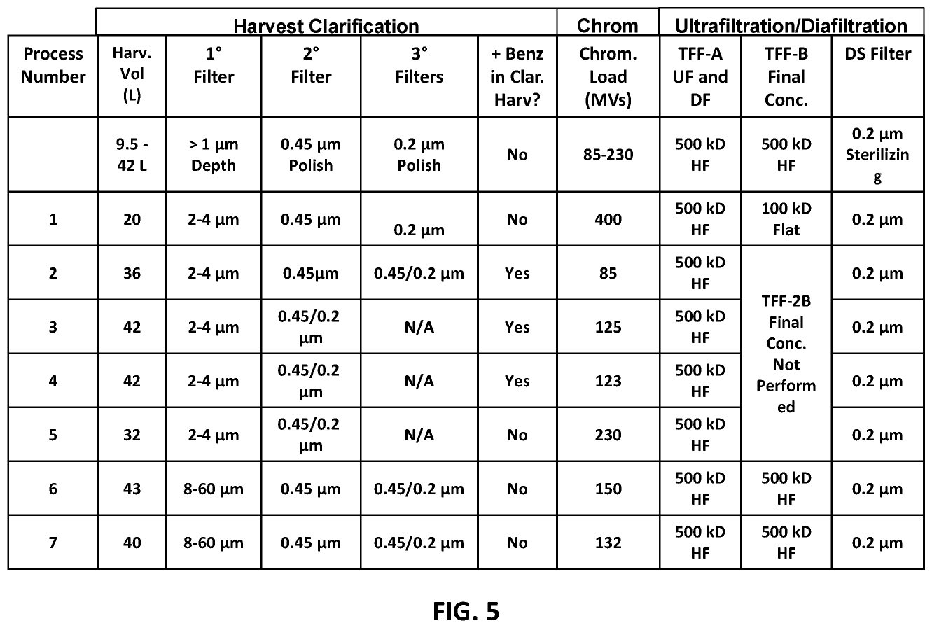

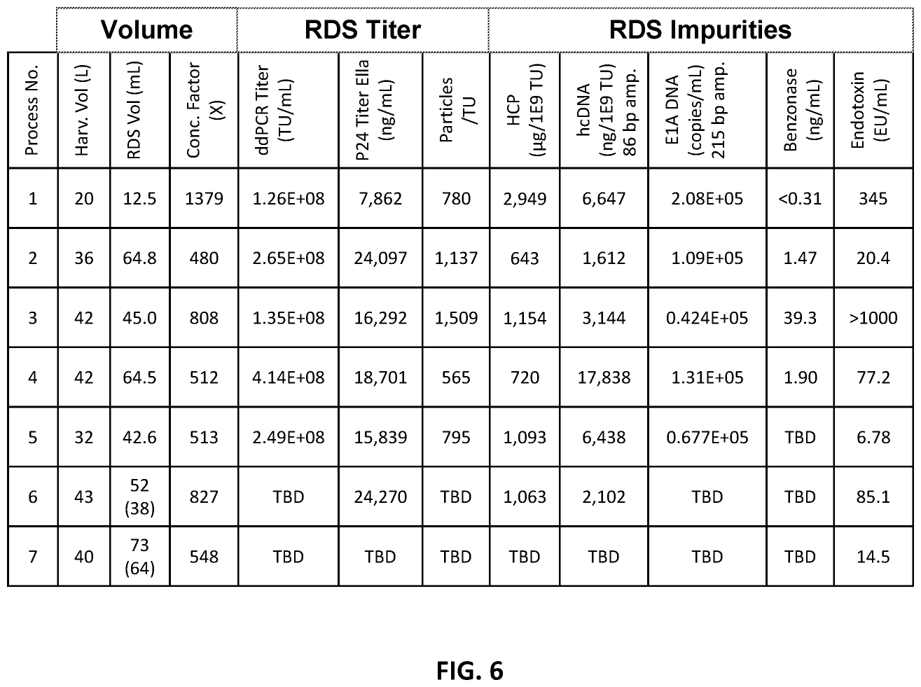

Seven downstream processes with varying conditions were performed (FIG. 5) to select for one or more process candidates. The downstream processes described herein were performed substantially as described in Example 1 but differed with respect to the starting harvest volume, the lack of endonuclease, and the UF/DF filter membrane molecular weight cutoffs. After downstream processing, the end volume, titer, and impurities were measured in the end product (FIG. 6). Briefly, hcDNA was measured by qPCR, HCP was measured by ELISA, E1DNA with 215 bp amplification region was measured by qPCR, endotoxin levels were measured using the Charles River Endosafe method, and titer was measured by a SUP-T1 cell transduction assay and a p24 ELISA method (Bio-Techne®) according to manufacturer's instructions. The end product after downstream processing is also referred to herein as research drug substance (RDS).

Process No. 1 had the lowest harvest volume (20 L) and high levels of endotoxin in the end product (FIG. 6).

In Process Nos. 2, 3, and 4, a benzonase addition step was performed after the third filtration step. Results showed that the additional benzonase step did not provide further host cell DNA (hcDNA) removal capability, and residual analysis showed detectable or abnormally high levels of benzonase.

Among Process Nos. 5, 6, and 7, Process No. 5 did not include a third filtration step in the harvest clarification process, while both Process No. 6 and Process No. 7 included a third filtration step using a 0.2 μm filter (for example, a Sartopore®2 0.2 μm). Impurities analysis showed Process No. 6 had significantly reduced amounts of hcDNA compared to Process No. 5 (FIG. 6).

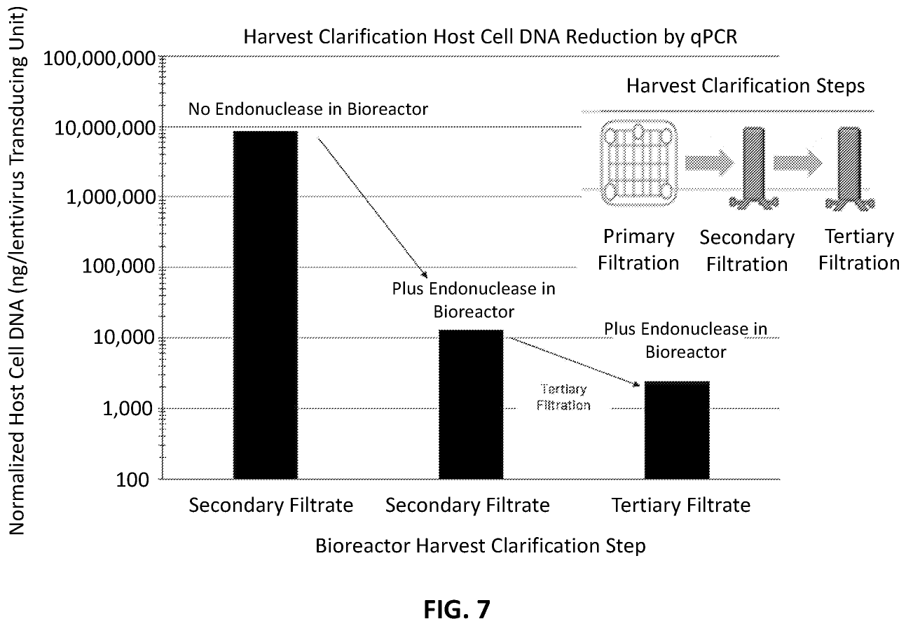

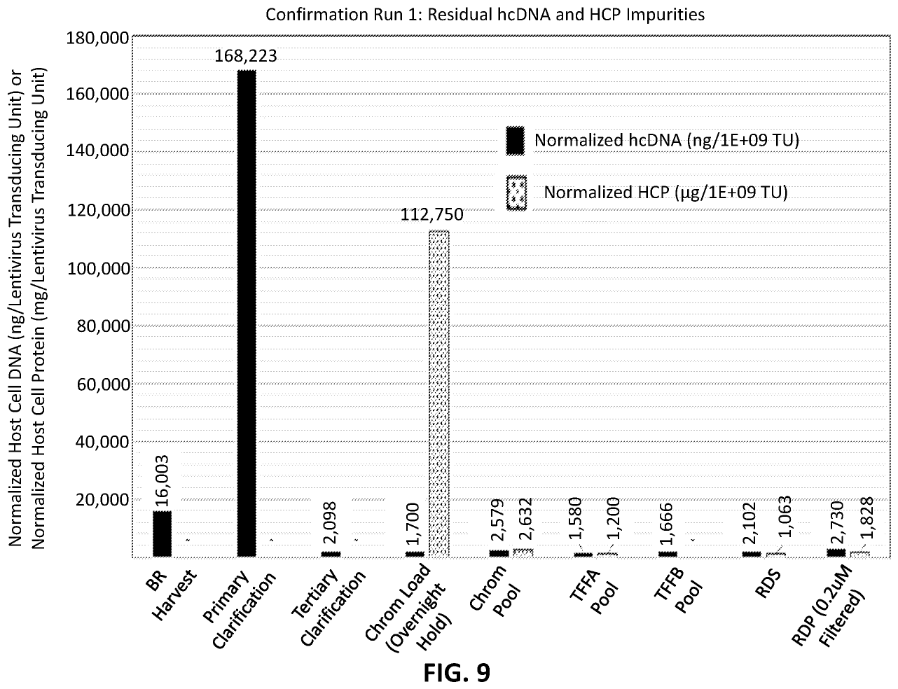

Endonuclease digestion in the bioreactor reduced hcDNA by ˜3 log in the secondary filtrate as measured by qPCR (FIG. 7). In the case where endonuclease was added to the bioreactor prior to harvest, the effect of the second and third filtration steps on hcDNA was investigated. The measured hcDNA amount (ng) was normalized to lentivirus amount (Transducing Unit, TU). The results showed the third filter reduced hcDNA in the tertiary filtrate by 5-fold compared to the secondary filtrate (FIG. 7) and more than 80-fold compared to the primary filtrate (FIG. 9). This was a surprising result because the threshold for the third filter was 0.2 μm, which is magnitudes of order larger than the size of DNA. These results indicate a significant amount of hcDNA that was not digested by the endonuclease associated with larger particles (e.g., cell debris and/or precipitate), and the third filtration step enabled the removal of these particles and particle-associated hcDNA.

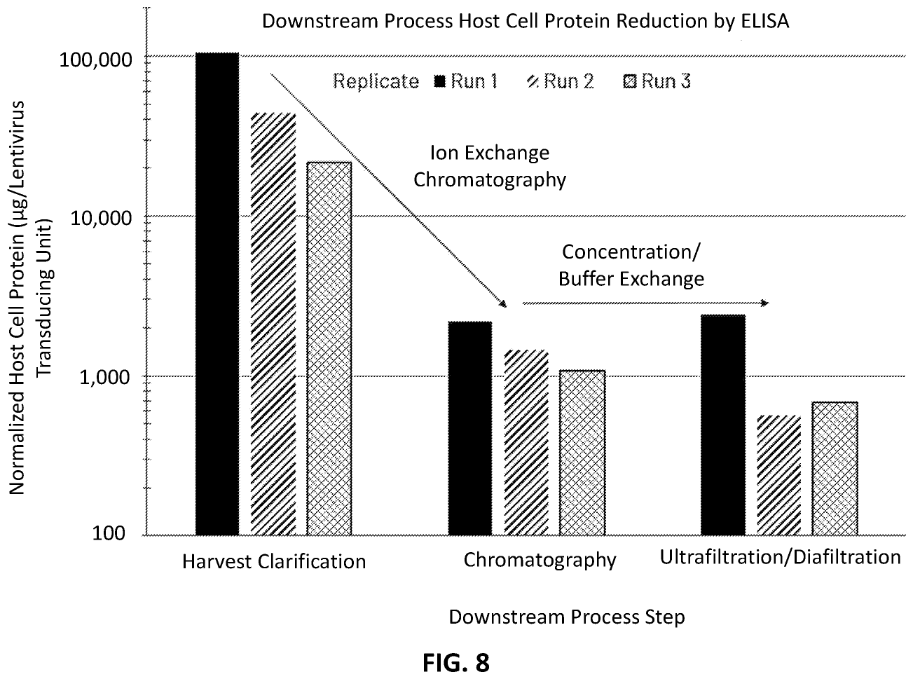

After clarification, ion-exchange chromatography reduced host cell protein (HCP) levels by 1-2 logs as measured by ELISA (FIG. 8). The measured HCP amount (μg) was normalized to lentivirus amount (TU). The effect of subsequent ultrafiltration/diafiltration (UF/DF) after chromatography was investigated, and results showed less reduction in HCP was achieved with the subsequent UF/DF as compared to that achieved in the chromatography step (FIG. 8). These results indicated some HCP were retained by the ultrafiltration membrane and may be associated with the lentivirus.

Process No. 5 did not include a final concentration step using UF/DF (step TFF-B in FIG. 5), while both Process No. 6 and Process No. 7 included the TFF-B step. Impurities analysis of the RDS showed the HCP levels between the Process No. 5 and Process No. 6 remain similar, while the hcDNA amount in Process No. 5 was about 3-fold higher than that of Process No. 6 (FIG. 5). Furthermore, while Process No. 5 did not include a third filtration step in the clarification process, it also used a 0.2 μm threshold filter (for example, a Sartopore®2 Pore size: 0.45|0.2 μm, heterogeneous PES double layer) in the second filtration step (FIG. 5). This indicated a potential importance of a final concentration (TFF-B) step in removing hcDNA.

The residual DNA (also referred to as resDNA) and HCP per dose was calculated for individual steps in Process No. 6, and results are presented in FIG. 9. After primary clarification (1^{st} filter), the amount of resDNA was 168,223 ng/dose. After tertiary clarification (3^{rd} filter), the amount of resDNA was 2,098 ng/dose, approximately 80-fold decrease. The largest decrease in HCP amount occurred after chromatography: from 112,750 μg/dose to 2,632 μg/dose, approximately 40-fold decrease. HCP amount further decreased to 1,200 μg/dose after the first UF/DF concentration step and was undetectable after the second UF/DF concentration.

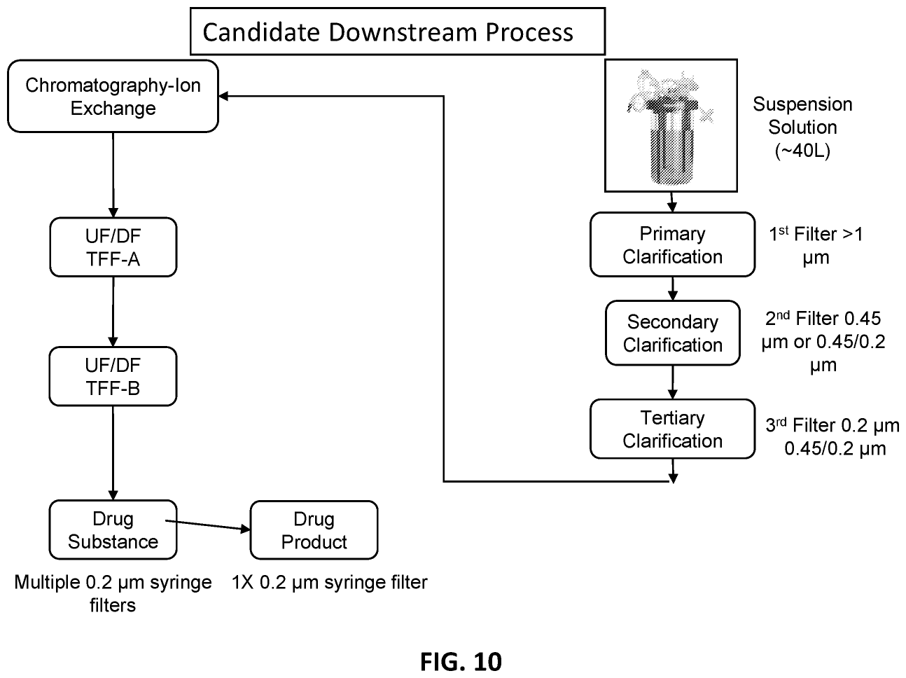

As a result of the experiments described above, Process No. 6 and No. 7 were selected as candidate processes to be used in the downstream process of lentiviral particle manufacturing. FIG. 10 provides a schematic drawing of an exemplary downstream process.

### Example 3—Assessment of Purity Following Lentiviral Manufacturing Process Purification

The lentiviral process purification method described in the above examples was assessed for purity of the preparation by monitoring the presence of host cell DNA and protein levels. Briefly, the upstream process described in Example 1 was performed, with the exception that in Step 3 a 5 L (for downstream Process Nos. 2 and 5) or 10 L (for downstream Process Nos. 6 and 7) seed bioreactor was inoculated from the seed train and grown under dissolved oxygen and pH control. Once a sufficient cell density was achieved (4 to 6×10⁶ cells/mL), the seed bioreactor was transferred to a 40 L bioreactor for the outgrowth stage.

After the upstream process was complete, the downstream process corresponding to Process Nos. 2, 5, 6 and 7 in Example 2 was performed. Following the downstream process, host cell impurities including host cell protein (HCP) and host cell DNA (hcDNA) were measured in the end product. hcDNA was measured by qPCR and HCP was measured by ELISA and LC-MS.

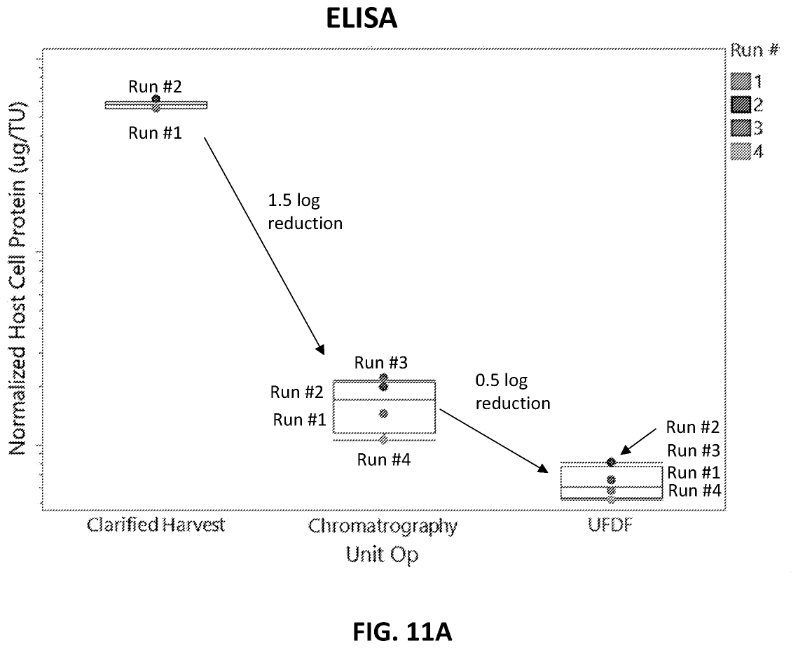

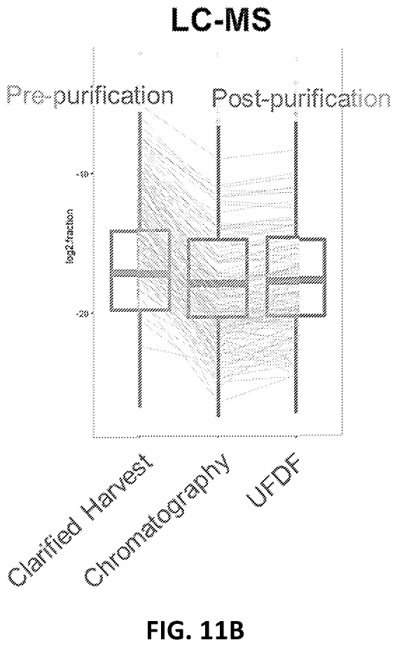

FIG. 11A and FIG. 11B depict the amount (μg/TU) of HCP protein present by ELISA and LC-MS, respectively. “Clarified Harvest” refers to a bioreactor harvest that was treated with an endonuclease, isolated from the bioreactor, filtered and treated with endonuclease again. “Chromatography” refers to a bioreactor harvest that was treated with an endonuclease, isolated from the bioreactor, filtered and purified by AEX chromatography. “UFDF” refers to a bioreactor harvest that was treated with an endonuclease, isolated from the bioreactor, filtered, purified by AEX chromatography and filtered by UF/DF. Purifying clarified bioreactor harvest by AEX chromatography (“Chromatography” sample in FIG. 11A) resulted in a 1.5 log reduction of HCP. Purifying clarified bioreactor harvest by UF/DF (“UF/DF” sample in FIG. 11A), resulted in a further 0.5 log reduction in HCP. Corresponding LC-MS HCP data is shown in FIG. 11B.

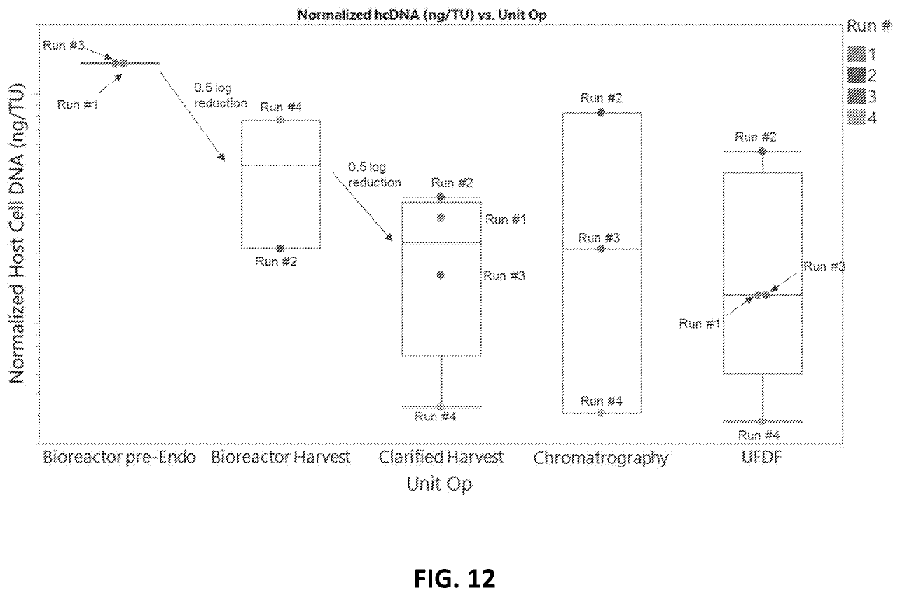

FIG. 12 depicts the amount (ng/TU) of hcDNA present by qPCR. From left to right, hcDNA (ng/TU) is provided for: bioreactor sample that was not treated with an endonuclease (“Bioreactor pre-Endo”); bioreactor harvest that was treated with an endonuclease (“Bioreactor Harvest”); bioreactor harvest that was treated with an endonuclease, isolated from the bioreactor, filtered and treated with endonuclease again (“Clarified Harvest”); bioreactor harvest that was treated with an endonuclease, isolated from the bioreactor, filtered and purified by AEX chromatography (“Chromatography”); and bioreactor harvest that was treated with an endonuclease, isolated from the bioreactor, filtered, purified by AEX chromatography and filtered by UF/DF (“UF/DF”).

Purifying bioreactor harvest treated with Benzonase resulted in a 0.5 log reduction of hcDNA. Purifying clarified bioreactor harvest treated with Benzonase further resulted in a 0.5 log reduction of hcDNA.

These data demonstrate that the upstream and downstream manufacturing processes demonstrated herein clear more than 99% of HCP and hcDNA from lentiviral particle formulations.

### Example 4—Assessment of Lentiviral Yield and Purity Following Salt-Dependent Elution

Lentivirus engineered to express surface molecules were assessed in the downstream process to determine the role of salt concentration in the elution step of the chromatography method. Specifically, a lentivirus expressing a single domain protein (LV-1), a lentivirus expressing at least three different single domain proteins+GFP (LV-2), a lentivirus expressing at least three different single domain proteins+CAR (LV-3), a lentivirus expressing a multi-domain fusion protein+CAR (LV-4), or a lentivirus expressing a multi-domain fusion protein (LV-5) were generated in virus producer cells. The downstream process #5 described in Example 2 was performed to purify and concentrate the LVs using AEX chromatography as described in step 8 of Example 1, except that the salt elution step of the chromatography method was performed at different salt (NaCl) concentrations (0.75M vs 2M).

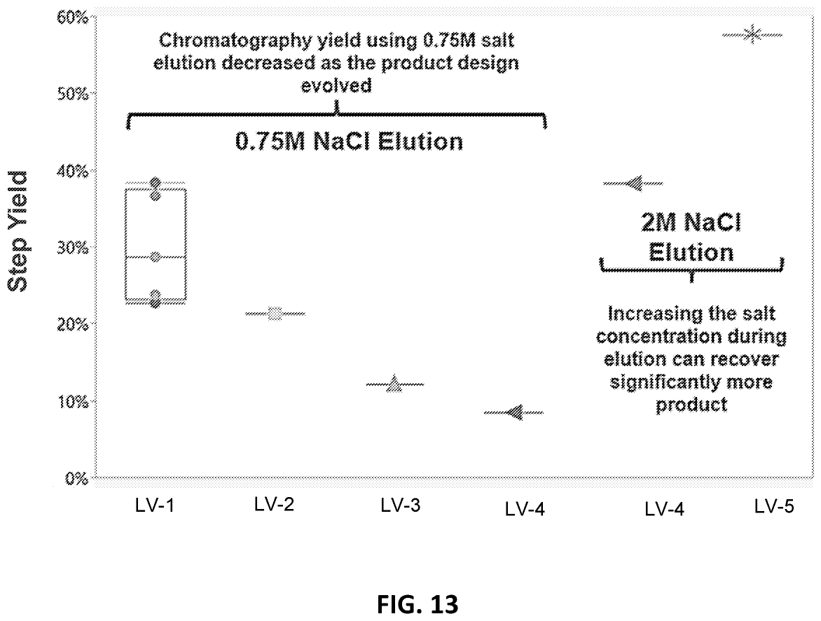

The results show LV was recovered in all conditions when eluted with 0.75M NaCl; however, there was a decreasing trend in the percent yield of LV comprising a higher transgene payload (FIG. 13). For example, yield was inversely related to the size of the surface molecules of the engineered LVs. However, increasing the salt concentration of the elution step to 2M in the chromatography method increased lentivirus yield as a percentage of the amount of virus loaded to the column in the chromatography step (FIG. 13) for LVs with larger transgene payloads (e.g. LV-4 and LV-5).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID | | |
|---|---|---|
| NO. | Annotation | Sequence |
| 1 | CD58 | MVAGSDAGRALGVLSVVCLLHCFGFISCFSQQIYGVVYGNVTFHVPSNVPLKEVLW |
| | | KKQKDKVAELENSEFRAFSSFKNRVYLDTVSGSLTIYNLTSSDEDEYEMESPNITD |
| | | TMKFFLYVLESLPSPTLTCALINGSIEVQCMIPEHYNSHRGLIMYSWDCPMEQCKR |
| | | NSTSIYFKMENDLPQKIQCTLSNPLENTTSSIILTTCIPSSGHSRHRYALIPIPLA |
| | | VITTCIVLYMNGILKCDRKPDRINSN |
| 2 | CD58 | ATGGTTGCTGGGAGCGACGCGGGGCGGGCCCTGGGGGTCCTCAGCGTGGTCTGCCT |
| | | GCTGCACTGCTTTGGTTTCATCAGCTGTTTTTCCCAACAAATATATGGTGTTGTGT |
| | | ATGGGAATGTAACTTTCCATGTACCAAGCAATGTGCCTTTAAAAGAGGTCCTATGG |
| | | AAAAAACAAAAGGATAAAGTTGCAGAACTGGAAAATTCTGAGTTCAGAGCTTTCTC |
| | | ATCTTTTAAAAATAGGGTTTATTTAGACACTGTGTCAGGTAGCCTCACTATCTACA |
| | | ACTTAACATCATCAGATGAAGATGAGTATGAAATGGAATCGCCAAATATTACTGAT |
| | | ACCATGAAGTTCTTTCTTTATGTGCTTGAGTCTCTTCCATCTCCCACACTAACTTG |
| | | TGCATTGACTAATGGAAGCATTGAAGTCCAATGCATGATACCAGAGCATTACAACA |
| | | GCCATCGAGGACTTATAATGTACTCATGGGATTGTCCTATGGAGCAATGTAAACGT |
| | | AACTCAACCAGTATATATTTTAAGATGGAAAATGATCTTCCACAAAAAATACAGTG |
| | | TACTCTTAGCAATCCATTATTTAATACAACATCATCAATCATTTTGACAACCTGTA |
| | | TCCCAAGCAGCGGTCATTCAAGACACAGATATGCACTTATACCCATACCATTAGCA |
| | | GTAATTACAACATGTATTGTGCTGTATATGAATGGTATTCTGAAATGTGACAGAAA |
| | | ACCAGACAGAACCAACTCCAAT |
| 3 | CD80 | MGHTRRQGTSPSKCPYLNFFQLLVLAGLSHFCSGVIHVTKEVKEVATLSCGHNVSV |
| | | EELAQTRIYWQKEKKMVLTMMSGDMNIWPEYKNRTIFDITNNLSIVILALRPSDEG |
| | | TYECVVLKYEKDAFKREHLAEVTLSVKADFPTPSISDFEIPTSNIRRIICSTSGGF |
| | | PEPHLSWLENGEELNAINTTVSQDPETELYAVSSKLDFNMTTNHSFMCLIKYGHLR |
| | | VNQTFNWNTTKQEHFPDNLLPSWAITLISVNGIFVICCLTYCFAPRCRERRRNERL |
| | | RRESVRPV |
| 4 | CD80 | ATGGGCCACACACGGAGGCAGGGAACATCACCATCCAAGTGTCCATACCTCAATTT |
| | | CTTTCAGCTCTTGGTGCTGGCTGGTCTTTCTCACTTCTGTTCAGGTGTTATCCACG |
| | | TGACCAAGGAAGTGAAAGAAGTGGCAACGCTGTCCTGTGGTCACAATGTTTCTGTT |
| | | GAAGAGCTGGCACAAACTCGCATCTACTGGCAAAAGGAGAAGAAAATGGTGCTGAC |
| | | TATGATGTCTGGGGACATGAATATATGGCCCGAGTACAAGAACCGGACCATCTTTG |
| | | ATATCACTAATAACCTCTCCATTGTGATCCTGGCTCTGCGCCCATCTGACGAGGGC |
| | | ACATACGAGTGTGTTGTTCTGAAGTATGAAAAAGACGCTTTCAAGCGGGAACACCT |
| | | GGCTGAAGTGACGTTATCAGTCAAAGCTGACTTCCCTACACCTAGTATATCTGACT |
| | | TTGAAATTCCAACTTCTAATATTAGAAGGATAATTTGCTCAACCTCTGGAGGTTTT |
| | | CCAGAGCCTCACCTCTCCTGGTTGGAAAATGGAGAAGAATTAAATGCCATCAACAC |
| | | AACAGTTTCCCAAGATCCTGAAACTGAGCTCTATGCTGTTAGCAGCAAACTGGATT |
| | | TCAATATGACAACCAACCACAGCTTCATGTGTCTCATCAAGTATGGACATTTAAGA |
| | | GTGAATCAGACCTTCAACTGGAATACAACCAAGCAAGAGCATTTTCCTGATAACCT |
| | | GCTCCCATCCTGGGCCATTACCTTAATCTCAGTAAATGGAATTTTTGTGATATGCT |
| | | GCCTGACCTACTGCTTTGCCCCAAGATGCAGAGAGAGAAGGAGGAATGAGAGATTG |
| | | AGAAGGGAAAGTGTACGCCCTGTA |
| 5 | CD86 | MDPQCTMGLSNILFVMAFLLSGAAPLKIQAYFNETADLPCQFANSQNQSLSELVVE |
| | | WQDQENLVLNEVYLGKEKFDSVHSKYMGRTSFDSDSWTLRLHNLQIKDKGLYQCII |
| | | HHKKPTGMIRIHQMNSELSVLANFSQPEIVPISNITENVYINLTCSSIHGYPEPKK |
| | | MSVLLRTKNSTIEYDGVMQKSQDNVTELYDVSISLSVSFPDVTSNMTIFCILETDK |
| | | TRLLSSPFSIELEDPQPPPDHIPWITAVLPTVIICVMVFCLILWKWKKKKRPRNSY |
| | | KCGTNTMEREESEQTKKREKIHIPERSDEAQRVFKSSKTSSCDKSDTCF |
| 6 | CD86 | ATGGATCCCCAGTGCACTATGGGACTGAGTAACATTCTCTTTGTGATGGCCTTCCT |
| | | GCTCTCTGGTGCTGCTCCTCTGAAGATTCAAGCTTATTTCAATGAGACTGCAGACC |
| | | TGCCATGCCAATTTGCAAACTCTCAAAACCAAAGCCTGAGTGAGCTAGTAGTATTT |
| | | TGGCAGGACCAGGAAAACTTGGTTCTGAATGAGGTATACTTAGGCAAAGAGAAATT |
| | | TGACAGTGTTCATTCCAAGTATATGGGCCGCACAAGTTTTGATTCGGACAGTTGGA |
| | | CCCTGAGACTTCACAATCTTCAGATCAAGGACAAGGGCTTGTATCAATGTATCATC |
| | | CATCACAAAAAGCCCACAGGAATGATTCGCATCCACCAGATGAACTCTGAACTGTC |
| | | AGTGCTTGCTAACTTCAGTCAACCTGAAATAGTACCAATTTCTAATATAACAGAAA |
| | | ATGTGTACATAAATTTGACCTGCTCATCTATACACGGTTACCCAGAACCTAAGAAG |
| | | ATGAGTGTTTTGCTAAGAACCAAGAACTCAACTATCGAGTATGATGGTGTTATGCA |
| | | GAAATCTCAAGATAATGTCACAGAACTGTACGACGTTTCCATCAGCTTGTCTGTTT |
| | | CATTCCCTGATGTTACGAGCAATATGACCATCTTCTGTATTCTGGAAACTGACAAG |
| | | ACGCGGCTTTTATCTTCACCTTTCTCTATAGAGCTTGAGGACCCTCAGCCTCCCCC |
| | | AGACCACATTCCTTGGATTACAGCTGTACTTCCAACAGTTATTATATGTGTGATGG |
| | | TTTTCTGTCTAATTCTATGGAAATGGAAGAAGAAGAAGCGGCCTCGCAACTCTTAT |
| | | AAATGTGGAACCAACACAATGGAGAGGGAAGAGAGTGAACAGACCAAGAAAAGAGA |
| | | AAAAATCCATATACCTGAAAGGTCTGATGAAGCCCAGCGTGTTTTTAAAAGTTCGA |
| | | AGACATCTTCATGCGACAAAAGTGATACATGTTTT |
| 7 | CD80 | VIHVTKEVKEVATLSCGHNVSVEELAQTRIYWQKEKKMVLTMMSGDMNIWPEYKNR |
| | Extracellular | TIFDITNNLSIVILALRPSDEGTYECVVLKYEKDAFKREHLAEVTLSVKADFPTPS |
| | Domain | ISDFEIPTSNIRRIICSTSGGFPEPHLSWLENGEELNAINTTVSQDPETELYAVSS |
| | | KLDFNMTTNHSFMCLIKYGHLRVNQTFNWNTTKQEHFPDN |
| 8 | CD86 | APLKIQAYFNETADLPCQFANSQNQSLSELVVFWQDQENLVLNEVYLGKEKFDSVH |
| | Extracellular | SKYMGRTSFDSDSWTLRLHNLQIKDKGLYQCIIHHKKPTGMIRIHQMNSELSVLAN |
| | Domain | FSQPEIVPISNITENVYINLTCSSIHGYPEPKKMSVLLRTKNSTIEYDGVMQKSQD |
| | | NVTELYDVSISLSVSFPDVTSNMTIFCILETDKTRLLSSPFSIELEDPQPPPDHIP |
| 9 | CD58 | FSQQIYGVVYGNVTFHVPSNVPLKEVLWKKQKDKVAELENSEFRAFSSFKNRVYLD |
| | Extracellular | TVSGSLTIYNLTSSDEDEYEMESPNITDTMKFFLYV |
| | Domain | |
| 10 | HHLA2 | QTSVSPSKVILPRGGSVLVTCSTSCDQPKLLGIETPLPKKELLLPGNNRKVYELSN |
| | Extracellular | VQEDSQPMCYSNCPDGQSTAKTFLTVYWTPERVELAPLPSWQPVGKNLTLRCQVEG |
| | Domain | GAPRANLTVVLLRGEKELKREPAVGEPAEVTTTVLVRRDHHGANFSCRTELDLRPQ |
| | | GLELFENTSAPYQLQTFVLPATPPQLVSPRVLEVDTQGTVVCSLDGLFPVSEAQVH |
| | | LALGDQRLNPTVTYGNDSFSAKASVSVTAEDEGTQRLTCAVILGNQSQETLQTVTI |
| | | YSFPAPNVILTKPEVSEGTEVTVKCEAHPRAKVTLNGVPAQPLGPRAQLLLKATPE |
| | | DNGRSFSCSATLEVAGQLIHKNQTRELRVLYGPRLDERDCPGNWTWPENSQQTPMC |
| | | QAWGNPLPELKCLKDGTFPLPIGESVTVTRDLEGTYLCRARSTQGEVTRKVTVNVL |
| | | SPRYE |
| 11 | ICAM-1 | QVSHRYPRIQSIKVQFTEYKKEKGFILTSQKEDEIMKVQNNSVIINCDGFYLISLK |
| | Extracellular | GYFSQEVNISLHYQKDEEPLFQLKKVRSVNSLMVASLTYKDKVYLNVTTDNTSLDD |
| | Domain | FHVNGGELILIHQNPGEFCVL |
| 12 | OX40-L | REGPELSPDDPAGLLDLRQGMFAQLVAQNVLLIDGPLSWYSDPGLAGVSLTGGLSY |
| | Extracellular | KEDTKELVVAKAGVYYVFFQLELRRVVAGEGSGSVSLALHLQPLRSAAGAAALALT |
| | Domain | VDLPPASSEARNSAFGFQGRLLHLSAGQRLGVHLHTEARARHAWQLTQGATVLGLF |
| | | RVTPEIPAGLPSPRSE |
| 13 | 4-1BBL | EPPTACREKQYLINSQCCSLCQPGQKLVSDCTEFTETECLPCGESEFLDTWNRETH |
| | Extracellular | CHQHKYCDPNLGLRVQQKGTSETDTICTCEEGWHCTSEACESCVLHRSCSPGFGVK |
| | Domain | QIATGVSDTICEPCPVGFFSNVSSAFEKCHPWTSCETKDLVVQQAGTNKTDVVCGP |
| | | QDRLR |
| 14 | CD40 | WPPPGTGDVVVQAPTQVPGFLGDSVTLPCYLQVPNMEVTHVSQLTWARHGESGSMA |
| | Extracellular | VFHQTQGPSYSESKRLEFVAARLGAELRNASLRMFGLRVEDEGNYTCLFVTFPQGS |
| | Domain | RSVDIWLRVLAKPQNTAEVQKVQLTGEPVPMARCVSTGGRPPAQITWHSDLGGMPN |
| | | TSQVPGFLSGTVTVTSLWILVPSSQVDGKNVTCKVEHESFEKPQLLTVNLTVYYPP |
| | | EVSISGYDNNWYLGQNEATLTCDARSNPEPTGYNWSTTMGPLPPFAVAQGAQLLIR |
| | | PVDKPINTTLICNVTNALGARQAELTVQVKEGPPSEHSGISRN |
| 15 | CD155 | QRFAQAQQQLPLESLGWDVAELQLNHTGPQQDPRLYWQGGPALGRSFLHGPELDKG |
| | Extracellular | QLRIHRDGIYMVHIQVTLAICSSTTASRHHPTTLAVGICSPASRSISLLRLSFHQG |
| | Domain | CTIASQRLTPLARGDTLCTNLTGTLLPSRNTDETFFGVQWVRP |
| 16 | CD70 | LPSCKEDEYPVGSECCPKCSPGYRVKEACGELTGTVCEPCPPGTYIAHLNGLSKCL |
| | Extracellular | QCQMCDPAMGLRASRNCSRTENAVCGCSPGHFCIVQDGDHCAACRAYATSSPGQRV |
| | Domain | QKGGTESQDTLCQNCPPGTFSPNGTLEECQHQTKCSWLVTKAGAGTSSS |
| 17 | HVEM | QLETAKEPCMAKFGPLPSKWQMASSEPPCVNKVSDWKLEILQNGLYLIYGQVAPNA |
| | Extracellular | NYNDVAPFEVRLYKNKDMIQTLINKSKIQNVGGTYELHVGDTIDLIFNSEHQVLKN |
| | Domain | NTYWGIILLANPQFIS |
| 18 | GITRL | QRTDSIPNSPDNVPLKGGNCSEDLLCILKRAPFKKSWAYLQVAKHLNKTKLSWNKD |
| | Extracellular | GILHGVRYQDGNLVIQFPGLYFIICQLQFLVQCPNNSVDLKLELLINKHIKKQALV |
| | Domain | TVCESGMQTKHVYQNLSQFLLDYLQVNTTISVNVDTFQYIDTSTFPLENVLSIFLY |
| | | SNSD |
| 19 | CD30L | QGHLVHMTVVSGSNVTLNISESLPENYKQLTWFYTFDQKIVEWDSRKSKYFESKFK |
| | Extracellular | GRVRLDPQSGALYISKVQKEDNSTYIMRVLKKTGNEQEWKIKLQVLDPVPKPVIKI |
| | Domain | EKIEDMDDNCYLKLSCVIPGESVNYTWYGDKRPFPKELQNSVLETTLMPHNYSRCY |
| | | TCQVSNSVSSKNGTVCLSPPCTLAR |
| 20 | SLAMF2 | KDSAPTVVSGILGGSVTLPLNISVDTEIENVIWIGPKNALAFARPKENVTIMVKSY |
| | Extracellular | LGRLDITKWSYSLCISNLTLNDAGSYKAQINQRNFEVTTEEEFTLFVYEQLQEPQV |
| | Domain | TMKSVKVSENFSCNITLMCSVKGAEKSVLYSWTPREPHASESNGGSILTVSRTPCD |
| | | PDLPYICTAQNPVSQRSSLPVHVGQFCTDPGASRGGTTGETVVGVLGEPVTLPLAL |
| | | PACRDTEKVVWLFNTSIISKEREEAATADPLIKSRDPYKNRVWVSSQDCSLKISQL |
| | | KIEDAGPYHAYVCSEASSVTSMTHVTLLIYRRLRKPKITWSLRHSEDGICRISLTC |
| | | SVEDGGNTVMYTWTPLQKEAVVSQGESHLNVSWRSSENHPNLTCTASNPVSRSSHQ |
| | | FLSENICSGPERNTK |
| 21 | Ly-9 | KDSEIFTVNGILGESVTFPVNIQEPRQVKIIAWTSKTSVAYVTPGDSETAPVVTVT |
| | Extracellular | HRNYYERIHALGPNYNLVISDLRMEDAGDYKADINTQADPYTTTKRYNLQIYRRLG |
| | Domain | KPKITQSLMASVNSTCNVTLTCSVEKEEKNVTYNWSPLGEEGNVLQIFQTPEDQEL |
| | | TYTCTAQNPVSNNSDSISARQLCADIAMGERTHHTG |
| 22 | CD84 | QSSLTPLMVNGILGESVTLPLEFPAGEKVNFITWLFNETSLAFIVPHETKSPEIHV |
| | Extracellular | TNPKQGKRLNFTQSYSLQLSNLKMEDTGSYRAQISTKTSAKLSSYTLRILRQLRNI |
| | Domain | QVTNHSQLFQNMTCELHLTCSVEDADDNVSFRWEALGNTLSSQPNLTVSWDPRISS |
| | | EQDYTCIAENAVSNLSFSVSAQKLCEDVKIQYTDTK |
| 23 | Ly108 | EPHSLRYNLTVLSWDGSVQSGFLTEVHLDGQPFLRCDRQKCRAKPQGQWAEDVLGN |
| | Extracellular | KTWDRETRDLTGNGKDLRMTLAHIKDQKEGLHSLQEIRVCEIHEDNSTRSSQHFYY |
| | Domain | DGELFLSQNLETKEWTMPQSSRAQTLAMNVRNFLKEDAMKTKTHYHAMHADCLQEL |
| | | RRYLKSGVVLRRTVPPMVNVTRSEASEGNITVTCRASGFYPWNITLSWRQDGVSLS |
| | | HDTQQWGDVLPDGNGTYQTWVATRICQGEEQRFTCYMEHSGNHSTHPVPSGKVLVL |
| | | QSH |
| 24 | MICA | EPHSLRYNLMVLSQDGSVQSGFLAEGHLDGQPFLRYDRQKRRAKPQGQWAEDVLGA |
| | Extracellular | ETWDTETEDLTENGQDLRRTLTHIKDQKGVPQSSRAQTLAMNVTNFWKEDAMKTKT |
| | Domain | HYRAMQADCLQKLQRYLKSGVAIRRTVPPMVNVTCSEVSEGNITVTCRASSFYPRN |
| | | ITLTWRQDGVSLSHNTQQWGDVLPDGNGTYQTWVATRIRQGEEQRFTCYMEHSGNH |
| | | GTHPVPSGKALVLQSQRTD |
| 25 | MICB | MAAAASPAFLLCLPLLHLLSGWSRAGWVDTHCLCYDFIITPKSRPEPQWCEVQGLV |
| | Extracellular | DERPFLHYDCVNHKAKAFASLGKKVNVTKTWEEQTETLRDVVDFLKGQLLDIQVEN |
| | Domain | LIPIEPLTLQARMSCEHEAHGHGRGSWQFLENGQKFLLFDSNNRKWTALHPGAKKM |
| | | TEKWEKNRDVTMFFQKISLGDCKMWLEEFLMYWEQMLDPTKPPSLAPGTTQPKAMA |
| | | TTLSPWSLLIIFLCFILAGR |
| 26 | ULBP1 | MAAAAATKILLCLPLLLLLSGWSRAGRADPHSLCYDITVIPKFRPGPRWCAVQGQV |
| | | DEKTFLHYDCGNKTVTPVSPLGKKLNVTTAWKAQNPVLREVVDILTEQLRDIQLEN |
| | | YTPKEPLTLQARMSCEQKAEGHSSGSWQFSFDGQIFLLEDSEKRMWTTVHPGARKM |
| | | KEKWENDKVVAMSFHYFSMGDCIGWLEDFLMGMDSTLEPSAGAPLAMSSGTTQLRA |
| | | TATTLILCCLLIILPCFILPGI |
| 27 | ULBP2 | MAAAASPAILPRLAILPYLLFDWSGTGRADAHSLWYNFTIIHLPRHGQQWCEVQSQ |
| | | VDQKNFLSYDCGSDKVLSMGHLEEQLYATDAWGKQLEMLREVGQRLRLELADTELE |
| | | DFTPSGPLTLQVRMSCECEADGYIRGSWQFSFDGRKELLFDSNNRKWTVVHAGARR |
| | | MKEKWEKDSGLTTFFKMVSMRDCKSWLRDFLMHRKKRLEPTAPPTMAPGLAQPKAI |
| | | ATTLSPWSFLIILCFILPGI |
| 28 | ULBP3 | HSLCFNFTIKSLSRPGQPWCEAQVFLNKNLFLQYNSDNNMVKPLGLLGKKVYATST |
| | | WGELTQTLGEVGRDLRMLLCDIKPQIKTSDPSTLQVEMFCQREAERCTGASWQFAT |
| | | NGEKSLLFDAMNMTWTVINHEASKIKETWKKDRGLEKYFRKLSKGDCDHWLREFLG |
| | | HWEAMPEPTVSPVNASDIHWSSSSLPD |
| 29 | ULBP4 | GLADPHSLCYDITVIPKFRPGPRWCAVQGQVDEKTFLHYDCGSKTVTPVSPLGKKL |
| | Extracellular | NVTTAWKAQNPVLREVVDILTEQLLDIQLENYIPKEPLTLQARMSCEQKAEGHGSG |
| | Domain | SWQLSFDGQIFLLEDSENRMWTTVHPGARKMKEKWENDKDMTMSFHYISMGDCTGW |
| | | LEDFLMGMDSTLEPSAGAPPTMSSGTAQPR |
| 30 | ULBP5 | MAAAAIPALLLCLPLLFLLFGWSRARRDDPHSLCYDITVIPKFRPGPRWCAVQGQV |
| | Extracellular | DEKTFLHYDCGNKTVTPVSPLGKKLNVTMAWKAQNPVLREVVDILTEQLLDIQLEN |
| | Domain | YTPKEPLTLQARMSCEQKAEGHSSGSWQFSIDGQTFLLFDSEKRMWTTVHPGARKM |
| | | KEKWENDKDVAMSFHYISMGDCIGWLEDFLMGMDSTLEPSAGAPLAMSSGTTQLRA |
| | | TATTLILCCLLIILPCFILPGI |
| 31 | ULBP6 | DTQEKEVRAMVGSDVELSCACPEGSRFDLNDVYVYWQTSESKTVVTYHIPQNSSLE |
| | | NVDSRYRNRALMSPAGMLRGDFSLRLFNVTPQDEQKFHCLVLSQSLGFQEVLSVEV |
| | | TLHVAANFSVPVVSAPHSPSQDELTFTCTSINGYPRPNVYWINKTDNSLLDQALQN |
| | | DTVFLNMRGLYDVVSVLRIARTPSVNIGCCIENVLLQQNLTVGSQTGNDIGERDKI |
| | | TENPVSTGEKNAAT |
| 32 | B7-H2 | DLKVEMMAGGTQITPLNDNVTIFCNIFYSQPLNITSMGITWFWKSLTFDKEVKVFE |
| | Extracellular | FFGDHQEAFRPGAIVSPWRLKSGDASLRLPGIQLEEAGEYRCEVVVTPLKAQGTVQ |
| | Domain | LEVVASPASRLLLDQVGMKENEDKYMCESSGFYPEAINITWEKQTQKFPHPIEISE |
| | | DVITGPTIKNMDGTFNVTSCLKLNSSQEDPGTVYQCVVRHASLHTPLRSNFTLTAA |
| | | RHSLSETEKTDNES |
| 33 | B7-H6 | FKVATPYSLYVCPEGQNVTLTCRLLGPVDKGHDVTFYKTWYRSSRGEVQTCSERRP |
| | Extracellular | IRNLTFQDLHLHHGGHQAANTSHDLAQRHGLESASDHHGNFSITMRNLTLLDSGLY |
| | Domain | CCLVVEIRHHHSEHRVHGAMELQVQTGKDAPSNCVVYPSSSQDSENITAA |
| 34 | B7-H5 | LEVQVPEDPVVALVGTDATLCCSFSPEPGFSLAQLNLIWQLTDTKQLVHSFAEGQD |
| | Extracellular | QGSAYANRTALFPDLLAQGNASLRLQRVRVADEGSFTCFVSIRDFGSAAVSLQVAA |
| | Domain | PYSKPSMTLEPNKDLRPGDTVTITCSSYRGYPEAEVFWQDGQGVPLTGNVTTSQMA |
| | | NEQGLFDVHSVLRVVLGANGTYSCLVRNPVLQQDAHGSVTITGQPMTEPPEA |
| 35 | B7-H3 | LIIGFGISGRHSITVTTVASAGNIGEDGILSCTFEPDIKLSDIVIQWLKEGVLGLV |
| | Extracellular | HEFKEGKDELSEQDEMFRGRTAVFADQVIVGNASLRLKNVQLTDAGTYKCYIITSK |
| | Domain | GKGNANLEYKTGAFSMPEVNVDYNASSETLRCEAPRWFPQPTVVWASQVDQGANFS |
| | | EVSNTSFELNSENVTMKVVSVLYNVTINNTYSCMIENDIAKATGDIKVTESEIKRR |
| | | SHLQLLNSKASTTENLYFQG |
| 36 | B7x | LSVQQGPNLLQVRQGSQATLVCQVDQATAWERLRVKWTKDGAILCQPYITNGSLSL |
| | Extracellular | GVCGPQGRLSWQAPSHLTLQLDPVSLNHSGAYVCWAAVEIPELEEAEGNITRLFVD |
| | Domain | PDDPTQNRNRIASFPG |
| 37 | TMIGD2 | APTSSSTKKTQLQLEHLLLDLQMILNGINNYKNPKLTRMLTFKFYMPKKATELKHL |
| | Extracellular | QCLEEELKPLEEVLNLAQSKNFHLRPRDLISNINVIVLELKGSETTFMCEYADETA |
| | Domain | TIVEFLNRWITFCQSIISTLT |
| 38 | IL-2 | DCDIEGKDGKQYESVLMVSIDQLLDSMKEIGSNCLNNEFNFFKRHICDANKEGMFL |
| | | FRAARKLRQFLKMNSTGDFDLHLLKVSEGTTILLNCTGQVKGRKPAALGEAQPTKS |
| | | LEENKSLKEQKKLNDLCFLKRLLQEIKTCWNKILMGTKEH |
| 39 | IL-7 | RNLPVATPDPGMFPCLHHSQNLLRAVSNMLQKARQTLEFYPCTSEEIDHEDITKDK |
| | | TSTVEACLPLELTKNESCLNSRETSFITNGSCLASRKTSFMMALCLSSIYEDLKMY |
| | | QVEFKTMNAKLLMDPKRQIFLDQNMLAVIDELMQALNFNSETVPQKSSLEEPDFYK |
| | | TKIKLCILLHAFRIRAVTIDRVMSYLNAS |
| 40 | IL-12 | IWELKKDVYVVELDWYPDAPGEMVVLTCDTPEEDGITWTLDQSSEVLGSGKTLTIQ |
| | subunit | VKEFGDAGQYTCHKGGEVLSHSLLLLHKKEDGIWSTDILKDQKEPKNKTFLRCEAK |
| | alpha | NYSGRFTCWWLTTISTDLTFSVKSSRGSSDPQGVTCGAATLSAERVRGDNKEYEYS |
| | | VECQEDSACPAAEESLPIEVMVDAVHKLKYENYTSSFFIRDIIKPDPPKNLQLKPL |
| | | KNSRQVEVSWEYPDTWSTPHSYFSLTFCVQVQGKSKREKKDRVFTDKTSATVICRK |
| | | NASISVRAQDRYYSSSWSEWASVPCS |
| 41 | IL-12 | NWVNVISDLKKIEDLIQSMHIDATLYTESDVHPSCKVTAMKCFLLELQVISLESGD |
| | subunit | ASIHDTVENLIILANNSLSSNGNVTESGCKECEELEEKNIKEFLQSFVHIVQMFIN |
| | beta | TS |
| 42 | IL-15 | YFGKLESKLSVIRNLNDQVLFIDQGNRPLFEDMTDSDCRDNAPRTIFIISMYKDSQ |
| | | PRGMAVTISVKCEKISTLSCENKIISFKEMNPPDNIKDTKSDIIFFQRSVPGHDNK |
| | | MQFESSSYEGYFLACEKERDLFKLILKKEDELGDRSIMFTVQNED |
| 43 | IL-18 | HKSSSQGQDRHMIRMRQLIDIVDQLKNYVNDLVPEFLPAPEDVETNCEWSAFSCFQ |
| | | KAQLKSANTGNNERIINVSIKKLKRKPPSTNAGRRQKHRLTCPSCDSYEKKPPKEF |
| | | LERFKSLLQKMIHQHLSSRTHGSEDS |
| 44 | IL-21 | QTSVSPSKVILPRGGSVLVTCSTSCDQPKLLGIETPLPKKELLLPGNNRKVYELSN |
| | | VQEDSQPMCYSNCPDGQSTAKTFLTVYWTPERVELAPLPSWQPVGKNLTLRCQVEG |
| | | GAPRANLTVVLLRGEKELKREPAVGEPAEVTTTVLVRRDHHGANFSCRTELDLRPQ |
| | | GLELFENTSAPYQLQTFVLPATPPQLVSPRVLEVDTQGTVVCSLDGLFPVSEAQVH |
| | | LALGDQRLNPTVTYGNDSFSAKASVSVTAEDEGTQRLTCAVILGNQSQETLQTVTI |
| | | YSFPAPNVILTKPEVSEGTEVTVKCEAHPRAKVTLNGVPAQPLGPRAQLLLKATPE |
| | | DNGRSFSCSATLEVAGQLIHKNQTRELRVLYGPRLDERDCPGNWTWPENSQQTPMC |
| | | QAWGNPLPELKCLKDGTFPLPIGESVTVTRDLEGTYLCRARSTQGEVTRKVTVNVL |
| | | SPRYE |
| 45 | E2 SCFV VL | TCCGTGCTGACCCAGCCTAGCTCCGTGTCTGCCGCACCAGGACAGAAGGTGACAAT |
| | nucleotide | CAGCTGTTCCGGCTCTACCAGCAACATCGGCAACAATTACGTGAGCTGGTACCAGC |
| | | AGCACCCTGGCAAGGCCCCAAAGCTGATGATCTACGACGTGTCCAAGAGGCCATCT |
| | | GGAGTGCCTGATCGGTTCTCCGGCTCTAAGAGCGGCAATTCCGCCTCTCTGGACAT |
| | | CAGCGGACTGCAGTCCGAGGACGAGGCAGATTACTATTGCGCCGCCTGGGACGATA |
| | | GCCTGTCCGAGTTTCTGTTCGGCACCGGCACAAAGCTGACCGTGCTG |
| 46 | Gag | MGARASVLSGGELDRWEKIRLRPGGKKKYKLKHIVWASRELERFAVNPGLLETSEG |
| | polypeptide | CRQILGQLQPSLQTGSEELRSLYNTVATLYCVHQRIEIKDTKEALDKIEEEQNKSK |
| | | KKAQQAAADTGHSNQVSQNYPIVQNIQGQMVHQAISPRTLNAWVKVVEEKAFSPEV |
| | | IPMFSALSEGATPQDLNTMLNTVGGHQAAMQMLKETINEEAAEWDRVHPVHAGPIA |
| | | PGQMREPRGSDIAGTTSTLQEQIGWMTHNPPIPVGEIYKRWIILGLNKIVRMYSPT |
| | | SILDIRQGPKEPFRDYVDRFYKTLRAEQASQEVKNWMTETLLVQNANPDCKTILKA |
| | | LGPGATLEEMMTACQGVGGPGHKARVLAEAMSQVINPATIMIQKGNFRNQRKTVKC |
| | | FNCGKEGHIAKNCRAPRKKGCWKCGKEGHQMKDCTERQANFLGKIWPSHKGRPGNF |
| | | LQSRPEPTAPPEESFRFGEETTTPSQKQEPIDKELYPLASLRSLFGSDPSSQ |
| 47 | E2 scFv VL | SVLTQPSSVSAAPGQKVTISCSGSTSNIGNNYVSWYQQHPGKAPKLMIYDVSKRPS |
| | polypeptide | GVPDRFSGSKSGNSASLDISGLQSEDEADYYCAAWDDSLSEFLFGTGTKLTVL |
| 48 | E2 scFv VH | CAGGTGCAGCTGGTGGAGAGCGGAGGAAACCTGGTGCAGCCAGGAGGCTCCCTGCG |
| | nucleotide | CCTGTCTTGTGCCGCCAGCGGCTTTACCTTCGGCTCTTTTAGCATGTCCTGGGTGC |
| | | GCCAGGCACCTGGAGGAGGACTGGAGTGGGTGGCCGGCCTGAGCGCCCGGTCTAGC |
| | | CTGACACACTATGCCGACTCCGTGAAGGGCCGCTTCACCATCTCCCGGGATAACGC |
| | | CAAGAATAGCGTGTACCTGCAGATGAATAGCCTGCGGGTGGAGGACACAGCCGTGT |
| | | ACTATTGCGCCAGGCGCTCCTATGATTCCTCTGGCTACTGGGGCCACTTTTACTCT |
| | | TATATGGACGTGTGGGGACAGGGCACCCTGGTGACAGTGAGCTCC |
| 49 | Pol | FFREDLAFPQGKAREFSSEQTRANSPTRRELQVWGRDNNSLSEAGADRQGTVSFSF |
| | polypeptide | PQITLWQRPLVTIKIGGQLKEALLDTGADDTVLEEMNLPGRWKPKMIGGIGGFIKV |
| | | RQYDQILIEICGHKAIGTVLVGPTPVNIIGRNLLTQIGCTLNFPISPIETVPVKLK |
| | | PGMDGPKVKQWPLTEEKIKALVEICTEMEKEGKISKIGPENPYNTPVFAIKKKDST |
| | | KWRKLVDFRELNKRTQDFWEVQLGIPHPAGLKQKKSVTVLDVGDAYFSVPLDKDER |
| | | KYTAFTIPSINNETPGIRYQYNVLPQGWKGSPAIFQCSMTKILEPFRKQNPDIVIY |
| | | QYMDDLYVGSDLEIGQHRTKIEELRQHLLRWGFTTPDKKHQKEPPFLWMGYELHPD |
| | | KWTVQPIVLPEKDSWTVNDIQKLVGKLNWASQIYAGIKVRQLCKLLRGTKALTEVV |
| | | PLTEEAELELAENREILKEPVHGVYYDPSKDLIAEIQKQGQGQWTYQIYQEPFKNL |
| | | KTGKYARMKGAHTNDVKQLTEAVQKIATESIVIWGKTPKFKLPIQKETWEAWWTEY |
| | | WQATWIPEWEFVNTPPLVKLWYQLEKEPIIGAETFYVDGAANRETKLGKAGYVTDR |
| | | GRQKVVPLTDTTNQKTELQAIHLALQDSGLEVNIVTDSQYALGIIQAQPDKSESEL |
| | | VSQIIEQLIKKEKVYLAWVPAHKGIGGNEQVDKLVSAGIRKVLFLDGIDKAQEEHE |
| | | KYHSNWRAMASDFNLPPVVAKEIVASCDKCQLKGEAMHGQVDCSPGIWQLDCTHLE |
| | | GKVILVAVHVASGYIEAEVIPAETGQETAYFLLKLAGRWPVKTVHTDNGSNFTSTT |
| | | VKAACWWAGIKQEFGIPYNPQSQGVIESMNKELKKIIGQVRDQAEHLKTAVQMAVF |
| | | IHNFKRKGGIGGYSAGERIVDIIATDIQTKELQKQITKIQNERVYYRDSRDPVWKG |
| | | PAKLLWKGEGAVVIQDNSDIKVVPRRKAKIIRDYGKQMAGDDCVASRQDED |
| 50 | E2 scFv VH | QVQLVESGGNLVQPGGSLRLSCAASGFTFGSFSMSWVRQAPGGGLEWVAGLSARSS |
| | polypeptide | LTHYADSVKGRFTISRDNAKNSVYLQMNSLRVEDTAVYYCARRSYDSSGYWGHFYS |
| | | YMDVWGQGTLVTVSS |
| 51 | E2 scFv | GGCTCTACAAGCGGATCCGGCAAGCCAGGATCTGGAGAGGGCAGCACAAAGGGA |
| | linker | |
| | nucleotide | |
| 52 | Gag-pol | atgggcgcccgcgccagcgtgctttctggcggcgagctggacaggtgggagaagat |
| | nucleotide | tcgcctgcggcctggaggaaagaaaaagtacaagctgaagcacatcgtgtgggctt |
| | | ctcgggaactggaaagattcgccgtgaaccctggactgctagagacctccgaaggc |
| | | tgcagacagatcctgggacagctgcaacctagcctgcagaccggcagcgaggagct |
| | | gagaagcctgtacaacaccgtcgccaccctgtattgtgtgcaccaaagaatcgaga |
| | | tcaaggacaccaaggaggctctggataagatcgaggaagagcagaacaagagcaag |
| | | aaaaaagcccagcaggccgccgctgataccggccattctaatcaggtgtcccagaa |
| | | ctaccccattgtgcaaaatatccagggccagatggtccaccaggccatcagcccta |
| | | gaaccctgaatgcctgggtgaaggtggtggaagagaaggccttttctccagaggtg |
| | | atccctatgttcagcgccctgagcgagggcgctacccctcaggacctgaacacaat |
| | | gctgaacaccgtgggcggccaccaggccgccatgcagatgctgaaagaaaccatca |
| | | acgaggaggccgccgaatgggaccgggtgcaccccgttcacgccggcccaatcgcc |
| | | cctggccagatgcgggaacctagaggcagcgacatcgccggcacaaccagcacact |
| | | gcaagagcagatcggatggatgacacacaacccccccatccccgtgggcgaaatct |
| | | acaagcggtggatcattctgggactgaacaaaatcgttagaatgtacagccctacc |
| | | agcatcctggatatcagacagggcccaaaggagcctttccgggactacgtggacag |
| | | attttacaagaccctgagagccgaacaggcctcccaagaggtgaagaactggatga |
| | | cagagaccctgctggtgcagaatgccaaccctgattgtaagacaatcctgaaggcc |
| | | ctcggacctggcgctacactggaagaaatgatgaccgcctgccagggcgtgggcgg |
| | | ccccggccacaaggccagagtgctggccgaggctatgagccaggtgacaaaccccg |
| | | ccacaatcatgatccagaagggcaacttcagaaaccagcggaagaccgtgaaatgc |
| | | ttcaactgcggcaaggaaggccacatcgcaaagaactgcagagcccctaggaagaa |
| | | aggctgttggaagtgcggaaaggaaggacaccaaatgaaagattgtactgagagac |
| | | aggctaattttttagggaagatctggccttcccacaagggaaggccagggaatttt |
| | | cttcagagcagaccagagccaacagccccaccagaagagagcttcaggtttgggga |
| | | agagacaacaactccctctcagaagcaggagccgatagacaaggaactgtatcctt |
| | | tagcttccctcagatcactctttggcagcgacccctcgtcacaataaagatcggcg |
| | | gacagctgaaagaggccttgctggacaccggagccgatgacaccgtgctggaagaa |
| | | atgaacctgcctggaagatggaaacctaagatgatcggtggcatcggcggatttat |
| | | caaagtgcgacagtatgaccagatcctgatcgagatttgcggccacaaagctatcg |
| | | gaacagtgctggtcggcccgacccccgtgaacatcattggccgcaacctgctgaca |
| | | cagatcggttgtacactgaactttcctatcagccctatcgaaaccgtgccggtcaa |
| | | gctgaagcccggcatggatggccctaaggtgaagcagtggcccctgacagaggaaa |
| | | agatcaaggcactggtggaaatctgcacagaaatggaaaaagagggcaagatttct |
| | | aaaatcggcccagagaacccctacaacacccctgttttcgccatcaagaagaaaga |
| | | ttccaccaagtggaggaagctggtggactttcgggaactgaacaagcggacccagg |
| | | atttctgggaggtgcagctgggcatcccccaccctgccggcctgaaacaaaaaaaa |
| | | agcgtgaccgtgctggacgtgggcgacgcctatttcagcgtgcctctggataagga |
| | | cttccggaaatacaccgcctttaccatccctagcatcaacaacgagacccctggca |
| | | tccggtaccagtacaacgtgctcccacagggctggaagggctcacccgccatcttc |
| | | cagtgcagcatgaccaagatcctggagcctttcagaaagcagaatcctgacatcgt |
| | | gatctaccagtacatggacgacctgtacgtgggctctgatctggagatcggacagc |
| | | acagaacaaagatcgaagagctgagacagcatctgctgagatggggtttcaccacc |
| | | cccgacaagaagcaccagaaggaacctccttttctgtggatgggctacgagctgca |
| | | ccccgataagtggacagtgcagcccatcgtgctgcccgagaaggactcctggaccg |
| | | tgaacgacattcagaagctggtcggaaagctgaattgggcttcccaaatctacgcc |
| | | ggcatcaaggtgcggcagctgtgcaagcttctgcgcggcacaaaggccctgacgga |
| | | agtcgtgccactgaccgaggaagccgaattagagctggccgaaaacagagaaattc |
| | | tgaaagaacctgtgcacggcgtttactacgacccttctaaggacctgatcgccgaa |
| | | atccagaaacaaggccagggccagtggacttaccaaatctaccaggagcctttcaa |
| | | aaacctcaagaccggcaagtacgccagaatgaagggagcccatacaaacgacgtga |
| | | agcagctgacagaggctgttcagaagatcgccacagaaagcatcgtgatctggggc |
| | | aagaccccaaagttcaagctgcctatccaaaaggaaacctgggaggcctggtggac |
| | | cgagtactggcaggccacctggattcctgaatgggagttcgtgaacacaccacctc |
| | | ttgtgaagctgtggtaccagctggaaaaggagccaatcatcggcgccgagacattc |
| | | tacgtggacggcgccgccaaccgggagaccaaactgggaaaggccggatatgtgac |
| | | cgacagaggcagacaaaaggtggtgcctctgaccgatacaactaaccagaaaacag |
| | | agctgcaggccattcacctggccctgcaagacagcggcctggaagtgaatatcgtg |
| | | acagacagtcagtacgccctgggcatcatccaggctcagcctgacaagagcgagag |
| | | cgagctggtgtcccagatcatcgagcagctgatcaaaaaggagaaggtttatctgg |
| | | cctgggtgcccgcccacaagggcatcggaggcaacgagcaggtggacaagctggtc |
| | | agcgccggcatccggaaggtgctgttcctggacggcatcgacaaggcccaggagga |
| | | acacgagaagtaccacagcaactggcgggccatggccagcgacttcaacctgccac |
| | | ctgtggttgctaaggagatcgtcgcctcttgtgataagtgccagctgaagggcgag |
| | | gccatgcacggccaagtggattgcagccctgggatctggcagctagactgtaccca |
| | | cctggagggcaaggtgatcctggtggcagtgcacgtggccagcggctacatcgagg |
| | | ctgaggtgatccccgccgaaacgggccaggagaccgcctactttctgctgaagcta |
| | | gccggccggtggcctgtgaagaccgtgcacaccgataacggcagcaatttcaccag |
| | | cacaaccgtgaaggctgcctgctggtgggctggaatcaagcaggagttcggcatcc |
| | | catacaatcctcagtctcagggcgtgatcgagagcatgaacaaggaactgaagaag |
| | | atcattggtcaggtcagagatcaggccgagcacctgaaaaccgccgttcaaatggc |
| | | tgtgttcatccataacttcaaaagaaaaggcggcatcggcggctacagcgccggcg |
| | | aaagaatcgtggatatcatcgcgaccgacatccaaacaaaagagctgcaaaagcaa |
| | | atcaccaagatccagaacttcagagtgtactacagagatagcagagatcctgtgtg |
| | | gaagggacctgccaagctgctgtggaagggcgagggcgccgtggtgatccaggaca |
| | | atagcgacatcaaggtcgtgcccagaagaaaggctaaaatcattagagactacggc |
| | | aaacagatggccggagatgattgcgtggcttctagacaggacgaggactga |
| 53 | E2 scFv | GSTSGSGKPGSGEGSTKG |
| | linker | |
| | polypeptide | |
| 54 | E2 scFv | TCCGTGCTGACCCAGCCTAGCTCCGTGTCTGCCGCACCAGGACAGAAGGTGACAAT |
| | nucleotide | CAGCTGTTCCGGCTCTACCAGCAACATCGGCAACAATTACGTGAGCTGGTACCAGC |
| | | AGCACCCTGGCAAGGCCCCAAAGCTGATGATCTACGACGTGTCCAAGAGGCCATCT |
| | | GGAGTGCCTGATCGGTTCTCCGGCTCTAAGAGCGGCAATTCCGCCTCTCTGGACAT |
| | | CAGCGGACTGCAGTCCGAGGACGAGGCAGATTACTATTGCGCCGCCTGGGACGATA |
| | | GCCTGTCCGAGTTTCTGTTCGGCACCGGCACAAAGCTGACCGTGCTGGGCTCTACA |
| | | AGCGGATCCGGCAAGCCAGGATCTGGAGAGGGCAGCACAAAGGGACAGGTGCAGCT |
| | | GGTGGAGAGCGGAGGAAACCTGGTGCAGCCAGGAGGCTCCCTGCGCCTGTCTTGTG |
| | | CCGCCAGCGGCTTTACCTTCGGCTCTTTTAGCATGTCCTGGGTGCGCCAGGCACCT |
| | | GGAGGAGGACTGGAGTGGGTGGCCGGCCTGAGCGCCCGGTCTAGCCTGACACACTA |
| | | TGCCGACTCCGTGAAGGGCCGCTTCACCATCTCCCGGGATAACGCCAAGAATAGCG |
| | | TGTACCTGCAGATGAATAGCCTGCGGGTGGAGGACACAGCCGTGTACTATTGCGCC |
| | | AGGCGCTCCTATGATTCCTCTGGCTACTGGGGCCACTTTTACTCTTATATGGACGT |
| | | GTGGGGACAGGGCACCCTGGTGACAGTGAGCTCC |
| 55 | E2 scFv | TCCGTGCTGACCCAGCCTAGCTCCGTGTCTGCCGCACCAGGACAGAAGGTGACAAT |
| | nucleotide | CAGCTGTTCCGGCTCTACCAGCAACATCGGCAACAATTACGTGAGCTGGTACCAGC |
| | | AGCACCCTGGCAAGGCCCCAAAGCTGATGATCTACGACGTGTCCAAGAGGCCATCT |
| | | GGAGTGCCTGATCGGTTCTCCGGCTCTAAGAGCGGCAATTCCGCCTCTCTGGACAT |
| | | CAGCGGACTGCAGTCCGAGGACGAGGCAGATTACTATTGCGCCGCCTGGGACGATA |
| | | GCCTGTCCGAGTTTCTGTTCGGCACCGGCACAAAGCTGACCGTGCTGGGCTCTACA |
| | | AGCGGATCCGGCAAGCCAGGATCTGGAGAGGGCAGCACAAAGGGACAGGTGCAGCT |
| | | GGTGGAGAGCGGAGGAAACCTGGTGCAGCCAGGAGGCTCCCTGCGCCTGTCTTGTG |
| | | CCGCCAGCGGCTTTACCTTCGGCTCTTTTAGCATGTCCTGGGTGCGCCAGGCACCT |
| | | GGAGGAGGACTGGAGTGGGTGGCCGGCCTGAGCGCCCGGTCTAGCCTGACACACTA |
| | | TGCCGACTCCGTGAAGGGCCGCTTCACCATCTCCCGGGATAACGCCAAGAATAGCG |
| | | TGTACCTGCAGATGAATAGCCTGCGGGTGGAGGACACAGCCGTGTACTATTGCGCC |
| | | AGGCGCTCCTATGATTCCTCTGGCTACTGGGGCCACTTTTACTCTTATATGGACGT |
| | | GTGGGGACAGGGCACCCTGGTGACAGTGAGCTCC |
| 56 | E2 scFv | SVLTQPSSVSAAPGQKVTISCSGSTSNIGNNYVSWYQQHPGKAPKLMIYDVSKRPS |
| | polypeptide | GVPDRFSGSKSGNSASLDISGLQSEDEADYYCAAWDDSLSEFLFGTGTKLTVLGST |
| | | SGSGKPGSGEGSTKGQVQLVESGGNLVQPGGSLRLSCAASGFTFGSFSMSWVRQAP |
| | | GGGLEWVAGLSARSSLTHYADSVKGRFTISRDNAKNSVYLQMNSLRVEDTAVYYCA |
| | | RRSYDSSGYWGHFYSYMDVWGQGTLVTVSS |
| 57 | CD8α Hinge | ACCACCACACCTGCTCCTAGACCACCTACACCCGCTCCTACCATCGCCAGCCAGCC |
| | nucleotide | TCTGTCTCTGAGACCTGAGGCCTGTAGACCTGCCGCTGGAGGCGCTGTGCACACCA |
| | | GAGGACTGGATTTCGCCTGCGACATCTAC |
| 58 | CD8α Hinge | TTTPAPRPPTPAPTIASQPLSLRPEACRPAAGGAVHTRGLDFACDIY |
| | polypeptide | |
| 59 | CD8 Hinge | GAVHTRGLDFACD |
| 60 | Modified | ESKYGPPSPPSPA |
| | IgG4 Hinge | |
| 61 | Modified | ESKYGPPSPPSP |
| | IgG4 Hinge | |
| 62 | IgG1 Hinge | EPKSCDKTHTCP |
| 63 | PD1 Hinge | PSPRPAGQFQTLV |
| 64 | CD28 Hinge | LCPSPLFPGPSKP |
| 65 | CD8 | ATCTGGGCTCCTCTGGCTGGAACATGCGGCGTGCTGCTCCTGAGCCTGGTGATCAC |
| | trans- | ACTGTACTGC |
| | membrane | |
| | nucleotide | |
| 66 | CD8 | IWAPLAGTCGVLLLSLVITLYC |
| | trans- | |
| | membrane | |
| | polypeptide | |
| 67 | 41BB | AAGCGCGGCCGGAAGAAGCTGCTCTACATCTTTAAGCAGCCATTCATGCGCCCCGT |
| | nucleotide | GCAGACCACACAGGAGGAGGACGGCTGCTCCTGTCGGTTTCCAGAGGAGGAGGAGG |
| | | GAGGATGTGAGCTG |
| 68 | 41BB | KRGRKKLLYIFKQPFMRPVQTTQEEDGCSCRFPEEEEGGCEL |
| | polypeptide | |
| 69 | CD3ζ | AGAGTGAAGTTCTCTAGGAGCGCCGATGCCCCTGCCTATCAGCAGGGACAGAACCA |
| | nucleotide | GCTGTACAACGAGCTGAATCTGGGCCGGAGAGAGGAGTACGACGTGCTGGATAAGA |
| | | GGAGGGGAAGAGACCCAGAGATGGGAGGCAAGCCTCGGAGAAAGAACCCACAGGAG |
| | | GGCCTGTATAATGAGCTGCAGAAGGACAAGATGGCCGAGGCCTACTCCGAGATCGG |
| | | CATGAAGGGAGAGAGGCGCCGGGGCAAGGGACACGATGGCCTGTATCAGGGCCTGA |
| | | GCACCGCCACAAAGGACACATACGATGCCCTGCACATGCAGGCCCTGCCTCCAAGG |
| 70 | CD33 | RVKFSRSADAPAYQQGQNQLYNELNLGRREEYDVLDKRRGRDPEMGGKPRRKNPQE |
| | polypeptide | GLYNELQKDKMAEAYSEIGMKGERRRGKGHDGLYQGLSTATKDTYDALHMQALPPR |
| 71 | CD19 CAR | CTGCTGCTGGTGACCTCCCTGCTGCTGTGCGAGCTGCCTCACCCAGCCTTTCTGCT |
| | nucleotide | GATCCCCGACATCCAGATGACACAGACCACAAGCTCCCTGTCTGCCAGCCTGGGCG |
| | | ACAGAGTGACCATCTCCTGTAGGGCCTCTCAGGATATCAGCAAGTACCTGAACTGG |
| | | TATCAGCAGAAGCCAGATGGCACAGTGAAGCTGCTGATCTACCACACCTCCAGGCT |
| | | GCACTCTGGAGTGCCAAGCCGGTTCTCCGGATCTGGAAGCGGCACCGACTATTCCC |
| | | TGACAATCTCTAACCTGGAGCAGGAGGATATCGCCACATACTTTTGCCAGCAGGGC |
| | | AATACCCTGCCATATACATTCGGCGGAGGAACCAAGCTGGAGATCACCGGATCCAC |
| | | ATCTGGAAGCGGCAAGCCAGGAAGCGGAGAGGGATCCACAAAGGGAGAGGTGAAGC |
| | | TGCAGGAGAGCGGACCAGGACTGGTGGCACCATCCCAGTCTCTGAGCGTGACCTGT |
| | | ACAGTGTCCGGCGTGTCTCTGCCTGACTACGGCGTGTCCTGGATCAGGCAGCCACC |
| | | TAGGAAGGGACTGGAGTGGCTGGGCGTGATCTGGGGCTCTGAGACCACATACTATA |
| | | ATTCTGCCCTGAAGAGCCGCCTGACCATCATCAAGGACAACTCCAAGTCTCAGGTG |
| | | TTTCTGAAGATGAATAGCCTGCAGACCGACGATACAGCCATCTACTATTGCGCCAA |
| | | GCACTACTATTACGGCGGCTCCTACGCCATGGATTATTGGGGCCAGGGCACCTCCG |
| | | TGACAGTGTCTAGCGGCGCTGTGCACACCAGAGGACTGGATTTCGCCTGCGACTTC |
| | | TGGGTGCTGGTGGTGGTGGGAGGCGTGCTGGCCTGTTACTCCCTGCTGGTGACCGT |
| | | GGCCTTTATCATCTTCTGGGTGAAGAGAGGCAGGAAGAAGCTGCTGTATATCTTTA |
| | | AGCAGCCCTTCATGCGCCCTGTGCAGACCACACAGGAGGAGGACGGCTGCAGCTGT |
| | | CGGTTTCCAGAGGAGGAGGAGGGAGGATGCGAGCTGCGCGTGAAGTTCAGCCGGTC |
| | | CGCCGATGCCCCTGCCTACCAGCAGGGCCAGAACCAGCTGTATAACGAGCTGAATC |
| | | TGGGCCGGAGAGAGGAGTACGACGTGCTGGATAAGAGGAGGGGAAGGGACCCAGAG |
| | | ATGGGAGGCAAGCCTCGGAGAAAGAACCCACAGGAGGGCCTGTACAATGAGCTGCA |
| | | GAAGGACAAGATGGCCGAGGCCTATTCTGAGATCGGCATGAAGGGAGAGAGGCGCC |
| | | GGGGCAAGGGACACGATGGCCTGTACCAGGGCCTGAGCACCGCCACAAAGGACACA |
| | | TATGATGCCCTGCACATGCAGGCCCTGCCACCTAGG |
| 72 | CD19 CAR | LLLVTSLLLCELPHPAFLLIPDIQMTQTTSSLSASLGDRVTISCRASQDISKYLNW |
| | polypeptide | YQQKPDGTVKLLIYHTSRLHSGVPSRFSGSGSGTDYSLTISNLEQEDIATYFCQQG |
| | | NTLPYTFGGGTKLEITGSTSGSGKPGSGEGSTKGEVKLQESGPGLVAPSQSLSVTC |
| | | TVSGVSLPDYGVSWIRQPPRKGLEWLGVIWGSETTYYNSALKSRLTIIKDNSKSQV |
| | | FLKMNSLQTDDTAIYYCAKHYYYGGSYAMDYWGQGTSVTVSSGAVHTRGLDFACDE |
| | | WVLVVVGGVLACYSLLVTVAFIIFWVKRGRKKLLYIFKQPFMRPVQTTQEEDGCSC |
| | | RFPEEEEGGCELRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLSTATKDT |
| | | YDALHMQALPPR |
| 73 | CD19 scFv | GACATCCAGATGACACAGACCACAAGCTCCCTGTCTGCCAGCCTGGGCGACAGAGT |
| | nucleotide | GACCATCTCCTGTAGGGCCTCTCAGGATATCAGCAAGTACCTGAACTGGTATCAGC |
| | | AGAAGCCAGATGGCACAGTGAAGCTGCTGATCTACCACACCTCCAGGCTGCACTCT |
| | | GGAGTGCCAAGCCGGTTCTCCGGATCTGGAAGCGGCACCGACTATTCCCTGACAAT |
| | | CTCTAACCTGGAGCAGGAGGATATCGCCACATACTTTTGCCAGCAGGGCAATACCC |
| | | TGCCATATACATTCGGCGGAGGAACCAAGCTGGAGATCACCGGATCCACATCTGGA |
| | | AGCGGCAAGCCAGGAAGCGGAGAGGGATCCACAAAGGGAGAGGTGAAGCTGCAGGA |
| | | GAGCGGACCAGGACTGGTGGCACCATCCCAGTCTCTGAGCGTGACCTGTACAGTGT |
| | | CCGGCGTGTCTCTGCCTGACTACGGCGTGTCCTGGATCAGGCAGCCACCTAGGAAG |
| | | GGACTGGAGTGGCTGGGCGTGATCTGGGGCTCTGAGACCACATACTATAATTCTGC |
| | | CCTGAAGAGCCGCCTGACCATCATCAAGGACAACTCCAAGTCTCAGGTGTTTCTGA |
| | | AGATGAATAGCCTGCAGACCGACGATACAGCCATCTACTATTGCGCCAAGCACTAC |
| | | TATTACGGCGGCTCCTACGCCATGGATTATTGGGGCCAGGGCACCTCCGTGACAGT |
| | | GTCTAGC |
| 74 | CD19 scFv | DIQMTQTTSSLSASLGDRVTISCRASQDISKYLNWYQQKPDGTVKLLIYHTSRLHS |
| | polypeptide | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGTSVTVSS |
| 75 | CD19 scFv | GACATCCAGATGACACAGACCACAAGCTCCCTGTCTGCCAGCCTGGGCGACAGAGT |
| | VL | GACCATCTCCTGTAGGGCCTCTCAGGATATCAGCAAGTACCTGAACTGGTATCAGC |
| | nucleotide | AGAAGCCAGATGGCACAGTGAAGCTGCTGATCTACCACACCTCCAGGCTGCACTCT |
| | | GGAGTGCCAAGCCGGTTCTCCGGATCTGGAAGCGGCACCGACTATTCCCTGACAAT |
| | | CTCTAACCTGGAGCAGGAGGATATCGCCACATACTTTTGCCAGCAGGGCAATACCC |
| | | TGCCATATACATTCGGCGGAGGAACCAAGCTGGAGATC |
| 76 | CD19 scFv | DIQMTQTTSSLSASLGDRVTISCRASQDISKYLNWYQQKPDGTVKLLIYHTSRLHS |
| | VL | GVPSRFSGSGSGTDYSLTISNLEQEDIATYFCQQGNTLPYTFGGGTKLEI |
| | polypeptide | |
| 77 | CD19 scFv | ACCGGATCCACATCTGGAAGCGGCAAGCCAGGAAGCGGAGAGGGATCCACAAAGGG |
| | linker | A |
| | nucleotide | |
| 78 | CD19 scFv | TGSTSGSGKPGSGEGSTKG |
| | linker | |
| | polypeptide | |
| 79 | CD19 scFv | GAGGTGAAGCTGCAGGAGAGCGGACCAGGACTGGTGGCACCATCCCAGTCTCTGAG |
| | VH | CGTGACCTGTACAGTGTCCGGCGTGTCTCTGCCTGACTACGGCGTGTCCTGGATCA |
| | nucleotide | GGCAGCCACCTAGGAAGGGACTGGAGTGGCTGGGCGTGATCTGGGGCTCTGAGACC |
| | | ACATACTATAATTCTGCCCTGAAGAGCCGCCTGACCATCATCAAGGACAACTCCAA |
| | | GTCTCAGGTGTTTCTGAAGATGAATAGCCTGCAGACCGACGATACAGCCATCTACT |
| | | ATTGCGCCAAGCACTACTATTACGGCGGCTCCTACGCCATGGATTATTGGGGCCAG |
| | | GGCACCTCCGTGACAGTGTCTAGC |
| 80 | CD19 scFv | GAGGTGAAGCTGCAGGAGAGCGGACCAGGACTGGTGGCACCATCCCAGTCTCTGAG |
| | VH | CGTGACCTGTACAGTGTCCGGCGTGTCTCTGCCTGACTACGGCGTGTCCTGGATCA |
| | polypeptide | GGCAGCCACCTAGGAAGGGACTGGAGTGGCTGGGCGTGATCTGGGGCTCTGAGACC |
| | | ACATACTATAATTCTGCCCTGAAGAGCCGCCTGACCATCATCAAGGACAACTCCAA |
| | | GTCTCAGGTGTTTCTGAAGATGAATAGCCTGCAGACCGACGATACAGCCATCTACT |
| | | ATTGCGCCAAGCACTACTATTACGGCGGCTCCTACGCCATGGATTATTGGGGCCAG |
| | | GGCACCTCCGTGACAGTGTCTAGC |
| 81 | CD8 hinge | GGCGCTGTGCACACCAGAGGACTGGATTTCGCCTGCGAC |
| | nucleotide | |
| 82 | CD8 hinge | GAVHTRGLDFACD |
| | polypeptide | |
| 83 | CD28 | TTCTGGGTGCTGGTGGTGGTGGGAGGCGTGCTGGCCTGTTACTCCCTGCTGGTGAC |
| | trans- | CGTGGCCTTTATCATCTTCTGGGTG |
| | membrane | |
| | domain | |
| | nucleotide | |
| 84 | CD28 | FWVLVVVGGVLACYSLLVTVAFIIFWV |
| | trans | |
| | membrane | |
| | domain | |
| | nucleotide | |
| 85 | 41BB | AAGAGAGGCAGGAAGAAGCTGCTGTATATCTTTAAGCAGCCCTTCATGCGCCCTGT |
| | nucleotide | GCAGACCACACAGGAGGAGGACGGCTGCAGCTGTCGGTTTCCAGAGGAGGAGGAGG |
| | | GAGGATGCGAGCTG |
| 86 | CD3ζ domain | CGCGTGAAGTTCAGCCGGTCCGCCGATGCCCCTGCCTACCAGCAGGGCCAGAACCA |
| | nucleotide | GCTGTATAACGAGCTGAATCTGGGCCGGAGAGAGGAGTACGACGTGCTGGATAAGA |
| | | GGAGGGGAAGGGACCCAGAGATGGGAGGCAAGCCTCGGAGAAAGAACCCACAGGAG |
| | | GGCCTGTACAATGAGCTGCAGAAGGACAAGATGGCCGAGGCCTATTCTGAGATCGG |
| | | CATGAAGGGAGAGAGGCGCCGGGGCAAGGGACACGATGGCCTGTACCAGGGCCTGA |
| | | GCACCGCCACAAAGGACACATATGATGCCCTGCACATGCAGGCCCTGCCACCTAGG |
| 87 | CD20 CAR | ATGCTGCTGCTGGTGACAAGCCTGCTGCTGTGCGAGCTGCCTCACCCAGCCTTTCT |
| | nucleotide | GCTGATCCCCGATATCGTGCTGACCCAGTCCCCCGCCATCCTGTCCGCCTCTCCTG |
| | (with flag) | GAGAGAAGGTGACCATGACATGTCGGGCCAGCTCCTCTGTGAACTACATGGACTGG |
| | | TATCAGAAGAAGCCTGGCAGCTCCCCCAAGCCTTGGATCTACGCCACCTCCAATCT |
| | | GGCCTCTGGAGTGCCAGCAAGATTCAGCGGATCCGGATCTGGCACAAGCTATTCCC |
| | | TGACCATCTCCAGGGTGGAGGCAGAGGATGCAGCAACATACTATTGCCAGCAGTGG |
| | | TCTTTCAACCCCCCTACATTTGGCGGCGGCACCAAGCTGGAGATCAAGGGCTCTAC |
| | | CAGCGGAGGAGGAAGCGGAGGAGGATCCGGAGGCGGCGGCTCTAGCGAGGTGCAGC |
| | | TGCAGCAGTCCGGAGCAGAGCTGGTGAAGCCTGGAGCCTCTGTGAAGATGAGCTGT |
| | | AAGGCCTCCGGCTACACCTTCACATCTTATAATATGCACTGGGTGAAGCAGACACC |
| | | AGGACAGGGACTGGAGTGGATCGGAGCAATCTACCCTGGCAACGGCGACACCAGCT |
| | | ATAATCAGAAGTTTAAGGGCAAGGCCACCCTGACAGCCGATAAGTCCTCTAGCACA |
| | | GCCTACATGCAGCTGTCCTCTCTGACCAGCGAGGACTCCGCCGATTACTATTGCGC |
| | | CCGGTCCAACTACTATGGCAGCTCCTATTGGTTCTTTGACGTGTGGGGAGCAGGAA |
| | | CAACCGTGACCGTGTCTAGCGCTGCAGGAGGCGGAGGATCTGGAGGCGGCGGCGGA |
| | | GACTACAAAGACGATGACGACAAGTTCGAAGCAAAGCCAACCACCACACCTGCTCC |
| | | TAGACCACCTACACCCGCTCCTACCATCGCCAGCCAGCCTCTGTCTCTGAGACCTG |
| | | AGGCCTGTAGACCTGCCGCTGGAGGCGCTGTGCACACCAGAGGACTGGATTTCGCC |
| | | TGCGACATCTACATCTGGGCTCCTCTGGCTGGAACATGCGGCGTGCTGCTCCTGAG |
| | | CCTGGTGATCACACTGTACTGCAAGAGAGGCAGGAAGAAGCTGCTGTATATCTTTA |
| | | AGCAGCCCTTCATGCGCCCTGTGCAGACCACACAGGAGGAGGACGGCTGCAGCTGT |
| | | CGGTTTCCAGAGGAGGAGGAGGGAGGATGCGAGCTGCGCGTGAAGTTCAGCCGGTC |
| | | CGCCGATGCCCCTGCCTACCAGCAGGGCCAGAACCAGCTGTATAACGAGCTGAATC |
| | | TGGGCCGGAGAGAGGAGTACGACGTGCTGGATAAGAGGAGGGGAAGGGACCCAGAG |
| | | ATGGGAGGCAAGCCTCGGAGAAAGAACCCACAGGAGGGCCTGTACAATGAGCTGCA |
| | | GAAGGACAAGATGGCCGAGGCCTATTCTGAGATCGGCATGAAGGGAGAGAGGCGCC |
| | | GGGGCAAGGGACACGATGGCCTGTACCAGGGCCTGAGCACCGCCACAAAGGACACC |
| | | TATGATGCCCTGCACATGCAGGCCCTGCCCCCTCGG |
| 88 | CD20 CAR | MLLLVTSLLLCELPHPAFLLIPDIVLTQSPAILSASPGEKVTMTCRASSSVNYMDW |
| | polypeptide | YQKKPGSSPKPWIYATSNLASGVPARFSGSGSGTSYSLTISRVEAEDAATYYCQQW |
| | (with flag | SFNPPTFGGGTKLEIKGSTSGGGSGGGSGGGGSSEVQLQQSGAELVKPGASVKMSC |
| | sequence) | KASGYTFTSYNMHWVKQTPGQGLEWIGAIYPGNGDTSYNQKFKGKATLTADKSSST |
| | | AYMQLSSLTSEDSADYYCARSNYYGSSYWFFDVWGAGTTVTVSSAAGGGGSGGGGG |
| | | DYKDDDDKFEAKPTTTPAPRPPTPAPTIASQPLSLRPEACRPAAGGAVHTRGLDFA |
| | | CDIYIWAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLSTATKDT |
| | | YDALHMQALPPR |
| 89 | CD20 scFv | GATATCGTGCTGACCCAGTCCCCCGCCATCCTGTCCGCCTCTCCTGGAGAGAAGGT |
| | nucleotide | GACCATGACATGTCGGGCCAGCTCCTCTGTGAACTACATGGACTGGTATCAGAAGA |
| | | AGCCTGGCAGCTCCCCCAAGCCTTGGATCTACGCCACCTCCAATCTGGCCTCTGGA |
| | | GTGCCAGCAAGATTCAGCGGATCCGGATCTGGCACAAGCTATTCCCTGACCATCTC |
| | | CAGGGTGGAGGCAGAGGATGCAGCAACATACTATTGCCAGCAGTGGTCTTTCAACC |
| | | CCCCTACATTTGGCGGCGGCACCAAGCTGGAGATCAAGGGCTCTACCAGCGGAGGA |
| | | GGAAGCGGAGGAGGATCCGGAGGCGGCGGCTCTAGCGAGGTGCAGCTGCAGCAGTC |
| | | CGGAGCAGAGCTGGTGAAGCCTGGAGCCTCTGTGAAGATGAGCTGTAAGGCCTCCG |
| | | GCTACACCTTCACATCTTATAATATGCACTGGGTGAAGCAGACACCAGGACAGGGA |
| | | CTGGAGTGGATCGGAGCAATCTACCCTGGCAACGGCGACACCAGCTATAATCAGAA |
| | | GTTTAAGGGCAAGGCCACCCTGACAGCCGATAAGTCCTCTAGCACAGCCTACATGC |
| | | AGCTGTCCTCTCTGACCAGCGAGGACTCCGCCGATTACTATTGCGCCCGGTCCAAC |
| | | TACTATGGCAGCTCCTATTGGTTCTTTGACGTGTGGGGAGCAGGAACAACCGTGAC |
| | | CGTGTCTAGC |
| 90 | CD20 scFv | DIVLTQSPAILSASPGEKVTMTCRASSSVNYMDWYQKKPGSSPKPWIYATSNLASG |
| | polypeptide | VPARFSGSGSGTSYSLTISRVEAEDAATYYCQQWSFNPPTFGGGTKLEIKGSTSGG |
| | | GSGGGSGGGGSSEVQLQQSGAELVKPGASVKMSCKASGYTFTSYNMHWVKQTPGQG |
| | | LEWIGAIYPGNGDTSYNQKFKGKATLTADKSSSTAYMQLSSLTSEDSADYYCARSN |
| | | YYGSSYWFFDVWGAGTTVTVSS |
| 91 | CD20 scFv | GATATCGTGCTGACCCAGTCCCCCGCCATCCTGTCCGCCTCTCCTGGAGAGAAGGT |
| | VL | GACCATGACATGTCGGGCCAGCTCCTCTGTGAACTACATGGACTGGTATCAGAAGA |
| | nucleotide | AGCCTGGCAGCTCCCCCAAGCCTTGGATCTACGCCACCTCCAATCTGGCCTCTGGA |
| | | GTGCCAGCAAGATTCAGCGGATCCGGATCTGGCACAAGCTATTCCCTGACCATCTC |
| | | CAGGGTGGAGGCAGAGGATGCAGCAACATACTATTGCCAGCAGTGGTCTTTCAACC |
| | | CCCCTACATTTGGCGGCGGCACCAAGCTGGAGATCAAG |
| 92 | CD20 scFv | DIVLTQSPAILSASPGEKVTMTCRASSSVNYMDWYQKKPGSSPKPWIYATSNLASG |
| | VL | VPARFSGSGSGTSYSLTISRVEAEDAATYYCQQWSENPPTFGGGTKLEIK |
| | polypeptide | |
| 93 | 218 linker | GGCTCTACCAGCGGAGGAGGAAGCGGAGGAGGATCCGGAGGCGGCGGCTCTAGC |
| | nucleotide | |
| 94 | 218 linker | GSTSGGGSGGGSGGGGSS |
| | polypeptide | |
| 95 | CD20 scFv | GAGGTGCAGCTGCAGCAGTCCGGAGCAGAGCTGGTGAAGCCTGGAGCCTCTGTGAA |
| | VH | GATGAGCTGTAAGGCCTCCGGCTACACCTTCACATCTTATAATATGCACTGGGTGA |
| | nucleotide | AGCAGACACCAGGACAGGGACTGGAGTGGATCGGAGCAATCTACCCTGGCAACGGC |
| | | GACACCAGCTATAATCAGAAGTTTAAGGGCAAGGCCACCCTGACAGCCGATAAGTC |
| | | CTCTAGCACAGCCTACATGCAGCTGTCCTCTCTGACCAGCGAGGACTCCGCCGATT |
| | | ACTATTGCGCCCGGTCCAACTACTATGGCAGCTCCTATTGGTTCTTTGACGTGTGG |
| | | GGAGCAGGAACAACCGTGACCGTGTCTAGC |
| 96 | CD20 scFv | EVQLQQSGAELVKPGASVKMSCKASGYTFTSYNMHWVKQTPGQGLEWIGAIYPGNG |
| | VH | DTSYNQKFKGKATLTADKSSSTAYMQLSSLTSEDSADYYCARSNYYGSSYWFFDVW |
| | polypeptide | GAGTTVTVSS |
| 97 | Flag | GACTACAAAGACGATGACGACAAG |
| | nucleotide | |
| 98 | Flag | DYKDDDDK |
| | polypeptide | |
| 99 | CD8 HTM | GCAAAGCCAACCACCACACCTGCTCCTAGACCACCTACACCCGCTCCTACCATCGC |
| | nucleotide | CAGCCAGCCTCTGTCTCTGAGACCTGAGGCCTGTAGACCTGCCGCTGGAGGCGCTG |
| | | TGCACACCAGAGGACTGGATTTCGCCTGCGACATCTACATCTGGGCTCCTCTGGCT |
| | | GGAACATGCGGCGTGCTGCTCCTGAGCCTGGTGATCACACTGTACTGC |
| 100 | CD8 HTM | AKPTTTPAPRPPTPAPTIASQPLSLRPEACRPAAGGAVHTRGLDFACDIYIWAPLA |
| | polypeptide | GTCGVLLLSLVITLYC |
| 101 | CD3ζ | CGCGTGAAGTTCAGCCGGTCCGCCGATGCCCCTGCCTACCAGCAGGGCCAGAACCA |
| | nucleotide | GCTGTATAACGAGCTGAATCTGGGCCGGAGAGAGGAGTACGACGTGCTGGATAAGA |
| | | GGAGGGGAAGGGACCCAGAGATGGGAGGCAAGCCTCGGAGAAAGAACCCACAGGAG |
| | | GGCCTGTACAATGAGCTGCAGAAGGACAAGATGGCCGAGGCCTATTCTGAGATCGG |
| | | CATGAAGGGAGAGAGGCGCCGGGGCAAGGGACACGATGGCCTGTACCAGGGCCTGA |
| | | GCACCGCCACAAAGGACACCTATGATGCCCTGCACATGCAGGCCCTGCCCCCTCGG |
| 102 | TagCAR | CTGCTGCTGGTGACAAGCCTGCTGCTGTGCGAGCTGCCTCACCCAGCCTTTCTGCT |
| | nucleotide | GATCCCCTCCGTGCTGACCCAGCCTAGCTCCGTGTCTGCCGCACCAGGACAGAAGG |
| | | TGACAATCAGCTGTTCCGGCTCTACCAGCAACATCGGCAACAATTACGTGAGCTGG |
| | | TACCAGCAGCACCCTGGCAAGGCCCCAAAGCTGATGATCTACGACGTGTCCAAGAG |
| | | GCCATCTGGAGTGCCTGATCGGTTCTCCGGCTCTAAGAGCGGCAATTCCGCCTCTC |
| | | TGGACATCAGCGGACTGCAGTCCGAGGACGAGGCAGATTACTATTGCGCCGCCTGG |
| | | GACGATAGCCTGTCCGAGTTTCTGTTCGGCACCGGCACAAAGCTGACCGTGCTGGG |
| | | CTCTACAAGCGGATCCGGCAAGCCAGGATCTGGAGAGGGCAGCACAAAGGGACAGG |
| | | TGCAGCTGGTGGAGAGCGGAGGAAACCTGGTGCAGCCAGGAGGCTCCCTGCGCCTG |
| | | TCTTGTGCCGCCAGCGGCTTTACCTTCGGCTCTTTTAGCATGTCCTGGGTGCGCCA |
| | | GGCACCTGGAGGAGGACTGGAGTGGGTGGCCGGCCTGAGCGCCCGGTCTAGCCTGA |
| | | CACACTATGCCGACTCCGTGAAGGGCCGCTTCACCATCTCCCGGGATAACGCCAAG |
| | | AATAGCGTGTACCTGCAGATGAATAGCCTGCGGGTGGAGGACACAGCCGTGTACTA |
| | | TTGCGCCAGGCGCTCCTATGATTCCTCTGGCTACTGGGGCCACTTTTACTCTTATA |
| | | TGGACGTGTGGGGACAGGGCACCCTGGTGACAGTGAGCTCCACCACCACACCTGCT |
| | | CCTAGACCACCTACACCCGCTCCTACCATCGCCAGCCAGCCTCTGTCTCTGAGACC |
| | | TGAGGCCTGTAGACCTGCCGCTGGAGGCGCTGTGCACACCAGAGGACTGGATTTCG |
| | | CCTGCGACATCTACATCTGGGCTCCTCTGGCTGGAACATGCGGCGTGCTGCTCCTG |
| | | AGCCTGGTGATCACACTGTACTGCAAGCGCGGCCGGAAGAAGCTGCTCTACATCTT |
| | | TAAGCAGCCATTCATGCGCCCCGTGCAGACCACACAGGAGGAGGACGGCTGCTCCT |
| | | GTCGGTTTCCAGAGGAGGAGGAGGGAGGATGTGAGCTGAGAGTGAAGTTCTCTAGG |
| | | AGCGCCGATGCCCCTGCCTATCAGCAGGGACAGAACCAGCTGTACAACGAGCTGAA |
| | | TCTGGGCCGGAGAGAGGAGTACGACGTGCTGGATAAGAGGAGGGGAAGAGACCCAG |
| | | AGATGGGAGGCAAGCCTCGGAGAAAGAACCCACAGGAGGGCCTGTATAATGAGCTG |
| | | CAGAAGGACAAGATGGCCGAGGCCTACTCCGAGATCGGCATGAAGGGAGAGAGGCG |
| | | CCGGGGCAAGGGACACGATGGCCTGTATCAGGGCCTGAGCACCGCCACAAAGGACA |
| | | CATACGATGCCCTGCACATGCAGGCCCTGCCTCCAAGGTGA |
| 103 | TagCAR | ATGCTGCTGCTGGTGACAAGCCTGCTGCTGTGCGAGCTGCCTCACCCAGCCTTTCT |
| | nucleotide | GCTGATCCCCTCCGTGCTGACCCAGCCTAGCTCCGTGTCTGCCGCACCAGGACAGA |
| | | AGGTGACAATCAGCTGTTCCGGCTCTACCAGCAACATCGGCAACAATTACGTGAGC |
| | | TGGTACCAGCAGCACCCTGGCAAGGCCCCAAAGCTGATGATCTACGACGTGTCCAA |
| | | GAGGCCATCTGGAGTGCCTGATCGGTTCTCCGGCTCTAAGAGCGGCAATTCCGCCT |
| | | CTCTGGACATCAGCGGACTGCAGTCCGAGGACGAGGCAGATTACTATTGCGCCGCC |
| | | TGGGACGATAGCCTGTCCGAGTTTCTGTTCGGCACCGGCACAAAGCTGACCGTGCT |
| | | GGGCTCTACAAGCGGATCCGGCAAGCCAGGATCTGGAGAGGGCAGCACAAAGGGAC |
| | | AGGTGCAGCTGGTGGAGAGCGGAGGAAACCTGGTGCAGCCAGGAGGCTCCCTGCGC |
| | | CTGTCTTGTGCCGCCAGCGGCTTTACCTTCGGCTCTTTTAGCATGTCCTGGGTGCG |
| | | CCAGGCACCTGGAGGAGGACTGGAGTGGGTGGCCGGCCTGAGCGCCCGGTCTAGCC |
| | | TGACACACTATGCCGACTCCGTGAAGGGCCGCTTCACCATCTCCCGGGATAACGCC |
| | | AAGAATAGCGTGTACCTGCAGATGAATAGCCTGCGGGTGGAGGACACAGCCGTGTA |
| | | CTATTGCGCCAGGCGCTCCTATGATTCCTCTGGCTACTGGGGCCACTTTTACTCTT |
| | | ATATGGACGTGTGGGGACAGGGCACCCTGGTGACAGTGAGCTCCACCACCACACCT |
| | | GCTCCTAGACCACCTACACCCGCTCCTACCATCGCCAGCCAGCCTCTGTCTCTGAG |
| | | ACCTGAGGCCTGTAGACCTGCCGCTGGAGGCGCTGTGCACACCAGAGGACTGGATT |
| | | TCGCCTGCGACATCTACATCTGGGCTCCTCTGGCTGGAACATGCGGCGTGCTGCTC |
| | | CTGAGCCTGGTGATCACACTGTACTGCAAGCGCGGCCGGAAGAAGCTGCTCTACAT |
| | | CTTTAAGCAGCCATTCATGCGCCCCGTGCAGACCACACAGGAGGAGGACGGCTGCT |
| | | CCTGTCGGTTTCCAGAGGAGGAGGAGGGAGGATGTGAGCTGAGAGTGAAGTTCTCT |
| | | AGGAGCGCCGATGCCCCTGCCTATCAGCAGGGACAGAACCAGCTGTACAACGAGCT |
| | | GAATCTGGGCCGGAGAGAGGAGTACGACGTGCTGGATAAGAGGAGGGGAAGAGACC |
| | | CAGAGATGGGAGGCAAGCCTCGGAGAAAGAACCCACAGGAGGGCCTGTATAATGAG |
| | | CTGCAGAAGGACAAGATGGCCGAGGCCTACTCCGAGATCGGCATGAAGGGAGAGAG |
| | | GCGCCGGGGCAAGGGACACGATGGCCTGTATCAGGGCCTGAGCACCGCCACAAAGG |
| | | ACACATACGATGCCCTGCACATGCAGGCCCTGCCTCCAAGG |
| 104 | TagCAR | LLLVTSLLLCELPHPAFLLIPSVLTQPSSVSAAPGQKVTISCSGSTSNIGNNYVSW |
| | polypeptide | YQQHPGKAPKLMIYDVSKRPSGVPDRFSGSKSGNSASLDISGLQSEDEADYYCAAW |
| | | DDSLSEFLFGTGTKLTVLGSTSGSGKPGSGEGSTKGQVQLVESGGNLVQPGGSLRL |
| | | SCAASGFTFGSFSMSWVRQAPGGGLEWVAGLSARSSLTHYADSVKGRFTISRDNAK |
| | | NSVYLQMNSLRVEDTAVYYCARRSYDSSGYWGHFYSYMDVWGQGTLVTVSSTTTPA |
| | | PRPPTPAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELRVKESR |
| | | SADAPAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 105 | TagCAR | MLLLVTSLLLCELPHPAFLLIPSVLTQPSSVSAAPGQKVTISCSGSTSNIGNNYVS |
| | polypeptidee | WYQQHPGKAPKLMIYDVSKRPSGVPDRFSGSKSGNSASLDISGLQSEDEADYYCAA |
| | | WDDSLSEFLFGTGTKLTVLGSTSGSGKPGSGEGSTKGQVQLVESGGNLVQPGGSLR |
| | | LSCAASGFTFGSFSMSWVRQAPGGGLEWVAGLSARSSLTHYADSVKGRFTISRDNA |
| | | KNSVYLQMNSLRVEDTAVYYCARRSYDSSGYWGHFYSYMDVWGQGTLVTVSSTTTP |
| | | APRPPTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 106 | E2 scFv | TCCGTGCTGACCCAGCCTAGCTCCGTGTCTGCCGCACCAGGACAGAAGGTGACAAT |
| | nucleotide | CAGCTGTTCCGGCTCTACCAGCAACATCGGCAACAATTACGTGAGCTGGTACCAGC |
| | | AGCACCCTGGCAAGGCCCCAAAGCTGATGATCTACGACGTGTCCAAGAGGCCATCT |
| | | GGAGTGCCTGATCGGTTCTCCGGCTCTAAGAGCGGCAATTCCGCCTCTCTGGACAT |
| | | CAGCGGACTGCAGTCCGAGGACGAGGCAGATTACTATTGCGCCGCCTGGGACGATA |
| | | GCCTGTCCGAGTTTCTGTTCGGCACCGGCACAAAGCTGACCGTGCTGGGCTCTACA |
| | | AGCGGATCCGGCAAGCCAGGATCTGGAGAGGGCAGCACAAAGGGACAGGTGCAGCT |
| | | GGTGGAGAGCGGAGGAAACCTGGTGCAGCCAGGAGGCTCCCTGCGCCTGTCTTGTG |
| | | CCGCCAGCGGCTTTACCTTCGGCTCTTTTAGCATGTCCTGGGTGCGCCAGGCACCT |
| | | GGAGGAGGACTGGAGTGGGTGGCCGGCCTGAGCGCCCGGTCTAGCCTGACACACTA |
| | | TGCCGACTCCGTGAAGGGCCGCTTCACCATCTCCCGGGATAACGCCAAGAATAGCG |
| | | TGTACCTGCAGATGAATAGCCTGCGGGTGGAGGACACAGCCGTGTACTATTGCGCC |
| | | AGGCGCTCCTATGATTCCTCTGGCTACTGGGGCCACTTTTACTCTTATATGGACGT |
| | | GTGGGGACAGGGCACCCTGGTGACAGTGAGCTCC |
| 107 | E2 scFv | SVLTQPSSVSAAPGQKVTISCSGSTSNIGNNYVSWYQQHPGKAPKLMIYDVSKRPS |
| | polypeptide | GVPDRFSGSKSGNSASLDISGLQSEDEADYYCAAWDDSLSEFLFGTGTKLTVLGST |
| | | SGSGKPGSGEGSTKGQVQLVESGGNLVQPGGSLRLSCAASGFTFGSFSMSWVRQAP |
| | | GGGLEWVAGLSARSSLTHYADSVKGRFTISRDNAKNSVYLQMNSLRVEDTAVYYCA |
| | | RRSYDSSGYWGHFYSYMDVWGQGTLVTVSS |
| 108 | E2 scFv VL | TCCGTGCTGACCCAGCCTAGCTCCGTGTCTGCCGCACCAGGACAGAAGGTGACAAT |
| | nucleotide | CAGCTGTTCCGGCTCTACCAGCAACATCGGCAACAATTACGTGAGCTGGTACCAGC |
| | | AGCACCCTGGCAAGGCCCCAAAGCTGATGATCTACGACGTGTCCAAGAGGCCATCT |
| | | GGAGTGCCTGATCGGTTCTCCGGCTCTAAGAGCGGCAATTCCGCCTCTCTGGACAT |
| | | CAGCGGACTGCAGTCCGAGGACGAGGCAGATTACTATTGCGCCGCCTGGGACGATA |
| | | GCCTGTCCGAGTTTCTGTTCGGCACCGGCACAAAGCTGACCGTGCTG |
| 109 | E2 scFv VL | SVLTQPSSVSAAPGQKVTISCSGSTSNIGNNYVSWYQQHPGKAPKLMIYDVSKRPS |
| | polypeptide | GVPDRFSGSKSGNSASLDISGLQSEDEADYYCAAWDDSLSEFLFGTGTKLTVL |
| 110 | E2 scFv | GGCTCTACAAGCGGATCCGGCAAGCCAGGATCTGGAGAGGGCAGCACAAAGGGA |
| | linker | |
| | nucleotide | |
| 111 | E2 scFv | GSTSGSGKPGSGEGSTKG |
| | linker | |
| | polypeptide | |
| 112 | E2 scFv VH | CAGGTGCAGCTGGTGGAGAGCGGAGGAAACCTGGTGCAGCCAGGAGGCTCCCTGCG |
| | nucleotide | CCTGTCTTGTGCCGCCAGCGGCTTTACCTTCGGCTCTTTTAGCATGTCCTGGGTGC |
| | | GCCAGGCACCTGGAGGAGGACTGGAGTGGGTGGCCGGCCTGAGCGCCCGGTCTAGC |
| | | CTGACACACTATGCCGACTCCGTGAAGGGCCGCTTCACCATCTCCCGGGATAACGC |
| | | CAAGAATAGCGTGTACCTGCAGATGAATAGCCTGCGGGTGGAGGACACAGCCGTGT |
| | | ACTATTGCGCCAGGCGCTCCTATGATTCCTCTGGCTACTGGGGCCACTTTTACTCT |
| | | TATATGGACGTGTGGGGACAGGGCACCCTGGTGACAGTGAGCTCC |
| 113 | E2 scFv VH | QVQLVESGGNLVQPGGSLRLSCAASGFTFGSFSMSWVRQAPGGGLEWVAGLSARSS |
| | polypeptide | LTHYADSVKGRFTISRDNAKNSVYLQMNSLRVEDTAVYYCARRSYDSSGYWGHFYS |
| | | YMDVWGQGTLVTVSS |
| 114 | CD8 HTM | ACCACCACACCTGCTCCTAGACCACCTACACCCGCTCCTACCATCGCCAGCCAGCC |
| | nucleotide | TCTGTCTCTGAGACCTGAGGCCTGTAGACCTGCCGCTGGAGGCGCTGTGCACACCA |
| | | GAGGACTGGATTTCGCCTGCGACATCTACATCTGGGCTCCTCTGGCTGGAACATGC |
| | | GGCGTGCTGCTCCTGAGCCTGGTGATCACACTGTACTGC |
| 115 | CD8 HTM | TTTPAPRPPTPAPTIASQPLSLRPEACRPAAGGAVHTRGLDFACDIYIWAPLAGTC |
| | polypeptide | GVLLLSLVITLYC |
| 116 | CD8 Hinge | ACCACCACACCTGCTCCTAGACCACCTACACCCGCTCCTACCATCGCCAGCCAGCC |
| | nucleotide | TCTGTCTCTGAGACCTGAGGCCTGTAGACCTGCCGCTGGAGGCGCTGTGCACACCA |
| | | GAGGACTGGATTTCGCCTGCGACATCTAC |
| 117 | CD8 Hinge | TTTPAPRPPTPAPTIASQPLSLRPEACRPAAGGAVHTRGLDFACDIY |
| | polypeptide | |
| 118 | CD8 | ATCTGGGCTCCTCTGGCTGGAACATGCGGCGTGCTGCTCCTGAGCCTGGTGATCAC |
| | trans- | ACTGTACTGC |
| | membrane | |
| | nucleotide | |
| 119 | CD8 | IWAPLAGTCGVLLLSLVITLYC |
| | trans | |
| | membrane | |
| | polypeptide | |
| 120 | Anti-CD3 | DIQMTQSPSSLSASVGDRVTITCSASSSVSYMNWYQQTPGKAPKRWIYDTSKLASG |
| | SCFv (VL- | VPSRFSGSGSGTDYTFTISSLQPEDIATYYCQQWSSNPFTFGQGTKLQITRTSGGG |
| | G4S x 3 | GSGGGGSGGGGSQVQLVQSGGGVVQPGRSLRLSCKASGYTFTRYTMHWVRQAPGKG |
| | linker- | LEWIGYINPSRGYTNYNQKVKDRFTISRDNSKNTAFLQMDSLRPEDIGVYFCARYY |
| | VH) | DDHYCLDYWGQGTPVTVSSAAAKP |
| 121 | Anti-CD3 | GATATCCAGATGACCCAGTCCCCAAGCTCCCTGAGCGCCTCCGTGGGCGACCGGGT |
| | SCFv (VL- | GACAATCACCTGCAGCGCCTCTAGCTCCGTGTCCTACATGAACTGGTATCAGCAGA |
| | G4S x 3 | CACCTGGCAAGGCCCCAAAGAGATGGATCTACGATACCAGCAAGCTGGCCTCCGGC |
| | linker- | GTGCCTTCTAGGTTTTCTGGCAGCGGCTCCGGCACAGATTATACATTCACCATCTC |
| | VH) | TAGCCTGCAGCCAGAGGACATCGCCACCTACTATTGCCAGCAGTGGTCCTCTAATC |
| | | CCTTTACATTCGGCCAGGGCACCAAGCTGCAGATCACAAGAACCTCTGGAGGAGGA |
| | | GGAAGCGGAGGAGGAGGATCCGGCGGCGGCGGCTCTCAGGTGCAGCTGGTGCAGAG |
| | | CGGAGGAGGAGTGGTGCAGCCAGGCAGAAGCCTGAGGCTGTCCTGTAAGGCCTCTG |
| | | GCTACACATTCACCAGATATACAATGCACTGGGTGAGGCAGGCACCAGGCAAGGGA |
| | | CTGGAGTGGATCGGCTACATCAACCCCTCCAGGGGCTACACCAACTATAATCAGAA |
| | | GGTGAAGGATCGGTTCACCATCAGCAGGGACAACTCCAAGAATACCGCCTTCCTGC |
| | | AGATGGACAGCCTGAGGCCAGAGGATACCGGCGTGTACTTTTGCGCCCGGTACTAT |
| | | GACGATCACTACTGTCTGGATTATTGGGGCCAGGGAACACCAGTGACCGTGAGCTC |
| | | CGCCGCAGCAAAGCCT |
| 122 | Anti-CD3 | DIQMTQSPSSLSASVGDRVTITCSASSSVSYMNWYQQTPGKAPKRWIYDTSKLASG |
| | SCFv VL-G4S | VPSRFSGSGSGTDYTFTISSLQPEDIATYYCQQWSSNPFTFGQGTKLQITRTSGGG |
| | x 3 linker- | GSGGGGSGGGGSQVQLVQSGGGVVQPGRSLRLSCKASGYTFTRYTMHWVRQAPGKG |
| | VH | LEWIGYINPSRGYTNYNQKVKDRFTISRDNSKNTAFLQMDSLRPEDIGVYFCARYY |
| | | DDHYCLDYWGQGTPVTVSSAS |
| 123 | Anti-CD3 | GACATCCAGATGACCCAGTCTCCTAGCAGCCTCAGCGCTAGCGTGGGCGATAGAGT |
| | SCFv VL-G4S | GACCATCACATGTAGCGCCAGCAGCAGCGTGTCCTACATGAACTGGTACCAGCAAA |
| | x 3 linker- | CACCTGGAAAGGCCCCTAAAAGGTGGATCTATGACACATCTAAGCTGGCTTCTGGA |
| | VH | GTGCCATCTAGATTTTCTGGCAGCGGCTCCGGCACTGATTATACATTCACCATCAG |
| | | CAGCCTGCAGCCCGAGGATATCGCCACCTACTACTGTCAGCAGTGGTCCTCTAATC |
| | | CCTTCACCTTCGGCCAGGGCACCAAGCTGCAGATCACCAGAACCAGCGGCGGGGGA |
| | | GGAAGCGGCGGGGGAGGATCTGGCGGCGGCGGCAGCCAGGTGCAGCTGGTGCAGAG |
| | | CGGCGGCGGCGTGGTGCAACCTGGCAGAAGCCTGAGACTGAGCTGCAAGGCCTCTG |
| | | GCTACACCTTCACCCGGTACACCATGCATTGGGTGCGGCAGGCCCCTGGCAAGGGC |
| | | CTGGAATGGATTGGATACATCAACCCCAGCAGAGGCTACACCAACTACAACCAGAA |
| | | GGTGAAGGACAGATTCACAATTTCTCGGGACAACAGCAAGAATACCGCCTTCCTGC |
| | | AAATGGACTCCCTGCGCCCAGAAGATACCGGCGTGTACTTCTGCGCTAGATATTAC |
| | | GACGACCACTACTGCCTGGACTACTGGGGCCAGGGCACCCCTGTGACCGTGTCCAG |
| | | CGCCTCC |
| 124 | Cocal | NFLLLTFIVLPLCSHAKFSIVFPQSQKGNWKNVPSSYHYCPSSSDQNWHNDLLGIT |
| | envelope | MKVKMPKTHKAIQADGWMCHAAKWITTCDFRWYGPKYITHSIHSIQPTSEQCKESI |
| | polypeptide | KQTKQGTWMSPGFPPQNCGYATVTDSVAVVVQATPHHVLVDEYTGEWIDSQFPNGK |
| | | CETEECETVHNSTVWYSDYKVTGLCDATLVDTEITFFSEDGKKESIGKPNTGYRSN |
| | | YFAYEKGDKVCKMNYCKHAGVRLPSGVWFEFVDQDVYAAAKLPECPVGATISAPTQ |
| | | TSVDVSLILDVERILDYSLCQETWSKIRSKQPVSPVDLSYLAPKNPGTGPAFTIIN |
| | | GTLKYFETRYIRIDIDNPIISKMVGKISGSQTERELWTEWFPYEGVEIGPNGILKT |
| | | PTGYKFPLFMIGHGMLDSDLHKTSQAEVFEHPHLAEAPKQLPEEETLFFGDTGISK |
| | | NPVELIEGWFSSWKSTVVTFFFAIGVFILLYVVARIVIAVRYRYQGSNNKRIYNDI |
| | | EMSRFRK |
| 125 | Cocal | ATGAACTTTCTGCTGCTGACCTTCATCGTGCTGCCTCTGTGCAGCCACGCCAAGTT |
| | envelope | TTCCATCGTGTTCCCACAGTCCCAGAAGGGCAACTGGAAGAATGTGCCCAGCTCCT |
| | nucleotide | ACCACTATTGTCCTTCTAGCTCCGACCAGAACTGGCACAATGATCTGCTGGGCATC |
| | | ACCATGAAGGTGAAGATGCCTAAGACACACAAGGCCATCCAGGCAGATGGATGGAT |
| | | GTGCCACGCAGCCAAGTGGATCACCACATGTGACTTTCGGTGGTACGGCCCCAAGT |
| | | ATATCACCCACAGCATCCACTCCATCCAGCCTACAAGCGAGCAGTGCAAGGAGTCC |
| | | ATCAAGCAGACCAAGCAGGGCACATGGATGTCTCCCGGCTTCCCCCCTCAGAACTG |
| | | TGGCTACGCCACCGTGACAGATAGCGTGGCAGTGGTGGTGCAGGCAACCCCACACC |
| | | ACGTGCTGGTGGATGAGTATACAGGCGAGTGGATCGACAGCCAGTTTCCCAACGGC |
| | | AAGTGCGAGACCGAGGAGTGTGAGACAGTGCACAATTCTACCGTGTGGTACAGCGA |
| | | TTATAAGGTGACCGGCCTGTGCGACGCCACACTGGTGGATACCGAGATCACATTCT |
| | | TTTCCGAGGACGGCAAGAAGGAGTCTATCGGCAAGCCCAACACCGGCTACAGGTCT |
| | | AATTACTTCGCCTATGAGAAGGGCGATAAGGTGTGCAAGATGAATTATTGTAAGCA |
| | | CGCCGGGGTGCGGCTGCCAAGCGGCGTGTGGTTTGAGTTCGTGGACCAGGACGTGT |
| | | ACGCAGCAGCAAAGCTGCCAGAGTGCCCAGTGGGAGCAACCATCAGCGCCCCCACC |
| | | CAGACATCTGTGGACGTGAGCCTGATCCTGGATGTGGAGAGAATCCTGGACTACTC |
| | | CCTGTGCCAGGAGACATGGTCCAAGATCCGCTCTAAGCAGCCCGTGAGCCCAGTGG |
| | | ACCTGTCTTACCTGGCACCAAAGAACCCTGGAACAGGACCTGCCTTTACCATCATC |
| | | AATGGCACACTGAAGTACTTCGAGACCCGGTATATCAGAATCGACATCGATAACCC |
| | | AATCATCTCCAAGATGGTGGGCAAGATCTCCGGCTCTCAGACCGAGAGAGAGCTGT |
| | | GGACAGAGTGGTTCCCATACGAGGGCGTGGAGATCGGCCCCAATGGCATCCTGAAG |
| | | ACCCCTACAGGCTATAAGTTTCCACTGTTCATGATCGGCCACGGCATGCTGGACTC |
| | | TGATCTGCACAAGACCAGCCAGGCCGAGGTGTTTGAGCACCCACACCTGGCAGAGG |
| | | CACCAAAGCAGCTGCCCGAGGAGGAGACCCTGTTCTTTGGCGATACAGGCATCTCC |
| | | AAGAACCCTGTGGAGCTGATCGAGGGCTGGTTTTCTAGCTGGAAGTCTACCGTGGT |
| | | GACATTCTTTTTCGCCATCGGCGTGTTCATCCTGCTGTACGTGGTGGCAAGGATCG |
| | | TGATCGCCGTGCGGTACAGATATCAGGGCAGCAACAATAAGAGAATCTATAATGAC |
| | | ATCGAGATGTCCAGGTTCCGCAAGTGA |
| 126 | Cocal | AATTTTCTGCTGCTGACCTTCATCGTGCTGCCTCTGTGCAGCCACGCCAAGTTTTC |
| | envelope | CATCGTGTTCCCACAGTCCCAGAAGGGCAACTGGAAGAATGTGCCCTCTAGCTACC |
| | nucleotide | ACTATTGCCCTTCCTCTAGCGACCAGAACTGGCACAATGATCTGCTGGGCATCACA |
| | | ATGAAGGTGAAGATGCCCAAGACCCACAAGGCCATCCAGGCAGATGGATGGATGTG |
| | | CCACGCAGCCAAGTGGATCACAACCTGTGACTTTCGGTGGTACGGCCCCAAGTATA |
| | | TCACACACTCCATCCACTCTATCCAGCCTACCTCCGAGCAGTGCAAGGAGTCTATC |
| | | AAGCAGACAAAGCAGGGCACCTGGATGAGCCCTGGCTTCCCACCCCAGAACTGTGG |
| | | CTACGCCACAGTGACCGACTCCGTGGCAGTGGTGGTGCAGGCAACACCTCACCACG |
| | | TGCTGGTGGATGAGTATACCGGCGAGTGGATCGACAGCCAGTTTCCAAACGGCAAG |
| | | TGCGAGACAGAGGAGTGTGAGACCGTGCACAATTCTACAGTGTGGTACAGCGATTA |
| | | TAAGGTGACAGGCCTGTGCGACGCCACCCTGGTGGATACAGAGATCACCTTCTTTT |
| | | CTGAGGACGGCAAGAAGGAGAGCATCGGCAAGCCCAACACCGGCTACAGATCCAAT |
| | | TACTTCGCCTATGAGAAGGGCGATAAGGTGTGCAAGATGAATTATTGTAAGCACGC |
| | | CGGGGTGCGGCTGCCTAGCGGCGTGTGGTTTGAGTTCGTGGACCAGGACGTGTACG |
| | | CAGCAGCAAAGCTGCCTGAGTGCCCAGTGGGAGCAACCATCTCCGCCCCAACACAG |
| | | ACCTCCGTGGACGTGTCTCTGATCCTGGATGTGGAGCGCATCCTGGACTACAGCCT |
| | | GTGCCAGGAGACCTGGAGCAAGATCCGGTCCAAGCAGCCCGTGTCCCCTGTGGACC |
| | | TGTCTTACCTGGCACCAAAGAACCCAGGAACCGGACCAGCCTTTACAATCATCAAT |
| | | GGCACCCTGAAGTACTTCGAGACCCGCTATATCCGGATCGACATCGATAACCCTAT |
| | | CATCAGCAAGATGGTGGGCAAGATCTCTGGCAGCCAGACAGAGAGAGAGCTGTGGA |
| | | CCGAGTGGTTCCCTTACGAGGGCGTGGAGATCGGCCCAAATGGCATCCTGAAGACA |
| | | CCAACCGGCTATAAGTTTCCCCTGTTCATGATCGGCCACGGCATGCTGGACAGCGA |
| | | TCTGCACAAGACCTCCCAGGCCGAGGTGTTTGAGCACCCACACCTGGCAGAGGCAC |
| | | CAAAGCAGCTGCCTGAGGAGGAGACACTGTTCTTTGGCGATACCGGCATCTCTAAG |
| | | AACCCCGTGGAGCTGATCGAGGGCTGGTTTTCCTCTTGGAAGAGCACAGTGGTGAC |
| | | CTTCTTTTTCGCCATCGGCGTGTTCATCCTGCTGTACGTGGTGGCCAGAATCGTGA |
| | | TCGCCGTGAGATACAGGTATCAGGGCTCCAACAATAAGAGGATCTATAATGACATC |
| | | GAGATGTCTCGCTTCCGGAAG |
| 127 | Rev | MAGRSGDSDEDLLKAVRLIKFLYQSNPPPNPEGTRQARRNRRRRWRERQRQIHSIS |
| | polypeptide | ERILSTYLGRSAEPVPLQLPPLERLTLDCNEDCGTSGTQGVGSPQILVESPTILES |
| | | GAKE* |
| 128 | Rev | ATGGCCGGCAGAAGCGGCGACAGCGACGAGGATCTGCTGAAAGCCGTGCGGCTGAT |
| | nucleotide | CAAGTTCCTGTACCAGAGCAACCCTCCTCCTAACCCCGAGGGCACCAGACAGGCTA |
| | | GACGGAACCGCAGAAGAAGGTGGCGGGAACGGCAAAGACAGATCCACTCTATCAGC |
| | | GAGAGAATCCTGAGCACCTACCTGGGAAGATCCGCCGAGCCTGTCCCCCTGCAGCT |
| | | GCCTCCACTGGAAAGACTGACCCTGGATTGTAATGAGGACTGCGGCACAAGCGGAA |
| | | CCCAGGGCGTGGGCAGCCCCCAGATTCTGGTGGAATCCCCTACAATCCTCGAGTCT |
| | | GGCGCCAAGGAATGA |
| 129 | Anti-CD20 | ATGCTGCTGCTGGTGACAAGCCTGCTGCTGTGCGAGCTGCCTCACCCAGCCTTTCT |
| | CAR | GCTGATCCCCGATATCGTGCTGACCCAGTCCCCCGCCATCCTGTCCGCCTCTCCTG |
| | (without | GAGAGAAGGTGACCATGACATGTCGGGCCAGCTCCTCTGTGAACTACATGGACTGG |
| | flag) | TATCAGAAGAAGCCTGGCAGCTCCCCCAAGCCTTGGATCTACGCCACCTCCAATCT |
| | nucleotide | GGCCTCTGGAGTGCCAGCAAGATTCAGCGGATCCGGATCTGGCACAAGCTATTCCC |
| | | TGACCATCTCCAGGGTGGAGGCAGAGGATGCAGCAACATACTATTGCCAGCAGTGG |
| | | TCTTTCAACCCCCCTACATTTGGCGGCGGCACCAAGCTGGAGATCAAGGGCTCTAC |
| | | CAGCGGAGGAGGAAGCGGAGGAGGATCCGGAGGCGGCGGCTCTAGCGAGGTGCAGC |
| | | TGCAGCAGTCCGGAGCAGAGCTGGTGAAGCCTGGAGCCTCTGTGAAGATGAGCTGT |
| | | AAGGCCTCCGGCTACACCTTCACATCTTATAATATGCACTGGGTGAAGCAGACACC |
| | | AGGACAGGGACTGGAGTGGATCGGAGCAATCTACCCTGGCAACGGCGACACCAGCT |
| | | ATAATCAGAAGITTAAGGGCAAGGCCACCCTGACAGCCGATAAGTCCTCTAGCACA |
| | | GCCTACATGCAGCTGTCCTCTCTGACCAGCGAGGACTCCGCCGATTACTATTGCGC |
| | | CCGGTCCAACTACTATGGCAGCTCCTATTGGTTCTTTGACGTGTGGGGAGCAGGAA |
| | | CAACCGTGACCGTGTCTAGCGCAAAGCCAACCACCACACCTGCTCCTAGACCACCT |
| | | ACACCCGCTCCTACCATCGCCAGCCAGCCTCTGTCTCTGAGACCTGAGGCCTGTAG |
| | | ACCTGCCGCTGGAGGCGCTGTGCACACCAGAGGACTGGATTTCGCCTGCGACATCT |
| | | ACATCTGGGCTCCTCTGGCTGGAACATGCGGCGTGCTGCTCCTGAGCCTGGTGATC |
| | | ACACTGTACTGCAAGAGAGGCAGGAAGAAGCTGCTGTATATCTTTAAGCAGCCCTT |
| | | CATGCGCCCTGTGCAGACCACACAGGAGGAGGACGGCTGCAGCTGTCGGTTTCCAG |
| | | AGGAGGAGGAGGGAGGATGCGAGCTGCGCGTGAAGTTCAGCCGGTCCGCCGATGCC |
| | | CCTGCCTACCAGCAGGGCCAGAACCAGCTGTATAACGAGCTGAATCTGGGCCGGAG |
| | | AGAGGAGTACGACGTGCTGGATAAGAGGAGGGGAAGGGACCCAGAGATGGGAGGCA |
| | | AGCCTCGGAGAAAGAACCCACAGGAGGGCCTGTACAATGAGCTGCAGAAGGACAAG |
| | | ATGGCCGAGGCCTATTCTGAGATCGGCATGAAGGGAGAGAGGCGCCGGGGCAAGGG |
| | | ACACGATGGCCTGTACCAGGGCCTGAGCACCGCCACAAAGGACACCTATGATGCCC |
| | | TGCACATGCAGGCCCTGCCCCCTCGG |
| 130 | Anti-CD20 | MLLLVTSLLLCELPHPAFLLIPDIVLTQSPAILSASPGEKVTMTCRASSSVNYMDW |
| | CAR | YQKKPGSSPKPWIYATSNLASGVPARFSGSGSGTSYSLTISRVEAEDAATYYCQQW |
| | (without | SFNPPTFGGGTKLEIKGSTSGGGSGGGSGGGGSSEVQLQQSGAELVKPGASVKMSC |
| | flag) | KASGYTFTSYNMHWVKQTPGQGLEWIGAIYPGNGDTSYNQKFKGKATLTADKSSST |
| | polypeptide | AYMQLSSLTSEDSADYYCARSNYYGSSYWFFDVWGAGTTVTVSSAKPTTTPAPRPP |
| | | TPAPTIASQPLSLRPEACRPAAGGAVHTRGLDFACDIYIWAPLAGTCGVLLLSLVI |
| | | TLYCKRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |

1. A method for preparing a lentivirus formulation, comprising
(i) clarifying a suspension mixture comprising a population of host cells and lentiviral particles to remove contaminants, wherein clarifying comprises:
(a) filtering the suspension mixture with a first filter, wherein the first filter is a depth filter, resulting in a first filtrate,
(b) filtering the first filtrate with a second filter, wherein the second filter has a retention threshold smaller than the first filter, resulting in a second filtrate, and
(c) filtering the second filtrate, with a third filter, wherein the third filter has a retention threshold smaller than the second filter, thereby producing a clarified formulation of lentiviral particles; and
(ii) concentrating the clarified formulation of lentiviral particles, wherein concentrating comprises chromatography and ultrafiltration.

2. A method for preparing a lentivirus formulation, comprising
(i) contacting a population of host cells in suspension with at least one plasmid encoding a lentiviral protein;
(ii) culturing the population of host cells of step (i) for a period of time sufficient to produce a suspension mixture comprising a population of host cells and lentiviral particles;
(iii) clarifying the suspension mixture to remove contaminants, comprising:
(a) contacting the suspension mixture with an endonuclease,
(b) filtering the suspension mixture with a first filter, wherein the first filter is a depth filter, resulting in a first filtrate
(c) filtering the first filtrate with a second filter, wherein the second filter has a retention threshold smaller than the first filter, resulting in a second filtrate, and
(d) filtering the second filtrate with a third filter, wherein the third filter has a retention threshold smaller than the second filter, thereby producing a clarified formulation of lentiviral particles; and
(iv) concentrating the clarified formulation of lentiviral particles, wherein concentrating comprises chromatography and ultrafiltration.

3. The method of claim 1, wherein the host cell comprises a human cell.

4. The method of claim 3, wherein the human cell comprises a HEK293 cell, a HEK293T cell, a HEK293F cell, a HEK293FT cell, a Te671 cell, a HT1080 cell, or a CEM cell.

5. (canceled)

6. (canceled)

7. The method of claim 1, wherein: the first filter has a retention threshold of 1-60 m; the second filter has a retention threshold of 0.4-4 μm; and/or the third filter has a retention threshold of 0.45 μm±0.2 μm.

8.-14. (canceled)

15. The method of claim 1, wherein an endonuclease is present through steps (i)(a)-(i)(c).

16. The method of claim 2, wherein the endonuclease is present through steps (iii)(b)-(iii)(d).

17-19. (canceled)

20. The method of claim 1, wherein the chromatography is anion exchange chromatography (AEX).

21. The method of claim 1, wherein the chromatography comprises eluting the lentiviral particles with a salt buffer comprising NaCl.

22. (canceled)

23. The method of claim 21, wherein the NaCl is at a concentration from about 0.5M to about 3M.

24.-26. (canceled)

27. The method of claim 22, wherein the NaCl is at a concentration from about 1.5 M to 2.5 M.

28. (canceled)

29. The method of claim 1, wherein the chromatography is performed before ultrafiltration.

30. The method of claim 1, wherein the ultrafiltration is ultrafiltration/diafiltration (UF/DF).

31.-43. (canceled)

44. The method of claim 1, comprising sterile filtering the clarified formulation after concentration, thereby producing a sterilized formulation.

45. (canceled)

46. (canceled)

47. The method of claim 1, comprising formulating the sterilized formulation in a buffer, thereby producing a drug substance.

48. (canceled)

49. The method of claim 1, wherein the lentivirus formulation is for in vivo administration to a subject.

50. The method of claim 1, wherein the amount of contaminants in the clarified formulation is reduced compared to the amount of contaminants in the second filtrate.

51. (canceled)

52. The method of claim 1, wherein the contaminants comprise host cells, host cell DNA (hcDNA), and/or host cell proteins (HCP).

53. The method of claim 52, wherein:
the amount of hcDNA is less than 10%, less than 9%, less than 8%, less than 7%, less than 6%, less than 5%, less than 4%, less than 3%, less than 2%, less than 1% or less than 0.5% of the sterilized formulation;
the amount of hcDNA is reduced greater than 90%, greater than 91%, greater than 92%, greater than 93%, greater than 94%, greater than 95%, greater than 96%, greater than 97%, greater than 98%, or greater than 99% in the sterilized formulation;
the amount of HCP is less than 10%, less than 9%, less than 8%, less than 7%, less than 6%, less than 5%, less than 4%, less than 3%, less than 2%, less than 1% or less than 0.5% of the sterilized formulation; and/or
the amount of HCP is reduced greater than 90%, greater than 91%, greater than 92%, greater than 93%, greater than 94%, greater than 95%, greater than 96%, greater than 97%, greater than 98%, or greater than 99% in the sterilized formulation.

54.-71. (canceled)

72. The method of claim 1, wherein the lentiviral particle comprises at least one payload.

73. (canceled)

74. The method of claim 72, wherein the at least payload is a polynucleotide encoding a polypeptide of interest.

75.-100. (canceled)

101. The method of claim 2, wherein the population of host cells is contacted with a mixture of plasmids comprising (i) a plasmid encoding a gene of interest; (ii) a plasmid encoding a rev viral protein; (iii) a plasmid encoding a gagpol viral protein; and (iv) a plasmid encoding a viral envelope protein.

102. (canceled)

103. A lentiviral formulation produced by the method of claim 1.

104.-115. (canceled)

116. A method for preparing a lentivirus formulation, comprising
(i) clarifying a suspension mixture comprising a population of host cells and lentiviral particles to remove contaminants, wherein clarifying comprises:
(a) filtering the suspension mixture with a first filter, wherein the first filter is a depth filter, resulting in a first filtrate, and
(b) filtering the first filtrate with a second filter, wherein the second filter has a retention threshold smaller than the first filter, thereby producing a clarified formulation of lentiviral particles; and
(ii) concentrating the clarified formulation of lentiviral particles, wherein concentrating comprises chromatography and ultrafiltration, and wherein the chromatography comprises eluting the lentiviral particles with a salt buffer comprising NaCl at a concentration from about 0.5M to about 3M.

117. A method for preparing a lentivirus formulation, comprising
(i) clarifying a suspension mixture comprising a population of host cells and lentiviral particles to remove contaminants, wherein clarifying comprises:
(a) centrifuging the suspension mixture, and
(b) filtering the centrifuged suspension with a membrane filter, thereby producing a clarified formulation of lentiviral particles; and
(ii) concentrating the clarified formulation of lentiviral particles, wherein concentrating comprises chromatography and ultrafiltration, and wherein the chromatography comprises eluting the lentiviral particles with a salt buffer comprising NaCl at a concentration from about 0.5M to about 3M.

118. A method for preparing a lentivirus formulation, comprising
(i) contacting a population of host cells in suspension with at least one plasmid encoding a lentiviral protein;
(ii) culturing the population of host cells of step (i) for a period of time sufficient to produce a suspension mixture comprising a population of host cells and lentiviral particles;
(iii) clarifying the suspension mixture to remove contaminants, comprising:
(a) contacting the suspension mixture with an endonuclease,
(b) filtering the suspension mixture with a first filter, wherein the first filter is a depth filter, resulting in a first filtrate, and
(c) filtering the first filtrate with a second filter, wherein the second filter has a retention threshold smaller than the first filter, thereby producing a clarified formulation of lentiviral particles; and
(iv) concentrating the clarified formulation of lentiviral particles, wherein concentrating comprises chromatography and ultrafiltration, and wherein the chromatography comprises eluting the lentiviral particles with a salt buffer comprising NaCl at a concentration from about 0.5M to about 3M.

119. The method of claim 118, wherein the NaCl is at a concentration from about 1.5 M to 2.5 M.

120. A method for preparing a lentivirus formulation, comprising
(i) contacting a population of host cells in suspension with at least one plasmid encoding a lentiviral protein;
(ii) culturing the population of host cells of step (i) for a period of time sufficient to produce a suspension mixture comprising a population of host cells and lentiviral particles;
(iii) clarifying the suspension mixture to remove contaminants, comprising:
(a) contacting the suspension mixture with an endonuclease,
(b) centrifuging the suspension mixture, and
(c) filtering the centrifuged suspension mixture with a membrane filter, thereby producing a clarified formulation of lentiviral particles; and
(iv) concentrating the clarified formulation of lentiviral particles, wherein concentrating comprises chromatography and ultrafiltration, and wherein the chromatography comprises eluting the lentiviral particles with a salt buffer comprising NaCl at a concentration from about 0.5M to about 3M.
